(19) United States
(12) Patent Application Publication
(10) Pub. No.: US 2024/0002416 A1
(43) Pub. Date: Jan. 4, 2024

(54) MRGPRX4 Agonists and Antagonists

(71) Applicant: Rheinische-Friedrich-Wilhelms-Universität Bonn, Bonn (DE)

(72) Inventors: Wessam Alnouri, Nidderau (DE); Jorg Hockemeyer, Bonn (DE); Daniel Marx, Linz am Rhein (DE); Christa Muller, Bonn (DE); Vigneshwaran Namasivayam, Bonn (DE); Yvonne Riedel, Freiburg (DE); Dominik Thimm, Bonn (DE); Sophie Clemens, Bonn (DE); Robin Gedschold, Bonn (DE); Thanigaimalai Pillaiyar, Reutlingen (DE)

(21) Appl. No.: 18/020,833

(22) PCT Filed: Oct. 15, 2021
(86) PCT No.: PCT/EP2021/078622

§ 371 Date: Feb. 10, 2023

(30) Foreign Application Priority Data
Oct. 15, 2020 (EP) 20202000.4

(51) Int. Cl. C07F 9/6561

(52) U.S. Cl. CPC C07F 9/65616

(57) ABSTRACT

The invention relates to MRGPRX4 receptor agonists and antagonists useful for treating, alleviating and/or preventing diseases and disorders related to MRGPRX4 receptor function as well as pharmaceutical compositions comprising such compounds and methods for preparing such compounds. The invention is further directed to the use of these compounds, alone or in combination with other therapeutic agents, for alleviating, preventing and/or treating diseases and disorders, especially the use as wound healing medicaments and for the treatment of chronic pain and itch.

The invention relates to MRGPRX4 receptor agonists and antagonists useful for treating, alleviating and/or preventing diseases and disorders related to MRGPRX4 receptor function as well as pharmaceutical compositions comprising such compounds and methods for preparing such compounds. The invention is further directed to the use of these compounds, alone or in combination with other therapeutic agents, for alleviating, preventing and/or treating diseases and disorders, especially the use as wound healing medicaments or medicaments for different types of pain including itching.

MRGPRX4 (in the literature also referred to as “MAS-related G protein-coupled receptor X4”, MRGX4), is a member of the MRGX receptor family (in the literature also referred to as “MAS-related G protein-coupled receptor X”, MRGX, MRGPRX, and the like).

MAS-related gene receptors (MRG receptors, MRGPR) form a large family of G protein-coupled receptors. The MRGPRX (MRGX) subfamily of MRGPRs is expressed in small-diameter sensory neurons of dorsal root ganglia, in keratinocytes and few other tissues. The MRGPRX family consists of 4 subtypes (MRGPRX1-X4) which are expressed in primates including humans, but not found, e.g., in rodents. Non-primate orthologs of MRGPRX4 have not been identified to date. Proliferative responses mediated by human MRGPRX4 are pertussis toxin- (PTX-) insensitive MRGPRX4 and primarily mediated by the stimulation of Gq-regulated pathways (E. S. Burnstein et al., Br J Pharmacol. 2006 January; 147(1):73-82. doi: 10.1038/sj.bjp.0706448). Besides, GPCRs induce β-arrestin recruitment followed by internalization of the receptor, and in fact, β-arrestin recruitment was shown to be induced by MRGPRX4 activation.

It is known that angiogenic peptide (AG)-30/5C activates human keratinocytes to produce cytokines/chemokines and to migrate and proliferate via MRGX receptors. There is evidence that AG-30/5C may be a useful therapeutic agent for wound healing by activating human keratinocytes (Ch. Kiatsurayanon et al., J Dermatol Sci. 2016 September; 83(3):190-9. doi: 10.1016/j.jdermsci.2016.05.006).

U.S. Pat. No. 5,719,279 A relates to new xanthine derivatives, processes for preparing them and their use as pharmaceutical compositions and their use as intermediates.

DE 198 16 857 A1 relates to novel unsymmetrically substituted xanthine derivatives, process for their preparation and their use as medicaments, in particular as medicinal products with adenosine antagonist effect.

Toperman, I. B. et al., “N-(ω-Carboxyalkyl) Ureas and their Cyclic Derivatives-II. Synthesis of 3-(ω-Carboxyalkyl)-Xanthines”, Pharmaceutical Chemistry Journal, No. 9 (1967), pp. 526-528, relates to the synthesis of 3-substituted xhantines and their methylated derivatives.

Maslankiewicz, A. et al., “Synthesis of N,N′-Dimethyl-7H-Xanthine-N″-Acetic Acids”, Acta Polon. Pharm., Vol. 39, Nr. 5 (1979), pp. 539-543, relates to alkylation reactions of N,N′-dimethyl-7H-xanthines by derivatives of chloroacetic acid.

Sauer, R. et al., “Water-soluble Phosphate Prodrugs of 1-Propargyl-8-styrylxanthine Derivatives, A_{2A}-Selective Adenosine Receptor Antagonists”, J. Med. Chem., Vol. 43, No. 3 (2000), pp. 440-448, relates to water-soluble prodrugs of potent A_{2A}-selective adenosine receptor (AR) antagonists.

Weyler, S. et al., “Improving Potency, Selectivity, and Water Solubility of Adenosine A₁ Receptor Antagonists: Xanthines Modified at Position 3 and Related Pyrimidio[1,2,3-cd]purinediones”, ChemMedChem., Vol. 1 (2006), pp. 891-902, relates to the structure-activity relationships of xanthine detivatives related to the adenosine A1 receptor antagonist 8-cyclopentyl-1,3-dipropylxanthine (DPCPX) and 1,3-dipropyl-8-(3-noradamantyl)xanthine (KW3902).

WO 2008/077557 A1 relates to 8-ethinylxanthine derivatives as selective A_{2A} receptor antagonists.

The human MRGPRX4 receptor represents a fundamentally new drug target, and the development of potent MRGPRX4 receptor agonists, partial agonists, and antagonists/inverse agonists to be used for the treatment of MRGPRX4-associated conditions, disorders or diseases requires the design of novel drugs targeting the MRGPRX4 receptor.

It is an object of the invention to provide compounds that have advantages compared to the compounds of the prior art. The compounds should act as potent and selective agonists or antagonists of the MRGPRX4 receptor, in particular of the human wildtype MRGPRX4 receptor, and thus may be useful as drugs or for the prevention or treatment of MRGPRX4-associated conditions, disorders or diseases. Moreover, it was an object of the invention to provide methods for preparing said compounds. It was furthermore an object of the invention to provide compounds and pharmaceutical formulations for the treatment, alleviation and/or prevention of MRGPRX4-associated conditions, disorders or diseases. It was a further object of the invention to provide the use of these compounds for alleviating, preventing and/or treating conditions, diseases and disorders connected to MRGPRX4 function, particularly for, but not limited to the use for treating open or closed wounds, e.g. for wound healing, and for the treatment of different types of pain and itch.

This object has been solved by the subject-matter of the patent claims.

The invention is directed to MRGPRX4 receptor agonists or antagonists, respectively, that are useful for preventing or treating MRGPRX4-associated conditions, disorders or diseases.

A first aspect of the invention relates to a compound according to general Formula 1

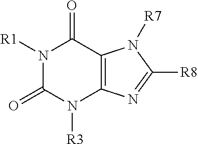

wherein
R1 represents —H, —C₁₋₁₀-alkyl, —C₁₋₁₀-alkyl-C₃₋₁₀-cycloalkyl, —C₁₋₁₀-alkyl-aryl, —C₁₋₁₀-alkyl-heteroaryl, —C₃₋₁₀-cycloalkyl, —C₁₋₁₀-alkyl-O-aryl, or —C₁₋₁₀-alkyl-O-heteroaryl;
R3 represents —C₁₋₁₀-alkyl-P(═O)(OC₁₋₁₀-alkyl)₂, —C₁₋₁₀-alkyl-P(═O)(OH)(OC₁₋₁₀-alkyl), —C₁₋₁₀-alkyl-S(═O)₂(OH), —C₁₋₁₀-alkyl-S(═O)₂(NH₂), —C₁₋₁₀-alkyl-C(═O)(OH), or —C₁₋₁₀-alkyl-P(═O)(OH)₂;
R7 represents —H, —C₁₋₁₀-alkyl, —C₁₋₁₀-alkyl-C₃₋₁₀-cycloalkyl, —C₁₋₁₀-alkyl-aryl, —C₁₋₁₀-alkyl-heteroaryl, —C₃₋₁₀-cycloalkyl, —C₁₋₁₀-alkyl-O-aryl, or —C₁₋₁₀-alkyl-O-heteroaryl; and R8 represents —H, —C₁₋₁₀-alkyl, —C₁₋₁₀-alkyl-C₃₋₁₀-cycloalkyl, —C₁₋₁₀-alkyl-aryl, —C₁₋₁₀-alkyl-heteroaryl, —C₃₋₁₀-cycloalkyl, —C₁₋₁₀-alkyl-O-aryl, —C₃₋₆-cycloalkyl-aryl, —C₃₋₆-cycloalkyl-heteroaryl, or —C₁₋₁₀-alkyl-O-heteroaryl;
wherein in each case “C₁₋₁₀-alkyl” may be linear or branched, unless expressly stated otherwise saturated or unsaturated, unsubstituted or monosubstituted with a substituent selected from —C≡CH, —C(═O)OH, —C(═O)O—C₁₋₁₀-alkyl, —OH, —O—C₁₋₁₀-alkyl, —NH₂, —NH—C₁₋₁₀-alkyl, —N(C₁₋₁₀-alkyl)₂, —N₃, —F, —Cl, —Br, and —I;
wherein in each case “C₃₋₁₀-cycloalkyl” may be linear or branched, unless expressly stated otherwise saturated or unsaturated, unsubstituted or monosubstituted with a substituent selected from —C≡CH, —C(═O)OH, —C(═O)O—C₁₋₆-alkyl, —OH, —O—C₁₋₁₀-alkyl, —NH₂, —NH—C₁₋₁₀-alkyl, —N(C₁₋₁₀-alkyl)₂, —N₃, —F, —Cl, —Br, and —I;
wherein in each case “aryl” is a 6-14-membered aryl moiety which may be unsubstituted, mono- or disubstituted with a substituent independently selected from —C≡CH, —CH₃, —CH₂—CH₃, —CH(CH₃)₂, -phenyl, —CF₃, —CCl₃, —CBr₃, —Cl₃, —C(═O)OH, —C(═O)O—C₁₋₁₀-alkyl, —OH, —O—C₁₋₁₀-alkyl, —NH₂, —NH—C₁₋₁₀-alkyl, —N(C₁₋₁₀-alkyl)₂, —N₃, —F, —Cl, —Br, and —I; preferably, in each case “aryl” is a 6-14-membered aryl moiety which may be unsubstituted, mono- or disubstituted with a substituent independently selected from —C≡CH, —CF₃, —CCl₃, —CBr₃, —Cl₃, —C(═O)OH, —C(═O)O—C₁₋₁₀-alkyl, —OH, —O—C₁₋₁₀-alkyl, —NH₂, —NH—C₁₋₁₀-alkyl, —N(C₁₋₁₀-alkyl)₂, —N₃, —F, —Cl, —Br, and —I;
wherein in each case “heteroaryl” is a 5-14-membered heteroaryl moiety which may be unsubstituted, mono- or disubstituted with a substituent independently selected from —C≡CH, —CF₃, —CCl₃, —CBr₃, —Cl₃, —C(═O)OH, —C(═O)O—C₁₋₁₀-alkyl, —OH, —O—C₁₋₁₀-alkyl, —NH₂, —NH—C₁₋₁₀-alkyl, —N(C₁₋₁₀-alkyl)₂, —N₃, —F, —Cl, —Br, and —I;
or a physiologically acceptable salt thereof;
with the proviso that the compound is not a compound selected from the group consisting of compounds J-1 to J-3:

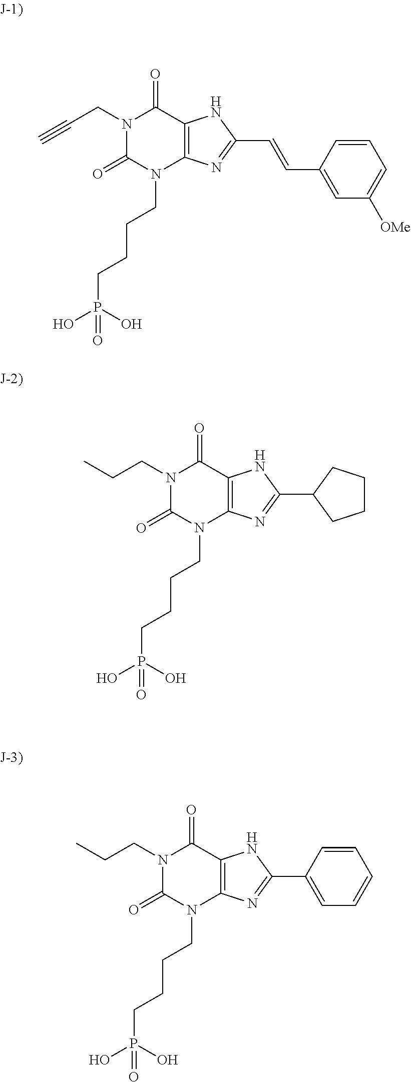 [SMILES: C#CCn1c(=O)c2[nH]c(/C=C/c3cccc(OC)c3)nc2n(CCCCP(=O)(O)O)c1=O ; CCCn1c(=O)c2[nH]c(C3CCCC3)nc2n(CCCCP(=O)(O)O)c1=O ; CCCn1c(=O)c2[nH]c(-c3ccccc3)nc2n(CCCCP(=O)(O)O)c1=O]

The compounds according to the invention are derivatives of xanthine (2,6-dihydroxypurine). The numbering of substituents R1, R3, R7 and R8 corresponds to the numbering of ring atoms of the xanthine scaffold.

The compounds according to the invention may reflect two substitution patterns, for the purpose of the specification indicated as (i) and (ii), respectively.

According to substitution pattern (i),
R1 represents —H, —C₁₋₁₀-alkyl, —C₁₋₁₀-alkyl-C₃₋₁₀-cycloalkyl, —C₁₋₁₀-alkyl-aryl, or —C₁₋₁₀-alkyl-heteroaryl;
R3 represents —C₁₋₁₀-alkyl-P(═O)(OH)₂;
R7 represents —H, —C₁₋₁₀-alkyl, —C₁₋₁₀-alkyl-C₃₋₁₀-cycloalkyl, —C₁₋₁₀-alkyl-aryl, or —C₁₋₁₀-alkyl-heteroaryl; and
R8 represents —C₃₋₆-cycloalkyl-aryl, —C₃₋₆-cycloalkyl-heteroaryl, —C₁₋₁₀-alkyl-aryl or —C₁₋₁₀-alkyl-heteroaryl (wherein in case of —C₂-alkyl-aryl, the alkyl-moiety is saturated).

According to substitution pattern (ii),
R1 represents —H, —C₁₋₁₀-alkyl, —C₁₋₁₀-alkyl-C₃₋₁₀-cycloalkyl, —C₁₋₁₀-alkyl-aryl, —C₁₋₁₀-alkyl-heteroaryl, —C₃₋₁₀-cycloalkyl, —C₁₋₁₀-alkyl-O-aryl, or —C₁₋₁₀-alkyl-O-heteroaryl;
R3 represents —C₁₋₁₀-alkyl-P(═O)(OC₁₋₁₀-alkyl)₂; —C₁₋₁₀-alkyl-P(═O)(OH)(OC₁₋₁₀-alkyl); —C₁₋₁₀-alkyl-S(═O)₂(OH); —C₁₋₁₀-alkyl-S(═O)₂(NH₂); or —C₁₋₁₀-alkyl-C(═O)(OH);
R7 represents —H, —C₁₋₁₀-alkyl, —C₁₋₁₀-alkyl-C₃₋₁₀-cycloalkyl, —C₁₋₁₀-alkyl-aryl, —C₁₋₁₀-alkyl-heteroaryl, —C₃₋₁₀-cycloalkyl, —C₁₋₁₀-alkyl-O-aryl, or —C₁₋₁₀-alkyl-O-heteroaryl; and
R8 represents —H, —C₁₋₁₀-alkyl, —C₁₋₁₀-alkyl-C₃₋₁₀-cycloalkyl, —C₁₋₁₀-alkyl-aryl, —C₁₋₁₀-alkyl-heteroaryl, —C₃₋₁₀-cycloalkyl, —C₁₋₁₀-alkyl-O-aryl, or —C₁₋₁₀-alkyl-O-heteroaryl.

According to substitution pattern (iii),
R1 represents —H, —C₁₋₁₀-alkyl, —C₁₋₁₀-alkyl-C₃₋₁₀-cycloalkyl, —C₁₋₁₀-alkyl-aryl, —C₁₋₁₀-alkyl-heteroaryl, —C₃₋₁₀-cycloalkyl, —C₁₋₁₀-alkyl-O-aryl, and —C₁₋₁₀-alkyl-O-heteroaryl;
R3 represents —C₁₋₁₀-alkyl-P(═O)(OC₁₋₁₀-alkyl)₂; —C₁₋₁₀-alkyl-P(═O)(OH)(OC₁₋₁₀-alkyl); —C₁₋₁₀-alkyl-S(═O)₂(OH); —C₁₋₁₀-alkyl-S(═O)₂(NH₂); —C₁₋₁₀-alkyl-C(═O)(OH);
R7 represents —H, —C₁₋₁₀-alkyl, —C₁₋₁₀-alkyl-C₃₋₁₀-cycloalkyl, —C₁₋₁₀-alkyl-aryl, —C₁₋₁₀-alkyl-heteroaryl, —C₃₋₁₀-cycloalkyl, —C₁₋₁₀-alkyl-O-aryl, and —C₁₋₁₀-alkyl-O-heteroaryl; and
R8 represents —C₃₋₆-cycloalkyl-aryl, —C₁₋₁₀-alkyl-aryl (wherein in case of —C₂-alkyl-aryl, the alkyl-moiety is saturated).

According to substitution pattern (iv),
R1 represents —H, —C₁₋₁₀-alkyl, —C₁₋₁₀-alkyl-C₃₋₁₀-cycloalkyl, —C₁₋₁₀-alkyl-aryl, —C₁₋₁₀-alkyl-heteroaryl, —C₃₋₁₀-cycloalkyl, —C₁₋₁₀-alkyl-O-aryl, and —C₁₋₁₀-alkyl-O-heteroaryl;
R3 represents C₁₋₁₀-alkyl-P(═O)(OC₁₋₁₀-alkyl)₂; —C₁₋₁₀-alkyl-P(═O)(OH)(OC₁₋₁₀-alkyl);
R7 represents —H, —C₁₋₁₀-alkyl, —C₁₋₁₀-alkyl-C₃₋₁₀-cycloalkyl, —C₁₋₁₀-alkyl-aryl, —C₁₋₁₀-alkyl-heteroaryl, —C₃₋₁₀-cycloalkyl, —C₁₋₁₀-alkyl-O-aryl, and —C₁₋₁₀-alkyl-O-heteroaryl; and
R8 represents —H, —C₁₋₁₀-alkyl, —C₁₋₁₀-alkyl-C₃₋₁₀-cycloalkyl, —C₁₋₁₀-alkyl-aryl, —C₁₋₁₀-alkyl-heteroaryl, —C₃₋₁₀-cycloalkyl, —C₁₋₁₀-alkyl-O-aryl, and —C₁₋₁₀-alkyl-O-heteroaryl.

Within the above definitions of the compounds according to substitution patterns (i), (ii), (iii) and (iv), in each case “C₁₋₁₀-alkyl” independently may be linear or branched, unless expressly stated otherwise saturated or unsaturated, unsubstituted or monosubstituted with a substituent selected from —C≡CH, —C(═O)OH, —C(═O)O—C₁₋₁₀-alkyl, —OH, —O—C₁₋₁₀-alkyl, —NH₂, —NH—C₁₋₁₀-alkyl, —N(C₁₋₁₀-alkyl)₂, —N₃, —F, —Cl, —Br, and —I.

Unless expressly stated otherwise, “alkyl” preferably refers to an aliphatic hydrocarbon including straight chain, or branched chain groups. Preferably, the alkyl group has 1 to 10 carbon atoms (C₁-C₁₀ alkyl), more preferably 1 to 6 carbon atoms (C₁-C₆ alkyl) and most preferably 1 to 4 carbon atoms (C₁-C₄ alkyl), e. g., methyl, ethyl, n-propyl, isopropyl, n-butyl, iso-butyl, sec-butyl, tert-butyl and the like.

The aliphatic hydrocarbon may be saturated or unsaturated. When it is unsaturated, it may contain one or more unsaturations, i.e., —C═C-double and/or —C≡C-triple bonds. If there is more than one unsaturation, the unsaturations may be conjugated or isolated. Thus, for the purpose of the specification the term “alkyl” encompasses saturated hydrocarbons as well as alkenyl, alkynyl and alkenynyl residues. “Alkenyl” preferably refers to an alkyl group, as defined above, consisting of at least two carbon atoms and at least one carbon-carbon double bond e.g., ethenyl, propenyl, butenyl or pentenyl and their structural isomeric forms such as 1- or 2-propenyl, 1-, 2-, or 3-butenyl and the like. “Alkynyl” preferably refers to an alkyl group, as defined above, consisting of at least two carbon atoms and at least one carbon-carbon triple bond e. g., acetylene, ethynyl, propynyl, butynyl, or pentynyl and their structural isomeric forms as described above. Alkyl may be substituted or unsubstituted. When substituted, the substituent group(s) is one or more, for example one or two groups, individually selected from the group consisting of —C≡CH, —C(═O)OH, —C(═O)O—C₁₋₁₀-alkyl, —OH, —O—C₁₋₁₀-alkyl, —NH₂, —NH—C₁₋₁₀-alkyl, —N(C₁₋₁₀-alkyl)₂, —N₃, —F, —Cl, —Br, and —I.

Within the above definitions of the compounds according to substitution patterns (i), (ii), (iii) and (iv), in each case “C₃₋₁₀-cycloalkyl” independently may be linear or branched, unless expressly stated otherwise saturated or unsaturated, unsubstituted or monosubstituted with a substituent selected from —C≡CH, —C(═O)OH, —C(═O)O—C₁₋₆-alkyl, —OH, —O—C₁₋₁₀-alkyl, —NH₂, —NH—C₁₋₁₀-alkyl, —N(C₁₋₁₀-alkyl)₂, —N₃, —F, —Cl, —Br, and —I.

Unless expressly stated otherwise, “cycloalkyl” preferably refers to cyclic hydrocarbon residue that contains no heteroatoms as ring members and that is not aromatic. “Cyclo-alkyl” may encompass a single cycle or more than one cycle. Preferably, cycloalkyl has 3 to 8 carbon atoms (—C₃-C₈ cycloalkyl). Cycloalkyl may be saturated, e.g., cyclopropane, cyclobutane, cyclopentane, cyclohexane, cycloheptane, adamantane; or unsaturated (e.g., cycloalkenyl, cycloalkynyl), e.g., cyclobutenyl, cyclopentenyl, cyclohexenyl, cyclo-hexadiene, cycloheptatriene and the like. Cycloalkyl may be substituted or unsubstituted.

When substituted, the substituent group(s) is one or more, for example one or two groups, individually selected from —C≡CH, —C(═O)OH, —C(═O)O—C₁₋₆-alkyl, —OH, —O—C₁₋₁₀-alkyl, —NH₂, —NH—C₁₋₁₀-alkyl, —N(C₁₋₁₀-alkyl)₂, —N₃, —F, —Cl, —Br, and —I.

Within the above definitions of the compounds according to substitution patterns (i), (ii), (iii) and (iv), in each case “aryl” independently is a 6-14-membered aryl moiety which may be unsubstituted, mono- or disubstituted with a substituent independently selected from —C≡CH, —CH₃, —CH₂—CH₃, —CH(CH₃)₂, -phenyl, —CF₃, —CCl₃, —CBr₃, —Cl₃, —C(═O)OH, —C(═O)O—C₁₋₁₀-alkyl, —OH, —O—C₁₋₁₀-alkyl, —NH₂, —NH—C₁₋₁₀-alkyl, —N(C₁₋₁₀-alkyl)₂, —N₃, —F, —Cl, —Br, and —I; preferably, in each case “aryl” is a 6-14-membered aryl moiety which may be unsubstituted, mono- or disubstituted with a substituent independently selected from —C≡CH, —CF₃, —CCl₃, —CBr₃, —Cl₃, —C(═O)OH, —C(═O)O—C₁₋₁₀-alkyl, —OH, —O—C₁₋₁₀-alkyl, —NH₂, —NH—C₁₋₁₀-alkyl, —N(C₁₋₁₀-alkyl)₂, —N₃, —F, —Cl, —Br, and —I;

Unless expressly stated otherwise, “aryl” preferably refers to an aromatic all-carbon monocyclic or fused-ring polycyclic group (i.e., rings which share adjacent pairs of carbon atoms) of 6 to 14 ring atoms and having a completely conjugated pi-electron system. Examples, without limitation, of aryl groups are phenyl, naphthalenyl and anthracenyl. The aryl group may be substituted or unsubstituted. When substituted, the substituted group(s) is one or more, for example one, two, or three substituents, independently selected from the group consisting of —C≡CH, —CH₃, —CH₂—CH₃, —CH(CH₃)₂, -phenyl, —CF₃, —CCl₃, —CBr₃, —Cl₃, —C(═O)OH, —C(═O)O—C₁₋₁₀-alkyl, —OH, —O—C₁₋₁₀-alkyl, —NH₂, —NH—C₁₋₁₀-alkyl, —N(C₁₋₁₀-alkyl)₂, —N₃, —F, —Cl, —Br, and —I; preferably —C≡CH, —CF₃, —CCl₃, —CBr₃, —Cl₃, —C(═O)OH, —C(═O)O—C₁₋₁₀-alkyl, —OH, —O—C₁₋₁₀-alkyl, —NH₂, —NH—C₁₋₁₀-alkyl, —N(C₁₋₁₀-alkyl)₂, —N₃, —F, —Cl, —Br, and —I.

Within the above definitions of the compounds according to substitution patterns (i) and (ii), in each case “heteroaryl” independently is a 5-14-membered heteroaryl moiety which may be unsubstituted, mono- or disubstituted with a substituent independently selected from —C≡CH, —CF₃, —CCl₃, —CBr₃, —Cl₃, —C(═O)OH, —C(═O)O—C₁₋₁₀-alkyl, —OH, —O—C₁₋₁₀-alkyl, —NH₂, —NH—C₁₋₁₀-alkyl, —N(C₁₋₁₀-alkyl)₂, —N₃, —F, —Cl, —Br, and —I.

Unless expressly stated otherwise, “heteroaryl” preferably refers to a monocyclic or fused aromatic ring (i.e., rings which share an adjacent pair of atoms) of 5 to 10 ring atoms in which one, two, three or four ring atoms are selected from the group consisting of N, O and S and the rest being carbon. Examples, without limitation, of heteroaryl groups are pyridyl, pyrrolyl, furyl, thienyl, imidazolyl, oxazolyl, isoxazolyl, thiazolyl, isothiazolyl, pyrazolyl, 1,2,3-triazolyl, 1,2,4-triazolyl, 1,2,3-oxadiazolyl, 1,2,4-oxadiazolyl, 1,2,5-oxadiazolyl, 1,3,4-oxadiazolyl, 1,3,4-triazinyl, 1,2,3-triazinyl, benzofuryl, isobenzofuryl, benzothienyl, benzotriazolyl, isobenzothienyl, indolyl, isoindolyl, 3H-indolyl, benzimidazolyl, benzothiazolyl, benzoxazolyl, quinolizinyl, quinazolinyl, pthalazinyl, quinoxalinyl, cinnnolinyl, napthyridinyl, quinolyl, isoquinolyl, tetrazolyl, 5,6,7,8-tetrahydroquinolyl, 5,6,7,8-tetra-hydroisoquinolyl, purinyl, pteridinyl, pyridinyl, pyrimidinyl, carbazolyl, xanthenyl or benzoquinolyl. The heteroaryl group may be substituted or unsubstituted. When substituted, the substituted group(s) is one or more, for example one or two substituents, independently selected from the group consisting of —C≡CH, —CF₃, —CCl₃, —CBr₃, —Cl₃, —C(═O)OH, —C(═O)O—C₁₋₁₀-alkyl, —OH, —O—C₁₋₁₀-alkyl, —NH₂, —NH—C₁₋₁₀-alkyl, —N(C₁₋₁₀-alkyl)₂, —N₃, —F, —Cl, —Br, and —I.

If not expressly stated otherwise, any residue, group or moiety defined herein that can be substituted is preferably substituted with one or more substituents independently selected from the group consisting of —C≡CH, —CF₃, —CCl₃, —CBr₃, —Cl₃, —C(═O)OH, —C(═O)O—C₁₋₁₀-alkyl, —OH, —O—C₁₋₁₀-alkyl, —NH₂, —NH—C₁₋₁₀-alkyl, —N(C₁₋₁₀-alkyl)₂, —N₃, —F, —Cl, —Br, and —I.

The invention also relates to the stereoisomers of the compounds according to general formula 1 e.g. the enantiomers or diastereomers in racemic, enriched or substantially pure form.

In preferred embodiments of the compounds according to substitution patterns (i), (ii), (iii) and (iv), R1 represents —C₁₋₁₀-alkyl, optionally substituted with —C≡CH; or —C₁₋₁₀-alkyl-C₃₋₁₀-cycloalkyl; preferably —C₁₋₆-alkyl, optionally substituted with —C≡CH; or —C₁₋₆-alkyl-C₃₋₆-cycloalkyl; more preferably —CH₂CH₃, —CH₂CH₂CH₃, —CH₂C≡CH, or —CH₂-cyclobutyl.

In preferred embodiments of the compounds according to substitution patterns (i), (ii), (iii) and (iv), R3 represents —C₃₋₅-alkyl-P(═O)(OH)₂; —C₃₋₅-alkyl-P(═O)(OC₁₋₆-alkyl)₂; —C₃₋₅-alkyl-P(═O)(OH)(OC₁₋₆-alkyl); —C₃₋₅-alkyl-S(═O)₂(OH); —C₃₋₅-alkyl-S(═O)₂(NH₂); or —C₃₋₅-alkyl-C(═O)(OH); preferably —C₃₋₅-alkyl-P(═O)(OH)₂; more preferably —C₄-alkyl-P(═O)(OH)₂.

In preferred embodiments of the compounds according to substitution patterns (i), (ii), (iii) and (iv), R7 represents —H; —C₁₋₁₀-alkyl, optionally substituted with —OH; —C₁₋₁₀-alkyl-C₃₋₁₀-cycloalkyl; or —C₁₋₁₀-alkyl-aryl;

preferably —H; —C₁₋₆-alkyl, optionally substituted with —OH; —C₁₋₆-alkyl-C₃₋₆-cycloalkyl; or —C₁₋₆-alkyl-aryl;

more preferably —H, —CH₃, —CH₂CH₃, —CH₂CH₂—OH, —CH₂CH₂CH₃, —CH₂-cyclopropyl, or —CH₂-phenyl.

In preferred embodiments of the compounds according to substitution patterns (i), (ii), (iii) and (iv), R8 represents —C₃₋₁₀-cycloalkyl, —C₁₋₁₀-alkyl-C₃₋₁₀-cycloalkyl, -aryl, —C₁₋₁₀-alkyl-aryl, —C₃₋₆-cycloalkyl-aryl, or —C₁₋₁₀-alkyl-O-aryl;

preferably -phenyl, optionally substituted with —F, —Br, —Cl, —CH₃, —CF₃, —OCH₃; —CH₂-phenyl, optionally substituted with —F, —Br, —Cl, —CH₃, —CF₃, —OCH₃; —CH₂CH₂-phenyl, optionally substituted with one or two substituents independently of one another selected from —F, —Br, —Cl, —CH₃, —CF₃, —OCH₃; —CH═CH— phenyl, optionally substituted with —F, —Br, —Cl, —CH₃, —CF₃, —OCH₃; —CH₂—O-phenyl, optionally substituted with —F, —Br, —Cl, —CH₃, —CF₃, —OCH₃; -cyclopropyl-phenyl, optionally substituted with —F, —Br, —Cl, —CH₃, —CF₃, —OCH₃; or —C₃₋₆-cycloalkyl;

more preferably -phenyl, optionally substituted with —OCH₃; —CH₂-phenyl, optionally substituted with —Cl; —CH₂CH₂-phenyl, optionally substituted with one or two substituents independently of one another selected from —F, —Br, —Cl, —CH₃, —CF₃, —OCH₃; —CH═CH-phenyl, optionally substituted with —OCH₃; —CH₂—O-phenyl; -cyclopropyl-phenyl; or -cyclopentyl.

In preferred embodiments of the compounds according to substitution pattern (i)
R1 represents —H, —C₁₋₆-alkyl, —C₁₋₆-alkyl-C₃₋₆-cycloalkyl, —C₁₋₆-alkyl-aryl, or —C₁₋₆-alkyl-heteroaryl; or
R3 represents —C₁₋₆-alkyl-P(═O)(OH)₂;
R7 represents —H, —C₁₋₆-alkyl, —C₁₋₆-alkyl-C₃₋₆-cycloalkyl, —C₁₋₆-alkyl-aryl, or —C₁₋₆-alkyl-heteroaryl; and
R8 represents —C₃₋₆-cycloalkyl-phenyl or —C₁₋₆-alkyl-phenyl (wherein in case of —C₂-alkyl-phenyl, the alkyl-moiety is saturated).

In preferred embodiments of the compounds according to substitution pattern (ii),
R1 represents —H, —C₁₋₆-alkyl, —C₁₋₆-alkyl-C₃₋₆-cycloalkyl, —C₁₋₆-alkyl-aryl, —C₁₋₆-alkyl-heteroaryl, —C₃₋₆-cycloalkyl, —C₁₋₆—O-aryl, or —C₁₋₆—O-heteroaryl;
R3 represents —C₁₋₆-alkyl-P(═O)(OC₁₋₆-alkyl)₂; —C₁₋₆-alkyl-P(═O)(OH)(OC₁₋₆-alkyl); —C₁₋₆-alkyl-S(═O)₂(OH); —C₁₋₆-alkyl-S(═O)₂(NH₂); or —C₁₋₆-alkyl-C(═O)(OH);
R7 represents —H, —C₁₋₆-alkyl, —C₁₋₆-alkyl-C₃₋₆-cycloalkyl, —C₁₋₆-alkyl-aryl, —C₁₋₆-alkyl-heteroaryl, —C₃₋₆-cycloalkyl, —C₁₋₆—O-aryl, or —C₁₋₆—O-heteroaryl; and
R8 represents —H, —C₁₋₆-alkyl, —C₁₋₆-alkyl-C₃₋₆-cycloalkyl, —C₁₋₆-alkyl-aryl, —C₁₋₆-alkyl-heteroaryl, —C₃₋₆-cycloalkyl, —C₁₋₆—O-aryl, or —C₁₋₆—O-heteroaryl.

In preferred embodiments of the compounds according to substitution pattern (iii),
R1 represents —H, —C₁₋₆-alkyl, —C₁₋₆-alkyl-C₃₋₆-cycloalkyl, —C₁₋₆-alkyl-aryl, —C₁₋₆-alkyl-heteroaryl, —C₃₋₆-cycloalkyl, —C₁₋₆—O-aryl, or —C₁₋₆—O-heteroaryl;
R3 represents —C₁₋₆-alkyl-P(═O)(OC₁₋₆-alkyl)₂; —C₁₋₆-alkyl-P(═O)(OH)(OC₁₋₆-alkyl); —C₁₋₆-alkyl-S(═O)₂(OH); —C₁₋₆-alkyl-S(═O)₂(NH₂); or —C₁₋₆-alkyl-C(═O)(OH);
R7 represents —H, —C₁₋₆-alkyl, —C₁₋₆-alkyl-C₃₋₆-cycloalkyl, —C₁₋₆-alkyl-aryl, —C₁₋₆-alkyl-heteroaryl, —C₃₋₆-cycloalkyl, —C₁₋₆—O-aryl, or —C₁₋₆—O-heteroaryl; and
R8 represents cyclopropyl-aryl, —CH₂-phenyl or —CH₂CH₂-phenyl (wherein in case of —CH₂CH₂-phenyl, the ethyl-moiety is saturated).

In preferred embodiments of the compounds according to substitution pattern (iv),
R1 represents —H, —C₁₋₆-alkyl, —C₁₋₆-alkyl-C₃₋₆-cycloalkyl, —C₁₋₆-alkyl-aryl, —C₁₋₆-alkyl-heteroaryl, —C₃₋₆-cycloalkyl, —C₁₋₆—O-aryl, or —C₁₋₆—O-heteroaryl;
R3 represents —C₁₋₆-alkyl-P(═O)(OC₁₋₆-alkyl)₂; —C₁₋₆-alkyl-P(═O)(OH)(OC₁₋₆-alkyl);
R7 represents —H, —C₁₋₆-alkyl, —C₁₋₆-alkyl-C₃₋₆-cycloalkyl, —C₁₋₆-alkyl-aryl, —C₁₋₆-alkyl-heteroaryl, —C₃₋₆-cycloalkyl, —C₁₋₆—O-aryl, or —C₁₋₆—O-heteroaryl; and
R8 represents —H, —C₁₋₆-alkyl, —C₁₋₆-alkyl-C₃₋₆-cycloalkyl, —C₁₋₆-alkyl-aryl, —C₁₋₆-alkyl-heteroaryl, —C₃₋₆-cycloalkyl, —C₁₋₆—O-aryl, or —C₁₋₆—O-heteroaryl.

In more preferred embodiments of the compounds according to substitution pattern (i),
R1 represents —H, —C₁₋₆-alkyl, —C₁₋₆-alkyl-C₃₋₆-cycloalkyl, —C₁₋₆-alkyl-aryl, or —C₁₋₆-alkyl-heteroaryl;
R3 represents —C₄-alkyl-P(═O)(OH)₂;
R7 represents —H, —C₁₋₆-alkyl, —C₁₋₆-alkyl-C₃₋₆-cycloalkyl, —C₁₋₆-alkyl-aryl, or —C₁₋₆-alkyl-heteroaryl; and
R8 represents —C₃-cycloalkyl-aryl or —C₁₋₆-alkyl-phenyl (wherein in case of —C₂-alkyl-phenyl, the alkyl-moiety is saturated).

In more preferred embodiments of the compounds according to substitution pattern (ii),
R1 represents —H, —C₁₋₆-alkyl, —C₁₋₆-alkyl-C₃₋₆-cycloalkyl, —C₁₋₆-alkyl-aryl, —C₁₋₆-alkyl-heteroaryl, —C₃₋₆-cycloalkyl, —C₁₋₆—O-aryl, or —C₁₋₆—O-heteroaryl;
R3 represents —C₄-alkyl-P(═O)(OC₁₋₆-alkyl)₂; —C₄-alkyl-P(═O)(OH)(OC₁₋₆-alkyl); —C₄-alkyl-S(═O)₂(OH); —C₄-alkyl-S(═O)₂(NH₂); or —C₄-alkyl-C(═O)(OH);
R7 represents —H, —C₁₋₆-alkyl, —C₁₋₆-alkyl-C₃₋₆-cycloalkyl, —C₁₋₆-alkyl-aryl, —C₁₋₆-alkyl-heteroaryl, —C₃₋₆-cycloalkyl, —C₁₋₆—O-aryl, or —C₁₋₆—O-heteroaryl; and
R8 represents —H, —C₁₋₆-alkyl, —C₁₋₆-alkyl-C₃₋₆-cycloalkyl, —C₁₋₆-alkyl-aryl, —C₁₋₆-alkyl-heteroaryl, —C₃₋₆-cycloalkyl, —C₁₋₆—O-aryl, or —C₁₋₆—O-heteroaryl.

In more preferred embodiments of the compounds according to substitution pattern (iii),
R1 represents —H, —C₁₋₆-alkyl, —C₁₋₆-alkyl-C₃₋₆-cycloalkyl, —C₁₋₆-alkyl-aryl, —C₁₋₆-alkyl-heteroaryl, —C₃₋₆-cycloalkyl, —C₁₋₆—O-aryl, or —C₁₋₆—O-heteroaryl;
R3 represents —C₄-alkyl-P(═O)(OC₁₋₆-alkyl)₂; —C₄-alkyl-P(═O)(OH)(OC₁₋₆-alkyl); —C₄-alkyl-S(═O)₂(OH); —C₄-alkyl-S(═O)₂(NH₂); or —C₄-alkyl-C(═O)(OH);
R7 represents —H, —C₁₋₆-alkyl, —C₁₋₆-alkyl-C₃₋₆-cycloalkyl, —C₁₋₆-alkyl-aryl, —C₁₋₆-alkyl-heteroaryl, —C₃₋₆-cycloalkyl, —C₁₋₆—O-aryl, or —C₁₋₆—O-heteroaryl; and
R8 represents —C₃-cycloalkyl-aryl or —C₁₋₆-alkyl-phenyl (wherein in case of —C₂-alkyl-phenyl, the alkyl-moiety is saturated).

In more preferred embodiments of the compounds according to substitution pattern (iv),
R1 represents —H, —C₁₋₆-alkyl, —C₁₋₆-alkyl-C₃₋₆-cycloalkyl, —C₁₋₆-alkyl-aryl, —C₁₋₆-alkyl-heteroaryl, —C₃₋₆-cycloalkyl, —C₁₋₆—O-aryl, or —C₁₋₆—O-heteroaryl;
R3 represents —C₄-alkyl-P(═O)(OC₁₋₆-alkyl)₂; —C₄-alkyl-P(═O)(OH)(OC₁₋₆-alkyl); —C₄-alkyl-;
R7 represents —H, —C₁₋₆-alkyl, —C₁₋₆-alkyl-C₃₋₆-cycloalkyl, —C₁₋₆-alkyl-aryl, —C₁₋₆-alkyl-heteroaryl, —C₃₋₆-cycloalkyl, —C₁₋₆—O-aryl, or —C₁₋₆—O-heteroaryl; and
R8 represents —H, —C₁₋₆-alkyl, —C₁₋₆-alkyl-C₃₋₆-cycloalkyl, —C₁₋₆-alkyl-aryl, —C₁₋₆-alkyl-heteroaryl, —C₃₋₆-cycloalkyl, —C₁₋₆—O-aryl, or —C₁₋₆—O-heteroaryl.

In still more preferred embodiments of the compounds according to substitution pattern (i),
R1 represents —H, —C₁₋₆-alkyl, —C₁₋₆-alkyl-C₃₋₆-cycloalkyl, —C₁₋₆-alkyl-aryl, or —C₁₋₆-alkyl-heteroaryl;
R3 represents —CH₂CH₂CH₂CH₂—P(═O)(OH)₂;
R7 represents —H, —C₁₋₆-alkyl, —C₁₋₆-alkyl-C₃₋₆-cycloalkyl, —C₁₋₆-alkyl-aryl, or —C₁₋₆-alkyl-heteroaryl; and
R8 represents -cyclopropyl-aryl, —CH₂-phenyl or —CH₂CH₂-phenyl.

In still more preferred embodiments of the compounds according to substitution pattern (ii),
R1 represents —H, —C₁₋₆-alkyl, —C₁₋₆-alkyl-C₃₋₆-cycloalkyl, —C₁₋₆-alkyl-aryl, —C₁₋₆-alkyl-heteroaryl, —C₃₋₆-cycloalkyl, —C₁₋₆—O-aryl, or —C₁₋₆—O-heteroaryl;
R3 represents —CH₂CH₂CH₂CH₂—P(═O)(OC₁₋₆-alkyl)₂; —CH₂CH₂CH₂CH₂—P(═O)(OH)(OC₁₋₆-alkyl); —CH₂CH₂CH₂CH₂—S(═O)₂(OH); —CH₂CH₂CH₂CH₂—S(═O)₂(NH₂); or —CH₂CH₂CH₂CH₂—C(═O)(OH);
R7 represents —H, —C₁₋₆-alkyl, —C₁₋₆-alkyl-C₃₋₆-cycloalkyl, —C₁₋₆-alkyl-aryl, —C₁₋₆-alkyl-heteroaryl, —C₃₋₆-cycloalkyl, —C₁₋₆—O-aryl, or —C₁₋₆—O-heteroaryl; and
R8 represents —H, —C₁₋₆-alkyl, —C₁₋₆-alkyl-C₃₋₆-cycloalkyl, —C₁₋₆-alkyl-aryl, —C₁₋₆-alkyl-heteroaryl, —C₃₋₆-cycloalkyl, —C₁₋₆—O-aryl, or —C₁₋₆—O-heteroaryl.

In still more preferred embodiments of the compounds according to substitution pattern (iii),
R1 represents —H, —C₁₋₆-alkyl, —C₁₋₆-alkyl-C₃₋₆-cycloalkyl, —C₁₋₆-alkyl-aryl, —C₁₋₆-alkyl-heteroaryl, —C₃₋₆-cycloalkyl, —C₁₋₆—O-aryl, or —C₁₋₆—O-heteroaryl;
R3 represents —CH₂CH₂CH₂CH₂—P(═O)(OC₁₋₆-alkyl)₂; —CH₂CH₂CH₂CH₂—P(═O)(OH)(OC₁₋₆-alkyl); —CH₂CH₂CH₂CH₂—S(═O)₂(OH); —CH₂CH₂CH₂CH₂—S(═O)₂(NH₂); or —CH₂CH₂CH₂CH₂—C(═O)(OH);
R7 represents —H, —C₁₋₆-alkyl, —C₁₋₆-alkyl-C₃₋₆-cycloalkyl, —C₁₋₆-alkyl-aryl, —C₁₋₆-alkyl-heteroaryl, —C₃₋₆-cycloalkyl, —C₁₋₆—O-aryl, or —C₁₋₆—O-heteroaryl; and
R8 represents -cyclopropyl-aryl, —CH₂-phenyl or —CH₂CH₂-phenyl.

In still more preferred embodiments of the compounds according to substitution pattern (iv),
R1 represents —H, —C₁₋₆-alkyl, —C₁₋₆-alkyl-C₃₋₆-cycloalkyl, —C₁₋₆-alkyl-aryl, —C₁₋₆-alkyl-heteroaryl, —C₃₋₆-cycloalkyl, —C₁₋₆—O-aryl, or —C₁₋₆—O-heteroaryl;
R3 represents —CH₂CH₂CH₂CH₂—P(═O)(OC₁₋₆-alkyl)₂; —CH₂CH₂CH₂CH₂—P(═O)(OH)(OC₁₋₆-alkyl);
R7 represents —H, —C₁₋₆-alkyl, —C₁₋₆-alkyl-C₃₋₆-cycloalkyl, —C₁₋₆-alkyl-aryl, —C₁₋₆-alkyl-heteroaryl, —C₃₋₆-cycloalkyl, —C₁₋₆—O-aryl, or —C₁₋₆—O-heteroaryl; and
R8 represents —H, —C₁₋₆-alkyl, —C₁₋₆-alkyl-C₃₋₆-cycloalkyl, —C₁₋₆-alkyl-aryl, —C₁₋₆-alkyl-heteroaryl, —C₃₋₆-cycloalkyl, —C₁₋₆—O-aryl, or —C₁₋₆—O-heteroaryl.

Within any of the above definitions of the preferred, more preferred and still more preferred embodiments of the compounds according to substitution patterns (i), (ii), (iii) and (iv), in each case “C₁₋₆-alkyl” may be linear or branched, unless expressly stated otherwise saturated or unsaturated, unsubstituted or monosubstituted with a substituent selected from —C≡CH, —C(═O)OH, —C(═O)O—C₁₋₁₀-alkyl, —OH, —O—C₁₋₁₀-alkyl, —NH₂, —NH—C₁₋₁₀-alkyl, —N(C₁₋₁₀-alkyl)₂, —N₃, —F, —Cl, —Br, and —I.

Within any of the above definitions of the preferred, more preferred and still more preferred embodiments of the compounds according to substitution patterns (i), (ii), (iii) and (iv), in each case “C₃₋₆-cycloalkyl” may be linear or branched, unless expressly stated otherwise saturated or unsaturated, unsubstituted or monosubstituted with a substituent selected from —C≡CH, —C(═O)OH, —C(═O)O—C₁₋₆-alkyl, —OH, —O—C₁₋₁₀-alkyl, —NH₂, —NH—C₁₋₁₀-alkyl, —N(C₁₋₁₀-alkyl)₂, —N₃, —F, —Cl, —Br, and —I.

Within any of the above definitions of the preferred, more preferred and still more preferred embodiments of the compounds according to substitution patterns (i), (ii), (iii) and (iv), in each case “aryl” and “heteroaryl” are as defined above.

In particularly preferred embodiments of the compounds according to general formula 1,
R1 represents —CH₂—C≡CH, —CH₂—CH₃, or —CH₂-cyclobutyl, unsubstituted;
R3 represents (—CH₂—)₄₋₆—P(═O)(OH)₂;
R7 represents (i) —H; (ii) —CH₃, —CH₂—CH₃, —CH₂—CH₂—CH₃, —CH(CH₃)₂, —CH(CH₃)(CH₂—CH₃), —CH₂—CH₂—OH, —CH₂—CH₂—Cl, —CH₂—CH₂—NH₂, —CH₂-cyclopropyl, unsubstituted, —CH₂-cyclobutyl, unsubstituted, —CH₂-cyclopentyl, unsubstituted, —CH₂-cyclohexyl, unsubstituted; or (iii) —CH₂-phenyl, unsubstituted;
R8 represents (i) —CH₂—CH₂-phenyl, unsubstituted; (ii) —CH₂—CH₂-phenyl, mono-, di- or trisubstituted with —OCH₃, —CH₃, —CH₂—CH₃, —CH(CH₃)₂, —CF₃, —F, —Cl, —Br, —I and/or unsubstituted-phenyl; or (iii) —CH₂-naphthyl, unsubstituted; or
R1 represents —CH₂—C≡CH, —CH₂—CH₃, or —CH₂-cyclobutyl, unsubstituted;
R3 represents (—CH₂—)₄—P(═O)(OCH₂CH₃)₂;
R7 represents —H or —CH₃;
R8 represents (i) —CH₂—O-phenyl, unsubstituted; (ii) —CH₂—CH₂-phenyl, mono-, di- or trisubstituted with —OCH₃, —CH₃, —CF₃ and/or —Br; or (iii) -cyclopropyl-phenyl, unsubstituted; or
R1 represents —CH₂—C≡CH;
R3 represents (—CH₂—)₄—P(═O)(OH)(OCH₂CH₃);
R7 represents —H or —CH₃;
R8 represents —CH₂—CH₂-phenyl, mono-, di- or trisubstituted with —OCH₃ and/or —Br; or
R1 represents —CH₂—C≡CH or —CH₂—CH₃;
R3 represents (—CH₂—)₄—SO₂—OH;
R7 represents —H;
R8 represents —CH₂—CH₂-phenyl, mono-, di- or trisubstituted with —OCH₃ and/or —Br; or
R1 represents —CH₂—C≡CH;
R3 represents (—CH₂—)₄—SO₂—NH₂;
R7 represents —H;
R8 represents —CH₂—CH₂-phenyl, mono-, di- or trisubstituted with —Br; or
R1 represents —CH₂—C≡CH or CH₂-cyclobutyl, unsubstituted;
R3 represents (—CH₂—)₄₋₅—CO₂H;
R7 represents —H;
R8 represents —CH₂—CH₂-phenyl, mono-, di- or trisubstituted with —OCH₃, CF₃ and/or —Br.

Particularly preferred compounds according to substitution pattern (i) are compounds B-1 to B-58:

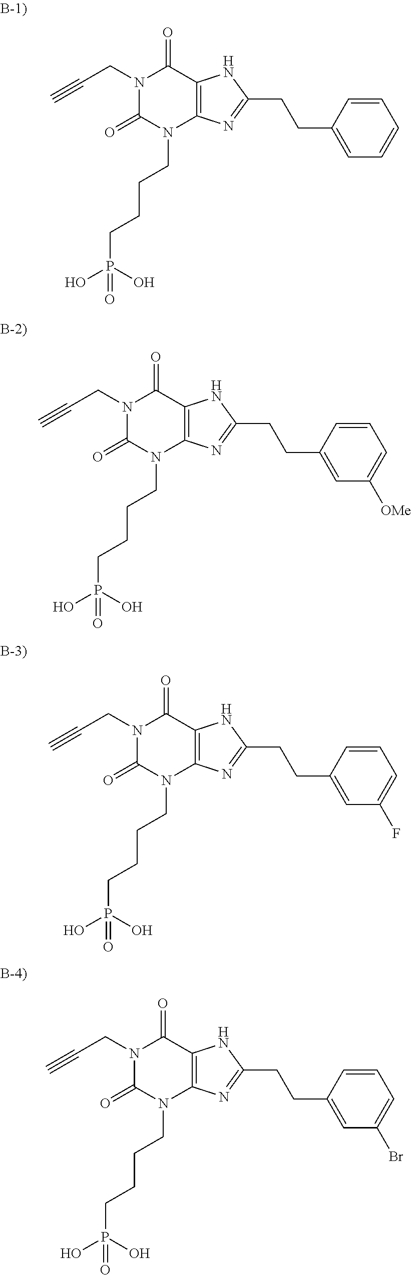 [SMILES: C#CCn1c(=O)c2[nH]c(CCc3ccccc3)nc2n(CCCCP(=O)(O)O)c1=O ; C#CCn1c(=O)c2[nH]c(CCc3cccc(OC)c3)nc2n(CCCCP(=O)(O)O)c1=O ; C#CCn1c(=O)c2[nH]c(CCc3cccc(F)c3)nc2n(CCCCP(=O)(O)O)c1=O ; C#CCn1c(=O)c2[nH]c(CCc3cccc(Br)c3)nc2n(CCCCP(=O)(O)O)c1=O]

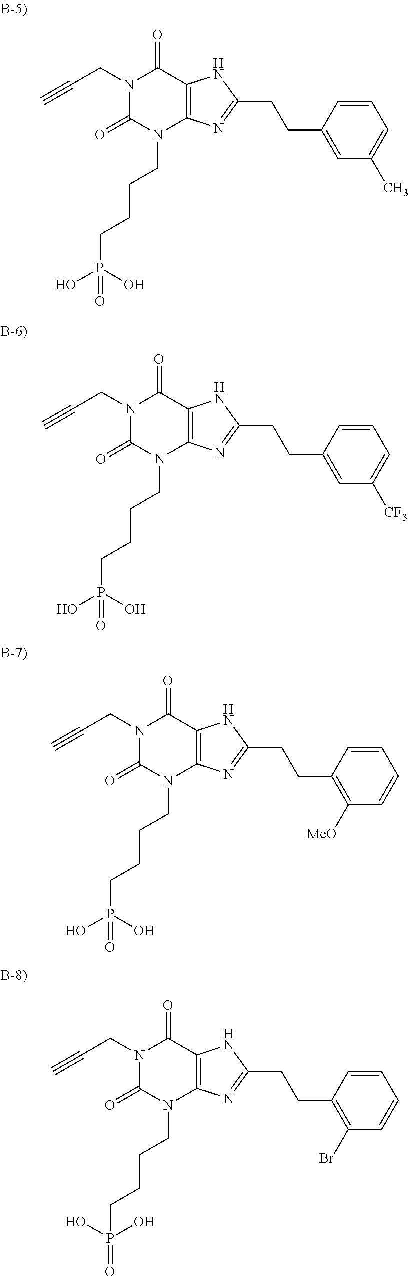

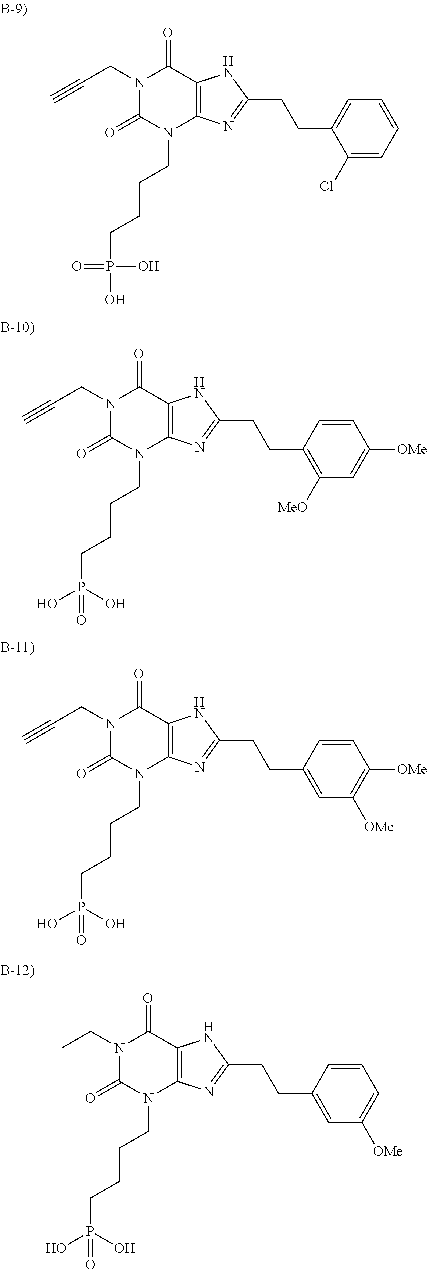 [SMILES: C#CCn1c(=O)c2[nH]c(CCc3ccccc3Cl)nc2n(CCCCP(=O)(O)O)c1=O ; C#CCn1c(=O)c2[nH]c(CCc3ccc(OC)cc3OC)nc2n(CCCCP(=O)(O)O)c1=O ; C#CCn1c(=O)c2[nH]c(CCc3ccc(OC)c(OC)c3)nc2n(CCCCP(=O)(O)O)c1=O ; CCn1c(=O)c2[nH]c(CCc3cccc(OC)c3)nc2n(CCCCP(=O)(O)O)c1=O]

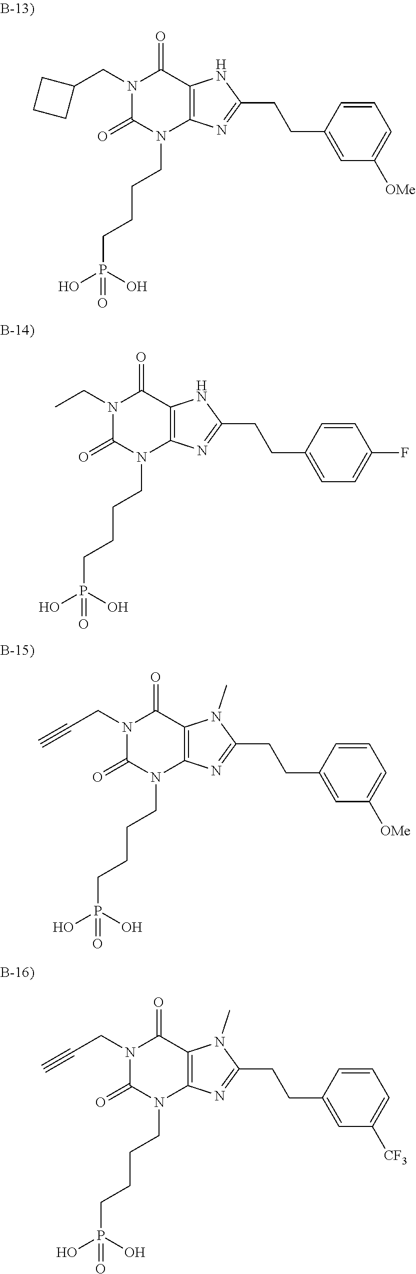 [SMILES: COc1cccc(CCc2nc3c([nH]2)c(=O)n(CC2CCC2)c(=O)n3CCCCP(=O)(O)O)c1 ; CCn1c(=O)c2[nH]c(CCc3ccc(F)cc3)nc2n(CCCCP(=O)(O)O)c1=O ; C#CCn1c(=O)c2c(nc(CCc3cccc(OC)c3)n2C)n(CCCCP(=O)(O)O)c1=O ; C#CCn1c(=O)c2c(nc(CCc3cccc(C(F)(F)F)c3)n2C)n(CCCCP(=O)(O)O)c1=O]

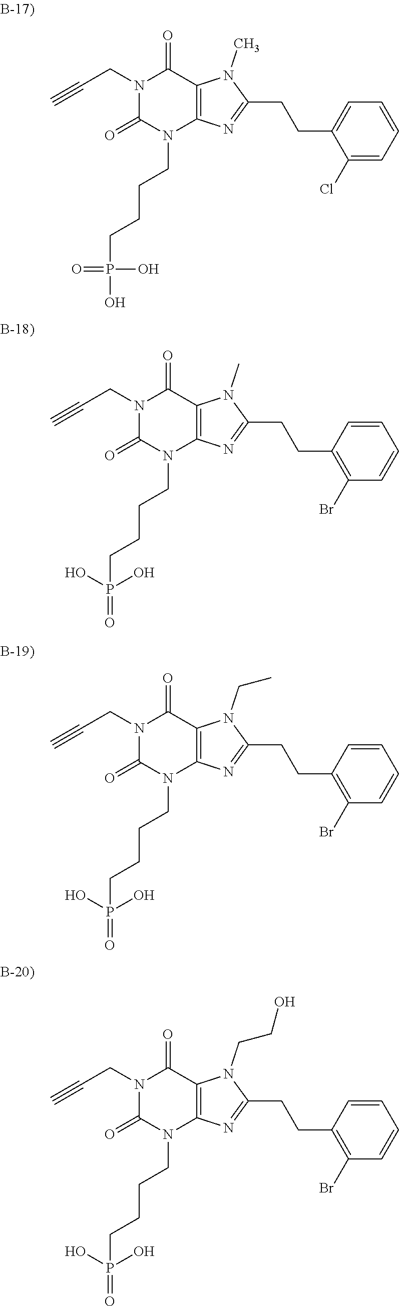 [SMILES: C#CCn1c(=O)c2c(nc(CCc3ccccc3Cl)n2C)n(CCCCP(=O)(O)O)c1=O ; C#CCn1c(=O)c2c(nc(CCc3ccccc3Br)n2C)n(CCCCP(=O)(O)O)c1=O ; C#CCn1c(=O)c2c(nc(CCc3ccccc3Br)n2CC)n(CCCCP(=O)(O)O)c1=O ; C#CCn1c(=O)c2c(nc(CCc3ccccc3Br)n2CCO)n(CCCCP(=O)(O)O)c1=O]

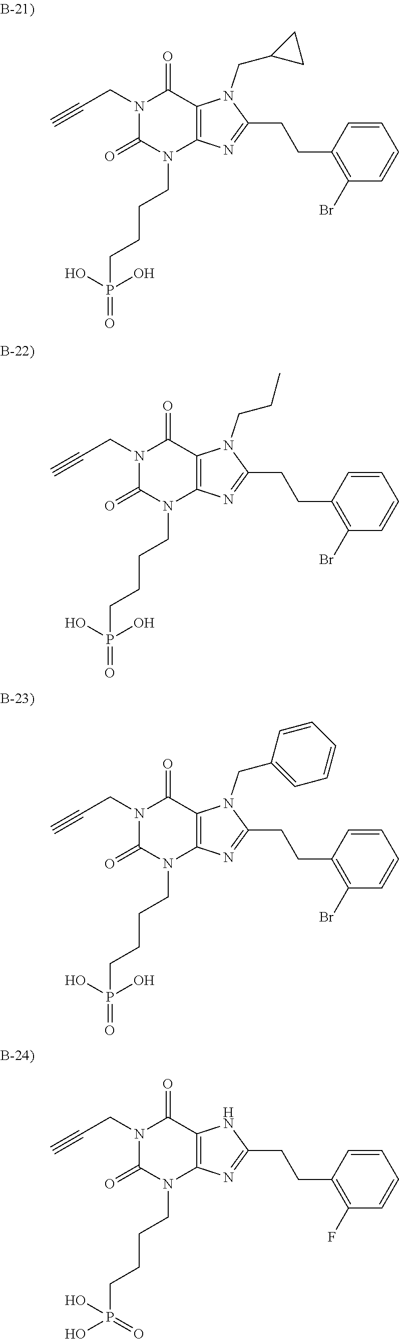 [SMILES: C#CCn1c(=O)c2c(nc(CCc3ccccc3Br)n2CC2CC2)n(CCCCP(=O)(O)O)c1=O ; C#CCn1c(=O)c2c(nc(CCc3ccccc3Br)n2CCC)n(CCCCP(=O)(O)O)c1=O ; C#CCn1c(=O)c2c(nc(CCc3ccccc3Br)n2Cc2ccccc2)n(CCCCP(=O)(O)O)c1=O ; C#CCn1c(=O)c2[nH]c(CCc3ccccc3F)nc2n(CCCCP(=O)(O)O)c1=O]

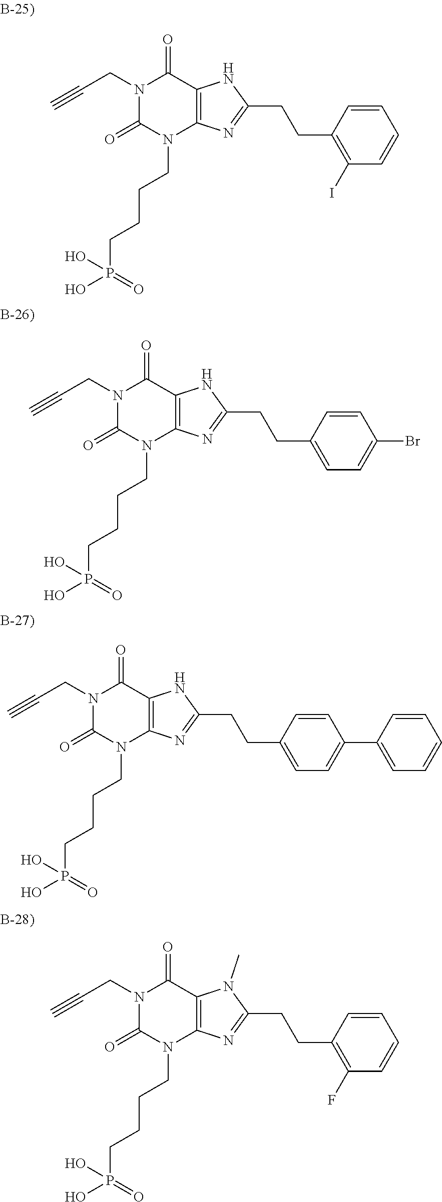 [SMILES: C#CCn1c(=O)c2[nH]c(CCc3ccccc3I)nc2n(CCCCP(=O)(O)O)c1=O ; C#CCn1c(=O)c2[nH]c(CCc3ccc(Br)cc3)nc2n(CCCCP(=O)(O)O)c1=O ; C#CCn1c(=O)c2[nH]c(CCc3ccc(-c4ccccc4)cc3)nc2n(CCCCP(=O)(O)O)c1=O ; C#CCn1c(=O)c2c(nc(CCc3ccccc3F)n2C)n(CCCCP(=O)(O)O)c1=O]

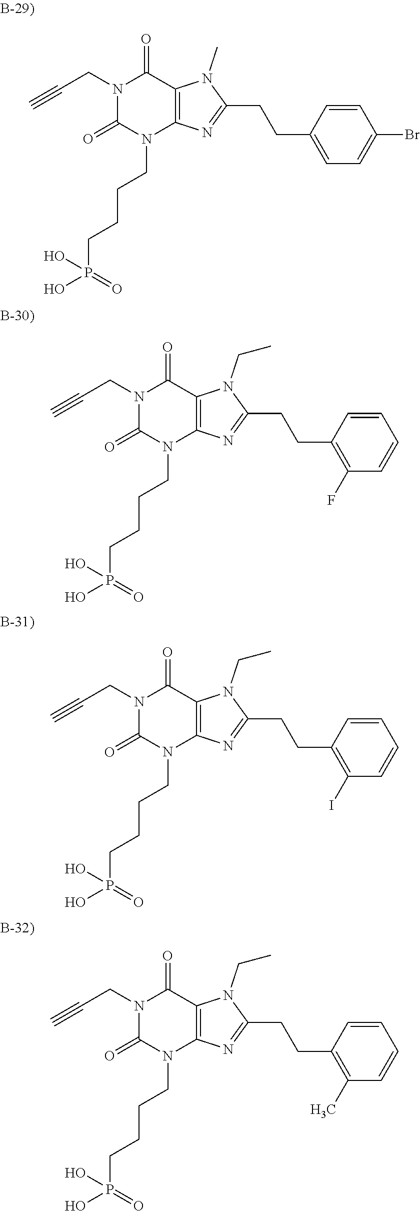 [SMILES: C#CCn1c(=O)c2c(nc(CCc3ccc(Br)cc3)n2C)n(CCCCP(=O)(O)O)c1=O ; C#CCn1c(=O)c2c(nc(CCc3ccccc3F)n2CC)n(CCCCP(=O)(O)O)c1=O ; C#CCn1c(=O)c2c(nc(CCc3ccccc3I)n2CC)n(CCCCP(=O)(O)O)c1=O ; C#CCn1c(=O)c2c(nc(CCc3ccccc3C)n2CC)n(CCCCP(=O)(O)O)c1=O]

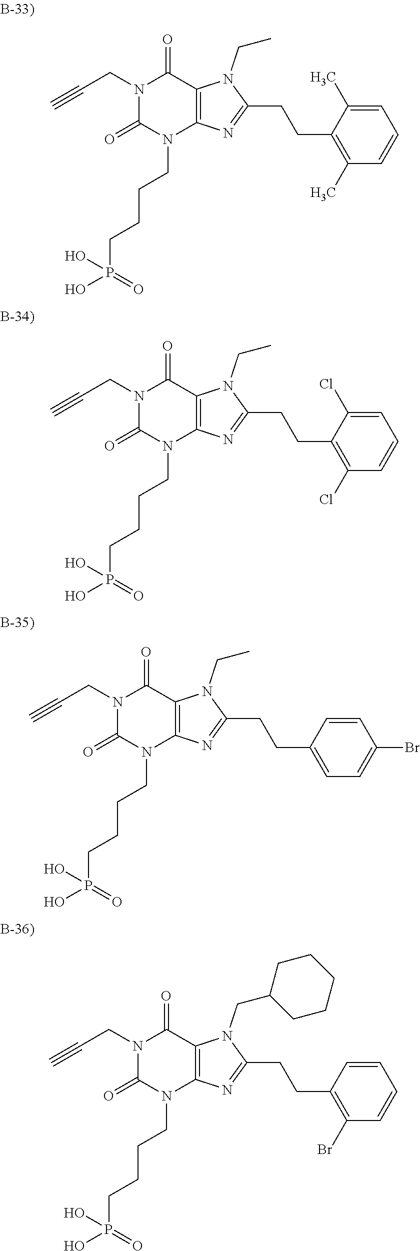 [SMILES: C#CCn1c(=O)c2c(nc(CCc3c(C)cccc3C)n2CC)n(CCCCP(=O)(O)O)c1=O ; C#CCn1c(=O)c2c(nc(CCc3c(Cl)cccc3Cl)n2CC)n(CCCCP(=O)(O)O)c1=O ; C#CCn1c(=O)c2c(nc(CCc3ccc(Br)cc3)n2CC)n(CCCCP(=O)(O)O)c1=O ; C#CCn1c(=O)c2c(nc(CCc3ccccc3Br)n2CC2CCCCC2)n(CCCCP(=O)(O)O)c1=O]

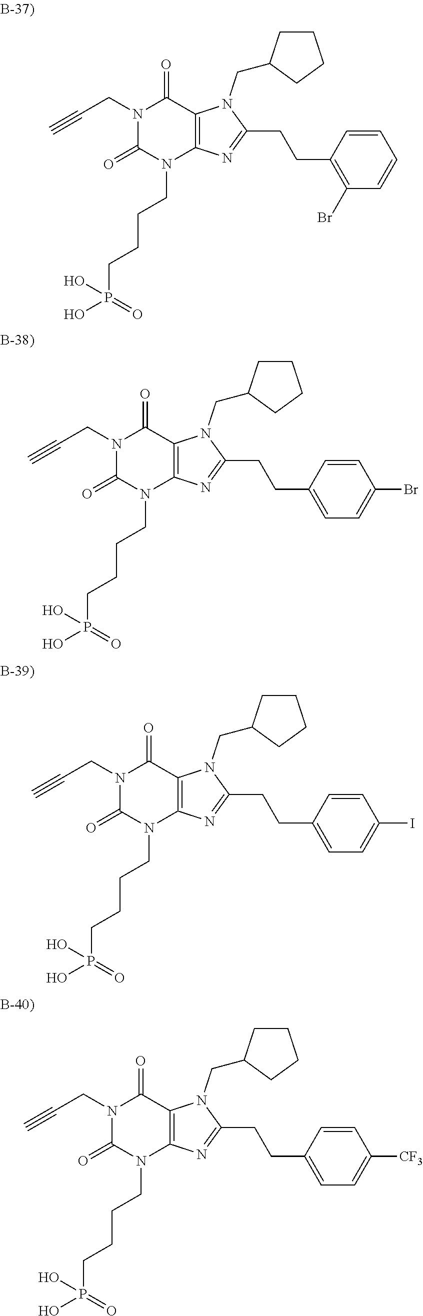 [SMILES: C#CCn1c(=O)c2c(nc(CCc3ccccc3Br)n2CC2CCCC2)n(CCCCP(=O)(O)O)c1=O ; C#CCn1c(=O)c2c(nc(CCc3ccc(Br)cc3)n2CC2CCCC2)n(CCCCP(=O)(O)O)c1=O ; C#CCn1c(=O)c2c(nc(CCc3ccc(I)cc3)n2CC2CCCC2)n(CCCCP(=O)(O)O)c1=O ; C#CCn1c(=O)c2c(nc(CCc3ccc(C(F)(F)F)cc3)n2CC2CCCC2)n(CCCCP(=O)(O)O)c1=O]

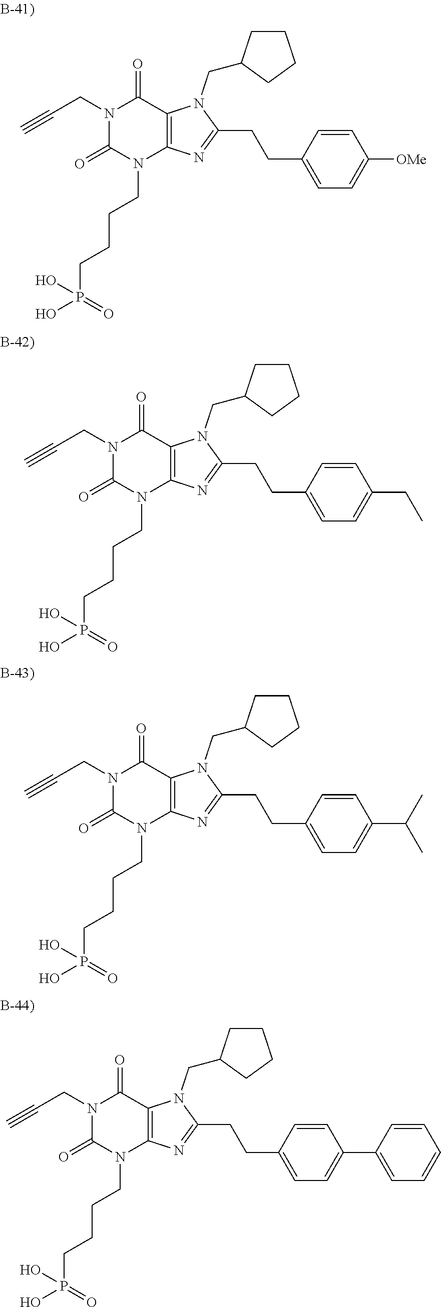

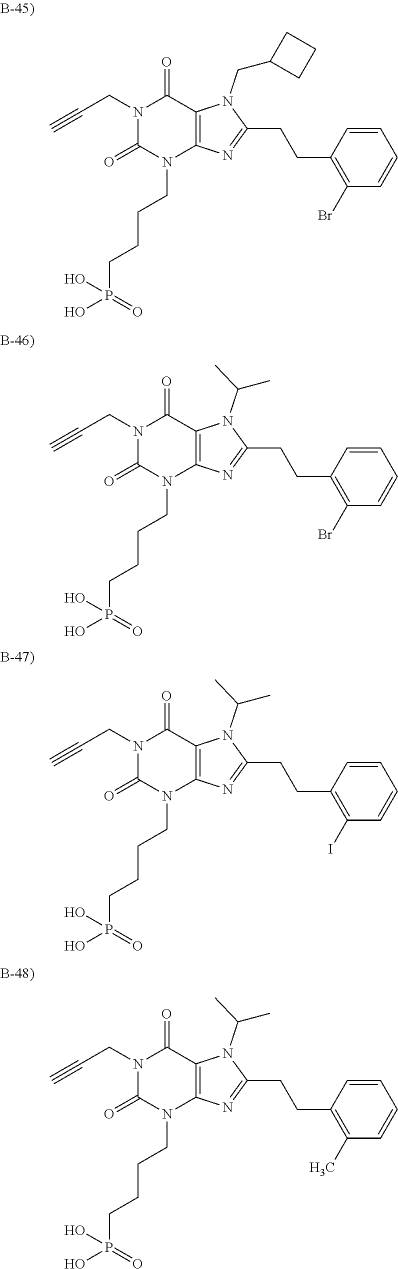 [SMILES: C#CCn1c(=O)c2c(nc(CCc3ccccc3Br)n2CC2CCC2)n(CCCCP(=O)(O)O)c1=O ; C#CCn1c(=O)c2c(nc(CCc3ccccc3Br)n2C(C)C)n(CCCCP(=O)(O)O)c1=O ; C#CCn1c(=O)c2c(nc(CCc3ccccc3I)n2C(C)C)n(CCCCP(=O)(O)O)c1=O ; C#CCn1c(=O)c2c(nc(CCc3ccccc3C)n2C(C)C)n(CCCCP(=O)(O)O)c1=O]

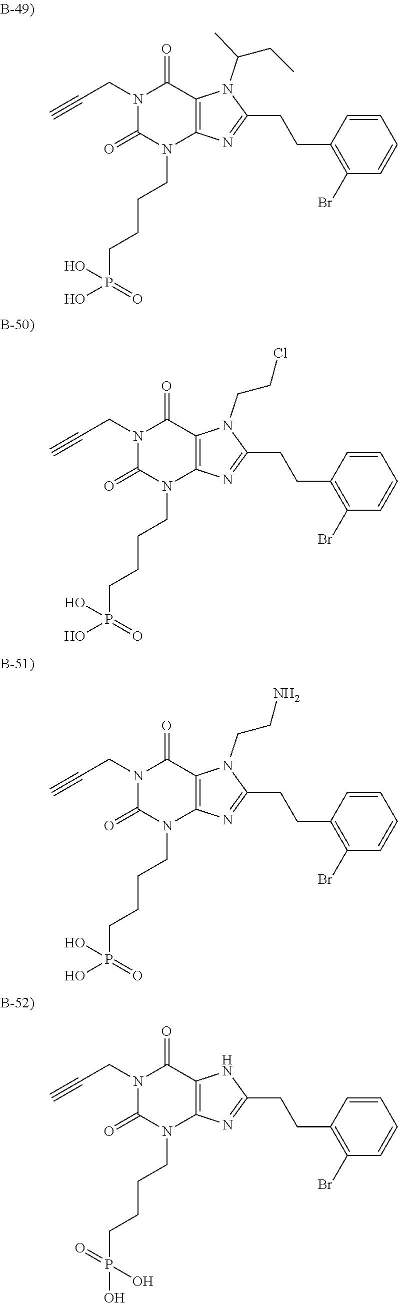 [SMILES: C#CCn1c(=O)c2c(nc(CCc3ccccc3Br)n2C(C)CC)n(CCCCP(=O)(O)O)c1=O ; C#CCn1c(=O)c2c(nc(CCc3ccccc3Br)n2CCCl)n(CCCCP(=O)(O)O)c1=O ; C#CCn1c(=O)c2c(nc(CCc3ccccc3Br)n2CCN)n(CCCCP(=O)(O)O)c1=O ; C#CCn1c(=O)c2[nH]c(CCc3ccccc3Br)nc2n(CCCCP(=O)(O)O)c1=O]

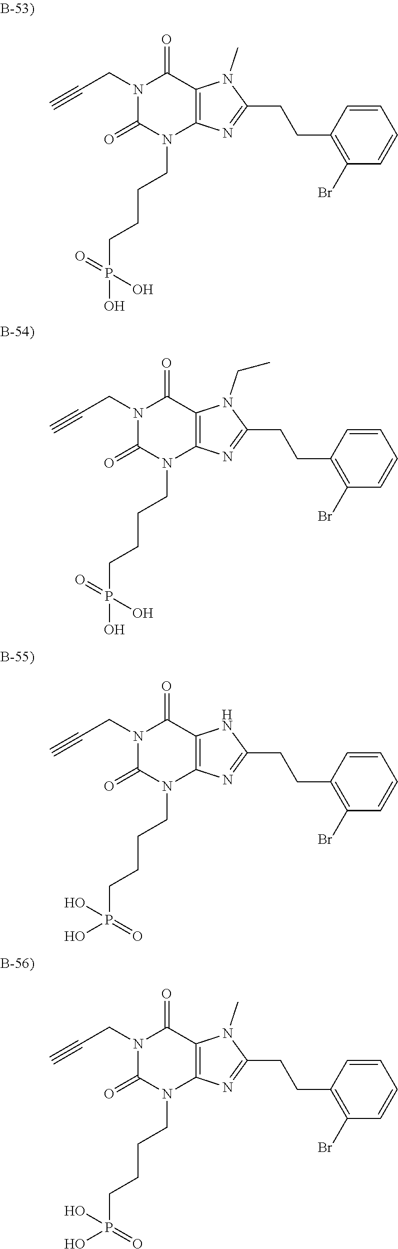 [SMILES: C#CCn1c(=O)c2c(nc(CCc3ccccc3Br)n2C)n(CCCCP(=O)(O)O)c1=O ; C#CCn1c(=O)c2c(nc(CCc3ccccc3Br)n2CC)n(CCCCP(=O)(O)O)c1=O ; C#CCn1c(=O)c2[nH]c(CCc3ccccc3Br)nc2n(CCCCP(=O)(O)O)c1=O ; C#CCn1c(=O)c2c(nc(CCc3ccccc3Br)n2C)n(CCCCP(=O)(O)O)c1=O]

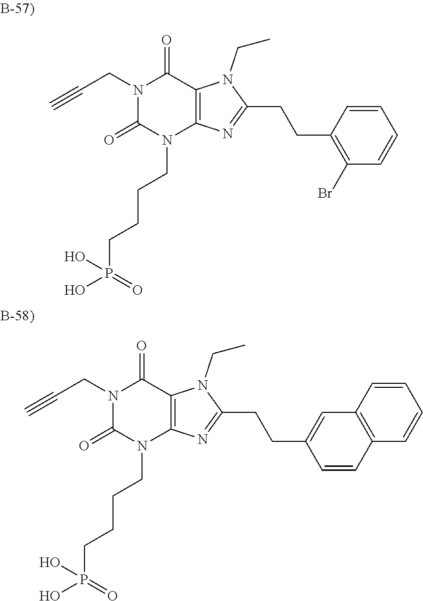 [SMILES: C#CCn1c(=O)c2c(nc(CCc3ccccc3Br)n2CC)n(CCCCP(=O)(O)O)c1=O ; C#CCn1c(=O)c2c(nc(CCc3ccc4ccccc4c3)n2CC)n(CCCCP(=O)(O)O)c1=O]

and the physiologically acceptable salts thereof.

Particularly preferred compounds according to substitution pattern (ii) where R3 represents —C₁₋₁₀-alkyl-P(═O)(OC₁₋₁₀-alkyl)₂ are compounds C-1 to C-10:

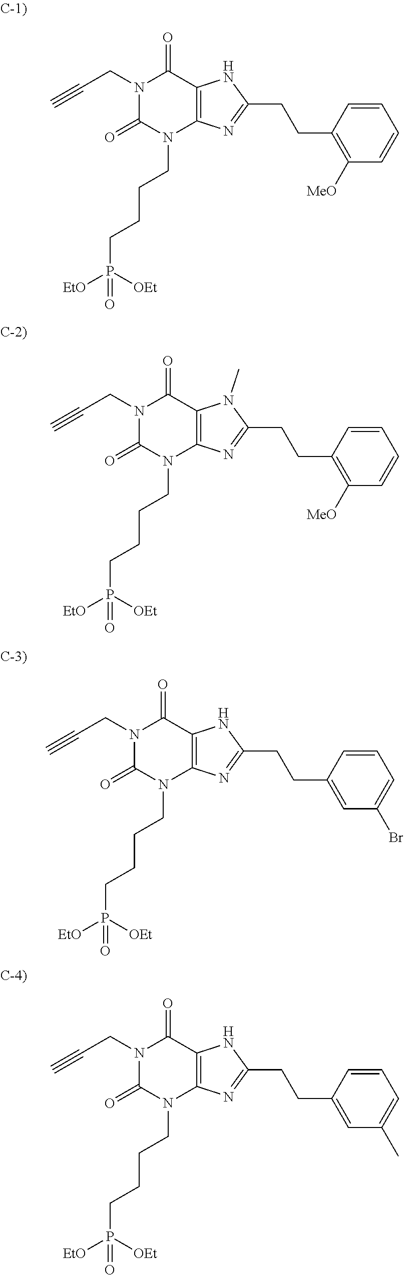 [SMILES: C#CCn1c(=O)c2[nH]c(CCc3ccccc3OC)nc2n(CCCCP(=O)(OCC)OCC)c1=O ; C#CCn1c(=O)c2c(nc(CCc3ccccc3OC)n2C)n(CCCCP(=O)(OCC)OCC)c1=O ; C#CCn1c(=O)c2[nH]c(CCc3cccc(Br)c3)nc2n(CCCCP(=O)(OCC)OCC)c1=O ; C#CCn1c(=O)c2[nH]c(CCc3cccc(C)c3)nc2n(CCCCP(=O)(OCC)OCC)c1=O]

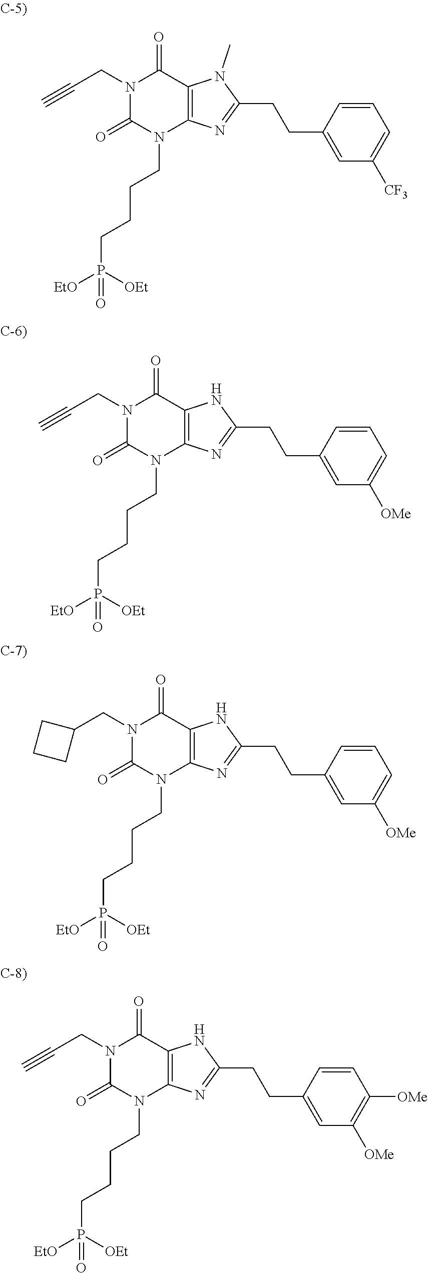 [SMILES: C#CCn1c(=O)c2c(nc(CCc3cccc(C(F)(F)F)c3)n2C)n(CCCCP(=O)(OCC)OCC)c1=O ; C#CCn1c(=O)c2[nH]c(CCc3cccc(OC)c3)nc2n(CCCCP(=O)(OCC)OCC)c1=O ; CCOP(=O)(CCCCn1c(=O)n(CC2CCC2)c(=O)c2[nH]c(CCc3cccc(OC)c3)nc21)OCC ; C#CCn1c(=O)c2[nH]c(CCc3ccc(OC)c(OC)c3)nc2n(CCCCP(=O)(OCC)OCC)c1=O]

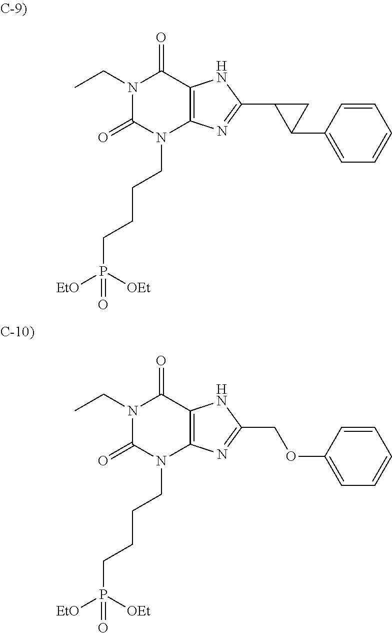 [SMILES: CCOP(=O)(CCCCn1c(=O)n(CC)c(=O)c2[nH]c(C3CC3c3ccccc3)nc21)OCC ; CCOP(=O)(CCCCn1c(=O)n(CC)c(=O)c2[nH]c(COc3ccccc3)nc21)OCC]

and the physiologically acceptable salts thereof.

Particularly preferred compounds according to substitution pattern (ii) where R3 represents —C₁₋₁₀-alkyl-P(═O)(OH)(OC₁₋₁₀-alkyl) are compounds D-1 to D-3:

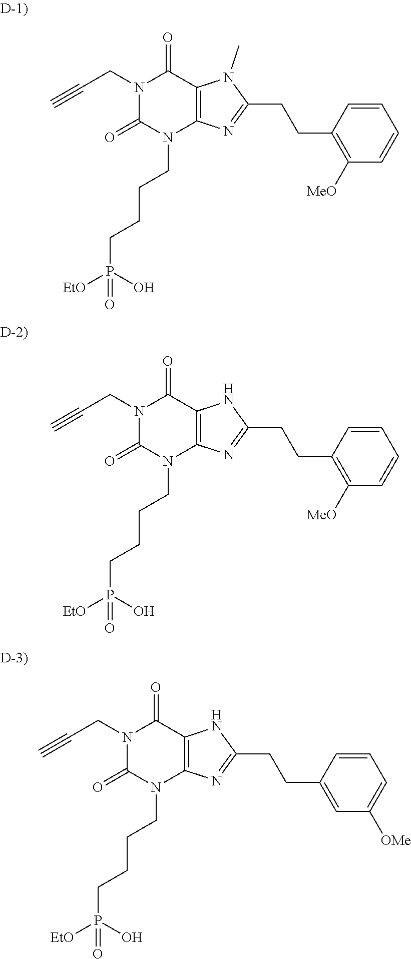 [SMILES: C#CCn1c(=O)c2c(nc(CCc3ccccc3OC)n2C)n(CCCCP(=O)(O)OCC)c1=O ; C#CCn1c(=O)c2[nH]c(CCc3ccccc3OC)nc2n(CCCCP(=O)(O)OCC)c1=O ; C#CCn1c(=O)c2[nH]c(CCc3cccc(OC)c3)nc2n(CCCCP(=O)(O)OCC)c1=O]

and the physiologically acceptable salts thereof.

Particularly preferred compounds according to substitution pattern (ii) where R3 represents —C₁₋₁₀-alkyl-S(═O)₂(OH) are compounds E-1 to E-3:

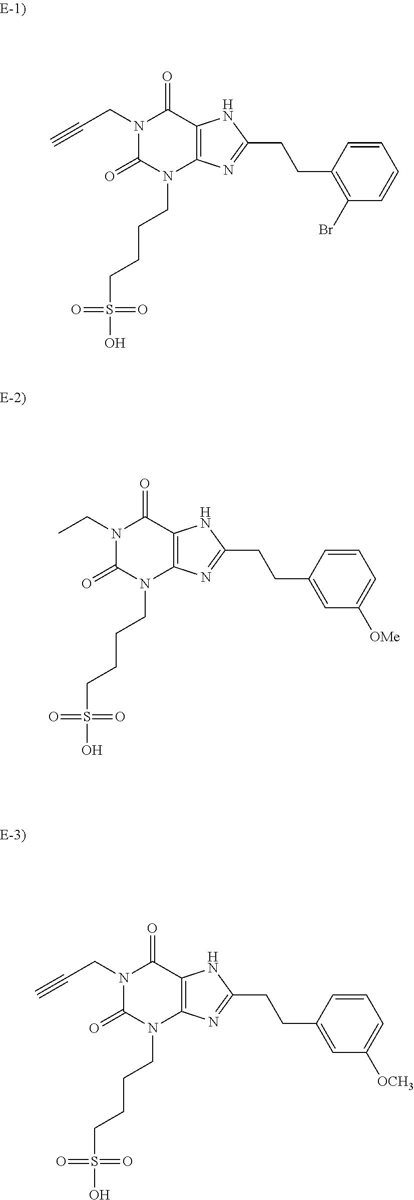 [SMILES: C#CCn1c(=O)c2[nH]c(CCc3ccccc3Br)nc2n(CCCCS(=O)(=O)O)c1=O ; CCn1c(=O)c2[nH]c(CCc3cccc(OC)c3)nc2n(CCCCS(=O)(=O)O)c1=O ; C#CCn1c(=O)c2[nH]c(CCc3cccc(OC)c3)nc2n(CCCCS(=O)(=O)O)c1=O]

and the physiologically acceptable salts thereof.

A particularly preferred compound according to substitution pattern (ii) where R3 represents —C₁₋₁₀-alkyl-S(═O)₂(NH₂) is compound F-1:

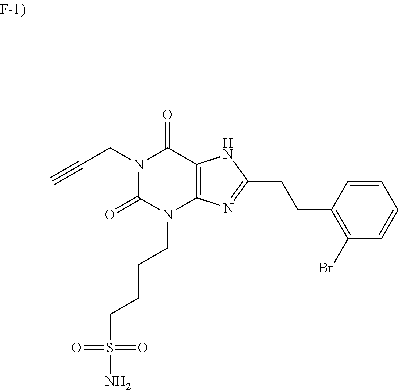 [SMILES: C#CCn1c(=O)c2[nH]c(CCc3ccccc3Br)nc2n(CCCCS(N)(=O)=O)c1=O]

and the physiologically acceptable salts thereof.

Particularly preferred compounds according to substitution pattern (ii) where R3 represents —C₁₋₁₀-alkyl-C(═O)(OH) are compounds G-1 to G-3:

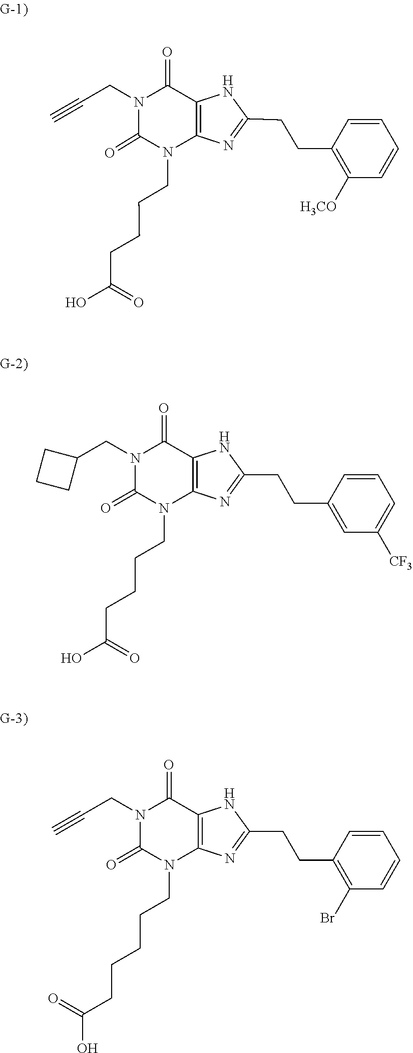 [SMILES: C#CCn1c(=O)c2[nH]c(CCc3ccccc3OC)nc2n(CCCCC(=O)O)c1=O ; O=C(O)CCCCn1c(=O)n(CC2CCC2)c(=O)c2[nH]c(CCc3cccc(C(F)(F)F)c3)nc21 ; C#CCn1c(=O)c2[nH]c(CCc3ccccc3Br)nc2n(CCCCCC(=O)O)c1=O]

and the physiologically acceptable salts thereof.

Another aspect of the invention relates to a compound of general formula 1

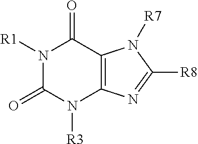

wherein
R1 represents —C₁₋₁₀-alkyl, optionally substituted with —C≡CH; or —C₁₋₁₀-alkyl-C₃₋₁₀-cycloalkyl; preferably —C₁₋₆-alkyl, optionally substituted with —C≡CH; or —C₁₋₆-alkyl-C₃₋₆-cycloalkyl; more preferably —CH₂CH₃, —CH₂CH₂CH₃, —CH₂C≡CH, or —CH₂-cyclobutyl;
R3 represents —C₃₋₅-alkyl-OH, —C₃₋₅-alkyl-O—C(═O)C₁₋₆-alkyl, —C₃₋₅-alkyl-P(═O)(OH)₂; —C₃₋₅-alkyl-P(═O)(OC₁₋₆-alkyl)₂; —C₃₋₅-alkyl-P(═O)(OH)(OC₁₋₆-alkyl); —C₃₋₅-alkyl-S(═O)₂(OH); —C₃₋₅-alkyl-S(═O)₂(NH₂); or —C₃₋₅-alkyl-C(═O)(OH); preferably —C₃₋₅-alkyl-P(═O)(OH)₂; more preferably —C₄-alkyl-P(═O)(OH)₂;
R7 represents —H; —C₁₋₁₀-alkyl, optionally substituted with —OH; —C₁₋₁₀-alkyl-C₃₋₁₀-cycloalkyl; or —C₁₋₁₀-alkyl-aryl; preferably —H; —C₁₋₆-alkyl, optionally substituted with —OH; —C₁₋₆-alkyl-C₃₋₆-cycloalkyl; or —C₁₋₆-alkyl-aryl; more preferably —H, —CH₃, —CH₂CH₃, —CH₂CH₂—OH, —CH₂CH₂CH₃, —CH₂-cyclopropyl, or —CH₂-phenyl;
R8 represents —C₃₋₁₀-cycloalkyl, —C₁₋₁₀-alkyl-C₃₋₁₀-cycloalkyl, -aryl, —C₁₋₁₀-alkyl-aryl, —C₃₋₆-cycloalkyl-aryl, or —C₁₋₁₀-alkyl-O-aryl; preferably -phenyl, optionally substituted with —F, —Br, —Cl, —CH₃, —CF₃, —OCH₃; —CH₂-phenyl, optionally substituted with —F, —Br, —Cl, —CH₃, —CF₃, —OCH₃; —CH₂CH₂-phenyl, optionally substituted with one or two substituents independently of one another selected from —F, —Br, —Cl, —CH₃, —CH₂—CH₃, —CH(CH₃)₂, -phenyl, —CF₃, —OCH₃; —CH═CH-phenyl, optionally substituted with —F, —Br, —Cl, —CH₃, —CF₃, —OCH₃; —CH₂—O-phenyl, optionally substituted with —F, —Br, —Cl, —CH₃, —CF₃, —OCH₃; -cyclopropyl-phenyl, optionally substituted with —F, —Br, —Cl, —CH₃, —CF₃, —OCH₃; or —C₃₋₆-cycloalkyl; more preferably -phenyl, optionally substituted with —OCH₃; —CH₂-phenyl, optionally substituted with —Cl; —CH₂CH₂-phenyl, optionally substituted with one or two substituents independently of one another selected from —F, —Br, —Cl, —CH₃, —CH₂—CH₃, —CH(CH₃)₂, -phenyl, —CF₃, —OCH₃; —CH═CH-phenyl, optionally substituted with —OCH₃; —CH₂—O-phenyl; -cyclopropyl-phenyl; or -cyclopentyl;
and the physiologically acceptable salts thereof.

Another aspect of the invention relates to a compound of general formula 1

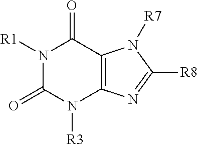

wherein
R1 represents —C₁₋₁₀-alkyl, optionally substituted with —C≡CH; or —C₁₋₁₀-alkyl-C₃₋₁₀-cycloalkyl; preferably —C₁₋₆-alkyl, optionally substituted with —C≡CH; or —C₁₋₆-alkyl-C₃₋₆-cycloalkyl; more preferably —CH₂CH₃, —CH₂CH₂CH₃, —CH₂C≡CH, or —CH₂-cyclobutyl;
R3 represents —C₃₋₅-alkyl-OH, —C₃₋₅-alkyl-O—C(═O)C₁₋₆-alkyl, —C₃₋₅-alkyl-P(═O)(OH)₂; —C₃₋₅-alkyl-P(═O)(OC₁₋₆-alkyl)₂; —C₃₋₅-alkyl-P(═O)(OH)(OC₁₋₆-alkyl); —C₃₋₅-alkyl-S(═O)₂(OH); —C₃₋₅-alkyl-S(═O)₂(NH₂); or —C₃₋₅-alkyl-C(═O)(OH); preferably —C₃₋₅-alkyl-P(═O)(OH)₂; more preferably —C₄-alkyl-P(═O)(OH)₂.
R7 represents —C₂₋₁₀-alkyl, optionally substituted with —OH; —C₂₋₁₀-alkyl-C₃₋₁₀-cycloalkyl; or —C₂₋₁₀-alkyl-aryl; preferably —C₂₋₆-alkyl, optionally substituted with —OH; —C₂₋₆-alkyl-C₃₋₆-cycloalkyl; or —C₂₋₆-alkyl-aryl; more preferably —CH₂CH₃, —CH₂CH₂—OH, or —CH₂CH₂CH₃;
R8 represents —C₃₋₁₀-cycloalkyl, —C₁₋₁₀-alkyl-C₃₋₁₀-cycloalkyl, -aryl, —C₁₋₁₀-alkyl-aryl, —C₃₋₆-cycloalkyl-aryl, or —C₁₋₁₀-alkyl-O-aryl; preferably -phenyl, optionally substituted with —F, —Br, —Cl, —CH₃, —CF₃, —OCH₃; —CH₂-phenyl, optionally substituted with —F, —Br, —Cl, —CH₃, —CF₃, —OCH₃; —CH₂CH₂-phenyl, optionally substituted with one or two substituents independently of one another selected from —F, —Br, —Cl, —CH₃, —CH₂—CH₃, —CH(CH₃)₂, -phenyl, —CF₃, —OCH₃; —CH═CH-phenyl, optionally substituted with —F, —Br, —Cl, —CH₃, —CF₃, —OCH₃; —CH₂—O-phenyl, optionally substituted with —F, —Br, —Cl, —CH₃, —CF₃, —OCH₃; -cyclopropyl-phenyl, optionally substituted with —F, —Br, —Cl, —CH₃, —CF₃, —OCH₃; or —C₃₋₆-cyclo-alkyl; more preferably -phenyl, optionally substituted with —OCH₃; —CH₂-phenyl, optionally substituted with —Cl; —CH₂CH₂-phenyl, optionally substituted with one or two substituents independently of one another selected from —F, —Br, —Cl, —CH₃, —CH₂—CH₃, —CH(CH₃)₂, -phenyl, —CF₃, —OCH₃; —CH═CH-phenyl, optionally substituted with —OCH₃; —CH₂—O-phenyl; -cyclopropyl-phenyl; or -cyclopentyl;
and the physiologically acceptable salts thereof.

Another aspect of the invention relates to a compound of general formula 1

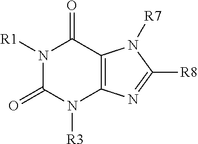

wherein
R1 represents —CH₂C≡CH;
R3 represents —C₃₋₅-alkyl-OH, —C₃₋₅-alkyl-O—C(═O)C₁₋₆-alkyl, —C₃₋₅-alkyl-P(═O)(OH)₂; —C₃₋₅-alkyl-P(═O)(OC₁₋₆-alkyl)₂; —C₃₋₅-alkyl-P(═O)(OH)(OC₁₋₆-alkyl); —C₃₋₅-alkyl-S(═O)₂(OH); —C₃₋₅-alkyl-S(═O)₂(NH₂); or —C₃₋₅-alkyl-C(═O)(OH); preferably —C₃₋₅-alkyl-P(═O)(OH)₂; more preferably —C₄-alkyl-P(═O)(OH)₂;
R7 represents —H; —C₁₋₁₀-alkyl, optionally substituted with —OH; —C₁₋₁₀-alkyl-C₃₋₁₀-cycloalkyl; or —C₁₋₁₀-alkyl-aryl; preferably —H; —C₁₋₆-alkyl, optionally substituted with —OH; —C₁₋₆-alkyl-C₃₋₆-cycloalkyl; or —C₁₋₆-alkyl-aryl; more preferably —H, —CH₃, —CH₂CH₃, —CH₂CH₂—OH, —CH₂CH₂CH₃, —CH₂-cyclopropyl, or —CH₂-phenyl;
R8 represents —C₂₋₁₀-alkyl-C₃₋₁₀-cycloalkyl, —C₂₋₁₀-alkyl-aryl, —C₃₋₆-cycloalkyl-aryl, or —C₁₋₁₀-alkyl-O-aryl; preferably —CH₂CH₂-phenyl, optionally substituted with one or two substituents independently of one another selected from —F, —Br, —Cl, —CH₃, —CH₂—CH₃, —CH(CH₃)₂, -phenyl, —CF₃, —OCH₃; —CH₂—O— phenyl, optionally substituted with —F, —Br, —Cl, —CH₃, —CF₃, —OCH₃; or -cyclopropyl-phenyl, optionally substituted with —F, —Br, —Cl, —CH₃, —CF₃, —OCH₃; more preferably —CH₂CH₂-phenyl, optionally substituted with one or two substituents independently of one another selected from —F, —Br, —Cl, —CH₃, —CH₂—CH₃, —CH(CH₃)₂, -phenyl, —CF₃, —OCH₃; —CH₂—O-phenyl; or -cyclopropyl-phenyl;
and the physiologically acceptable salts thereof.

Another aspect of the invention relates to a compound of general formula 1

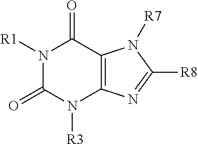

wherein
R1 represents —C≡CH;
R3 represents —C₃₋₅-alkyl-OH, —C₃₋₅-alkyl-O—C(═O)C₁₋₆-alkyl, —C₃₋₅-alkyl-P(═O)(OH)₂; —C₃₋₅-alkyl-P(═O)(OC₁₋₆-alkyl)₂; —C₃₋₅-alkyl-P(═O)(OH)(OC₁₋₆-alkyl); —C₃₋₅-alkyl-S(═O)₂(OH); —C₃₋₅-alkyl-S(═O)₂(NH₂); or —C₃₋₅-alkyl-C(═O)(OH); preferably —C₃₋₅-alkyl-P(═O)(OH)₂; more preferably —C₄-alkyl-P(═O)(OH)₂.
R7 represents —C₂₋₁₀-alkyl, optionally substituted with —OH; —C₂₋₁₀-alkyl-C₃₋₁₀-cycloalkyl; or —C₂₋₁₀-alkyl-aryl; preferably —C₂₋₆-alkyl, optionally substituted with —OH; —C₂₋₆-alkyl-C₃₋₆-cycloalkyl; or —C₂₋₆-alkyl-aryl; more preferably —CH₂CH₃, —CH₂CH₂—OH, or —CH₂CH₂CH₃;
R8 represents —C₃₋₁₀-cycloalkyl, —C₁₋₁₀-alkyl-C₃₋₁₀-cycloalkyl, -aryl, —C₁₋₁₀-alkyl-aryl, —C₃₋₆-cycloalkyl-aryl, or —C₁₋₁₀-alkyl-O-aryl; preferably -phenyl, optionally substituted with —F, —Br, —Cl, —CH₃, —CF₃, —OCH₃; —CH₂-phenyl, optionally substituted with —F, —Br, —Cl, —CH₃, —CF₃, —OCH₃; —CH₂CH₂-phenyl, optionally substituted with one or two substituents independently of one another selected from —F, —Br, —Cl, —CH₃, —CH₂—CH₃, —CH(CH₃)₂, -phenyl, —CF₃, —OCH₃; —CH═CH-phenyl, optionally substituted with —F, —Br, —Cl, —CH₃, —CF₃, —OCH₃; —CH₂—O-phenyl, optionally substituted with —F, —Br, —Cl, —CH₃, —CF₃, —OCH₃; -cyclopropyl-phenyl, optionally substituted with —F, —Br, —Cl, —CH₃, —CF₃, —OCH₃; or —C₃₋₆-cyclo-alkyl; more preferably -phenyl, optionally substituted with —OCH₃; —CH₂-phenyl, optionally substituted with —Cl; —CH₂CH₂-phenyl, optionally substituted with one or two substituents independently of one another selected from —F, —Br, —Cl, —CH₃, —CH₂—CH₃, —CH(CH₃)₂, -phenyl, —CF₃, —OCH₃; —CH═CH— phenyl, optionally substituted with —OCH₃; —CH₂—O-phenyl; -cyclopropyl-phenyl; or -cyclopentyl; and the physiologically acceptable salts thereof.

As used herein, the terms “physiologically acceptable salt” refer to those salts which retain the biological effectiveness and properties of the compound according to general formula 1. Such salts include, but are not restricted to: (1) an acid addition salt which is obtained by reaction of the free base of the compound according to general formula 1 with inorganic acids such as hydrochloric acid, hydrobromic acid, nitric acid, phosphoric acid, sulfuric acid, and perchloric acid and the like, or with organic acids such as acetic acid, oxalic acid, (D)- or (L)-malic acid, maleic acid, methanesulfonic acid, ethanesulfonic acid, p-toluenesulfonic acid, salicylic acid, tartaric acid, citric acid, succinic acid or malonic acid and the like, preferably hydrochloric acid or (L)-malic acid; or (2) salts formed when an acidic proton present in the compound according to general formula 1 either is replaced by a metal ion, e. g., an alkali metal ion, such as sodium or potassium, an alkaline earth ion, such as magnesium or calcium, or an aluminum ion; or coordinates with an organic base such as ethanolamine, diethanolamine, triethanolamine, tromethamme, N-methylglucamine, and the like.

The compound of general Formula 1 may also act as a prodrug. A “prodrug” preferably refers to an agent which is converted into the parent drug in vivo. Prodrugs are often useful because, in some situations, they may be easier to administer than the parent drug. They may, for instance, be bioavailable by oral administration whereas the parent drug is not, or they may penetrate into the central nervous system (CNS) while the parent drug does not. The prodrug may also have improved solubility in pharmaceutical compositions over the parent drug or otherwise increased lipophilicity to show better solubility in dermal ointments, to penetrate better into the skin, or to permeate into the CNS. An example, without limitation, of a prodrug would be a compound of the present invention which is administered as an ester (the “prodrug”) to facilitate transmittal across a cell membrane where water solubility is detrimental to mobility but then is metabolically hydrolyzed e.g. to the carboxylic acid or phosphonic acid, the active entity, once inside the cell where water solubility is beneficial. A prodrug may be converted into the parent drug by various mechanisms, including enzymatic processes and metabolic hydrolysis.

A further example of a prodrug might be a short polypeptide, for example, without limitation, a 2-10 amino acid polypeptide, bonded through a terminal amino group to a carboxy group of a compound of this invention wherein the polypeptide is hydrolyzed or metabolized in vivo to release the active molecule. The prodrugs of compounds of general formula 1 are within the scope of this invention.

Additionally, it is contemplated that compounds of general formula 1 would be metabolized by enzymes in the body of the organism such as a human being to generate a metabolite that can modulate the activity of the MRGPRX4 receptor. Such metabolites are within the scope of the present invention.

Another aspect of the invention relates to a pharmaceutical composition comprising any of the compounds or salts of the present invention and, optionally, a pharmaceutically acceptable carrier or excipient. This composition may additionally comprise further compounds or medicaments, such as, for example, neuroprotective or antinociceptive, anti-inflammatory or antibiotic agents besides the compounds according to general formula 1.

“Pharmaceutical composition” preferably refers to a mixture of one or more of the compounds described herein, or physiologically/pharmaceutically acceptable salts or prodrugs thereof, with other chemical components, such as physiologically/pharmaceutically acceptable carriers and excipients. The purpose of a pharmaceutical composition is to facilitate administration of a compound to an organism.

As used herein, a “physiologically/pharmaceutically acceptable carrier” refers to a carrier or diluent that does not cause significant irritation to an organism and does not abrogate the biological activity and properties of the administered compound.

A “pharmaceutically acceptable excipient” refers to an inert substance added to a pharmaceutical composition to further facilitate administration of a compound. Examples, without limitation, of excipients include calcium carbonate, calcium phosphate, various sugars and types of starch, cellulose derivatives, gelatin, vegetable oils and polyethylene glycols.

Physiologically or pharmaceutically acceptable carriers and excipients are known to the skilled person. In this regard it can be referred to, e.g., H. P. Fiedler, Lexikon der Hilfsstoffe für Pharmazie, Kosmetik und angrenzende technische Gebiete, Editio Cantor Aulendorf, 2001.

The pharmaceutical composition according to the invention can be, e.g., solid, liquid or pasty.

A further aspect of the invention relates to a pharmaceutical dosage form comprising the pharmaceutical composition according to the invention.

The pharmaceutical dosage form according to the invention may be adapted for various routes of administration (e.g. systemic, parenteral, subcutaneous, topic, local), such as oral administration, infusion, injection and the like. The pharmaceutical dosage form is preferably adapted for oral, local or subcutaneous administration, or combinations thereof.

Pharmaceutical dosage forms that are adapted for oral administration include tablets, pellets, capsules, powders, granules and the like.

The pharmaceutical dosage form is preferably adapted for administration once daily, twice daily or thrice daily. The pharmaceutical dosage form may release the compound according to general formula 1 immediately (immediate release formulation) or over an extended period of time (retarded release, delayed release, prolonged release, sustained release, and the like).

Another aspect of the invention relates to the compounds according to the invention as described above for use as a medicament.

Another aspect of the invention relates to the compounds according to the invention as described above for use in the prevention or treatment of a condition, disease or disorder that is associated with the MRGPRX4 receptor, preferably that can be prevented or treated by modulating, preferably agonizing the MRGPRX4 receptor, preferably the human wildtype MRGPRX4.

Another aspect of the invention relates to the use of the compounds according to the invention as described above for the manufacture of a medicament for the prevention or treatment of a condition, disease or disorder that is associated with the MRGPRX4 receptor, preferably that can be prevented or treated by modulating, preferably agonizing the MRGPRX4 receptor, preferably the human wildtype MRGPRX4.

Another aspect of the invention relates to a method for preventing or treating a condition, disease or disorder that is associated with the MRGPRX4, preferably that can be prevented or treated by modulating, preferably agonizing the MRGPRX4, preferably the human wildtype MRGPRX4, the method comprising the step of administering an effective amount of a compound according to the invention as described above to a subject in need thereof.

Preferably, the disease or disorder that is associated with the MRGPRX4 receptor is selected from
open wounds, e.g. incisions or incised wounds, lacerations, abrasions (grazes), avulsions, puncture wounds, penetration wounds and gunshot wounds; and
closed wounds, e.g. hematomas and crash injuries; and
painful states, itching, neuropathic pain, chronic pain.

Preferably, the disease or disorder that is associated with the MRGPRX4 receptor is associated with the wildtype or a variant of the MRGPRX4 receptor (83S or 83L).

Preferably, the compound, the pharmaceutical composition or the pharmaceutical dosage form is administered topically and/or locally.

The compounds according to the invention show agonistic or antagonistic effects at the MRGPRX4 receptor.

In another aspect, the invention relates to the use of the compounds according to general formula 1 for activating or blocking MRGPRX4 function. In a further aspect, the compounds according to general formula 1 may thus also be used for the prevention, alleviation and/or treatment of a condition, disease or disorder related to MRGPRX4 receptor activity.

“Treat”, “treating” and “treatment” preferably refer to a method of alleviating or abrogating an MRGPRX4 receptor related disease or disorder and/or its attendant symptoms. “Prevent”, “preventing” and “prevention” preferably refer to a method of hindering an MRGPRX4 receptor related disease or disorder from occurring, i.e. a prophylactic method.

The compounds according to the invention are useful for treating various organisms. “Organism” preferably refers to any living entity comprised of at least one cell. A living organism can be as simple as, for example, a single eukaryotic cell or as complex as a mammal, including a human being.

The compounds according to the invention are used in therapeutically effective amounts. “Therapeutically effective amount” preferably refers to that amount of the compound being administered which will relieve to some extent one or more of the symptoms of the disorder being treated.

Preferably, the subject afflicted by a disease treated, alleviated or prevented according to the invented use is a human.

Another aspect of the invention relates to a compound according to general Formula 1

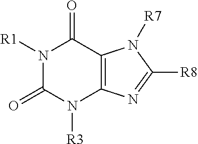

wherein
R1 represents —H, —C₁₋₁₀-alkyl, —C₁₋₁₀-alkyl-C₃₋₁₀-cycloalkyl, —C₁₋₁₀-alkyl-aryl, —C₁₋₁₀-alkyl-heteroaryl, —C₃₋₁₀-cycloalkyl, —C₁₋₁₀-alkyl-O-aryl, or —C₁₋₁₀-alkyl-O-heteroaryl;
R3 represents —C₁₋₁₀-alkyl-P(═O)(OC₁₋₁₀-alkyl)₂, —C₁₋₁₀-alkyl-P(═O)(OH)(OC₁₋₁₀-alkyl), —C₁₋₁₀-alkyl-S(═O)₂(OH), —C₁₋₁₀-alkyl-S(═O)₂(NH₂), —C₁₋₁₀-alkyl-C(═O)(OH), or —C₁₋₁₀-alkyl-P(═O)(OH)₂;
R7 represents —H, —C₁₋₁₀-alkyl, —C₁₋₁₀-alkyl-C₃₋₁₀-cycloalkyl, —C₁₋₁₀-alkyl-aryl, —C₁₋₁₀-alkyl-heteroaryl, —C₃₋₁₀-cycloalkyl, —C₁₋₁₀-alkyl-O-aryl, or —C₁₋₁₀-alkyl-O-heteroaryl; and
R8 represents —H, —C₁₋₁₀-alkyl, —C₁₋₁₀-alkyl-C₃₋₁₀-cycloalkyl, —C₁₋₁₀-alkyl-aryl, —C₁₋₁₀-alkyl-heteroaryl, —C₃₋₁₀-cycloalkyl, —C₁₋₁₀-alkyl-aryl, —C₁₋₁₀-alkyl-O-aryl, —C₃₋₆-cycloalkyl-aryl, —C₃₋₆-cycloalkyl-heteroaryl, —C₁₋₁₀-alkyl-heteroaryl, or —C₁₋₁₀-alkyl-O-heteroaryl;
wherein in each case “C₁₋₁₀-alkyl” may be linear or branched, unless expressly stated otherwise saturated or unsaturated, unsubstituted or monosubstituted with a substituent selected from —C≡CH, —C(═O)OH, —C(═O)O—C₁₋₁₀-alkyl, —OH, —O—C₁₋₁₀-alkyl, —NH₂, —NH—C₁₋₁₀-alkyl, —N(C₁₋₁₀-alkyl)₂, —N₃, —F, —Cl, —Br, and —I;
wherein in each case “C₃₋₁₀-cycloalkyl” may be linear or branched, unless expressly stated otherwise saturated or unsaturated, unsubstituted or monosubstituted with a substituent selected from —C≡CH, —C(═O)OH, —C(═O)O—C₁₋₆-alkyl, —OH, —O—C₁₋₁₀-alkyl, —NH₂, —NH—C₁₋₁₀-alkyl, —N(C₁₋₁₀-alkyl)₂, —N₃, —F, —Cl, —Br, and —I;
wherein in each case “aryl” is a 6-14-membered aryl moiety which may be unsubstituted, mono- or disubstituted with a substituent independently selected from —C≡CH, —CF₃, —CH₃, —CH₂—CH₃, —CH(CH₃)₂, -phenyl, —CCl₃, —CBr₃, —Cl₃, —C(═O)OH, —C(═O)O—C₁₋₁₀-alkyl, —OH, —O—C₁₋₁₀-alkyl, —NH₂, —NH—C₁₋₁₀-alkyl, —N(C₁₋₁₀-alkyl)₂, —N₃, —F, —Cl, —Br, and —I;
wherein in each case “heteroaryl” is a 5-14-membered heteroaryl moiety which may be unsubstituted, mono- or disubstituted with a substituent independently selected from —C≡CH, —CF₃, —CCl₃, —CBr₃, —Cl₃, —C(═O)OH, —C(═O)O—C₁₋₁₀-alkyl, —OH, —O—C₁₋₁₀-alkyl, —NH₂, —NH—C₁₋₁₀-alkyl, —N(C₁₋₁₀-alkyl)₂, —N₃, —F, —Cl, —Br, and —I;
or a physiologically acceptable salt thereof,
for use in the prevention or treatment of a condition, disease or disorder that is associated with the MRGPRX4 receptor, preferably associated with the wildtype of the MRGPRX4 receptor.

The above compounds for use according to the invention may again reflect two substitution patterns, for the purpose of the specification indicated as (iii) and (iv), respectively.

According to substitution pattern (iii) of the compounds for use according to the invention,
R1 represents —H, —C₁₋₁₀-alkyl, —C₁₋₁₀-alkyl-C₃₋₁₀-cycloalkyl, —C₁₋₁₀-alkyl-aryl, or —C₁₋₁₀-alkyl-heteroaryl;
R3 represents —C₁₋₁₀-alkyl-P(═O)(OH)₂;
R7 represents —H, —C₁₋₁₀-alkyl, —C₁₋₁₀-alkyl-C₃₋₁₀-cycloalkyl, —C₁₋₁₀-alkyl-aryl, or —C₁₋₁₀-alkyl-heteroaryl; and
R8 represents —C₃₋₆-cycloalkyl, -aryl, -heteroaryl, —C₁₋₁₀-alkyl-aryl, or —C₁₋₁₀-alkyl-heteroaryl (wherein in case of —C₂-alkyl-aryl, the alkyl-moiety is unsaturated);

According to substitution pattern (iv) of the compounds for use according to the invention,
R1 represents —H, —C₁₋₁₀-alkyl, —C₁₋₁₀-alkyl-C₃₋₁₀-cycloalkyl, —C₁₋₁₀-alkyl-aryl, —C₁₋₁₀-alkyl-heteroaryl, —C₃₋₁₀-cycloalkyl, —C₁₋₁₀-alkyl-O-aryl, or —C₁₋₁₀-alkyl-O-heteroaryl;
R3 represents —C₁₋₁₀-alkyl-OH; or —C₁₋₁₀-alkyl-O—C(═O)C₁₋₁₀-alkyl;
R7 represents —H, —C₁₋₁₀-alkyl, —C₁₋₁₀-alkyl-C₃₋₁₀-cycloalkyl, —C₁₋₁₀-alkyl-aryl, —C₁₋₁₀-alkyl-heteroaryl, —C₃₋₁₀-cycloalkyl, —C₁₋₁₀-alkyl-O-aryl, or —C₁₋₁₀-alkyl-O-heteroaryl; and
R8 represents —H, —C₁₋₁₀-alkyl, —C₁₋₁₀-alkyl-C₃₋₁₀-cycloalkyl, —C₁₋₁₀-alkyl-aryl, —C₁₋₁₀-alkyl-heteroaryl, —C₃₋₁₀-cycloalkyl, —C₁₋₁₀-alkyl-O-aryl, or —C₁₋₁₀-alkyl-O-heteroaryl.

Within the above definitions of the compounds for use according to substitution patterns (iii) and (iv), in each case “C₁₋₁₀-alkyl”, “C₃₋₁₀-cycloalkyl”, “aryl”, and “heteroaryl” independently have the same meaning as defined above.

In preferred embodiments of the compounds for use according to substitution pattern (iii),
R1 represents —H, —C₁₋₁₀-alkyl, —C₁₋₁₀-alkyl-C₃₋₁₀-cycloalkyl, —C₁₋₁₀-alkyl-aryl, or —C₁₋₁₀-alkyl-heteroaryl;
R3 represents —C₁₋₆-alkyl-P(═O)(OH)₂;
R7 represents —H, —C₁₋₁₀-alkyl, —C₁₋₁₀-alkyl-C₃₋₁₀-cycloalkyl, —C₁₋₁₀-alkyl-aryl, or —C₁₋₁₀-alkyl-heteroaryl; and
R8 represents —C₃₋₆-cycloalkyl, -aryl, -heteroaryl, —C₁₋₆-alkyl-aryl, or —C₁₋₆-alkyl-heteroaryl (wherein in case of —C₂-alkyl-aryl, the alkyl-moiety is unsaturated).

In preferred embodiments of the compounds for use according to substitution pattern (iv),
R1 represents —H, —C₁₋₁₀-alkyl, —C₁₋₁₀-alkyl-C₃₋₁₀-cycloalkyl, —C₁₋₁₀-alkyl-aryl, —C₁₋₁₀-alkyl-heteroaryl, —C₃₋₁₀-cycloalkyl, —C₁₋₁₀-alkyl-O-aryl, or —C₁₋₁₀-alkyl-O-heteroaryl;
R3 represents —C₁₋₆-alkyl-OH; or —C₁₋₆-alkyl-O—C(═O)C₁₋₆-alkyl;
R7 represents —H, —C₁₋₁₀-alkyl, —C₁₋₁₀-alkyl-C₃₋₁₀-cycloalkyl, —C₁₋₁₀-alkyl-aryl, —C₁₋₁₀-alkyl-heteroaryl, —C₃₋₁₀-cycloalkyl, —C₁₋₁₀-alkyl-O-aryl, or —C₁₋₁₀-alkyl-O-heteroaryl; and
R8 represents —H, —C₁₋₁₀-alkyl, —C₁₋₁₀-alkyl-C₃₋₁₀-cycloalkyl, —C₁₋₁₀-alkyl-aryl, —C₁₋₁₀-alkyl-heteroaryl, —C₃₋₁₀-cycloalkyl, —C₁₋₁₀-alkyl-O-aryl, or —C₁₋₁₀-alkyl-O-heteroaryl.

In more preferred embodiments of the compounds for use according to substitution pattern (iii),
R1 represents —H, —C₁₋₁₀-alkyl, —C₁₋₁₀-alkyl-C₃₋₁₀-cycloalkyl, —C₁₋₁₀-alkyl-aryl, or —C₁₋₁₀-alkyl-heteroaryl;
R3 represents —CH₂CH₂CH₂CH₂—P(═O)(OH)₂;
R7 represents —H, —C₁₋₁₀-alkyl, —C₁₋₁₀-alkyl-C₃₋₁₀-cycloalkyl, —C₁₋₁₀-alkyl-aryl, or —C₁₋₁₀-alkyl-heteroaryl; and
R8 represents -cyclopentyl, -phenyl, or —CH═CH-phenyl.

In more preferred embodiments of the compounds for use according to substitution pattern (iv),
R1 represents —H, —C₁₋₁₀-alkyl, —C₁₋₁₀-alkyl-C₃₋₁₀-cycloalkyl, —C₁₋₁₀-alkyl-aryl, —C₁₋₁₀-alkyl-heteroaryl, —C₃₋₁₀-cycloalkyl, —C₁₋₁₀-alkyl-O-aryl, or —C₁₋₁₀-alkyl-O-heteroaryl;
R3 represents —CH₂CH₂CH₂—OH; or —CH₂CH₂CH₂—O—C(═O)C₁₋₆-alkyl;
R7 represents —H, —C₁₋₁₀-alkyl, —C₁₋₁₀-alkyl-C₃₋₁₀-cycloalkyl, —C₁₋₁₀-alkyl-aryl, —C₁₋₁₀-alkyl-heteroaryl, —C₃₋₁₀-cycloalkyl, —C₁₋₁₀-alkyl-O-aryl, or —C₁₋₁₀-alkyl-O-heteroaryl; and
R8 represents —H, —C₁₋₁₀-alkyl, —C₁₋₁₀-alkyl-C₃₋₁₀-cycloalkyl, —C₁₋₁₀-alkyl-aryl, —C₁₋₁₀-alkyl-heteroaryl, —C₃₋₁₀-cycloalkyl, —C₁₋₁₀-alkyl-O-aryl, or —C₁₋₁₀-alkyl-O-heteroaryl.

Within any of the above definitions of the preferred and more preferred embodiments of the compounds for use according to substitution patterns (iii) and (iv), in each case “C₁₋₆-alkyl”, “C₃₋₆-cycloalkyl”, “aryl”, and “heteroaryl” have the same meaning as defined above.

In particularly preferred embodiments of the compounds according to general formula 1 for use according to the invention,
R1 represents —CH₂—C≡CH, —CH₂—CH₃, —CH₂—CH₂—CH₃, or —CH₂-cyclobutyl, unsubstituted;
R3 represents (—CH₂—)₄₋₆—P(═O)(OH)₂;
R7 represents (i) —H; (ii) —CH₃, —CH₂—CH₃, —CH₂—CH₂—CH₃, —CH(CH₃)₂, —CH(CH₃)(CH₂—CH₃), —CH₂—CH₂—OH, —CH₂—CH₂—Cl, —CH₂—CH₂—NH₂, —CH₂-cyclopropyl, unsubstituted, —CH₂-cyclobutyl, unsubstituted, —CH₂-cyclopentyl, unsubstituted, —CH₂-cyclohexyl, unsubstituted; or (iii) —CH₂-phenyl, unsubstituted;
R8 represents (i) -phenyl or —CH₂—CH₂-phenyl, in either case unsubstituted; (ii) —CH₂—CH₂-phenyl, mono-, di- or trisubstituted with —OCH₃, —CH₃, —CH₂—CH₃, —CH(CH₃)₂, —CF₃, —F, —Cl, —Br, —I and/or unsubstituted-phenyl; (iii) —CH═CH-phenyl, mono-, di- or trisubstituted with —OCH₃; (iv) -cyclopentyl, unsubstituted; or (v) —CH₂-naphthyl, unsubstituted; or
R1 represents —CH₂—C≡CH, —CH₂—CH₃, or —CH₂-cyclobutyl, unsubstituted;
R3 represents (—CH₂—)₄—P(═O)(OCH₂CH₃)₂;
R7 represents —H or —CH₃;
R8 represents (i) —CH₂—O-phenyl, unsubstituted; (ii) —CH₂—CH₂-phenyl, mono-, di- or trisubstituted with —OCH₃, —CH₃, —CF₃ and/or —Br; or (iii) -cyclopropyl-phenyl, unsubstituted; or
R1 represents —CH₂—C≡CH;
R3 represents (—CH₂—)₄—P(═O)(OH)(OCH₂CH₃);
R7 represents —H or —CH₃;
R8 represents —CH₂—CH₂-phenyl, mono-, di- or trisubstituted with —OCH₃ and/or —Br; or
R1 represents —CH₂—C≡CH or —CH₂—CH₃;
R3 represents (—CH₂—)₄—SO₂—OH;
R7 represents —H;
R8 represents —CH₂—CH₂-phenyl, mono-, di- or trisubstituted with —OCH₃ and/or —Br; or
R1 represents —CH₂—C≡CH;
R3 represents (—CH₂—)₄—SO₂—NH₂;
R7 represents —H;
R8 represents —CH₂—CH₂-phenyl, mono-, di- or trisubstituted with —Br;
or
R1 represents —CH₂—C≡CH or CH₂-cyclobutyl, unsubstituted;
R3 represents (—CH₂—)₄₋₅—CO₂H;
R7 represents —H;
R8 represents —CH₂—CH₂-phenyl, mono-, di- or trisubstituted with —OCH₃, CF₃ and/or —Br.

Particularly preferred compounds for use according to substitution pattern (iii) are compounds J-1 to J-3:

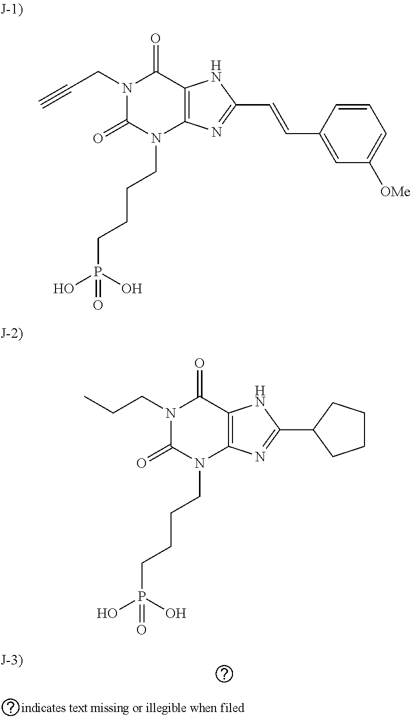 [SMILES: C#CCn1c(=O)c2[nH]c(/C=C/c3cccc(OC)c3)nc2n(CCCCP(=O)(O)O)c1=O ; CCCn1c(=O)c2[nH]c(C3CCCC3)nc2n(CCCCP(=O)(O)O)c1=O]

and the physiologically acceptable salts thereof.

Preferably, the disease or disorder that is associated with the MRGPRX4 receptor is selected from
open wounds, e.g. incisions or incised wounds, lacerations, abrasions (grazes), avulsions, puncture wounds, penetration wounds and gunshot wounds; and
closed wounds, e.g. hematomas and crash injuries; and
painful states, itching, neuropathic pain, chronic pain.

Preferably, the disease or disorder that is associated with the MRGPRX4 receptor is associated with the wildtype of the MRGPRX4 (83S) or its variant 83L.

Preferably, the compound is administered topically and/or locally.

Particularly preferred embodiments of the invention are summarized as embodiments 1 to 15 (Emb. 1 to 15) hereinafter:
Emb. 1: A compound according to general Formula 1

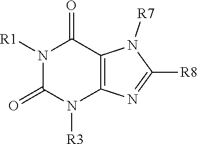

wherein
R1 represents —H, —C₁₋₁₀-alkyl, —C₁₋₁₀-alkyl-C₃₋₁₀-cycloalkyl, —C₁₋₁₀-alkyl-aryl, —C₁₋₁₀-alkyl-heteroaryl, —C₃₋₁₀-cycloalkyl, —C₁₋₁₀-alkyl-O-aryl, or —C₁₋₁₀-alkyl-O-heteroaryl;
R3 represents —C₁₋₁₀-alkyl-P(═O)(OC₁₋₁₀-alkyl)₂, —C₁₋₁₀-alkyl-P(═O)(OH)(OC₁₋₁₀-alkyl), —C₁₋₁₀-alkyl-S(═O)₂(OH), —C₁₋₁₀-alkyl-S(═O)₂(NH₂), —C₁₋₁₀-alkyl-C(═O)(OH), or —C₁₋₁₀-alkyl-P(═O)(OH)₂;
R7 represents —H, —C₁₋₁₀-alkyl, —C₁₋₁₀-alkyl-C₃₋₁₀-cycloalkyl, —C₁₋₁₀-alkyl-aryl, —C₁₋₁₀-alkyl-heteroaryl, —C₃₋₁₀-cycloalkyl, —C₁₋₁₀-alkyl-O-aryl, or —C₁₋₁₀-alkyl-O-heteroaryl; and
R8 represents —H, —C₁₋₁₀-alkyl, —C₁₋₁₀-alkyl-C₃₋₁₀-cycloalkyl, —C₁₋₁₀-alkyl-aryl, —C₁₋₁₀-alkyl-heteroaryl, —C₃₋₁₀-cycloalkyl, —C₁₋₁₀-alkyl-aryl, —C₁₋₁₀-alkyl-O-aryl, —C₃₋₆-cycloalkyl-aryl, —C₃₋₆-cycloalkyl-heteroaryl, —C₁₋₁₀-alkyl-heteroaryl, or —C₁₋₁₀-alkyl-O-heteroaryl;
wherein in each case “C₁₋₁₀-alkyl” may be linear or branched, unless expressly stated otherwise saturated or unsaturated, unsubstituted or monosubstituted with a substituent selected from —C≡CH, —C(═O)OH, —C(═O)O—C₁₋₁₀-alkyl, —OH, —O—C₁₋₁₀-alkyl, —NH₂, —NH—C₁₋₁₀-alkyl, —N(C₁₋₁₀-alkyl)₂, —N₃, —F, —Cl, —Br, and —I;
wherein in each case “C₃₋₁₀-cycloalkyl” may be linear or branched, unless expressly stated otherwise saturated or unsaturated, unsubstituted or monosubstituted with a substituent selected from —C≡CH, —C(═O)OH, —C(═O)O—C₁₋₆-alkyl, —OH, —O—C₁₋₁₀-alkyl, —NH₂, —NH—C₁₋₁₀-alkyl, —N(C₁₋₁₀-alkyl)₂, —N₃, —F, —Cl, —Br, and —I;
wherein in each case “aryl” is a 6-14-membered aryl moiety which may be unsubstituted, mono- or disubstituted with a substituent independently selected from —C≡CH, —CF₃, —CCl₃, —CBr₃, —Cl₃, —C(═O)OH, —C(═O)O—C₁₋₁₀-alkyl, —OH, —O—C₁₋₁₀-alkyl, —NH₂, —NH—C₁₋₁₀-alkyl, —N(C₁₋₁₀-alkyl)₂, —N₃, —F, —Cl, —Br, and —I;
wherein in each case “heteroaryl” is a 5-14-membered heteroaryl moiety which may be unsubstituted, mono- or disubstituted with a substituent independently selected from —C≡CH, —CF₃, —CCl₃, —CBr₃, —Cl₃, —C(═O)OH, —C(═O)O—C₁₋₁₀-alkyl, —OH, —O—C₁₋₁₀-alkyl, —NH₂, —NH—C₁₋₁₀-alkyl, —N(C₁₋₁₀-alkyl)₂, —N₃, —F, —Cl, —Br, and —I;
or a physiologically acceptable salt thereof,
with the proviso that the compound is not a compound selected from the group consisting of compounds J-1 to J-3:

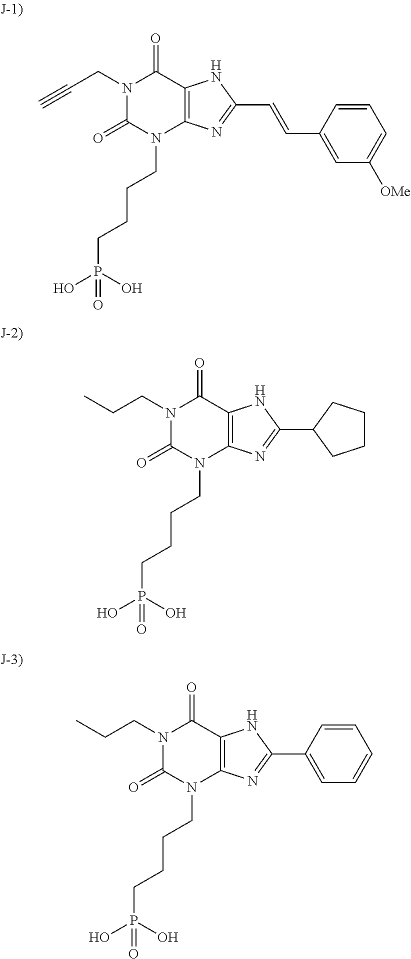 [SMILES: C#CCn1c(=O)c2[nH]c(/C=C/c3cccc(OC)c3)nc2n(CCCCP(=O)(O)O)c1=O ; CCCn1c(=O)c2[nH]c(C3CCCC3)nc2n(CCCCP(=O)(O)O)c1=O ; CCCn1c(=O)c2[nH]c(-c3ccccc3)nc2n(CCCCP(=O)(O)O)c1=O]

Emb. 2: The compound according to embodiment 1, wherein
   (i) R3 represents —C₁₋₁₀-alkyl-P(═O)(OH)₂ and R8 represents —C₃₋₆-cycloalkyl-aryl, —C₃₋₆-cycloalkyl-heteroaryl, —C₁₋₁₀-alkyl-aryl or —C₁₋₁₀-alkyl-heteroaryl (wherein in case of —C₂-alkyl-aryl, the alkyl-moiety is saturated);
      and wherein in each case R1 and R7 independently from one another are selected from —H, —C₁₋₁₀-alkyl, —C₁₋₁₀-alkyl-C₃₋₁₀-cycloalkyl, —C₁₋₁₀-alkyl-aryl, and —C₁₋₁₀-alkyl-heteroaryl; or
   wherein
   (ii) R3 represents —C₁₋₁₀-alkyl-P(═O)(OC₁₋₁₀-alkyl)₂; or
      R3 represents —C₁₋₁₀-alkyl-P(═O)(OH)(OC₁₋₁₀-alkyl); or
      R3 represents —C₁₋₁₀-alkyl-S(═O)₂(OH); or
      R3 represents —C₁₋₁₀-alkyl-S(═O)₂(NH₂); or
      R3 represents —C₁₋₁₀-alkyl-C(═O)(OH);
      and wherein in each case R1, R7, and R8 independently from one another are selected from —H, —C₁₋₁₀-alkyl, —C₁₋₁₀-alkyl-C₃₋₁₀-cycloalkyl, —C₁₋₁₀-alkyl-aryl, —C₁₋₁₀-alkyl-heteroaryl, —C₃₋₁₀-cycloalkyl, —C₁₋₁₀-alkyl-O-aryl, and —C₁₋₁₀-alkyl-O-heteroaryl.
Emb. 3: The compound according to embodiment 1 or 2, wherein
   R1 represents —C₁₋₁₀-alkyl, optionally substituted with —C≡CH; or —C₁₋₁₀-alkyl-C₃₋₁₀-cycloalkyl;
      preferably —C₁₋₆-alkyl, optionally substituted with —C≡CH; or —C₁₋₆-alkyl-C₃₋₆-cycloalkyl;
      more preferably —CH₂CH₃, —CH₂CH₂CH₃, —CH₂C≡CH, or —CH₂-cyclobutyl; and/or
   R3 represents —C₃₋₅-alkyl-P(═O)(OH)₂; —C₃₋₅-alkyl-O—P(═O)(OH)₂; —C₃₋₅-alkyl-P(═O)(OC₁₋₆-alkyl)₂; —C₃₋₅-alkyl-P(═O)(OH)(OC₁₋₆- alkyl); —C₃₋₅-alkyl-S(═O)₂(OH); —C₃₋₅-alkyl-S(═O)₂(NH₂); or —C₃₋₅-alkyl-C(═O)(OH);
      preferably —C₃₋₅-alkyl-P(═O)(OH)₂;
      more preferably —C₄-alkyl-P(═O)(OH)₂; and/or
   R7 represents —H; —C₁₋₁₀-alkyl, optionally substituted with —OH; —C₁₋₁₀-alkyl-C₃₋₁₀-cycloalkyl; or —C₁₋₁₀-alkyl-aryl;
      preferably —H; —C₁₋₆-alkyl, optionally substituted with —OH; —C₁₋₆-alkyl-C₃₋₆-cycloalkyl; or —C₁₋₆-alkyl-aryl;
      more preferably —H, —CH₃, —CH₂CH₃, —CH₂CH₂—OH, —CH₂CH₂CH₃, —CH₂-cyclopropyl, or —CH₂-phenyl; and/or
   R8 represents —C₃₋₁₀-cycloalkyl, —C₁₋₁₀-alkyl-C₃₋₁₀-cycloalkyl, -aryl, —C₁₋₁₀-alkyl-aryl, —C₃₋₆-cycloalkyl-aryl, or —C₁₋₁₀-alkyl-O-aryl;
      preferably -phenyl, optionally substituted with —F, —Br, —Cl, —CH₃, —CF₃, —OCH₃; —CH₂-phenyl, optionally substituted with —F, —Br, —Cl, —CH₃, —CF₃, —OCH₃; —CH₂CH₂-phenyl, optionally substituted with one or two substituents independently of one another selected from —F, —Br, —Cl, —CH₃, —CF₃, —OCH₃; —CH═CH-phenyl, optionally substituted with —F, —Br, —Cl, —CH₃, —CF₃, —OCH₃; —CH₂—O-phenyl, optionally substituted with —F, —Br, —Cl, —CH₃, —CF₃, —OCH₃; -cyclopropyl-phenyl, optionally substituted with —F, —Br, —Cl, —CH₃, —CF₃, —OCH₃; or —C₃₋₆-cycloalkyl;
      more preferably -phenyl, optionally substituted with —OCH₃; —CH₂-phenyl, optionally substituted with —Cl; —CH₂CH₂-phenyl, optionally substituted with one or two substituents independently of one another selected from —F, —Br, —Cl, —CH₃, —CF₃, —OCH₃; —CH═CH-phenyl, optionally substituted with —OCH₃; —CH₂—O-phenyl; -cyclopropyl-phenyl; or -cyclopentyl.
Emb. 4: The compound according to any of the preceding embodiments,
   wherein
   (i) R3 represents —C₁₋₆-alkyl-P(═O)(OH)₂ and R8 represents —C₃₋₆-cycloalkyl-phenyl or —C₁₋₆-alkyl-phenyl (wherein in case of —C₂-alkyl-phenyl, the alkyl-moiety is saturated);
      and wherein in each case R1 and R7 independently from one another are selected from —H, —C₁₋₆-alkyl, —C₁₋₆-alkyl-C₃₋₆-cycloalkyl, —C₁₋₆-alkyl-aryl, and —C₁₋₆-alkyl-heteroaryl; or
   wherein
   (ii) R3 represents —C₁₋₆-alkyl-P(═O)(OC₁₋₆-alkyl)₂; or
      R3 represents —C₁₋₆-alkyl-P(═O)(OH)(OC₁₋₆-alkyl); or
      R3 represents —C₁₋₆-alkyl-S(═O)₂(OH); or
      R3 represents —C₁₋₆-alkyl-S(═O)₂(NH₂); or
      R3 represents —C₁₋₆-alkyl-C(═O)(OH);
      and wherein in each case R1, R7, and R8 independently from one another are selected from —H, —C₁₋₆-alkyl, —C₁₋₆-alkyl-C₃₋₆-cycloalkyl, —C₁₋₆-alkyl-aryl, —C₁₋₆-alkyl-heteroaryl, —C₃₋₆-cycloalkyl, —C₁₋₆—O-aryl, and —C₁₋₆—O-heteroaryl;
   wherein in each case “C₁₋₆-alkyl” may be linear or branched, unless expressly stated otherwise saturated or unsaturated, unsubstituted or monosubstituted with a substituent selected from —C≡CH, —C(═O)OH, —C(═O)O—C₁₋₁₀-alkyl, —OH, —O—C₁₋₁₀-alkyl, —NH₂, —NH—C₁₋₁₀-alkyl, —N(C₁₋₁₀-alkyl)₂, —N₃, —F, —Cl, —Br, and —I;
   wherein in each case “C₃₋₆-cycloalkyl” may be linear or branched, unless expressly stated otherwise saturated or unsaturated, unsubstituted or monosubstituted with a substituent selected from —C≡CH, —C(═O)OH, —C(═O)O—C₁₋₆-alkyl, —OH, —O—C₁₋₁₀-alkyl, —NH₂, —NH—C₁₋₁₀-alkyl, —N(C₁₋₁₀-alkyl)₂, —N₃, —F, —Cl, —Br, and —I;
   or a physiologically acceptable salt thereof.
Emb. 5: The compound according to any of the preceding embodiments,
   wherein
   (i) R3 represents —C₄-alkyl-P(═O)(OH)₂ and R8 represents —C₃-cycloalkyl-aryl or —C₁₋₆-alkyl-phenyl (wherein in case of —C₂-alkyl-phenyl, the alkyl-moiety is saturated);
      and wherein in each case R1 and R7 are independently from one another are selected from —H, —C₁₋₆-alkyl, —C₁₋₆-alkyl-C₃₋₆-cycloalkyl, —C₁₋₆-alkyl-aryl, and —C₁₋₆-alkyl-heteroaryl; or
   wherein
   (ii) R3 represents —C₄-alkyl-P(═O)(OC₁₋₆-alkyl)₂; or
      R3 represents —C₄-alkyl-P(═O)(OH)(OC₁₋₆-alkyl); or
      R3 represents —C₄-alkyl-S(═O)₂(OH); or
      R3 represents —C₄-alkyl-S(═O)₂(NH₂); or
      R3 represents —C₄-alkyl-C(═O)(OH);
      and wherein in each case R1, R7, and R8 independently from one another are selected from —H, —C₁₋₆-alkyl, —C₁₋₆-alkyl-C₃₋₆-cycloalkyl, —C₁₋₆-alkyl-aryl, —C₁₋₆-alkyl-heteroaryl, —C₃₋₆-cycloalkyl, —C₁₋₆—O-aryl, and —C₁₋₆—O-heteroaryl;
   wherein in each case “C₁₋₆-alkyl” may be linear or branched, unless expressly stated otherwise saturated or unsaturated, unsubstituted or monosubstituted with a substituent selected from —C≡CH, —C(═O)OH, —C(═O)O—C₁₋₁₀-alkyl, —OH, —O—C₁₋₁₀-alkyl, —NH₂, —NH—C₁₋₁₀-alkyl, —N(C₁₋₁₀-alkyl)₂, —N₃, —F, —Cl, —Br, and —I;
   wherein in each case “C₃₋₆-cycloalkyl” may be linear or branched, unless expressly stated otherwise saturated or unsaturated, unsubstituted or monosubstituted with a substituent selected from —C≡CH, —C(═O)OH, —C(═O)O—C₁₋₆-alkyl, —OH, —O—C₁₋₁₀-alkyl, —NH₂, —NH—C₁₋₁₀-alkyl, —N(C₁₋₁₀-alkyl)₂, —N₃, —F, —Cl, —Br, and —I;
   or a physiologically acceptable salt thereof.
Emb. 6: The compound according any of the preceding embodiments,
   wherein
   (i) R3 represents —CH₂CH₂CH₂CH₂—P(═O)(OH)₂ and R8 represents -cyclopropyl-aryl, —CH₂-phenyl or —CH₂CH₂-phenyl (wherein in case of —CH₂CH₂-phenyl, the ethyl-moiety is saturated);
      and wherein in each case R1 and R7 independently from one another are selected from —H, —C₁₋₆-alkyl, —C₁₋₆-alkyl-C₃₋₆-cycloalkyl, —C₁₋₆-alkyl-aryl, and —C₁₋₆-alkyl-heteroaryl; or
   wherein
   (ii) R3 represents —CH₂CH₂CH₂CH₂—P(═O)(OC₁₋₆-alkyl)₂; or
      R3 represents —CH₂CH₂CH₂CH₂—P(═O)(OH)(OC₁₋₆-alkyl); or
      R3 represents —CH₂CH₂CH₂CH₂—S(═O)₂(OH); or
      R3 represents —CH₂CH₂CH₂CH₂—S(═O)₂(NH₂); or
      R3 represents —CH₂CH₂CH₂CH₂—C(═O)(OH);
      and wherein in each case R1, R7, and R8 independently from one another are selected from —H, —C₁₋₆-alkyl, —C₁₋₆-alkyl-C₃₋₆-cycloalkyl, —C₁₋₆-alkyl-aryl, —C₁₋₆-alkyl-heteroaryl, —C₃₋₆-cycloalkyl, —C₁₋₆—O-aryl, and —C₁₋₆—O-heteroaryl;
   wherein in each case “C₁₋₆-alkyl” may be linear or branched, unless expressly stated otherwise saturated or unsaturated, unsubstituted or monosubstituted with a substituent selected from —C≡CH, —C(═O)OH, —C(═O)O—C₁₋₁₀-alkyl, —OH, —O—C₁₋₁₀-alkyl, —NH₂, —NH—C₁₋₁₀-alkyl, —N(C₁₋₁₀-alkyl)₂, —N₃, —F, —Cl, —Br, and —I;
   wherein in each case “C₃₋₆-cycloalkyl” may be linear or branched, unless expressly stated otherwise saturated or unsaturated, unsubstituted or monosubstituted with a substituent selected from —C≡CH, —C(═O)OH, —C(═O)O—C₁₋₆-alkyl, —OH, —O—C₁₋₁₀-alkyl, —NH₂, —NH—C₁₋₁₀-alkyl, —N(C₁₋₁₀-alkyl)₂, —N₃, —F, —Cl, —Br, and —I;
   or a physiologically acceptable salt thereof.
Emb. 7: The compound according any of the preceding embodiments which is selected from compounds
   (I) B-1 to B-23 and the physiologically acceptable salts thereof:

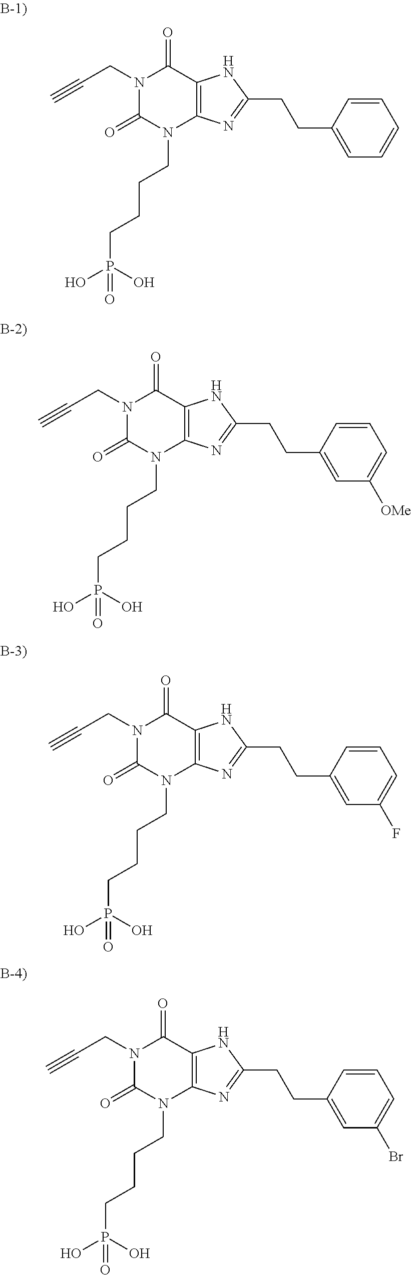 [SMILES: C#CCn1c(=O)c2[nH]c(CCc3ccccc3)nc2n(CCCCP(=O)(O)O)c1=O ; C#CCn1c(=O)c2[nH]c(CCc3cccc(OC)c3)nc2n(CCCCP(=O)(O)O)c1=O ; C#CCn1c(=O)c2[nH]c(CCc3cccc(F)c3)nc2n(CCCCP(=O)(O)O)c1=O ; C#CCn1c(=O)c2[nH]c(CCc3cccc(Br)c3)nc2n(CCCCP(=O)(O)O)c1=O]

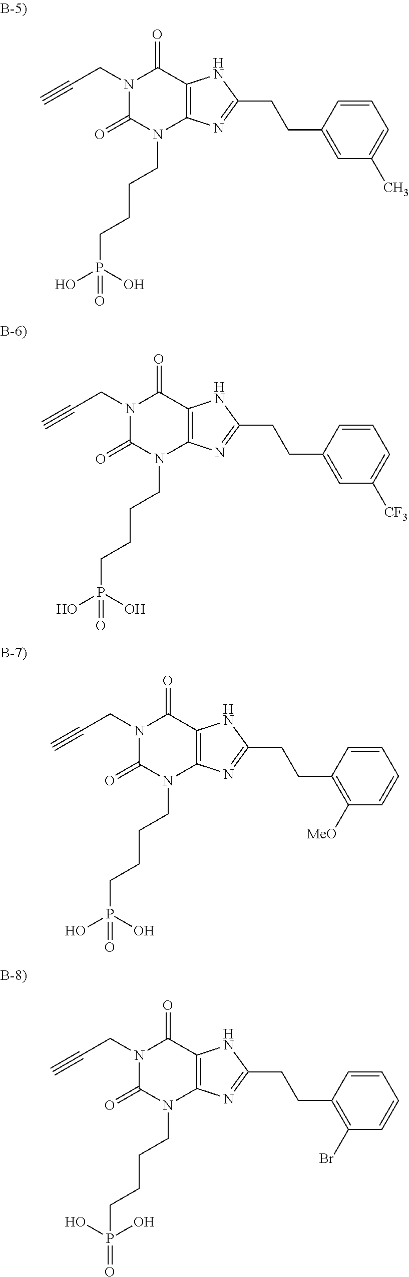

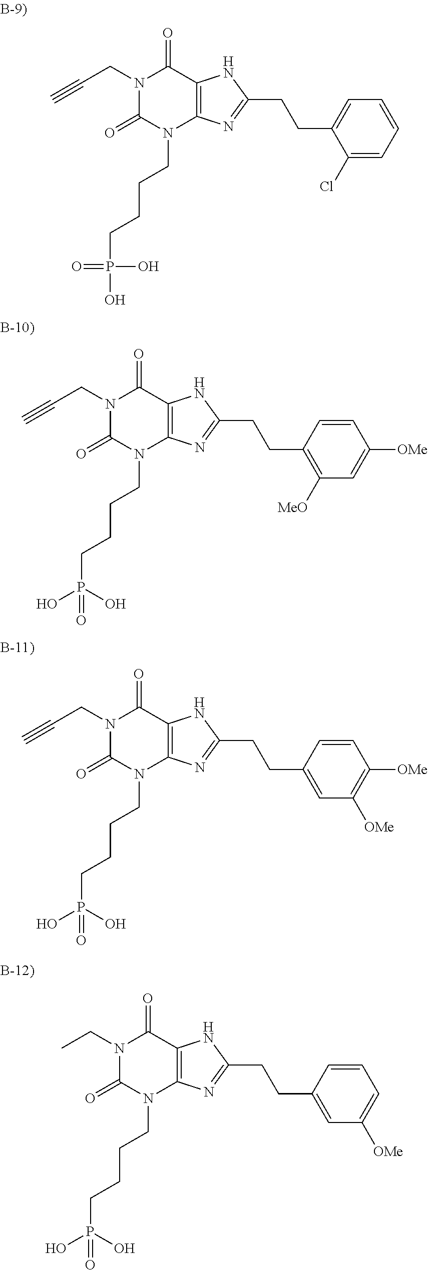 [SMILES: C#CCn1c(=O)c2[nH]c(CCc3ccccc3Cl)nc2n(CCCCP(=O)(O)O)c1=O ; C#CCn1c(=O)c2[nH]c(CCc3ccc(OC)cc3OC)nc2n(CCCCP(=O)(O)O)c1=O ; C#CCn1c(=O)c2[nH]c(CCc3ccc(OC)c(OC)c3)nc2n(CCCCP(=O)(O)O)c1=O ; CCn1c(=O)c2[nH]c(CCc3cccc(OC)c3)nc2n(CCCCP(=O)(O)O)c1=O]

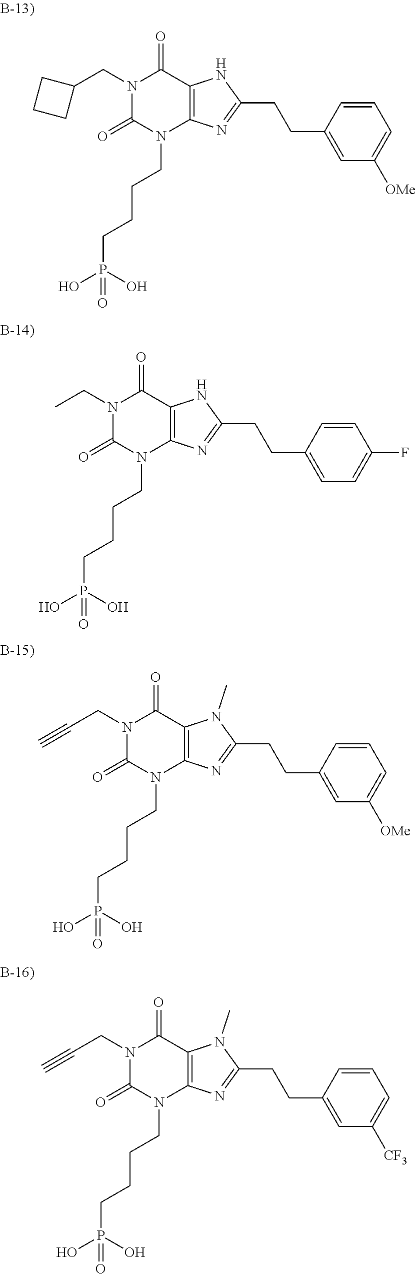 [SMILES: COc1cccc(CCc2nc3c([nH]2)c(=O)n(CC2CCC2)c(=O)n3CCCCP(=O)(O)O)c1 ; CCn1c(=O)c2[nH]c(CCc3ccc(F)cc3)nc2n(CCCCP(=O)(O)O)c1=O ; C#CCn1c(=O)c2c(nc(CCc3cccc(OC)c3)n2C)n(CCCCP(=O)(O)O)c1=O ; C#CCn1c(=O)c2c(nc(CCc3cccc(C(F)(F)F)c3)n2C)n(CCCCP(=O)(O)O)c1=O]

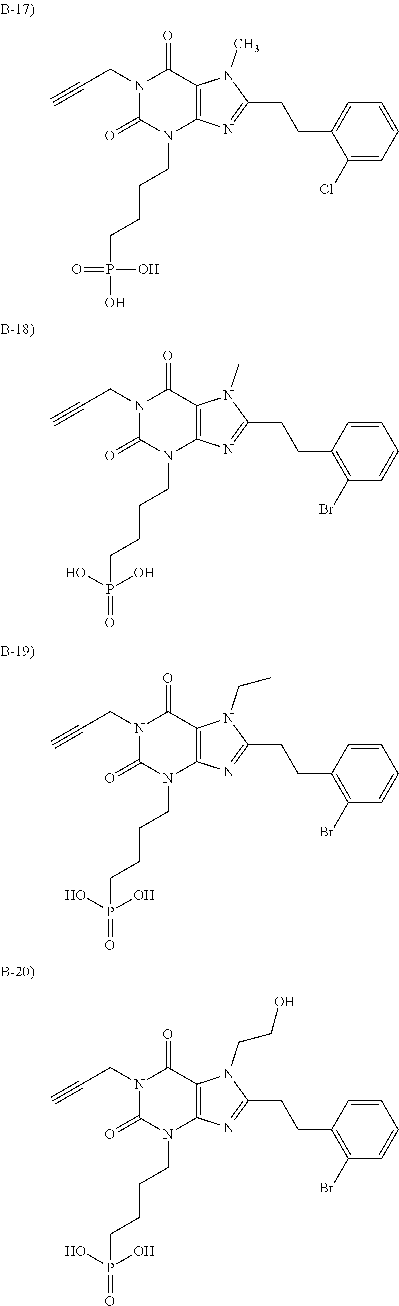 [SMILES: C#CCn1c(=O)c2c(nc(CCc3ccccc3Cl)n2C)n(CCCCP(=O)(O)O)c1=O ; C#CCn1c(=O)c2c(nc(CCc3ccccc3Br)n2C)n(CCCCP(=O)(O)O)c1=O ; C#CCn1c(=O)c2c(nc(CCc3ccccc3Br)n2CC)n(CCCCP(=O)(O)O)c1=O ; C#CCn1c(=O)c2c(nc(CCc3ccccc3Br)n2CCO)n(CCCCP(=O)(O)O)c1=O]

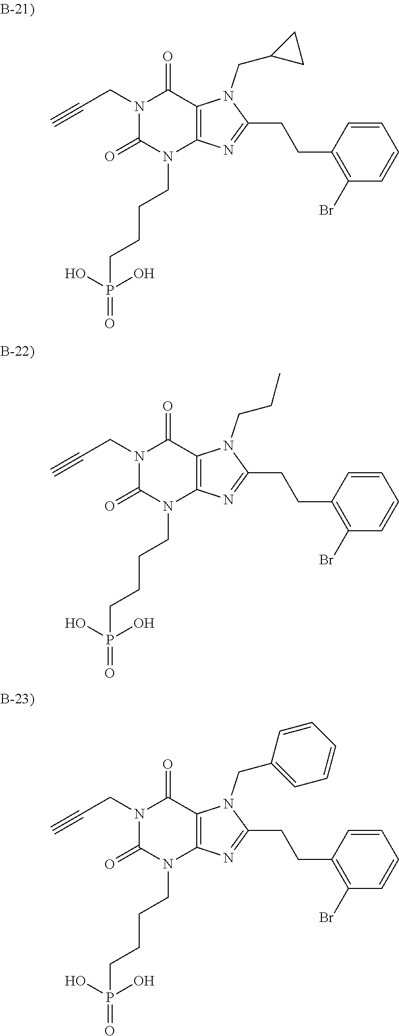 [SMILES: C#CCn1c(=O)c2c(nc(CCc3ccccc3Br)n2CC2CC2)n(CCCCP(=O)(O)O)c1=O ; C#CCn1c(=O)c2c(nc(CCc3ccccc3Br)n2CCC)n(CCCCP(=O)(O)O)c1=O ; C#CCn1c(=O)c2c(nc(CCc3ccccc3Br)n2Cc2ccccc2)n(CCCCP(=O)(O)O)c1=O]

and
(II) C-1 to C-10 and the physiologically acceptable salts thereof:

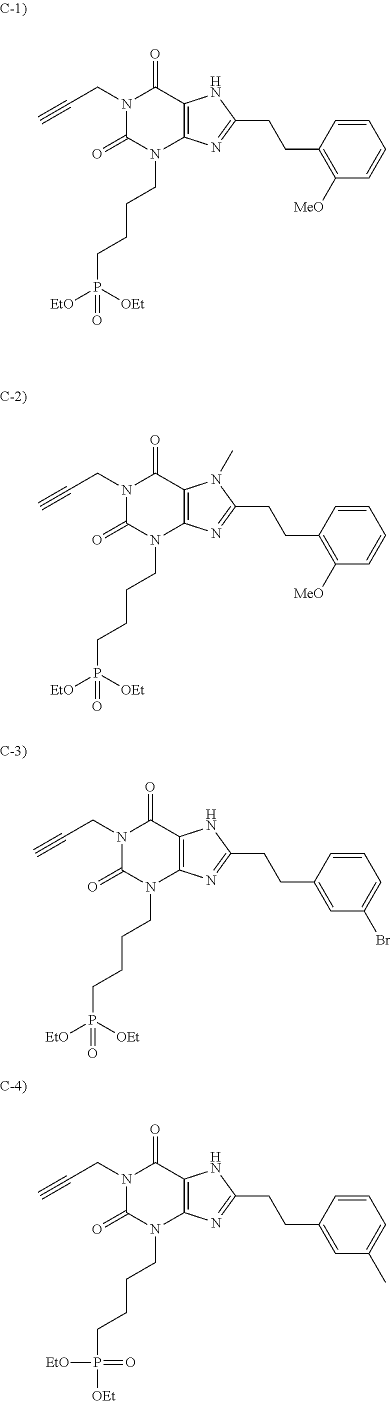 [SMILES: C#CCn1c(=O)c2[nH]c(CCc3ccccc3OC)nc2n(CCCCP(=O)(OCC)OCC)c1=O ; C#CCn1c(=O)c2c(nc(CCc3ccccc3OC)n2C)n(CCCCP(=O)(OCC)OCC)c1=O ; C#CCn1c(=O)c2[nH]c(CCc3cccc(Br)c3)nc2n(CCCCP(=O)(OCC)OCC)c1=O ; C#CCn1c(=O)c2[nH]c(CCc3cccc(C)c3)nc2n(CCCCP(=O)(OCC)OCC)c1=O]

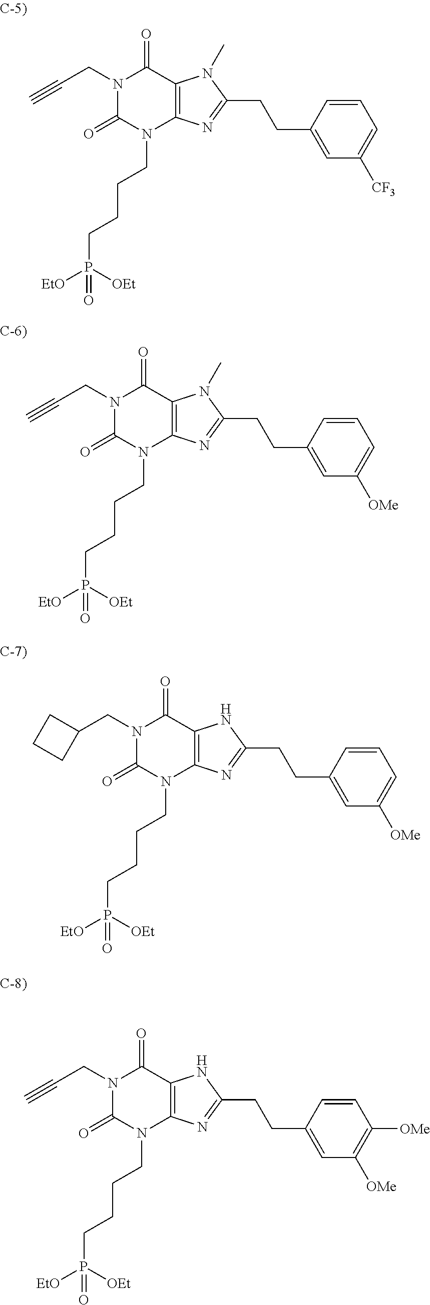 [SMILES: C#CCn1c(=O)c2c(nc(CCc3cccc(C(F)(F)F)c3)n2C)n(CCCCP(=O)(OCC)OCC)c1=O ; C#CCn1c(=O)c2c(nc(CCc3cccc(OC)c3)n2C)n(CCCCP(=O)(OCC)OCC)c1=O ; CCOP(=O)(CCCCn1c(=O)n(CC2CCC2)c(=O)c2[nH]c(CCc3cccc(OC)c3)nc21)OCC ; C#CCn1c(=O)c2[nH]c(CCc3ccc(OC)c(OC)c3)nc2n(CCCCP(=O)(OCC)OCC)c1=O]

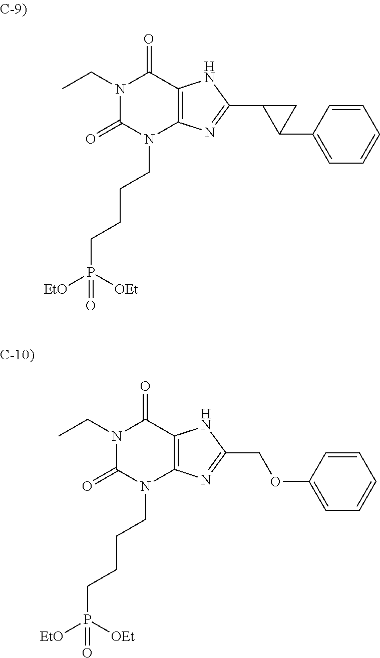 [SMILES: CCOP(=O)(CCCCn1c(=O)n(CC)c(=O)c2[nH]c(C3CC3c3ccccc3)nc21)OCC ; CCOP(=O)(CCCCn1c(=O)n(CC)c(=O)c2[nH]c(COc3ccccc3)nc21)OCC]

and
(III) D-1 to D-3 and the physiologically acceptable salts thereof:

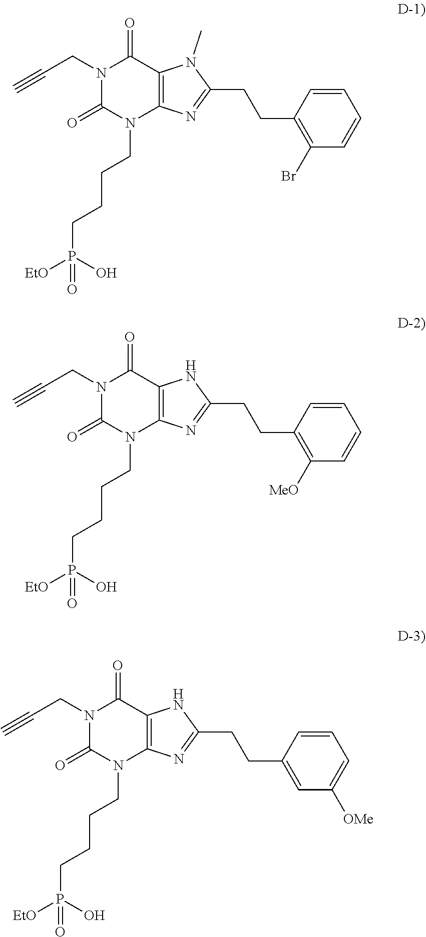 [SMILES: C#CCn1c(=O)c2c(nc(CCc3ccccc3Br)n2C)n(CCCCP(=O)(O)OCC)c1=O ; C#CCn1c(=O)c2[nH]c(CCc3ccccc3OC)nc2n(CCCCP(=O)(O)OCC)c1=O ; C#CCn1c(=O)c2[nH]c(CCc3cccc(OC)c3)nc2n(CCCCP(=O)(O)OCC)c1=O]

and
(IV) E-1 to E-3 and the physiologically acceptable salts thereof:

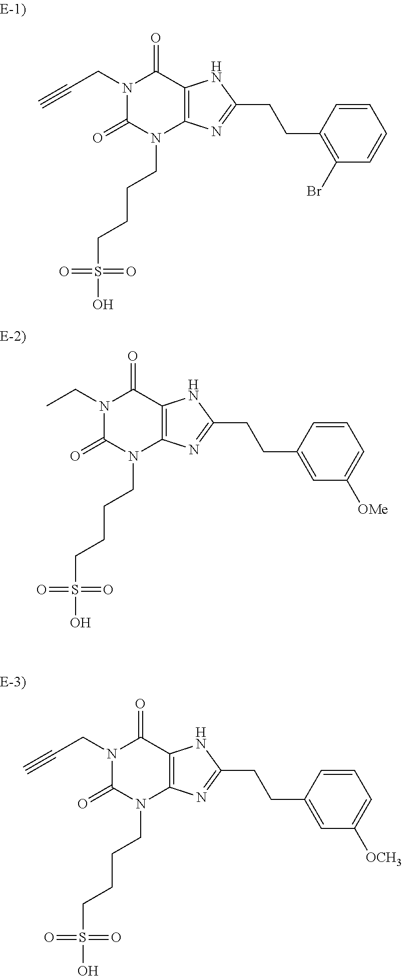 [SMILES: C#CCn1c(=O)c2[nH]c(CCc3ccccc3Br)nc2n(CCCCS(=O)(=O)O)c1=O ; CCn1c(=O)c2[nH]c(CCc3cccc(OC)c3)nc2n(CCCCS(=O)(=O)O)c1=O ; C#CCn1c(=O)c2[nH]c(CCc3cccc(OC)c3)nc2n(CCCCS(=O)(=O)O)c1=O]

and
(V) F-1 and the physiologically acceptable salts thereof:

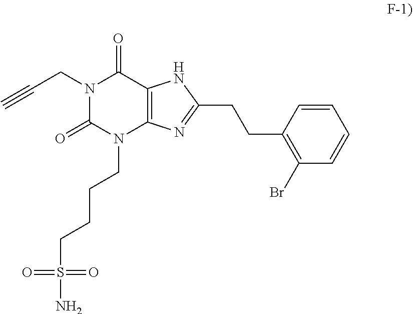 [SMILES: C#CCn1c(=O)c2[nH]c(CCc3ccccc3Br)nc2n(CCCCS(N)(=O)=O)c1=O]

and
(VI) G-1 and G-2 and the physiologically acceptable salts thereof:

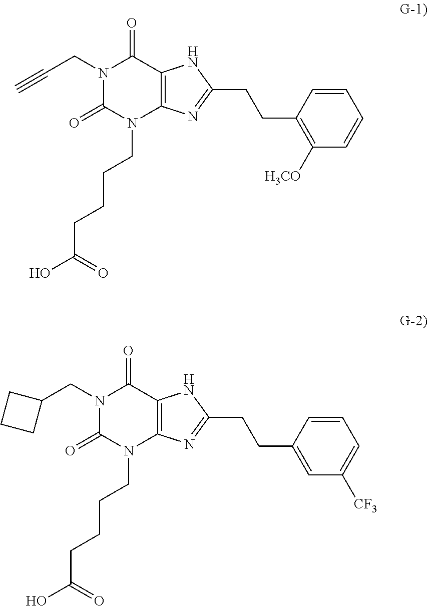 [SMILES: C#CCn1c(=O)c2[nH]c(CCc3ccccc3OC)nc2n(CCCCC(=O)O)c1=O ; O=C(O)CCCCn1c(=O)n(CC2CCC2)c(=O)c2[nH]c(CCc3cccc(C(F)(F)F)c3)nc21]

Emb. 8: The compound according to any of the preceding embodiments for use as a medicament.
Emb. 9: The compound according to any of embodiments 1 to 7 for use in the prevention or treatment of a condition, disease or disorder that is associated with the MRGPRX4 receptor, preferably associated with the wildtype of the MRGPRX4 receptor.
Emb. 10: A compound according to general Formula 1

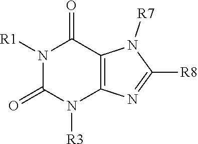

wherein
R1 represents —H, —C₁₋₁₀-alkyl, —C₁₋₁₀-alkyl-C₃₋₁₀-cycloalkyl, —C₁₋₁₀-alkyl-aryl, —C₁₋₁₀-alkyl-heteroaryl, —C₃₋₁₀-cycloalkyl, —C₁₋₁₀-alkyl-O-aryl, or —C₁₋₁₀-alkyl-O-heteroaryl;
R3 represents —C₁₋₁₀-alkyl-P(═O)(OC₁₋₁₀-alkyl)₂, —C₁₋₁₀-alkyl-P(═O)(OH)(OC₁₋₁₀-alkyl), —C₁₋₁₀-alkyl-S(═O)₂(OH), —C₁₋₁₀-alkyl-S(═O)₂(NH₂), —C₁₋₁₀-alkyl-C(═O)(OH), or —C₁₋₁₀-alkyl-P(═O)(OH)₂;
R7 represents —H, —C₁₋₁₀-alkyl, —C₁₋₁₀-alkyl-C₃₋₁₀-cycloalkyl, —C₁₋₁₀-alkyl-aryl, —C₁₋₁₀-alkyl-heteroaryl, —C₃₋₁₀-cycloalkyl, —C₁₋₁₀-alkyl-O-aryl, or —C₁₋₁₀-alkyl-O-heteroaryl; and
R8 represents —H, —C₁₋₁₀-alkyl, —C₁₋₁₀-alkyl-C₃₋₁₀-cycloalkyl, —C₁₋₁₀-alkyl-aryl, —C₁₋₁₀-alkyl-heteroaryl, —C₃₋₁₀-cycloalkyl, —C₁₋₁₀-alkyl-aryl, —C₁₋₁₀-alkyl-O-aryl, —C₃₋₆-cycloalkyl-aryl, —C₃₋₆-cycloalkyl-heteroaryl, —C₁₋₁₀-alkyl-heteroaryl, or —C₁₋₁₀-alkyl-O-heteroaryl;
wherein in each case “C₁₋₁₀-alkyl” may be linear or branched, unless expressly stated otherwise saturated or unsaturated, unsubstituted or monosubstituted with a substituent selected from —C≡CH, —C(═O)OH, —C(═O)O—C₁₋₁₀-alkyl, —OH, —O—C₁₋₁₀-alkyl, —NH₂, —NH—C₁₋₁₀-alkyl, —N(C₁₋₁₀-alkyl)₂, —N₃, —F, —Cl, —Br, and —I;
wherein in each case “C₃₋₁₀-cycloalkyl” may be linear or branched, unless expressly stated otherwise saturated or unsaturated, unsubstituted or monosubstituted with a substituent selected from —C≡CH, —C(═O)OH, —C(═O)O—C₁₋₆-alkyl, —OH, —O—C₁₋₁₀-alkyl, —NH₂, —NH—C₁₋₁₀-alkyl, —N(C₁₋₁₀-alkyl)₂, —N₃, —F, —Cl, —Br, and —I;
wherein in each case “aryl” is a 6-14-membered aryl moiety which may be unsubstituted, mono- or disubstituted with a substituent independently selected from —C≡CH, —CF₃, —CCl₃, —CBr₃, —Cl₃, —C(═O)OH, —C(═O)O—C₁₋₁₀-alkyl, —OH, —O—C₁₋₁₀-alkyl, —NH₂, —NH—C₁₋₁₀-alkyl, —N(C₁₋₁₀-alkyl)₂, —N₃, —F, —Cl, —Br, and —I;
wherein in each case “heteroaryl” is a 5-14-membered heteroaryl moiety which may be unsubstituted, mono- or disubstituted with a substituent independently selected from —C≡CH, —CF₃, —CCl₃, —CBr₃, —Cl₃, —C(═O)OH, —C(═O)O—C₁₋₁₀-alkyl, —OH, —O—C₁₋₁₀-alkyl, —NH₂, —NH—C₁₋₁₀-alkyl, —N(C₁₋₁₀-alkyl)₂, —N₃, —F, —Cl, —Br, and —I;
or a physiologically acceptable salt thereof,
for use in the prevention or treatment of a condition, disease or disorder that is associated with the MRGPRX4 receptor, preferably associated with the wildtype of the MRGPRX4 receptor.
Emb. 11: The compound for use according to embodiment 10, wherein the compound is according to any of embodiments 2 to 7.
Emb. 12: The compound according to embodiment 10, wherein
   (iii) R3 represents —C₁₋₁₀-alkyl-P(═O)(OH)₂ and R8 represents —C₃₋₆-cycloalkyl, -aryl, -heteroaryl, —C₁₋₁₀-alkyl-aryl, or —C₁₋₁₀-alkyl-heteroaryl (wherein in case of —C₂-alkyl-aryl, the alkyl-moiety is unsaturated);
      and wherein in each case R1 and R7 independently from one another are selected from —H, —C₁₋₁₀-alkyl, —C₁₋₁₀-alkyl-C₃₋₁₀-cycloalkyl, —C₁₋₁₀-alkyl-aryl, and —C₁₋₁₀-alkyl-heteroaryl; or
   wherein
   (iv) R3 represents —C₁₋₁₀-alkyl-OH; or
      R3 represents —C₁₋₁₀-alkyl-O—C(═O)C₁₋₁₀-alkyl;
      and wherein in each case R1, R7, and R8 independently from one another are selected from —H, —C₁₋₁₀-alkyl, —C₁₋₁₀-alkyl-C₃₋₁₀-cycloalkyl, —C₁₋₁₀-alkyl-aryl, —C₁₋₁₀-alkyl-heteroaryl, —C₃₋₁₀-cycloalkyl, —C₁₋₁₀-alkyl-O-aryl, and —C₁₋₁₀-alkyl-O-heteroaryl;
   for use in the prevention or treatment of a condition, disease or disorder that is associated with the MRGPRX4 receptor, preferably associated with the wildtype of the MRGPRX4 receptor.
Emb. 13: The compound for use according to any of embodiments 10 or 11, which is selected from compounds
   J-1 to J-3 and the physiologically acceptable salts thereof:

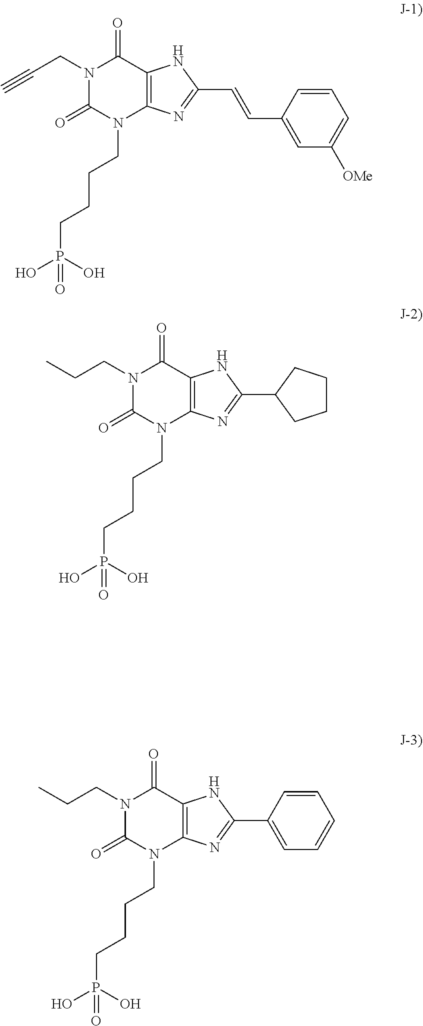 [SMILES: C#CCn1c(=O)c2[nH]c(/C=C/c3cccc(OC)c3)nc2n(CCCCP(=O)(O)O)c1=O ; CCCn1c(=O)c2[nH]c(C3CCCC3)nc2n(CCCCP(=O)(O)O)c1=O ; CCCn1c(=O)c2[nH]c(-c3ccccc3)nc2n(CCCCP(=O)(O)O)c1=O]

Emb. 14: The compound for use according to any of embodiments 8 to 13, wherein the disease or disorder that is associated with the MRGPRX4 receptor, preferably associated with the wildtype of the MRGPRX4 receptor, is selected from
   open wounds; preferably selected from the group consisting of incisions or incised wounds, lacerations, abrasions (grazes), avulsions, puncture wounds, penetration wounds and gunshot wounds; and
   closed wounds; preferably selected from the group consisting of hematomas and crash injuries; and
   painful states, itching, neuropathic pain, chronic pain.
Emb. 15: The compound for use according to any of embodiments 8 to 14, wherein the compound is administered topically and/or locally.

The inventions illustratively described herein may suitably be practiced in the absence of any element or elements, limitation or limitations, not specifically disclosed herein. Thus, for example, the term “includes”shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the inventions embodied therein herein disclosed may be resorted to by those skilled in the art, and that such modifications and variations are considered to be within the scope of this invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

Other embodiments are within the following claims and non-limiting examples. In addition, where features or aspects of the invention are described in terms of Markush groups, those skilled in the art will recognize that the invention is also thereby described in terms of any individual member or subgroup of members of the Markush group.

The present inventions will be explained in more detail in the following examples. However, the examples are only used for illustration and do not limit the scope of the present invention.

### Experimental Part

### Synthesis of Reference Compounds A-1 and H-1-H-9

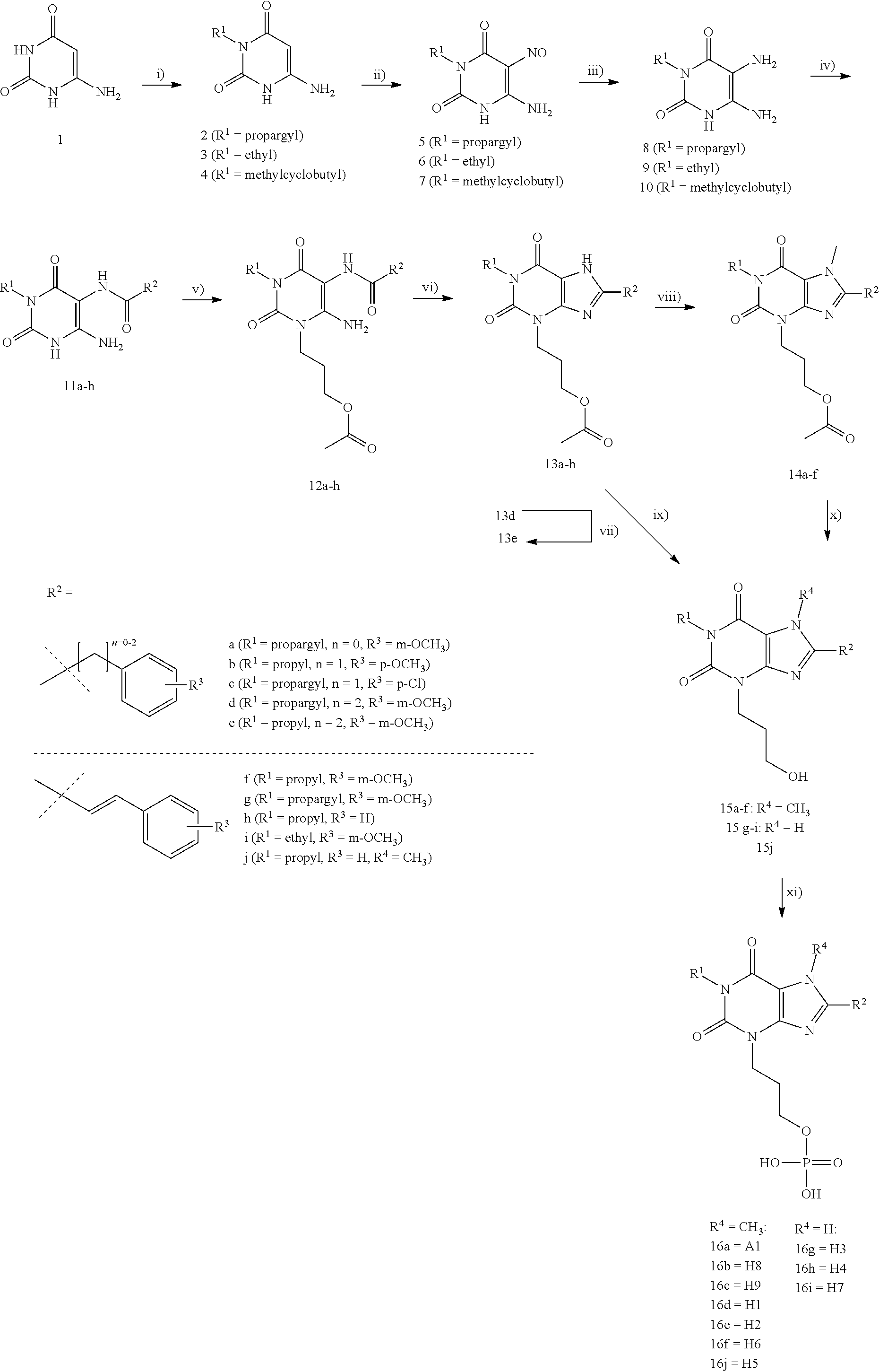

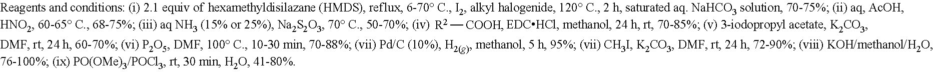

### Synthesis of Inventive Compounds G-1-G-2

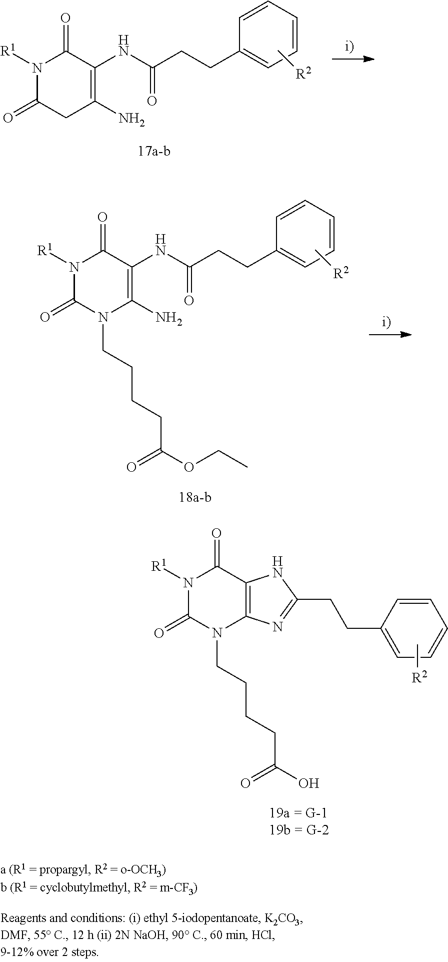

### Synthesis of Inventive Compound G-3

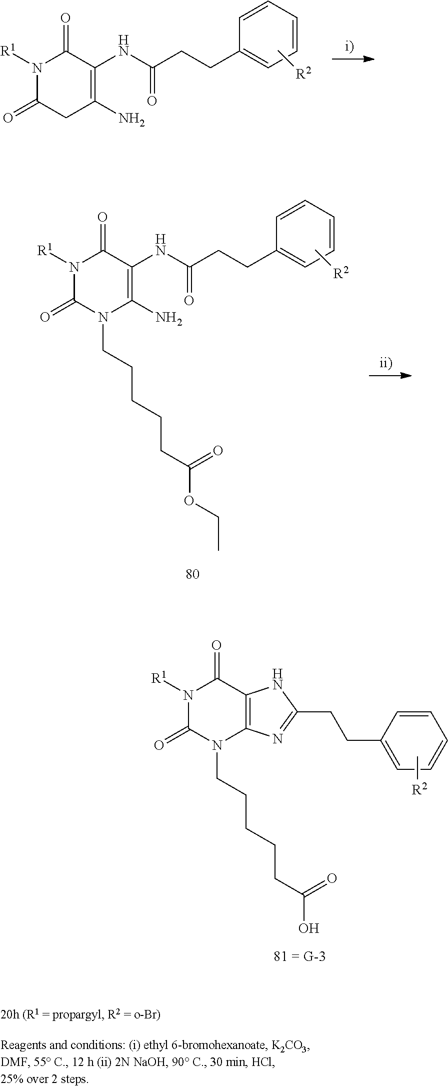

### Synthesis of Inventive Compounds B-1-B-23, C-1-C-10, D-1-D-3 and J-1-J-3

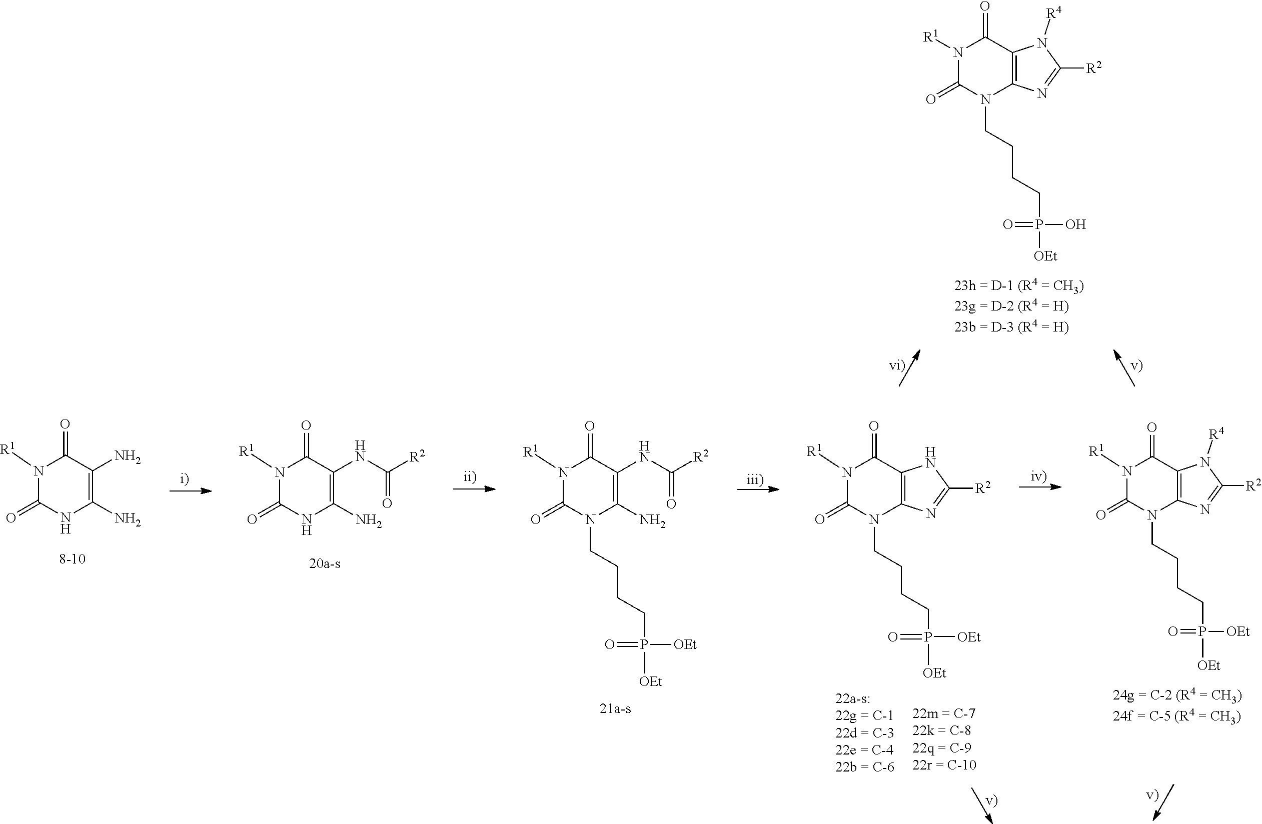

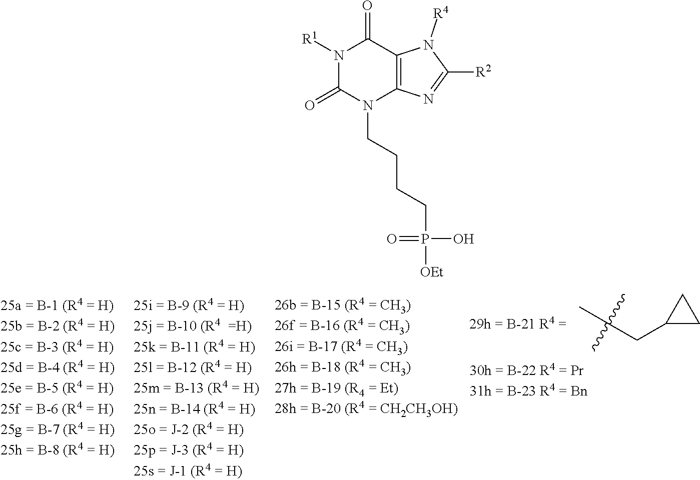

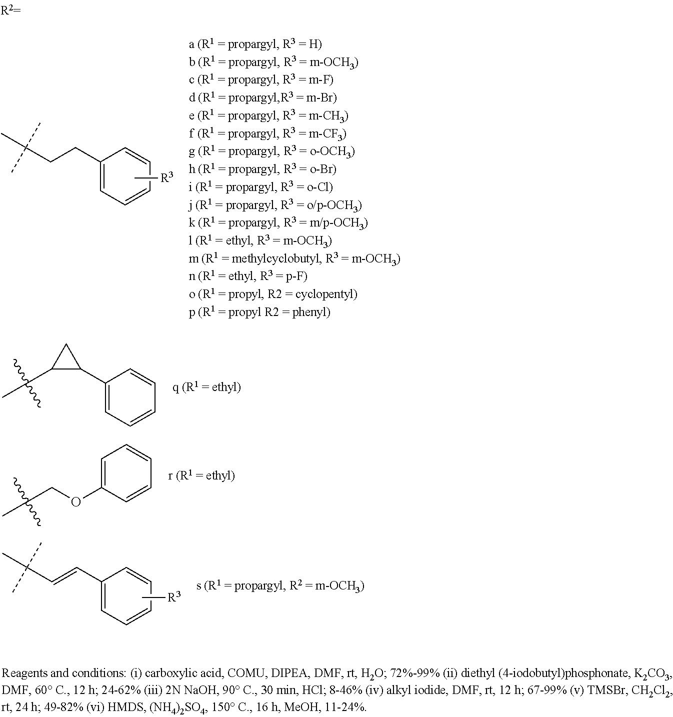

### Synthesis of Inventive Compounds B-24-B-57

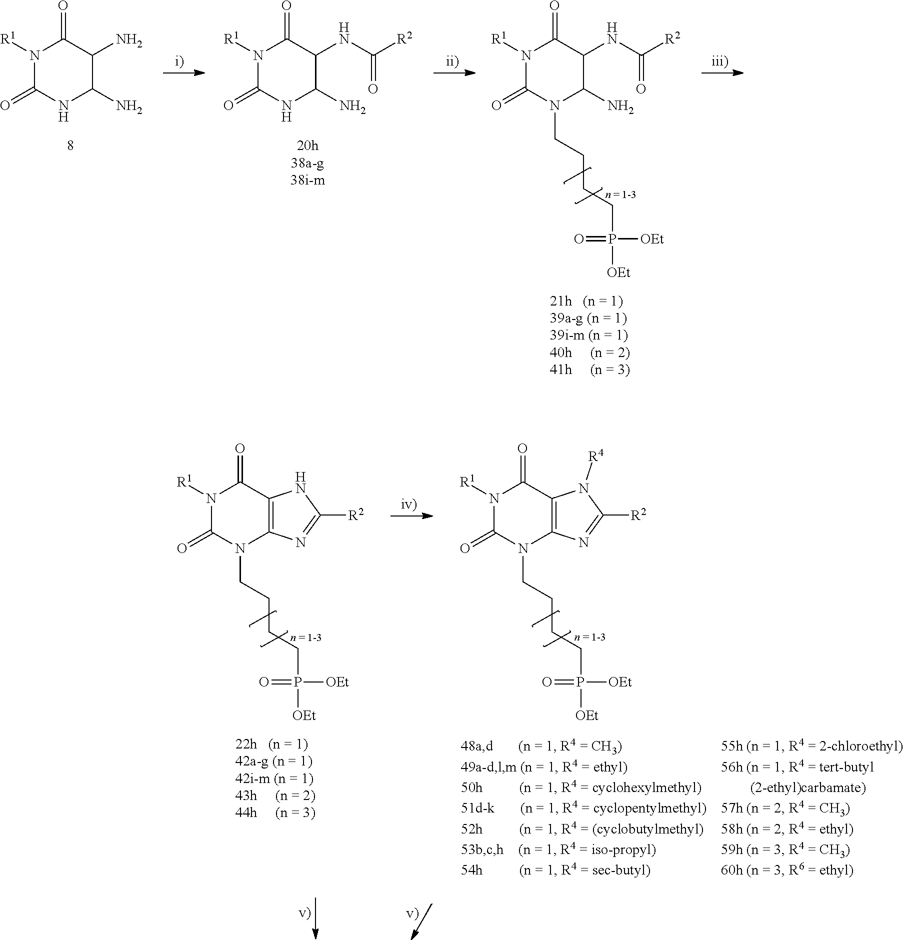

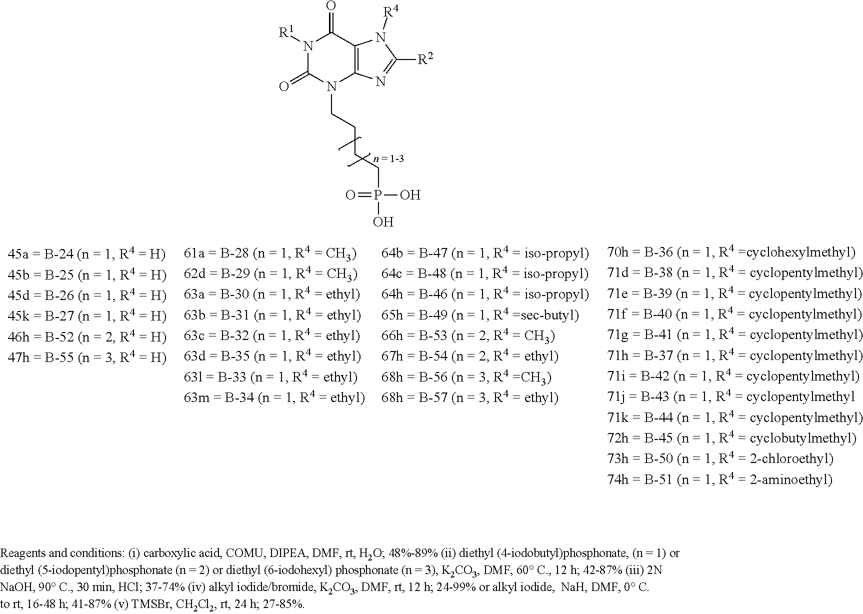

### Synthesis of Inventive Compound B-58 (=79

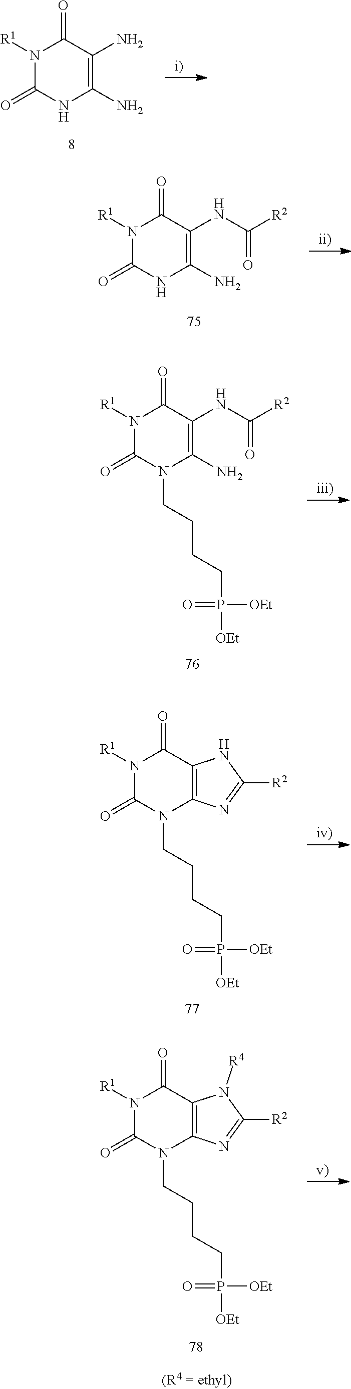

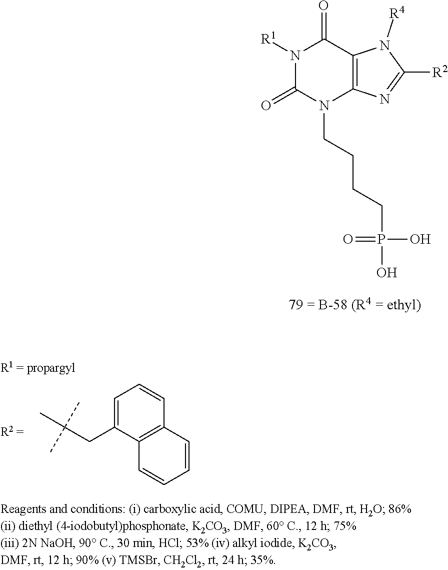

### Synthesis of Compounds E-1-E-3 and F-1

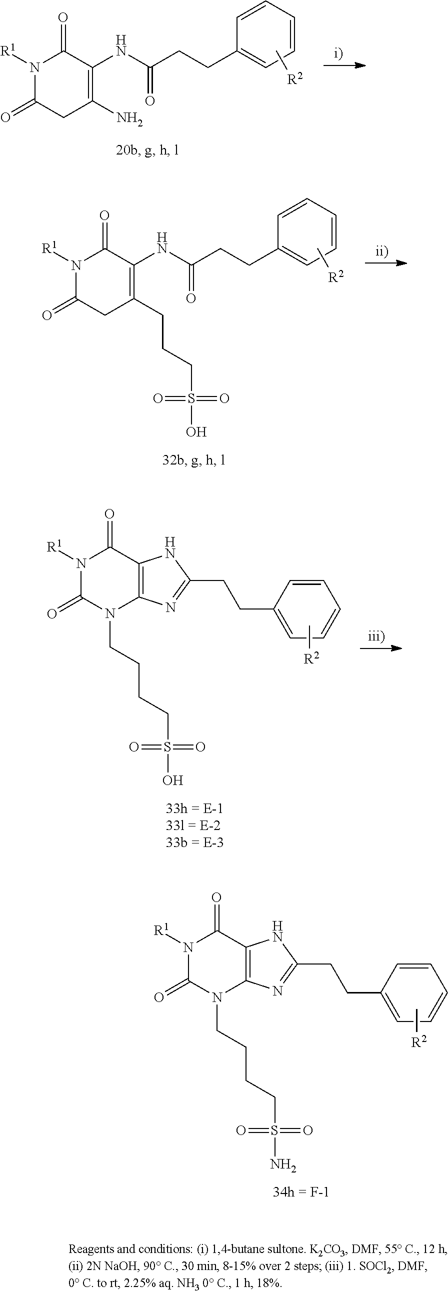

General Procedures:

The synthesis of 3-substituted 5,6-diaminouracil derivatives (8-10) was performed according to literature procedures (Maxwell, L. C. E., and Salivar, C. J. Method of preparing 4-aminouracils. U.S. Pat. No. 2,715,625A, 1952, Müller et al., J. Med. Chem. 1993, 36: 3341-3349)

General Procedure A: Amide Coupling Reaction to Produce N3-Substituted 5-Amino-6-Carboxamidouracil Derivatives (11a-h; 17a-b; 20a-s; 38a-g; 38i-m; 75)

To a solution of the appropriate carboxylic acid (1.0 equiv) and COMU (1.1 equiv) dissolved in a minimum amount of dimethylformamide (DMF) a mixture of the substituted diaminouracil (8-10, 1.1 equiv) and N,N-Diisopropylethylamine (DIPEA 1.1 equiv) dissolved in DMF was added dropwise. The mixture was stirred at room temperature for 5-10 min. After complete conversion of the starting material water (30 ml) was added and the resulting precipitate was filtered off, washed with water and dried under reduced pressure to yield the pure 6-amino-5-carboxamidouracil derivatives 11a-h; 17a-b; 20a-s in high isolated yields (75-99%) and 38a-g; 38i-m; 75 in moderate to high isolated yields (48-89%).

General Procedure B: Alkylation of N1 of the 3-Substituted 6-Amino-5-Carboxamidouracil Derivatives (12a-h; 18a-b; 21a-s; 32b, g, h, 1; 39a-g; 39i-m; 40h; 41h; 76; 80)

6-Amino-5-carboxamidouracil derivative (11a-h; 17a-b; 20a-s; 35a-g; 35i-m; 72; 1.0 equiv), 1.1 equiv of diethyl (4-iodobutyl)phosphonate or 1,4-butansultone or ethyl 5-bromopentanoate (in case of compounds 11a-h; 17a-b; 20a-s); or 1.1 equiv of diethyl (4-iodobutyl)phosphonate or diethyl (5-iodopentyl)phosphonate or diethyl (6-iodohexyl)phosphonate or ethyl 6-bromohexanoate (in case of compounds 35a-g; 35i-m; 72); and K₂CO₃ (1.2 equiv) were dissolved in a minimum amount of DMF (1-2 ml). After stirring for 12 h at 55° C., DMF was removed, water (15 ml) was added, and the resulting mixture extracted with dichloromethane (3×15 ml). The combined organic phases were washed with brine (20 ml), dried over MgSO₄ and the solvent was removed in vacuo. The residue was purified by flash column chromatography on silica gel 60 (eluent:dichloromethane/methanol, 9:1 in case of compounds 12a-h; 18a-b; 21a-s; 32b, g, h, 1; and eluent:dichloromethane/methanol, 95:5 in case of compounds 39a-g; 39i-m; 40h; 41h; 80) to yield the desired substituted uracil derivatives (12a-h; 18a-b; and 21a-s; 32b, g, h, 1; 39a-g; 39i-m; 40h; 41h; 80).

General Procedure D: Ring Closure Reaction to the Corresponding Xanthines Using NaOH (13a-h; 42a-g; 42i-m; 43h; 44h; 77; C-1; C-3; C-4; C-6 to C-10, E-1 to E-3, G-1 to G-3)

The 1,3-disubstituted-6-amino-5-carboxamidouracil derivatives were dissolved in a minimum amount of NaOH (2N) and stirred at 90° C. until complete conversion of the starting material (TLC:dichloro-methane/methanol, 9:1) was observed (20-30 min). The mixture was cooled to 0° C. in an ice bath, and the desired product was precipitated by the addition of concentrated HCl solution. The precipitate was filtered off and washed with water to yield the xanthine derivatives as off-white solids.

General Procedure E: Alkylation of the Xanthine N7-Position (14a-f; 23h, g, b; 24g, f; 48a, d; 49a-d, 1, m; 50h; 51d-k; 52h; 55h; 56h; 57h; 58h; 59h; 60h; 78; C-2; C-5)

The xanthine derivatives (13a-h; 22a-s; 22h; 42a-g; 42i-m; 43h; 44h; 77; C-2; C-5; 1.0 equiv), K₂CO₃ (1.2 equiv) and alkyl iodide/or alkyl bromide (1.1 equiv) were dissolved in a minimum of DMF (1-2 ml) and stirred at room temperature overnight. After complete conversion of the starting material (TLC:dichloromethane/methanol, 9:1) DMF was removed, water was added, and extracted three times with EtOAc (5-10 ml). The combined organic phases were washed with brine, dried over MgSO₄, filtered, and the solvent was removed in vacuo to yield the N7-alkylated xanthine derivatives (14a-f; 23h, g, b; 24g, f; 48a, d; 49a-d, 1, m; 50h; 51d-k; 52h; 55h; 56h; 57h; 58h; 59h; 60h; 78; C-2; C-5).

General Procedure E1: Alkylation of the Xanthine N7-Position (53b, c, h and 54h)

The xanthine derivatives (22h; 42b, c: 1.0 equiv) were dissolved in a minimum of DMF (1-2 ml), cooled to 0° C., NaH (1.1 equiv) was added and the solution stirred for 30 min. Alkyl iodide (5.0 equiv) was added, and the solution was stirred at room temperature for 16h. DMF was removed, water was added, and extracted three times with EtOAc (5-10 ml). The combined organic phases were washed with brine, dried over MgSO₄, filtered, and the solvent was removed in vacuo. Purification of the residue by flash column chromatography on silica gel 60 (eluent:dichloromethane/methanol, 95:5) yielded the N7-alkylated xanthine derivatives (53b, c, h and 54h).

General Procedure F: Deprotection of the Phosphonic Acid Esters Using Trimethylsilyl Bromide (A-1; B-1 to B-58; J-1 to J-3; H-1 to H-10)

The phosphonic acid ester derivatives were dissolved in dichloromethane (5-10 ml), TMSBr (3 equiv) was added, and the resulting solution was stirred for 12 h at rt. The solvent was removed in vacuo and the residue was dissolved in 2N NaOH (2-3 ml), the product was then precipitated by addition of a minimum amount of concentrated HCl solution and washed with water (3×5 ml) to yield the final xanthine derivatives (A-1; B-1 to B-58, J-1 to J-3; H-1 to H-10).

A-1) 3-(8-(3-Methoxyphenyl)-7-methyl-2,6-dioxo-1-(prop-2-yn-1-yl)-1,2,6,7-tetrahydro-3H-purin-3-yl)propyl dihydrogen phosphate: White solid; Yield 76%; ¹H-NMR (600 MHz, DMSO-d₆) δ [ppm]=7.49-7.44 (m, 1H, Hₐᵣₒₘ), 7.33 (d, J=7.6 Hz, 1H, Hₐᵣₒₘ), 7.31-7.27 (m, 1H, Hₐᵣₒₘ), 7.15-7.08 (m, 1H, Hₐᵣₒₘ), 4.61 (d, J=2.4 Hz, 2H, N1-CH₂), 4.10 (t, J=7.3 Hz, 2H, N3-CH₂), 3.97 (s, 3H, OCH₃), 3.87 (m, 2H, OCH₂), 3.82 (s, 3H, N7-CH₃), 3.11-3.03 (m, 1H, H_{propargyl}), 2.00-1.98 (m, 2H, H_{alkyl}). ¹³C-NMR (151 MHz, DMSO-d₆) δ [ppm]=159.44 (Cₐᵣₒₘ—OCH₃), 153.81 (C8), 151.65 (C6), 149.98 (C2), 147.62 (C4), 130.16 (Cₐᵣₒₘ), 129.48 (Cₐᵣₒₘ), 128.18 (Cₐᵣₒₘ), 125.66 (Cₐᵣₒₘ), 121.62 (Cₐᵣₒₘ), 108.00 (C5), 79.77 (C_{propargyl}), 72.96 (C_{propargyl}), 63.13 (OCH₂), 55.57 (OCH₃), 40.49 (N3-CH₂), 33.88 (N1-CH₂), 30.21 (N7-CH₃), 28.93 (CH₂). ³¹P-NMR (243 MHz, DMSO-d₆) δ [ppm]=0.27.

B-1) (4-(2,6-Dioxo-8-phenethyl-1-(prop-2-yn-1-yl)-1,2,6,7-tetrahydro-3H-purin-3-yl)butyl)phosphonic acid: Yield 57%; ¹H NMR (500 MHz, DMSO-d₆) δ 13.27 (br s, 1H, N7-H), 7.30-7.22 (m, 2H, Hₐᵣₒₘ) 7.12-7.11 (m, 3H, Hₐᵣₒₘ), 4.58 (d, J=2.5 Hz, 2H, N1-CH₂), 3.97 (t, J=7.2 Hz, 2H, N3-CH₂), 3.07 (t, J=2.1 Hz, 1H, H_{propargyl}), 3.05-2.95 (m, 4H, C8-CH₂CH₂), 1.73-170 (m, 2H, H_{alkyl}), 1.52-1.47 (m, 4H, H_{alkyl}). ¹³C NMR (126 MHz, DMSO-d₆) δ 154.14 (C8), 153.02 (C6), 150.12 (C2), 148.14 (C4), 140.58 (Cₐᵣₒₘ), 128.45 (Cₐᵣₒₘ), 128.42 (Cₐᵣₒₘ), 126.22 (C5), 79.89 (C_{propargyl}), 72.81 (C_{propargyl}), 42.91 (N3-CH₂), 33.36 (C8-CH₂CH₂), 30.20 (C8-CH₂CH₂), 28.78, 28.66, 28.12 (1C, PCH₂CH₂), 27.04 1C, PCH₂), 20.28 (C_{alkyl}). ³¹P NMR (243 MHz, DMSO) δ 26.73. ³¹P-NMR (243 MHz, DMSO-d₆) δ [ppm]26.73. HRMS (ESI-QTOF) calculated for C₂₀H₂₃N₄O₅P [M+H]⁺: 431.1484; found: 431.1480).

B-2) (4-(8-(3-Methoxyphenethyl)-2,6-dioxo-1-(prop-2-yn-1-yl)-1,2,6,7-tetrahydro-3H-purin-3-yl)butyl)phosphonic acid: White solid; m.p. 240-242° C. ¹H-NMR (600 MHz, DMSO-d₆) δ [ppm]=13.28 (br s, 1H, N7-H), 7.16 (t, J=7.8 Hz, 1H, Hₐᵣₒₘ), 6.79-6.71 (m, 3H, Hₐᵣₒₘ), 4.6 (d, J=1.6 Hz, 2H, N1-CH₂), 3.97 (t, J=7.2 Hz, 2H, N3-CH₂), 3.70 (s, 3H, OCH₃), 3.05 (t, J=2.1 Hz, 1H, H_{propargyl}), 2.99 (s, 4H, C8-CH₂CH₂), 1.76-1.70 (m, 2H, H_{alkyl}), 1.58-1.44 (m, 4H, H_{alkyl}). ¹³C-NMR (151 MHz, DMSO-d₆) δ [ppm]=159.4 (Cₐᵣₒₘ—OCH3), 153.0 (C8), 150.2 (C2 or C6), 148.2 (C2 or C6), 142.2 (C4), 129.5 (Cₐᵣₒₘ), 120.7 (Cₐᵣₒₘ), 117.5 (Cₐᵣₒₘ), 114.1 (Cₐᵣₒₘ), 111.8 (Cₐᵣₒₘ), 106.0 (C5), 79.9 (C_{propargyl}), 72.9 (C_{propargyl}), 55.0 (OCH₃), 42.9 (N3-CH₂), 33.4 (CH₂), 30.2 (CH₂), 30.1 (N1-CH₂) 28.7 (d, ²J_{C,P}=14.9 Hz, 1C, PCH₂CH₂), 27.5 (d, ¹J_{C,P}=138.8 Hz, 1C, PCH₂), 20.3 (C_{alkyl}). ³¹P-NMR (243 MHz, DMSO-d₆) δ [ppm]=27.0. HRMS (ESI-QTOF) calculated for C₂₁H₂₅N₄O₆P [M+H]⁺: 461.1584; found: 461.1587.

B-3) (4-(8-(3-Fluorophenethyl)-2,6-dioxo-1-(prop-2-yn-1-yl)-1,2,6,7-tetrahydro-3H-purin-3-yl)butyl)phosphonic acid: White solid: m.p. 154-156° C. ¹H-NMR (600 MHz, DMSO-d₆) δ [ppm]=13.32 (br s, 1H, N7-H), 7.29 (q, J=7.8 Hz, 1H, Hₐᵣₒₘ), 7.07 (d, J=10.1 Hz, 1H, Hₐᵣₒₘ), 7.03 (d, J=7.6 Hz, 1H, Hₐᵣₒₘ), 6.99 (td, J=8.7, 2.4 Hz, 1H, Hₐᵣₒₘ), 4.58 (d, J=2.1 Hz, 2H, N1-CH₂), 3.96 (t, J=7.1 Hz, 2H, N3-CH₂), 3.06 (t, J=2.4 Hz, 1H, H_{propargyl}), 3.05-2.99 (m, 4H, C8-CH₂CH₂), 1.72 (p, J=7.5 Hz, 2H, H_{alkyl}), 1.58-1.51 (m, 2H, H_{alkyl}), 1.47 (dp, J=15.6, 9.0, 7.9 Hz, 2H, H_{alkyl}). ¹³C-NMR (151 MHz, DMSO-d₆) δ [ppm]=162.4 (d, ¹J_{C,F}=240.5 Hz, 1C, Cₐᵣₒₘ—F), 154.0 (Cₓₐₙₜₕᵢₙₑ), 153.0 (Cₓₐₙₜₕᵢₙₑ), 150.2 (Cₓₐₙₜₕᵢₙₑ), 148.2 (Cₓₐₙₜₕᵢₙₑ), 143.6 (d, ³J_{C,F}=7.5 Hz, 1C, Cₐᵣₒₘ), 130.4 (d, ³J_{C,F}=8.9 Hz, 1C, Cₐᵣₒₘ), 124.7 (d, ⁴J_{C,F}=3.0 Hz, 1C, Cₐᵣₒₘ), 115.3 (d, ²J_{C,F}=20.7 Hz, 1C, Cₐᵣₒₘ), 113.0 (d, ²J_{C,F}=20.7 Hz, 1C, Cₐᵣₒₘ), 106.0 (C5), 79.9 (C_{propargyl}), 72.9 (C_{propargyl}), 42.9 (N3-CH₂), 33.0 (CH₂), 30.3 (CH₂), 29.8 (N1-CH₂), 28.8 (d, ²J_{C,P}=16.1 Hz, 1C, PCH₂CH₂), 27.4 (d, ¹J_{C,P}=135.7 Hz, 1C, PCH₂), 20.2 (d, ³J_{C,P}=4.4 Hz, 1C, PCH₂CH₂CH₂). ³¹P-NMR (243 MHz, DMSO-d₆) δ [ppm]=27.2. HRMS (ESI-QTOF) calculated for C₂₀H₂₂FN₄O₅P [M+H]⁺: 449.1385; found: 449.1389.

B-4) (4-(8-(3-Bromophenethyl)-2,6-dioxo-1-(prop-2-yn-1-yl)-1,2,6,7-tetrahydro-3H-purin-3-yl)butyl)phosphonic acid: White solid; m.p. 240-242° C. ¹H-NMR (600 MHz, DMSO-d₆) δ [ppm]=13.31 (s, 1H, N7-H), 7.45 (s, 1H, Hₐᵣₒₘ), 7.37 (d, J=7.4 Hz, 1H, Hₐᵣₒₘ), 7.22 (dt, J=12.6, 7.5 Hz, 2H, Hₐᵣₒₘ), 4.59 (s, 2H, N1-CH₂), 3.98 (t, J=6.9 Hz, 2H, N3-CH₂), 3.05 (s, 1H, H_{propargyl}), 3.02 (s, 4H, C8-CH₂CH₂), 1.78-1.68 (m, 2H, H_{alkyl}), 1.57 (dt, J=16.8, 7.6 Hz, 2H, H_{alkyl}), 1.52-1.44 (m, 2H, H_{alkyl}). ¹³C-NMR (151 MHz, DMSO-d₆) δ [ppm]=153.7 (Cₓₐₙₜₕᵢₙₑ), 152.9 (Cₓₐₙₜₕᵢₙₑ), 150.0 (Cₓₐₙₜₕᵢₙₑ), 147.9 (Cₓₐₙₜₕᵢₙₑ), 143.3 (Cₓₐₙₜₕᵢₙₑ), 131.2 (Cₐᵣₒₘ), 130.5 (Cₐᵣₒₘ), 129.0 (Cₐᵣₒₘ), 127.5 (Cₐᵣₒₘ), 121.6 (Cₐᵣₒₘ), 105.9 (C5), 79.7 (C_{propargyl}), 72.7 (C_{propargyl}), 42.7 (C_{alkyl}), 32.6 (C_{alkyl}), 30.1 (C_{alkyl}), 29.6 (C_{alkyl}), 28.5 (d, ²J_{C,P}=15.9 Hz, 1C, PCH₂CH₂), 27.2 (d, ¹J_{C,P}=136.7 Hz, 1C, PCH₂), 20.0 (d, ³J_{C,P}=4.1 Hz, 1C, P(CH₂)₂CH₂). ³¹P-NMR (243 MHz, DMSO-d₆) δ [ppm]=27.2. HRMS (ESI-QTOF) calculated for C₂₀H₂₃BrN₄O₅P [M+H]⁺: 509.0584; found: 509.0578.

B-5) (4-(8-(3-Methylphenethyl)-2,6-dioxo-1-(prop-2-yn-1-yl)-1,2,6,7-tetrahydro-3H-purin-3-yl)butyl)phosphonic acid: White solid; m.p. 244-246° C. ¹H-NMR (600 MHz, DMSO-d₆) δ [ppm]=13.29 (s, 1H, N7-H), 7.14 (t, J=7.5 Hz, 1H, Hₐᵣₒₘ), 7.04 (s, 1H, Hₐᵣₒₘ), 6.98 (d, J=7.5 Hz, 2H, Hₐᵣₒₘ), 4.59 (d, J=2.2 Hz, 2H, N1-CH₂), 3.97 (t, J=7.2 Hz, 2H, N3-CH₂), 3.05 (t, J=2.4 Hz, 1H, H_{propargyl}), 2.98 (s, 4H, C8-CH₂CH₂), 2.26 (s, 3H, CH₃), 1.74 (p, J=7.5 Hz, 2H, H_{alkyl}), 1.61-1.54 (m, 2H, H_{alkyl}), 1.51-1.45 (m, 2H, H_{alkyl}). ¹³C-NMR (151 MHz, DMSO-d₆) δ [ppm]=154.1 (Cₓₐₙₜₕᵢₙₑ), 152.9 (Cₓₐₙₜₕᵢₙₑ), 150.0 (Cₓₐₙₜₕᵢₙₑ), 148.0 (Cₓₐₙₜₕᵢₙₑ), 140.3 (Cₐᵣₒₘ), 137.3 (Cₐᵣₒₘ), 129.0 (Cₐᵣₒₘ), 128.2 (Cₐᵣₒₘ), 126.8 (Cₐᵣₒₘ), 125.3 (Cₐᵣₒₘ), 105.8 (C5), 79.8 (C_{propargyl}), 72.7 (C_{propargyl}), 42.7 (C_{alkyl}), 33.2 (C_{alkyl}), 30.1 (C_{alkyl}), 30.0 (C_{alkyl}), 28.6 (d, ²J_{C,P}=15.3 Hz, 1C, PCH₂CH₂), 27.3 (d, ¹J_{C,P}=136.3 Hz, 1C, PCH₂), 21.0 (CH₃), 20.0 (d, ³J_{C,P}=3.9 Hz, 1C, P(CH₂)₂CH₂). ³¹P-NMR (243 MHz, DMSO-d₆) δ [ppm]=27.2. HRMS (ESI-QTOF) calculated for C₂₁H₂₅N₄O₅P [M+H]⁺: 445.1635; found: 445.1650.

B-6) (4-(2,6-Dioxo-1-(prop-2-yn-1-yl)-8-(3-(trifluoromethyl)phenethyl)-1,2,6,7-tetrahydro-3H-purin-3-yl)butyl)phosphonic acid: White solid; m.p. 223-226° C. ¹H-NMR (500 MHz, DMSO-d₆) δ [ppm]=13.29 (br s, 1H, N7-H), 7.55 (s, 1H, Hₐᵣₒₘ), 7.51 (q, J=6.8 Hz, 3H, Hₐᵣₒₘ), 4.57 (d, 2H, N1-CH₂), 3.95 (t, 2H, N3-CH₂), 3.14-3.10 (m, 2H, C8-CH₂), 3.05-3.02 (m, 3H, C8-CH₂CH₂ and H_{propargyl}), 1.71 (dq, J=13.1, 6.8 Hz, 2H, H_{alkyl}), 1.55-1.45 (m, 4H, H_{alkyl}). ¹³C-NMR (151 MHz, DMSO-d₆) δ [ppm]=153.8 (C8), 153.2 (C2 or C6), 150.1 (C2 or C6), 148.1 (C4), 142.1 (Cₐᵣₒₘ), 132.8 (Cₐᵣₒₘ), 129.5 (Cₐᵣₒₘ), 129.2 (q, ²J_{C,F}=32.8 Hz, 1C, Cₐᵣₒₘ—CF₃), 125.1 (Cₐᵣₒₘ), 124.4 (q, ¹J_{C,F}=272.1 Hz, 1C, CF₃), 122.9 (Cₐᵣₒₘ), 123.1 (C5), 79.9 (C_{propargyl}), 72.9 (C_{propargyl}), 43.0 (N3-CH₂), 33.0 (CH₂), 30.3 (N1-CH₂), 29.8 (CH₂), 28.8 (d, ²J_{C,P}=15.3 Hz, 1C, PCH₂CH₂), 27.7 (d, ¹J_{C,P}=138.1 Hz, 1C, PCH₂), 20.4 (d, ³J_{C,P}=4.5 Hz, 1C, PCH₂CH₂CH₂). ³¹P-NMR (243 MHz, DMSO-d₆) δ [ppm]=26.3. HRMS (ESI-QTOF) calculated for C₂₁H₂₂F₃N₄O₅P [M+H]⁺: 499.1280; found: 449.1283.

B-7) (4-(8-(2-Methoxyphenethyl)-2,6-dioxo-1-(prop-2-yn-1-yl)-1,2,6,7-tetrahydro-3H-purin-3-yl)butyl)phosphonic acid: White solid; m.p. 169-172° C. ¹H-NMR (600 MHz, DMSO-d₆) δ [ppm]=13.25 (br s, 1H, N7-H), 7.19-7.15 (m, 1H, Hₐᵣₒₘ), 7.08 (d, J=7.3 Hz, 1H, Hₐᵣₒₘ), 6.94 (d, J=8.1 Hz, 1H, Hₐᵣₒₘ), 6.82 (t, J=7.3 Hz, 1H, Hₐᵣₒₘ), 4.58 (d, J=2.0 Hz, 2H, N1-CH₂), 3.96 (t, J=7.2 Hz, 2H, N3-CH₂), 3.77 (s, 3H, OCH₃), 3.04 (t, J=2.3 Hz, 1H, H_{propargyl}), 2.93-2.97 (m, 4H, C8-CH₂CH₂), 1.72 (p, J=7.4 Hz, 2H, H_{alkyl}), 1.49-1.53 (m, 4H, H_{alkyl}). ¹³C-NMR (151 MHz, DMSO-d₆) δ [ppm]=157.3 (Cₐᵣₒₘ—OCH3), 154.5 (Cₓₐₙₜₕᵢₙₑ), 153.0 (Cₓₐₙₜₕᵢₙₑ), 150.1 (Cₓₐₙₜₕᵢₙₑ), 148.2 (Cₓₐₙₜₕᵢₙₑ), 129.8 (Cₐᵣₒₘ), 128.3 (Cₐᵣₒₘ), 127.8 (Cₐᵣₒₘ), 120.4 (Cₐᵣₒₘ), 110.8 (Cₐᵣₒₘ), 106.0 (C5), 79.9 (C_{propargyl}), 72.8 (C_{propargyl}), 55.4 (OCH₃), 42.9 (N3-CH₂), 30.2 (N1-CH₂), 28.7 (CH₂), 28.7 (d, ²J_{C,P}=16.1 Hz, 1C, PCH₂CH₂), 28.6 (CH₂), 27.5 (d, ¹J_{C,P}=136.6 Hz, 1C, PCH₂), 20.3 (d, ³J_{C,P}=4.3 Hz, 1C, PCH₂CH₂CH₂). ³¹P-NMR (243 MHz, DMSO-d₆) δ [ppm]=26.9. HRMS (ESI-QTOF) calculated for C₂₁H₂₅N₄O₆P [M+H]⁺: 461.1584; found: 461.1586.

B-8) (4-(8-(2-Bromophenethyl)-2,6-dioxo-1-(prop-2-yn-1-yl)-1,2,6,7-tetrahydro-3H-purin-3-yl)butyl)phosphonic acid: White solid; m.p. 209-212° C.; ¹H-NMR (500 MHz, DMSO-d₆) δ [ppm]=13.33 (s, 1H, N7-H), 7.58 (d, J=8.0 Hz, 1H, Hₐᵣₒₘ), 7.29 (d, J=4.4 Hz, 2H, Hₐᵣₒₘ), 7.15 (dt, J=8.7, 4.6 Hz, 1H, Hₐᵣₒₘ), 4.59 (d, J=2.4 Hz, 2H, N1-CH₂), 3.14 (t, J=7.6 Hz, 2H, —CH₂), 3.07-2.99 (m, 3H, —CH₂ and H_{propargyl}), 1.74 (p, J=7.5 Hz, 2H, —CH₂), 1.64-1.43 (m, 4H, —CH₂CH₂). ¹³C-NMR (126 MHz, DMSO-d₆) δ [ppm]=153.4 (C8 or C4), 152.9 (C8 or C4), 150.0 (CO), 148.0 (CO), 139.3 (Cₐᵣₒₘ), 132.5 (Cₐᵣₒₘ), 130.7 (Cₐᵣₒₘ), 128.4 (Cₐᵣₒₘ), 127.9 (Cₐᵣₒₘ), 123.7 (Cₐᵣₒₘ), 106.0 (C5), 79.7 (C_{propargyl}), 72.7 (C_{propargyl}), 42.7 (N—CH₂), 33.5 (N—CH₂), 30.1 (—CH₂), 28.5 (d, ²J_{C,P}=15.6 Hz, 1C, PCH₂CH₂), 28.3 (—CH₂), 27.2 (d, ¹J_{C,P}=136.2 Hz, 1C, PCH₂), 20.0 (d, 3J_{C,P}=4.3 Hz, PCH₂CH₂CH₂). ³¹P-NMR (243 MHz, DMSO-d₆): δ [ppm]=27.3. HRMS (ESI-QTOF) calculated for C₂₀H₂₃BrN₄O₅P [M+H]⁺: 509.0584; found: 509.0566.

B-9) (4-(8-(2-Chlorophenethyl)-2,6-dioxo-1-(prop-2-yn-1-yl)-1,2,6,7-tetrahydro-3H-purin-3-yl)butyl)phosphonic acid: White solid; m.p. 207-209° C.; ¹H-NMR (600 MHz, DMSO-d₆) δ [ppm]=13.51-12.94 (m, 1H, N7-H), 7.41 (dd, J=7.0, 2.1 Hz, 1H, Hₐᵣₒₘ), 7.29 (dd, J=7.0, 2.4 Hz, 1H, Hₐᵣₒₘ), 7.24 (tt, J=7.4, 5.3 Hz, 2H, Hₐᵣₒₘ), 4.59 (d, J=2.5 Hz, 2H, N1-CH₂), 3.97 (t, J=7.2 Hz, 2H, N3-CH₂), 3.14 (t, J=7.6 Hz, 2H, CH₂), 3.09-3.04 (m, 1H, H_{propargyl}), 3.02 (t, J=7.6 Hz, 2H, CH₂), 1.73 (p, J=7.3 Hz, 2H, CH₂), 1.52 (m, 4H, 2×CH₂). ¹³C-NMR (151 MHz, DMSO-d₆) δ [ppm]=153.5 (C8 or C4), 152.9 (C8 or C4), 150.0 (CO), 148.0 (CO), 137.6 (Cₐᵣₒₘ), 132.9 (Cₐᵣₒₘ), 130.7 (Cₐᵣₒₘ), 129.2 (Cₐᵣₒₘ), 128.2 (Cₐᵣₒₘ), 127.3 (Cₐᵣₒₘ), 105.9 (C5), 79.7 (C_{propargyl}), 72.7 (C_{propargyl}), 42.7 (—CH₂), 31.1 (—CH₂), 30.1 (—CH₂), 28.5 (d, ²J_{C,P}=15.6 Hz, 1C, PCH₂CH₂), 28.2 (—CH₂), 27.3 (d, ¹J_{C,P}=137.0 Hz, 1C, PCH₂), 20.0 (d, ³J_{C,P}=4.2 Hz, PCH₂CH₂CH₂). ³¹P-NMR (243 MHz, DMSO-d₆) δ [ppm]=27.0. HRMS (ESI-QTOF) n.d.

B-10) (4-(8-(2,4-Dimethoxyphenethyl)-2,6-dioxo-1-(prop-2-yn-1-yl)-1,2,6,7-tetrahydro-3H-purin-3-yl)butyl)phosphonic acid: White solid; m.p. 255-257° C. ¹H-NMR (600 MHz, DMSO-d₆) δ [ppm]=7.02 (d, J=8.1 Hz, 1H, Hₐᵣₒₘ), 6.50 (d, J=2.3 Hz, 1H, Hₐᵣₒₘ), 6.39 (dd, J=8.3, 2.3 Hz, 1H, Hₐᵣₒₘ), 4.58 (d, J=1.9 Hz, 2H, N1-CH₂), 4.01-3.92 (m, 2H, N3-CH₂), 3.77 (s, 3H, OCH₃), 3.71 (s, 3H, OCH₃), 3.03 (t, J=2.3 Hz, 1H, H_{propargyl}), 2.93-2.83 (m, 4H, C8-CH₂CH₂), 1.77-1.68 (m, 2H, H_{alkyl}), 1.57-1.48 (m, 2H, H_{alkyl}), 1.40 (dt, J=14.5, 6.7 Hz, 2H, H_{alkyl}). ¹³C-NMR (151 MHz, DMSO-d₆) δ [ppm]=163.8 (Cₐᵣₒₘ—OCH₃), 160.0 (Cₐᵣₒₘ—OCH₃), 158.9 (Cₓₐₙₜₕᵢₙₑ), 157.9 (Cₓₐₙₜₕᵢₙₑ), 154.6 (Cₓₐₙₜₕᵢₙₑ), 149.9 (Cₓₐₙₜₕᵢₙₑ), 129.8 (Cₐᵣₒₘ), 121.3 (Cₐᵣₒₘ), 105.4 (C5) 104.3 (Cₐᵣₒₘ), 98.3 (Cₐᵣₒₘ), 80.4 (C_{propargyl}), 72.2 (C_{propargyl}), 55.2 (OCH₃), 55.03 (OCH₃), 43.5 (N3-CH₂), 32.1 (—CH₂) 29.8 (N1-CH₂), 29.4 (—CH₂), 27.7 (PCH₂CH₂), 21.0 (PCH₂), 16.7 (P(CH₂)₂CH₂), 15.8 (POCH₂CH₃). ³¹P-NMR (243 MHz, DMSO-d₆) δ [ppm]=26.9. HRMS (ESI-QTOF) calculated for C₂₂H₂₇N₄O₇P [M+H]⁺: 491.1690; found: 491.1690.

B-11) (4-(8-(3,4-Dimethoxyphenethyl)-2,6-dioxo-1-(prop-2-yn-1-yl)-1,2,6,7-tetrahydro-3H-purin-3-yl)butyl)phosphonic acid: White solid; m.p. 254-256° C. ¹H-NMR (600 MHz, DMSO-d₆) δ [ppm]=7.02 (d, J=8.1 Hz, 1H, Hₐᵣₒₘ), 6.51 (d, J=2.3 Hz, 1H, Hₐᵣₒₘ), 6.38 (dd, J=8.3, 2.3 Hz, 1H, Hₐᵣₒₘ), 4.58 (d, J=1.9 Hz, 2H, N1-CH₂), 4.02-3.93 (m, 2H, N3-CH₂), 3.77 (s, 3H, OCH₃), 3.71 (s, 3H, OCH₃), 3.02 (t, J=2.3 Hz, 1H, H_{propargyl}), 2.93-2.83 (m, 4H, C8-CH₂CH₂), 1.77-1.68 (m, 2H, H_{alkyl}), 1.57-1.48 (m, 2H, H_{alkyl}), 1.40 (dt, J=14.5, 6.7 Hz, 2H, H_{alkyl}). ¹³C-NMR (151 MHz, DMSO-d₆) δ [ppm]=163.6 (Cₐᵣₒₘ—OCH₃), 160.2 (Cₐᵣₒₘ—OCH₃), 158.9 (Cₓₐₙₜₕᵢₙₑ), 157.9 (Cₓₐₙₜₕᵢₙₑ), 154.6 (Cₓₐₙₜₕᵢₙₑ), 149.9 (Cₓₐₙₜₕᵢₙₑ), 129.8 (Cₐᵣₒₘ), 121.3 (Cₐᵣₒₘ), 105.4 (C5) 104.3 (Cₐᵣₒₘ), 98.3 (Cₐᵣₒₘ), 80.4 (C_{propargyl}), 72.2 (C_{propargyl}), 55.2 (OCH₃), 55.03 (OCH₃), 43.5 (N3-CH₂), 32.1 (—CH₂) 29.7 (N1-CH₂), 29.4 (—CH₂), 27.7 (PCH₂CH₂), 21.0 (PCH₂), 16.7 (P(CH₂)₂CH₂), 15.8 (POCH₂CH₃). ³¹P-NMR (243 MHz, DMSO-d₆) δ [ppm]=26.7. HRMS (ESI-QTOF) calculated for C₂₂H₂₇N₄O₇P [M+H]⁺: 491.1690; found: 491.1690.

B-12) (4-(1-Ethyl-8-(3-methoxyphenethyl)-2,6-dioxo-1,2,6,7-tetrahydro-3H-purin-3-yl)butyl)phosphonic acid: White solid; m.p. 243-245° C. ¹H-NMR (600 MHz, DMSO-d₆) δ [ppm]=7.15 (t, J=7.8 Hz, 1H, Hₐᵣₒₘ), 6.78-6.75 (m, 2H, Hₐᵣₒₘ), 6.73-6.69 (m, 1H, Hₐᵣₒₘ), 3.93 (t, J=7.6 Hz, 2H, N1-CH₂), 3.89-3.86 (m, 2H, N3-CH₂), 3.69 (s, 3H, OCH₃), 2.96 (s, 4H, C8-CH₂CH₂), 1.69 (dq, J=15.2, 7.9, 6.9 Hz, 2H, H_{alkyl}), 1.51-1.44 (m, 2H, H_{alkyl}), 1.40 (dt, J=15.8, 7.4 Hz, 2H, H_{alkyl}), 1.07 (t, J=7.0 Hz, 3H, CH₂CH₃). ¹³C-NMR (151 MHz, DMSO-d₆) δ [ppm]=159.4 (Cₐᵣₒₘ—OCH₃), 154.0 (Cₓₐₙₜₕᵢₙₑ), 153.3 (Cₓₐₙₜₕᵢₙₑ), 150.5 (Cₓₐₙₜₕᵢₙₑ), 147.0 (Cₓₐₙₜₕᵢₙₑ), 142.3 (Cₐᵣₒₘ), 129.4 (Cₐᵣₒₘ), 120.7 (Cₐᵣₒₘ), 114.0 (Cₐᵣₒₘ), 111.8 (Cₐᵣₒₘ), 106.7 (C5), 55.0 (OCH₃), 43.0 (N3-CH₂), 35.6 (CH₂) 33.5 (CH₂), 30.1 (N1-CH₂), 29.0 (d, ²J_{C,P}=15.1 Hz, 1C, PCH₂CH₂), 28.8 (d, ¹J_{C,P}=135.7 Hz, 1C, PCH₂), 21.1 (d, ³J_{C,P}=4.1 Hz, 1C, PCH₂CH₂CH₂), 13.3 (N1-CH₂CH₃). ³¹P-NMR (243 MHz, DMSO-d₆) δ [ppm]=23.8. HRMS (ESI-QTOF) calculated for C₂₀H₂₇N₄O₆P [M+H]⁺: 451.1741; found: 451.1743.

B-13) (4-(1-(Cyclobutylmethyl)-8-(3-methoxyphenethyl)-2,6-dioxo-1,2,6,7-tetrahydro-3H-purin-3-yl)butyl)phosphonic acid: White solid; m.p. 205-207° C. ¹H-NMR (600 MHz, DMSO-d₆) δ [ppm]=13.16 (br s, 1H, N7-H), 7.17 (t, J=8.0 Hz, 1H, Hₐᵣₒₘ), 6.77 (d, J=6.0 Hz, 2H, Hₐᵣₒₘ), 6.74-6.72 (m, 1H, Hₐᵣₒₘ), 3.95 (d, J=7.1 Hz, 2H, N1-CH₂), 3.92 (d, J=7.2 Hz, 2H, N3-CH₂), 3.69 (s, 3H, OCH₃), 2.98 (s, 4H, C8-CH₂CH₂), 2.62 (dt, J=15.0, 7.5 Hz, 1H, H_{alkyl}), 1.91-1.86 (m, 2H, H_{alkyl}), 1.72-1.76 (m, 6H, H_{alkyl}), 1.55 (dt, J=16.8, 7.8 Hz, 2H, H_{alkyl}), 1.50-1.42 (m, 2H, H_{alkyl}). ¹³C-NMR (151 MHz, DMSO-d₆) δ [ppm]=159.4 (Cₐᵣₒₘ—OCH₃), 154.2 (Cₓₐₙₜₕᵢₙₑ), 153.8 (Cₓₐₙₜₕᵢₙₑ), 151.0 (Cₓₐₙₜₕᵢₙₑ), 147.8 (Cₓₐₙₜₕᵢₙₑ), 142.2 (Cₐᵣₒₘ), 129.5 (Cₐᵣₒₘ), 120.7 (Cₐᵣₒₘ), 114.0 (Cₐᵣₒₘ), 111.8 (Cₐᵣₒₘ), 106.2 (C5), 55.0 (OCH₃), 45.2 (N1-CH₂), 42.6 (N3-CH₂), 34.2 (C_{alkyl}), 33.5 (C_{alkyl}), 30.1 (C_{alkyl}), 28.7 (d, ²J_{C,P}=16.2 Hz, 1C, PCH₂CH₂), 27.4 (d, ¹J_{C,P}=136.5 Hz, 1C, PCH₂), 25.7 (2C, C_{cyclobutyle}), 20.2 (d, ³J_{C,P}=3.9 Hz, 1C, P(CH₂)₂CH₂), 17.9 (C_{cyclobutyle}). ³¹P-NMR (243 MHz, DMSO-d₆) δ [ppm]=27.3. HRMS (ESI-QTOF) calculated for C₂₃H₃₁N₄O₆P [M+H]⁺: 491.2054; found: 491.2068.

B-14) (4-(1-Ethyl-8-(4-fluorophenethyl)-2,6-dioxo-1,2,6,7-tetrahydro-3H-purin-3-yl)butyl)phosphonic acid: White solid; m.p. 256-260° C. ¹H-NMR (600 MHz, DMSO-d₆) δ [ppm]=13.19 (br s, 1H, N7-H), 7.26-7.18 (m, 2H, Hₐᵣₒₘ), 7.10-7.03 (m, 2H, Hₐᵣₒₘ), 3.95-3.91 (m, 2H, N1-CH₂), 3.90-3.85 (m, 2H, N3-CH₂), 3.00-2.94 (m, 4H, C8-CH₂CH₂), 1.73-1.66 (m, 2H, H_{alkyl}), 1.52-1.42 (m, 4H, H_{alkyl}), 1.08 (t, J=5.8 Hz, 3H, CH₂CH₃). ¹³C-NMR (151 MHz, DMSO-d₆) δ [ppm]=161.2 (d, 1 J=245 Hz, 1C, Cₐᵣₒₘ—F), 154.1 (Cₓₐₙₜₕᵢₙₑ), 153.8 (Cₓₐₙₜₕᵢₙₑ), 150.8 (Cₓₐₙₜₕᵢₙₑ), 137.0 (d, J_{C,F}=3.0 Hz, 1C, Cₐᵣₒₘ), 130.6 (d, ³J_{C,F}=7.8 Hz, 2C, Cₐᵣₒₘ), 115.5 (d, ²J_{C,F}=20.6 Hz, 2C, Cₐᵣₒₘ), 106.7 (C5), 43.0 (N3-CH₂), 36.1 (CH₂), 32.9 (CH₂), 30.5 (N1-CH₂), 29.1 (d, ²J_{C,P}=16.0 Hz, 1C, P—CH₂CH₂), 28.0 (d, ¹J_{C,P}=135.5 Hz, 1C, PCH₂), 20.7 (d, ³J_{C,P}=4.5 Hz, 1C, PCH₂CH₂CH₂), 13.6 (N1-CH₂CH₃). ³¹P-NMR (243 MHz, DMSO-d₆) δ [ppm]=26.3. HRMS (ESI-QTOF) calculated for C₁₉H₂₄FN₄O₅P [M+H]⁺: 439.1541; found: 439.1543.

B-15) (4-(8-(3-Methoxyphenethyl)-7-methyl-2,6-dioxo-1-(prop-2-yn-1-yl)-1,2,6,7-tetrahydro-3H-purin-3-yl)butyl)phosphonic acid: white solid; m.p. n.d.; ¹H-NMR (600 MHz, DMSO-d₆) δ [ppm]=6.82-6.79 (m, 2H, Hₐᵣₒₘ), 6.76-6.74 (m, 1H, Hₐᵣₒₘ), 6.66-6.62 (m, 1H, Hₐᵣₒₘ), 4.58 (d, 2H, N1-CH₂), 3.97 (t, J=7.0 Hz, 2H, N3-CH₂), 3.71 (s, 3H, OCH₃ or NCH₃), 3.70 (s, 3H, OCH₃ or NCH₃), 3.06-3.04 (m, 2H, C8-CH₂), 3.04-3.02 (m, 1H, H_{propargyl}), 2.99-2.96 (m, 2H, C8-CH₂CH₂), 1.74-1.69 (m, 2H, H_{alkyl}), 1.58-1.52 (m, 2H, H_{alkyl}), 1.51-1.45 (m, 2H, H_{alkyl}). ¹³C-NMR (151 MHz, DMSO-d₆) δ [ppm]=171.6 (COCH₃), 159.4 (C8), 154.3 (C2 or C6), 153.4 (C2 or C6), 149.9 (C4), 147.5 (Cₐᵣₒₘ), 142.1 (Cₐᵣₒₘ), 129.5 (Cₐᵣₒₘ), 120.8 (Cₐᵣₒₘ), 114.2 (Cₐᵣₒₘ), 111.9 (Cₐᵣₒₘ), 106.4 (C5), 79.8 (C_{propargyl}), 72.9 (C_{propargyl}), 55.0 (OCH₃), 42.5 (N3-CH₂), 32.8 (CH₂), 31.4 (CH₂), 30.0 (N1-CH₂), 28.7 (d, ²J_{C,P}=14.9 Hz, 1C, PCH₂CH₂), 27.9 (N7-CH₃) 27.4 (d, ¹J_{C,P}=137.2 Hz, 1C, PCH₂), 20.2 (d, ³J_{C,P}=3.9 Hz, 1C, PCH₂CH₂CH₂). ³¹P-NMR (243 MHz, DMSO-d₆) δ [ppm]=27.0. HRMS (ESI-QTOF) calculated for C₂₂H₂₈N₄O₆P [M+H]⁺: 475.4618; found: 475.4620.

B-16) (4-(7-Methyl-2,6-dioxo-1-(prop-2-yn-1-yl)-8-(3-(trifluoromethyl)phenethyl)-1,2,6,7-tetrahydro-3H-purin-3-yl)butyl)phosphonic acid: White solid; m.p. n.d. ° C.; ¹H-NMR (600 MHz, DMSO-d₆) δ [ppm]=7.63 (s, 1H, Hₐᵣₒₘ), 7.58 (d, J=7.5 Hz, 1H, Hₐᵣₒₘ), 7.54 (d, J=7.8 Hz, 1H, Hₐᵣₒₘ), 7.50 (t, J=7.6 Hz, 1H, Hₐᵣₒₘ), 4.58 (d, J=2.3 Hz, 2H, N1-CH₂), 3.95 (t, J=7.1 Hz, 2H, N3-CH₂), 3.76 (s, 3H, N7-CH₃), 3.11 (d, J=3.4 Hz, 4H, C8-CH₂CH₂), 3.05 (t, J=2.4 Hz, 1H, H_{propargyl}), 1.71 (p, J=7.4 Hz, 2H, C_{alkyl}), 1.58-1.52 (m, 2H, C_{alkyl}), 1.50-1.43 (m, 2H, C_{alkyl}). ¹³C-NMR (151 MHz, DMSO-d₆) δ [ppm]=154.0 (Cₓₐₙₜₕᵢₙₑ), 153.5 (Cₓₐₙₜₕᵢₙₑ), 150.0 (Cₓₐₙₜₕᵢₙₑ), 147.5 (C4), 142.1 (Cₐᵣₒₘ), 133.1 (Cₐᵣₒₘ), 129.5 (Cₐᵣₒₘ), 129.2 (q, ²J_{C,F}=32.9 Hz, 1C, CCF₃), 125.4 (q, ³J_{C,F}=3.4 Hz, 1C, CHCF₃), 124.5 (q, J_{C,F}=272.0 Hz, 1C, CF₃), 123.1 (q, ³J_{C,F}=3.7 Hz, 1C, CHCF₃), 106.5 (C5), 79.9 (C_{propargyl}), 72.9 (C_{propargyl}), 42.5 (N1-CH₂), 32.2 (C_{alkyl}), 31.6 (C_{alkyl}), 30.1 (C_{alkyl}), 28.7 (d, ²J_{C,P}=16.5 Hz, 1C, PCH₂), 27.6 (N7-CH₃), 27.4 (d, ¹J_{C,P}=136.6 Hz, 1C, PCH₂), 20.2 (d, ³J_{C,P}=4.7 Hz, 1C, PCH₂CH₂CH₂). ³¹P-NMR (243 MHz, DMSO-d₆) δ [ppm]=27.3. HRMS (ESI-QTOF) calculated for C₂₂H₂₅F₃N₄O₅P [M+H]⁺: 513.1509; found: 513.1491.

B-17) (4-(8-(2-Chlorophenethyl)-7-methyl-2,6-dioxo-1-(prop-2-yn-1-yl)-1,2,6,7-tetrahydro-3H-purin-3-yl)butyl)phosphonic acid: White solid; m.p. n.d. ° C.; ¹H-NMR (600 MHz, DMSO-d₆) δ [ppm]=7.42 (dd, J=6.6, 2.5 Hz, 1H), 7.36 (dd, J=6.7, 2.7 Hz, 1H), 7.28-7.23 (m, 2H), 4.59 (d, J=2.7 Hz, 2H), 3.96 (t, J=7.1 Hz, 2H), 3.74 (s, 3H), 3.13 (t, J=7.4 Hz, 2H), 3.10-3.03 (m, 3H), 1.71 (p, J=7.4 Hz, 2H), 1.59-1.50 (m, 2H), 1.51-1.43 (m, 2H). ¹³C-NMR (151 MHz, DMSO-d₆) δ [ppm]=153.6 (C8 or C4), 153.3 (C8 or C4), 149.7 (CO), 147.3 (CO), 137.6 (Cₐᵣₒₘ), 133.0 (Cₐᵣₒₘ), 131.0 (Cₐᵣₒₘ), 129.2 (Cₐᵣₒₘ), 128.3 (Cₐᵣₒₘ), 127.3 (Cₐᵣₒₘ), 106.4 (C5), 79.7 (C_{propargyl}), 72.7 (C_{propargyl}), 42.4 (—CH₂), 31.3 (—CH₂), 30.4 (—CH₂), 29.9 (—CH₂), 28.5 (d, ²J_{C,P}=15.6 Hz, 1C, PCH₂CH₂), 27.3 (d, ¹J_{C,P}=136.5 Hz, 1C, PCH₂), 26.1 (—CH₂), 20.0 (d, ³J_{C,P}=3.6 Hz, PCH₂CH₂CH₂). HRMS (ESI-QTOF) n.d.

B-18) (4-(8-(2-Bromophenethyl)-7-methyl-2,6-dioxo-1-(prop-2-yn-1-yl)-1,2,6,7-tetrahydro-3H-purin-3-yl)butyl)phosphonic acid: White solid; m.p. n.d.; ¹H-NMR (600 MHz, DMSO-d₆) δ [ppm]=7.59 (dd, J=7.9, 1.2 Hz, 1H, Hₐᵣₒₘ), 7.35 (dd, J=7.7, 1.8 Hz, 1H, Hₐᵣₒₘ), 7.31 (td, J=7.4, 1.3 Hz, 1H, Hₐᵣₒₘ), 7.17 (td, J=7.6, 1.8 Hz, 1H, Hₐᵣₒₘ), 4.59 (d, J=2.5 Hz, 2H, N1-CH₂), 3.96 (t, J=7.1 Hz, 2H, N3-CH₂), 3.74 (s, 3H, N7-CH₃), 3.13 (t, J=7.1 Hz, 2H, —CH₂), 3.10-3.02 (m, 3H, —CH₂ and H_{propargyl}), 1.76-1.67 (m, 2H, —CH₂), 1.59-1.51 (m, 2H, —CH₂), 1.51-1.43 (m, 2H, —CH₂). ¹³C-NMR (151 MHz, DMSO-d₆) δ [ppm]=153.5 (C8 or C4), 153.3 (C8 or C4), 149.8 (CO), 147.3 (CO), 139.3 (Cₐᵣₒₘ), 132.5 (Cₐᵣₒₘ), 131.0 (Cₐᵣₒₘ), 128.6 (Cₐᵣₒₘ), 127.9 (Cₐᵣₒₘ), 123.7 (Cₐᵣₒₘ), 106.4 (C5), 79.7 (C_{propargyl}), 72.7 (C_{propargyl}), 42.4 (N—CH₂), 33.0 (N—CH₂), 31.3 (N7-CH₃), 29.9 (CH₂), 28.5 (d, ²J_{C,P}=15.6 Hz, 1C, PCH₂CH₂), 27.2 (d, ¹J_{C,P}=137.5 Hz, 1C, PCH₂), 26.3 (CH₂), 20.0 (d, ³J_{C,P}=4.4 Hz, PCH₂CH₂CH₂). ³¹P-NMR (243 MHz, DMSO-d₆) δ [ppm]=27.1.

B-19) (4-(8-(2-Bromophenethyl)-7-ethyl-2,6-dioxo-1-(prop-2-yn-1-yl)-1,2,6,7-tetrahydro-3H-purin-3-yl)butyl)phosphonic acid: White solid; m.p. 192-195° C.; ¹H-NMR (600 MHz, DMSO-d₆) δ [ppm]=7.59 (d, J=7.9 Hz, 1H, Hₐᵣₒₘ), 7.36 (d, J=7.5 Hz, 1H, Hₐᵣₒₘ), 7.30 (t, J=7.4 Hz, 1H, Hₐᵣₒₘ), 7.16 (t, J=7.5 Hz, 1H, Hₐᵣₒₘ), 4.59 (d, J=2.5 Hz, 2H, N1-CH₂), 4.18 (q, J=7.1 Hz, 2H, N7-CH₂), 3.96 (t, J=7.2 Hz, 2H, N3-CH₂), 3.15 (t, J=7.6 Hz, 2H, —CH₂), 3.09-3.05 (m, 3H, —CH₂ and H_{propargyl}), 1.72 (p, J=7.4 Hz, 2H, —CH₂), 1.58-1.44 (m, 4H, —CH₂CH₂), 1.22 (t, J=7.1 Hz, 3H, N7-CH₂CH₃). ¹³C-NMR (151 MHz, DMSO-d₆) δ [ppm]=152.9 (C8 or C4), 152.7 (C8 or C4), 149.8 (CO), 147.7 (CO), 139.3 (Cₐᵣₒₘ), 132.5 (Cₐᵣₒₘ), 131.1 (Cₐᵣₒₘ), 128.6 (Cₐᵣₒₘ), 127.9 (Cₐᵣₒₘ), 123.8 (Cₐᵣₒₘ), 105.5 (C5), 79.7 (C_{propargyl}), 72.8 (C_{propargyl}), 42.5 (N—CH₂), 39.5 (N—CH₂), 33.3 (N—CH₂), 30.0 (—CH₂), 28.6 (d, ²J_{C,P}=15.7 Hz, 1C, PCH₂CH₂), 27.5 (d, ¹J_{C,P}=136.1 Hz, 1C, PCH₂), 26.1 (—CH₂), 20.2 (d, ³J_{C,P}=4.2 Hz, PCH₂CH₂CH₂), 15.9 (N7-CH₂CH₃). ³¹P-NMR (243 MHz, DMSO-d₆): δ [ppm]=26.6. HRMS (ESI-QTOF) calculated for C₂₂H₂₆BrN₄O₅P [M+H]⁺: 537.0897; found: 537.0888.

B-20) (4-(8-(2-Bromophenethyl)-7-(2-hydroxyethyl)-2,6-dioxo-1-(prop-2-yn-1-yl)-1,2,6,7-tetrahydro-3H-purin-3-yl)butyl)phosphonic acid: White solid; m.p. 157-160° C.; ¹H-NMR (600 MHz, DMSO-d₆) δ [ppm]=7.59 (d, J=7.7 Hz, 1H, Hₐᵣₒₘ), 7.38 (dd, J=7.6, 1.7 Hz, 1H, Hₐᵣₒₘ), 7.33-7.28 (m, 1H, Hₐᵣₒₘ), 7.17 (td, J=7.7, 1.8 Hz, 1H, Hₐᵣₒₘ), 4.59 (d, J=2.5 Hz, 2H, N1-CH₂), 4.19 (t, J=5.3 Hz, 2H, N—CH₂), 3.98 (t, J=7.2 Hz, 2H, N—CH₂), 3.67-3.66 (m, 1H, —CH₂OH), 3.18-3.13 (m, 2H, —CH₂), 3.14-3.09 (m, 2H, —CH₂), 3.06 (q, J=2.6 Hz, 1H, H_{propargyl}), 1.73 (p, J=7.3 Hz, 2H, —CH₂), 1.61-1.53 (m, 2H, —CH₂), 1.53-1.45 (m, 2H, —CH₂). ¹³C-NMR (151 MHz, DMSO-d₆) δ [ppm]=154.3 (C8 or C4), 153.0 (C8 or C4), 149.8 (CO), 147.7 (CO), 139.6 (Cₐᵣₒₘ), 132.5 (Cₐᵣₒₘ), 131.0 (Cₐᵣₒₘ), 128.5 (Cₐᵣₒₘ), 127.9 (Cₐᵣₒₘ), 123.8 (Cₐᵣₒₘ), 105.7 (C5), 79.7 (C_{propargyl}), 72.8 (C_{propargyl}), 60.1 (—CH₂OH), 47.3 (N7-CH₂), 42.4 (N—CH₂), 33.1 (N—CH₂), 30.0 (—CH₂), 28.5 (d, ²J_{C,P}=15.8 Hz, 1C, PCH₂CH₂), 27.2 (d, ¹J_{C,P}=136.5 Hz, 1C, PCH₂), 26.5 (—CH₂), 20.0 (d, ³J_{C,P}=4.1 Hz, PCH₂CH₂CH₂). ³¹P-NMR (243 MHz, DMSO-d₆): δ [ppm]=27.2. HRMS (ESI-QTOF) calculated for C₂₂H₂₆BrN₄O₆P [M+H]⁺: 553.0846; found: 553.0829.

B-21) (4-(8-(2-Bromophenethyl)-7-(cyclopropylmethyl)-2,6-dioxo-1-(prop-2-yn-1-yl)-1,2,6,7-tetrahydro-3H-purin-3-yl)butyl)phosphonic acid: White solid; m.p. 181-184° C.; ¹H-NMR (600 MHz, DMSO-d₆) δ [ppm]=7.59 (dd, J=8.1, 1.2 Hz, 1H, Hₐᵣₒₘ), 7.39 (dd, J=7.7, 1.7 Hz, 1H, Hₐᵣₒₘ), 7.31 (td, J=7.4, 1.2 Hz, 1H, Hₐᵣₒₘ), 7.16 (td, J=7.6, 1.7 Hz, 1H, Hₐᵣₒₘ), 4.59 (d, J=2.4 Hz, 2H, N1-CH₂), 4.08 (d, J=7.1 Hz, 2H, N7-CH₂), 3.98 (t, J=7.2 Hz, 2H, N3-CH₂), 3.18 (t, J=7.5 Hz, 2H, —CH₂), 3.10 (dd, J=8.0, 6.6 Hz, 2H, —CH₂), 3.06 (t, J=2.4 Hz, 1H, H_{propargyl}), 1.74 (p, J=7.4 Hz, 2H, —CH₂), 1.61-1.46 (m, 4H, —CH₂CH₂), 1.23-1.14 (m, 1H, N7-CH₂CH), 0.45-0.41 (m, 2H, —CH_{2 cyclopropyl}), 0.40-0.36 (m, 2H, —CH_{2 cyclopropyl}). ¹³C-NMR (151 MHz, DMSO-d₆) δ [ppm]=153.1 (C8 or C4), 153.0 (C8 or C4), 149.8 (CO), 147.7 (CO), 139.4 (Cₐᵣₒₘ), 132.5 (Cₐᵣₒₘ), 131.1 (Cₐᵣₒₘ), 128.6 (Cₐᵣₒₘ), 127.9 (Cₐᵣₒₘ), 123.8 (Cₐᵣₒₘ), 105.8 (C5), 79.7 (C_{propargyl}), 72.8 (C_{propargyl}), 48.3 (N—CH₂), 42.5 (N—CH₂), 33.1 (N—CH₂), 30.0 (—CH₂), 28.6 (d, ²J_{C,P}=15.7 Hz, 1C, PCH₂CH₂), 27.3 (d, ¹J_{C,P}=136.7 Hz, 1C, PCH₂), 26.3 (—CH₂), 20.1 (d, ³J_{C,P}=4.0 Hz, PCH₂CH₂CH₂), 11.7 (—CH_{cyclopropyl}), 3.3 (—CH_{2 cyclopropyl}). ³¹P-NMR (243 MHz, DMSO-d₆) δ [ppm]=27.2. HRMS (ESI-QTOF) calculated for C₂₄H₂₉BrN₄O₅P [M+H]⁺: 563.1053; found: 563.1038.

B-22) (4-(8-(2-Bromophenethyl)-2,6-dioxo-1-(prop-2-yn-1-yl)-7-propyl-1,2,6,7-tetrahydro-3H-purin-3-yl)butyl)phosphonic acid: White solid; m.p. 178-181° C.; ¹H-NMR (600 MHz, DMSO-d₆) δ [ppm]=7.59 (d, J=7.9 Hz, 1H, Hₐᵣₒₘ), 7.37 (d, J=7.6 Hz, 1H, Hₐᵣₒₘ), 7.30 (t, J=7.4 Hz, 1H, Hₐᵣₒₘ), 7.16 (t, J=7.5 Hz, 1H, Hₐᵣₒₘ), 4.59 (d, J=2.4 Hz, 2H, N1-CH₂), 4.09 (t, J=7.4 Hz, 2H, N—CH₂), 3.98 (t, J=7.1 Hz, 2H, N—CH₂), 3.20-3.14 (m, 2H, —CH₂), 3.10-3.04 (m, 3H, —CH₂ and H_{propargyl}), 1.74 (p, J=7.3 Hz, 2H, —CH₂), 1.66-1.53 (m, 4H, 2×—CH₂), 1.53-1.45 (m, 2H, —CH₂), 0.81 (t, J=7.4 Hz, 3H, —CH₃). ¹³C-NMR (151 MHz, DMSO-d₆) δ [ppm]=153.0 (C8 or C4), 152.9 (C8 or C4), 149.8 (CO), 147.7 (CO), 139.3 (Cₐᵣₒₘ), 132.5 (Cₐᵣₒₘ), 131.1 (Cₐᵣₒₘ), 128.6 (Cₐᵣₒₘ), 127.9 (Cₐᵣₒₘ), 123.8 (Cₐᵣₒₘ), 105.8 (C5), 79.7 (C_{propargyl}), 72.8 (C_{propargyl}), 46.0 (N—CH₂), 42.4 (N—CH₂), 33.3 (N—CH₂), 30.0 (—CH₂), 28.5 (d, ²J_{C,P}=15.8 Hz, 1C, PCH₂CH₂), 27.3 (d, ¹J_{C,P}=136.8 Hz, 1C, PCH₂), 26.2 (—CH₂), 23.6 (—CH₂), 20.1 (d, ³J_{C,P}=4.1 Hz, PCH₂CH₂CH₂), 10.6 (—CH₃). ³¹P-NMR (243 MHz, DMSO-d₆): δ [ppm]=27.2. HRMS (ESI-QTOF) calculated for C₂₃H₂₉BrN₄O₅P [M+H]⁺: 551.1053; found: 553.1038.

B-23) (4-(7-Benzyl-8-(2-bromophenethyl)-2,6-dioxo-1-(prop-2-yn-1-yl)-1,2,6,7-tetrahydro-3H-purin-3-yl)butyl)phosphonic acid: White solid; m.p. 126-129° C.; ¹H-NMR (600 MHz, DMSO-d₆) δ [ppm]=7.55 (d, J=7.9 Hz, 1H), 7.32 (t, J=7.4 Hz, 2H), 7.29-7.25 (m, 3H), 7.17-7.10 (m, 3H), 5.50 (s, 2H), 4.59 (d, J=2.5 Hz, 2H), 4.00 (t, J=7.2 Hz, 2H), 3.08-3.04 (m, 3H), 3.01 (dd, J=8.2, 5.8 Hz, 2H), 1.75 (p, J=7.4 Hz, 2H), 1.62-1.46 (m, 4H). ¹³C-NMR (151 MHz, DMSO-d₆) δ [ppm]=153.6 (C8 or C4), 153.3 (C8 or C4), 149.8 (CO), 147.7 (CO), 139.2 (Cₐᵣₒₘ), 136.4 (Cₐᵣₒₘ), 132.5 (Cₐᵣₒₘ), 130.9 (Cₐᵣₒₘ), 128.8 (2C, Cₐᵣₒₘ), 128.6 (Cₐᵣₒₘ), 127.9 (Cₐᵣₒₘ), 127.7 (Cₐᵣₒₘ), 126.6 (2C, Cₐᵣₒₘ), 123.7 (Cₐᵣₒₘ), 106.0 (C5), 79.6 (C_{propargyl}), 72.8 (C_{propargyl}), 47.2 (N7-CH₂), 42.5 (N—CH₂), 32.9 (N—CH₂), 30.0 (—CH₂), 28.5 (d, ²J_{C,P}=15.5 Hz, 1C, PCH₂CH₂), 27.2 (d, ¹J_{C,P}=137.1 Hz, 1C, PCH₂), 26.3 (—CH₂), 20.1 (d, ³J_{C,P}=4.2 Hz, PCH₂CH₂CH₂). ³¹P-NMR (243 MHz, DMSO-d₆): δ [ppm]=27.2. HRMS (ESI-QTOF) calculated for C27H₂₈BrN₄O₅P [M+H]⁺: 599.1053; found: 5991051.

C-1) Diethyl (4-(8-(2-Methoxyphenethyl)-2,6-dioxo-1-(prop-2-yn-1-yl)-1,2,6,7-tetrahydro-3H-purin-3-yl)butyl)phosphonate: White solid; m.p. 104-106° C. ¹H-NMR (500 MHz, DMSO-d₆) δ [ppm]=13.26 (s, 1H, N7-H), 7.18 (td, J=7.8, 1.7 Hz, 1H, Hₐᵣₒₘ), 7.09 (dd, J=7.5, 1.7 Hz, 1H, Hₐᵣₒₘ), 6.96 (dd, J=8.3, 1.0 Hz, 1H, Hₐᵣₒₘ), 6.83 (td, J=7.4, 1.1 Hz, 1H, Hₐᵣₒₘ), 4.59 (d, J=2.5 Hz, 2H, N1-CH₂), 4.00 (t, J=6.8 Hz, 2H, N3-CH₂), 3.98-3.90 (m, 4H, POCH₂), 3.78 (s, 3H, OCH₃), 3.05 (t, J=2.4 Hz, 1H, H_{propargyl}), 3.01-2.91 (m, 4H, CH₂CH₂), 1.83-1.73 (m, 4H, CH₂CH₂), 1.48 (m, 2H, CH₂), 1.19 (t, J=7.0 Hz, 6H, POCH₂CH₃). ¹³C-NMR (151 MHz, DMSO-d₆) δ [ppm]=157.1 (Cₐᵣₒₘ—OCH₃), 154.4 (C8), 152.9 (CO), 150.0 (CO), 148.1 (C4), 129.5 (Cₐᵣₒₘ), 128.1 (Cₐᵣₒₘ), 127.6 (Cₐᵣₒₘ), 120.2 (Cₐᵣₒₘ), 110.6 (Cₐᵣₒₘ), 105.9 (C5), 79.7 (C_{propargyl}), 72.6 (C_{propargyl}), 60.7 (d, ²J_{C,P}=6.7 Hz, 1C, POCH₂), 55.2 (OCH₃), 42.4 (N3-CH₂), 30.0 (N1-CH₂), 28.4 (CH₂), 28.3 (CH₂), 28.2 (d, ²J_{C,P}=15.1 Hz, 1C, PCH₂CH₂), 24.1 (d, ¹J_{C,P}=138.8 Hz, 1C, PCH₂), 19.3 (d, ³J_{C,P}=5.2 Hz, 1C, P(CH₂)₂CH₂), 16.2 (d, ³J_{C,P}=5.6 Hz, 2C, POCH₂CH₃). ³¹P-NMR (243 MHz, DMSO-d₆) δ [ppm]=32.8. HRMS (ESI-QTOF) calculated for C₂₅H₃₃N₄O₆P [M+H]⁺: 517.2210; found: 517.2213.

B-24) (4-(8-(2-Fluorophenethyl)-2,6-dioxo-1-(prop-2-yn-1-yl)-1,2,6,7-tetrahydro-3H-purin-3-yl)butyl) phosphonic acid: White solid; m.p. 239-241° C. ¹H NMR (600 MHz, DMSO-d₆) δ [ppm]=13.33 (s, 1H, N7-H), 7.29-7.21 (m, 2H, 2Hₐᵣₒₘ), 7.16-7.07 (m, 2H, 2Hₐᵣₒₘ), 4.59 (d, J=2.5 Hz, 2H, N1-CH₂), 3.97 (t, J=7.2 Hz, 2H, N3-CH₂), 3.06 (t, J=2.4 Hz, 1H, H_{propargyl}), 3.08-3.03 (m, 2H, CH₂), 3.01-2.96 (m, 2H, CH₂), 1.73 (p, J=7.3 Hz, 2H, CH₂), 1.56 (dt, J=16.9, 7.7 Hz, 2H, CH₂), 1.48 (qd, J=9.8, 9.0, 4.3 Hz, 2H, CH₂). ¹³C NMR (151 MHz, DMSO-d₆) δ [ppm]=160.5 (d, ¹J_{C,F}=244.2 Hz, Cₐᵣₒₘ), 153.6 (Cₓₐₙₜₕᵢₙₑ), 152.8 (Cₓₐₙₜₕᵢₙₑ), 150.0 (Cₓₐₙₜₕᵢₙₑ), 148.0 (Cₓₐₙₜₕᵢₙₑ), 130.8 (d, J_{C,F}=4.5 Hz, Cₐᵣₒₘ), 128.3 (d, J_{C,F}=8.1 Hz, Cₐᵣₒₘ), 126.9 (d, 2J_{C,F}=15.5 Hz, Cₐᵣₒₘ), 124.4 (d, J_{C,F}=3.3 Hz, Cₐᵣₒₘ), 115.1 (d, 2J_{C,F}=21.7 Hz, Cₐᵣₒₘ), 105.9 (C5), 79.7 (C_{propargyl}), 72.7 (C_{propargyl}), 42.7 (N3-CH₂), 30.1 (CH₂), 28.5 (d, ³J_{C,P}=17.1 Hz. CH₂), 27.3 (d, ¹J_{C,P}=136.5 Hz, PCH₂), 26.7 (2CH₂), 20.0 (d, ²J_{C,P}=4.5 Hz, CH₂). ³¹P NMR (243 MHz, DMSO-d₆) δ [ppm]=27.1. HRMS (ESI-QTOF) calculated for C₂₀H₂₂FN₄O₅P [M+H]⁺: 449.139; found: 449.138.

B-25) (4-(8-(2-Iodophenethyl)-2,6-dioxo-1-(prop-2-yn-1-yl)-1,2,6,7-tetrahydro-3H-purin-3-yl)butyl) phosphonic acid: White solid; m.p. 227-229° C. ¹H NMR (600 MHz, DMSO-d₆) δ [ppm]=13.34 (s, 1H, N7-H), 7.83 (dd, J=7.9, 1.3 Hz, 1H, Hₐᵣₒₘ), 7.31 (td, J=7.4, 1.3 Hz, 1H, Hₐᵣₒₘ), 7.25 (dd, J=7.7, 1.7 Hz, 1H, Hₐᵣₒₘ), 6.96 (td, J=7.6, 1.8 Hz, 1H, Hₐᵣₒₘ), 4.59 (d, J=2.5 Hz, 2H, N1-CH₂), 3.98 (t, J=7.2 Hz, 2H, N3-CH₂), 3.12 (t, J=7.6 Hz, 2H, CH₂), 3.06 (t, J=2.4 Hz, 1H, H_{propargyl}), 3.00 (t, J=7.6 Hz, 2H, CH₂), 1.74 (p, J=7.4 Hz, 2H, CH₂), 1.60-1.53 (m, 2H, CH₂), 1.49 (tt, J=8.9, 4.7 Hz, 2H, CH₂). ¹³C NMR (151 MHz, DMSO-d₆) δ [ppm]=153.4 (Cₓₐₙₜₕᵢₙₑ), 152.9 (Cₓₐₙₜₕᵢₙₑ), 150.0 (Cₓₐₙₜₕᵢₙₑ), 148.1 (Cₓₐₙₜₕᵢₙₑ), 142.7 (Cₐᵣₒₘ), 139.1 (Cₐᵣₒₘ), 129.6 (Cₐᵣₒₘ), 128.5 (Cₐᵣₒₘ), 128.5 (Cₐᵣₒₘ), 105.9 (C5), 100.7 (Cₐᵣₒₘ), 79.7 (C_{propargyl}), 72.7 (C_{propargyl}), 42.7 (N3-CH₂), 38.0 (CH₂), 30.1 (N1-CH₂), 28.6 (d, ³J_{C,P}=16.2 Hz, CH₂), 28.6 (CH₂), 27.3 (d, ¹J_{C,P}=136.2 Hz, PCH₂), 20.1 (d, ²J_{C,P}=4.5 Hz, CH₂). ³¹P NMR (243 MHz, DMSO-d₆) δ [ppm]=27.1. HRMS (ESI-QTOF) calculated for C₂₀H₂₂1N₄O₅P [M+2Na]⁺: 601.0084; found: 601.0091.

B-26) (4-(8-(4-Bromophenethyl)-2,6-dioxo-1-(prop-2-yn-1-yl)-1,2,6,9-tetrahydro-3H-purin-3-yl)butyl) phosphonic acid: White solid; m.p. 206-208° C. ¹H NMR (600 MHz, DMSO-d₆) δ [ppm]=13.29 (s, 1H, N7-H), 7.47-7.43 (m, 2H, 2Hₐᵣₒₘ), 7.19-7.14 (m, 2H, 2Hₐᵣₒₘ), 4.59 (d, J=2.5 Hz, 2H, N1-CH₂), 3.97 (t, J=7.2 Hz, 2H, N3-CH₂), 3.06 (t, J=2.4 Hz, 1H, H_{propargyl}), 3.00 (d, J=3.5 Hz, 4H, 2CH₂), 1.74 (p, J=7.3 Hz, 2H, CH₂), 1.57 (ddd, J=16.7, 9.5, 5.7 Hz, 2H, CH₂), 1.49 (tdd, J=8.8, 6.0, 3.4 Hz, 2H, CH₂). ¹³C NMR (126 MHz, DMSO-d₆) δ [ppm]=153.8 (Cₓₐₙₜₕᵢₙₑ), 152.9 (Cₓₐₙₜₕᵢₙₑ), 150.0 (Cₓₐₙₜₕᵢₙₑ), 148.1 (Cₓₐₙₜₕᵢₙₑ), 139.9 (Cₐᵣₒₘ), 131.2 (2Cₐᵣₒₘ), 130.7 (2Cₐᵣₒₘ), 119.2 (Cₐᵣₒₘ), 105.9 (C5), 79.8 (C_{propargyl}), 72.7 (C_{propargyl}), 42.8 (N3-CH₂), 32.5 (CH₂), 30.1 (N1-CH₂), 29.8 (CH₂), 28.6 (d, J=15.5 Hz, CH₂), 27.3 (d, J=136.2 Hz, PCH₂), 20.0 (d, J=4.6 Hz, CH₂). ³¹P NMR (243 MHz, DMSO-d₆) δ [ppm]=27.2. HRMS (ESI-QTOF) calculated for C₂₀H₂₂BrN₄O₅P [M+H]⁺: 509.0584; found: 509.0589.

B-27) (4-(8-(2-([1,1′-Biphenyl]-4-yl)ethyl)-2,6-dioxo-1-(prop-2-yn-1-yl)-1,2,6,7-tetrahydro-3H-purin-3-yl)butyl)phosphonic acid: White solid; m.p. 212-214° C. ¹H NMR (600 MHz, DMSO-d₆) δ [ppm]=13.33 (s, 1H, N7-H), 7.63 (d, J=7.6 Hz, 2H, 2Hₐᵣₒₘ), 7.57 (d, J=7.8 Hz, 2H, 2Hₐᵣₒₘ), 7.44 (t, J=7.6 Hz, 2H, 2Hₐᵣₒₘ), 7.34 (d, J=7.3 Hz, 1H, Hₐᵣₒₘ), 7.31 (d, J=7.8 Hz, 2H, 2Hₐᵣₒₘ), 4.59 (d, J=2.5 Hz, 2H, N1-CH₂), 3.99 (t, J=7.2 Hz, 2H, N3-CH₂), 3.11-3.00 (m, 5H, H_{propargyl} and 2CH₂), 1.83-1.71 (m, 2H, CH₂), 1.54 (dddd, J=33.3, 18.9, 12.6, 7.8 Hz, 4H, 2CH₂). ¹³C NMR (151 MHz, DMSO-d₆) δ [ppm]=154.0 (Cₓₐₙₜₕᵢₙₑ), 152.9 (Cₓₐₙₜₕᵢₙₑ), 150.0 (Cₓₐₙₜₕᵢₙₑ), 148.1 (Cₓₐₙₜₕᵢₙₑ), 139.9 (Cₐᵣₒₘ), 139.7 (Cₐᵣₒₘ), 138.0 (Cₐᵣₒₘ), 128.9 (2Cₐᵣₒₘ), 128.9 (2Cₐᵣₒₘ), 127.2 (Cₐᵣₒₘ), 126.6 (2Cₐᵣₒₘ), 126.5 (2Cₐᵣₒₘ), 105.8 (C5), 79.7 (C_{propargyl}), 72.7 (C_{propargyl}), 42.8 (N3-CH₂), 34.4, 32.8 (CH₂), 30.1 (N1-CH₂), 29.9 (CH₂), 28.6 (d, ³J_{C,P}=15.7 Hz, CH₂), 27.4 (d, ¹J_{C,P}=136.5 Hz, PCH₂), 20.1 (d, ²J_{C,P}=4.7 Hz, CH₂). ³¹P NMR (243 MHz, DMSO-d₆) δ [ppm]=26.8. HRMS (ESI-QTOF) calculated for C₂₋₆H27N₄O₅P [M+H]⁺: 507.180; found: 507.180.

B-28) (4-(8-(2-Fluorophenethyl)-7-methyl-2,6-dioxo-1-(prop-2-yn-1-yl)-1,2,6,7-tetrahydro-3H-purin-3-yl)butyl)phosphonic acid: White solid; m.p. 217-219° C. ¹H NMR (600 MHz, DMSO-d₆) δ [ppm]=7.33 (td, J=7.7, 1.7 Hz, 1H, Hₐᵣₒₘ), 7.26 (tdd, J=7.4, 5.3, 1.8 Hz, 1H, Hₐᵣₒₘ), 7.16-7.14 (m, 1H, Hₐᵣₒₘ), 7.12 (td, J=7.4, 1.2 Hz, 1H, Hₐᵣₒₘ), 4.58 (d, J=2.4 Hz, 2H, N1-CH₂), 3.96 (t, J=7.2 Hz, 2H, N3-CH₂), 3.76 (s, 3H, N7-CH₃), 3.08-3.03 (m, 5H, H_{propargyl} and 2CH₂), 1.70 (q, J=7.4 Hz, 2H, CH₂), 1.59-1.52 (m, 2H, CH₂), 1.51-1.42 (m, 2H, CH₂). ¹³C NMR (151 MHz, DMSO-d₆) δ [ppm]=160.6 (d, ¹J_{C,F}=243.4 Hz, Cₐᵣₒₘ), 153.7 (Cₓₐₙₜₕᵢₙₑ), 153.3 (Cₓₐₙₜₕᵢₙₑ), 149.8 (Cₓₐₙₜₕᵢₙₑ), 147.3 (Cₓₐₙₜₕᵢₙₑ), 131.1 (d, ³J_{C,F}=4.4 Hz, Cₐᵣₒₘ), 128.4 (d, ¹J_{C,F}=8.2 Hz, Cₐᵣₒₘ), 126.9 (d, ²J_{C,F}=15.5 Hz, Cₐᵣₒₘ), 124.4 (d, ³J_{C,F}=3.2 Hz, Cₐᵣₒₘ), 115.1 (d, J_{C,F}=21.7 Hz, Cₐᵣₒₘ), 106.4 (C5), 79.7 (C_{propargyl}), 72.7 (C_{propargyl}), 42.4 (N3-CH₂), 31.3 (N7-CH₃), 29.9 (N1-CH₂), 28.5 (d, ³J_{C,P}=15.8 Hz, CH₂), 27.2 (d, ¹J_{C,P}=136.4 Hz, PCH₂), 26.4 (CH₂), 25.9 (d, ³J_{C,F}=1.5 Hz, CH₂), 12.0 (d, ²J_{C,P}=4.4 Hz, CH₂). ³¹P NMR (243 MHz, DMSO-d₆) δ [ppm]=27.2. HRMS (ESI-QTOF) calculated for C₂₁H₂₄FN₄O₅P [M+H]⁺: 463.1547; found: 463.30. HRMS (ESI-QTOF) calculated for C₂₁H₂₄FN₄O₅P [M+H]⁺: 463.154; found: 463.155.

B-29) (4-(8-(4-Bromophenethyl)-7-methyl-2,6-dioxo-1-(prop-2-yn-1-yl)-1,2,6,7-tetrahydro-3H-purin-3-yl)butyl)phosphonic acid: White solid; m.p. 227-229° C. ¹H NMR (600 MHz, DMSO-d₆) δ [ppm]=7.45 (d, J=8.1 Hz, 2H, 2Hₐᵣₒₘ), 7.23 (d, J=8.1 Hz, 2H, 2Hₐᵣₒₘ), 4.58 (d, J=2.5 Hz, 2H, N1-CH₂), 3.96 (t, J=7.1 Hz, 2H, N3-CH₂), 3.74 (s, 3H, N7-CH₃), 3.06 (t, J=2.3 Hz, 1H, H_{propargyl}), 3.04 (t, J=8.1 Hz, 2H, CH₂), 2.99 (t, J=8.2 Hz, 2H, CH₂), 1.76-1.68 (m, 2H, CH₂), 1.57-1.42 (m, 4H, 2CH₂). ¹³C NMR (151 MHz, DMSO-d₆) δ [ppm]=153.9 (Cₓₐₙₜₕᵢₙₑ), 153.3 (Cₓₐₙₜₕᵢₙₑ), 149.7 (Cₓₐₙₜₕᵢₙₑ), 147.3 (Cₓₐₙₜₕᵢₙₑ), 139.9 (Cₐᵣₒₘ), 131.1 (2Cₐᵣₒₘ), 130.8 (2Cₐᵣₒₘ), 119.2 (Cₐᵣₒₘ), 106.3 (C5), 79.7 (C_{propargyl}), 72.7 (C_{propargyl}), 42.4 (N3-CH₂), 31.8 (CH₂), 31.3 (N1-CH₂), 29.9 (CH₂), 28.6 (d, ³J_{C,P}=15.4 Hz, CH₂), 27.5 (d, ¹J_{C,P}=136.7 Hz, PCH₂), 27.4 (N7-CH₃), 20.2 (d, ²J_{C,P}=4.2 Hz, CH₂). ³¹P NMR (243 MHz, DMSO-d₆) δ [ppm]=26.6. HRMS (ESI-QTOF) calculated for C₂₁H₂₄BrN₄O₅P [M+H]⁺: 523.0740; found: 523.0735.

B-30) (4-(7-Ethyl-8-(2-fluorophenethyl)-2,6-dioxo-1-(prop-2-yn-1-yl)-1,2,6,7-tetrahydro-3H-purin-3-yl)butyl)phosphonic acid: White solid; m.p. 197-199° C. ¹H NMR (600 MHz, DMSO-d₆) δ [ppm]=7.34 (td, J=7.8, 1.8 Hz, 1H, Hₐᵣₒₘ), 7.26 (tdd, J=7.5, 5.3, 1.8 Hz, 1H, Hₐᵣₒₘ), 7.18-7.09 (m, 2H, 2Hₐᵣₒₘ), 4.59 (d, J=2.5 Hz, 2H, N1-CH₂), 4.20 (q, J=7.1 Hz, 2H, N7-CH₂), 3.96 (t, J=7.2 Hz, 2H, N3-CH₂), 3.40 (bs, 20H), 3.08-3.06 (m, 4H, 2CH₂), 3.07 (t, J=2.7 Hz, 1H, H_{propargyl}), 1.72 (p, J=7.2 Hz, 2H, CH₂), 1.60-1.53 (m, 2H, CH₂), 1.48 (tt, J=9.1, 4.2 Hz, 2H, CH₂), 1.23 (t, J=7.1 Hz, 3H, N7-CH₂CH₃). ¹³C NMR (151 MHz, DMSO-d₆) δ [ppm]=160.6 (d, ¹J_{C,F}=243.2 Hz, Cₐᵣₒₘ), 152.9 (Cxamthine), 152.9 (Cxamhiine), 149.8 (Cxamhiine), 147.7 (Cxamthine), 131.1 (d, J_{C,F}=4.7 Hz, Cₐᵣₒₘ), 128.4 (d, ²J_{C,F}=8.2 Hz, Cₐᵣₒₘ), 126.9 (d, ²J_{C,F}=14.4 Hz, Cₐᵣₒₘ), 124.3 (d, ⁴J_{C,F}=3.1 Hz, Cₐᵣₒₘ), 115.1 (d, 2J_{C,F}=21.9 Hz, Cₐᵣₒₘ), 105.5 (C5), 79.7 (C_{propargyl}), 72.8 (C_{propargyl}), 42.4 (N3-CH₂), 40.1 (N7-CH₂), 29.9 (N1-CH₂), 28.5 (d, ³J_{C,P}=15.6 Hz, CH₂), 27.2 (d, ¹J_{C,P}=136.1 Hz, PCH₂), 26.3 (2CH₂), 20.0 (d, ²J_{C,P}=3.7 Hz, CH₂), 15.8 (N7-CH₂CH₃). ³¹P NMR (243 MHz, DMSO-d₆) δ [ppm]=27.2. HRMS (ESI-QTOF) calculated for C₂₂H₂₆FN₄O₅P [M+H]⁺: 477.17; found: 477.20. HRMS (ESI-QTOF) calculated for C₂₂H₂₆FN₄O₅P [M+H]⁺: 477.170; found: 477.170.

B-31) (4-(7-Ethyl-8-(2-iodophenethyl)-2,6-dioxo-1-(prop-2-yn-1-yl)-1,2,6,7-tetrahydro-3H-purin-3-yl)butyl)phosphonic acid: White solid; m.p. 202-204° C. ¹H NMR (600 MHz, DMSO-d₆) δ [ppm]=7.84 (d, J=7.8 Hz, 1H, Hₐᵣₒₘ), 7.36-7.28 (m, 2H, 2Hₐᵣₒₘ), 6.99-6.94 (m, 1H, Hₐᵣₒₘ), 4.59 (d, J=2.4 Hz, 2H, N1-CH₂), 4.19 (q, J=7.1 Hz, 2H, N7-CH₂), 3.98 (t, J=7.2 Hz, 2H, N3-CH₂), 3.14 (t, J=7.5 Hz, 2H, CH₂), 3.07 (t, J=2.5 Hz, 1H, H_{propargyl}), 3.0 (t, J=7.5 Hz, 2H, CH₂), 1.74 (p, J=7.4 Hz, 2H, CH₂), 1.57 (ddd, J=16.9, 9.4, 5.9 Hz, 2H, CH₂), 1.53-1.44 (m, 2H, CH₂), 1.23 (t, J=7.1 Hz, 3H, N7-CH₂CH₃). ¹³C NMR (151 MHz, DMSO-d₆) δ [ppm]=152.9 (Cₓₐₙₜₕᵢₙₑ), 152.6 (Cₓₐₙₜₕᵢₙₑ), 149.8 (Cₓₐₙₜₕᵢₙₑ), 147.7 (Cₓₐₙₜₕᵢₙₑ), 142.6 (Cₐᵣₒₘ), 139.1 (Cₐᵣₒₘ), 130.0 (Cₐᵣₒₘ), 128.5 (Cₐᵣₒₘ), 128.5 (Cₐᵣₒₘ), 105.5 (C5), 100.7 (Cₐᵣₒₘ), 79.7 (C_{propargyl}), 72.7 (C_{propargyl}), 42.4 (N3-CH₂), 40.1 (N7-CH₂), 37.9 (CH₂), 29.9 (N1-CH₂), 28.5 (d, ³J_{C,P}=15.7 Hz, CH₂), 27.3 (d, ¹J_{C,P}=136.4 Hz, CH₂), 26.4 (CH₂), 20.1 (d, ²J_{C,P}=4.3 Hz, CH₂), 15.9 (N7-CH₂CH₃). ³¹P NMR (243 MHz, DMSO-d₆) δ [ppm]=27.1. HRMS (ESI-QTOF) calculated for C₂₂H₂₆IN₄O₅P [M+Na]⁺: 607.0578; found: 607.0593.

B-32) (4-(7-Ethyl-8-(2-methylphenethyl)-2,6-dioxo-1-(prop-2-yn-1-yl)-1,2,6,7-tetrahydro-3H-purin-3-yl)butyl)phosphonic acid: White solid; m.p. 152-154° C. ¹H NMR (600 MHz, DMSO-d₆) δ [ppm]=7.15 (ddd, J=9.8, 5.5, 3.5 Hz, 2H, 2Hₐᵣₒₘ), 7.09 (dd, J=5.6, 3.4 Hz, 2H, 2Hₐᵣₒₘ), 4.59 (d, J=2.4 Hz, 2H, N1-CH₂), 4.14 (q, J=7.1 Hz, 2H, N7-CH₂), 3.99 (t, J=7.2 Hz, 2H, N3-CH₂), 3.06 (t, J=2.4 Hz, 1H, H_{propargyl}), 3.03 (s, 4H, 2CH₂), 2.29 (s, 3H, CH₃), 1.75 (p, J=7.4 Hz, 2H, CH₂), 1.61-1.54 (m, 2H, CH₂), 1.54-1.44 (m, 2H, CH₂), 1.17 (t, J=7.1 Hz, 3H, N7-CH₂CH₃). ¹³C NMR (151 MHz, DMSO-d₆) δ [ppm]=153.4 (Cₓₐₙₜₕᵢₙₑ), 152.8 (Cₓₐₙₜₕᵢₙₑ), 149.8 (Cₓₐₙₜₕᵢₙₑ), 147.7 (Cₓₐₙₜₕᵢₙₑ), 138.6 (Cₐᵣₒₘ), 135.7 (Cₐᵣₒₘ), 130.0 (Cₐᵣₒₘ), 128.8 (Cₐᵣₒₘ), 126.3 (Cₐᵣₒₘ), 125.9 (Cₐᵣₒₘ), 105.4 (C5), 79.7 (C_{propargyl}), 72.7 (C_{propargyl}), 40.1 (N3-CH₂), 30.3 (CH₂), 29.9 (N1-CH₂), 28.5 (d, ³J_{C,P}=15.6 Hz, CH₂), 27.3 (d, ¹J_{C,P}=136.5 Hz, PCH₂), 26.5 (CH₂), 20.0 (d, ²J_{C,P}=4.3 Hz, CH₂), 18.8 (CH₃), 15.8 (N7-CH₂CH₃). ³¹P NMR (243 MHz, DMSO-d₆) δ [ppm]=27.1. HRMS (ESI-QTOF) calculated for C₂₃H₂₉N₄O₅P [M+Na]⁺: 495.1768; found: 495.1788.

B-33) (4-(8-(2,6-Dimethylphenethyl)-7-ethyl-2,6-dioxo-1-(prop-2-yn-1-yl)-1,2,6,7-tetrahydro-3H-purin-3-yl)butyl)phosphonic acid: White solid; m.p. n.d. ¹H NMR (600 MHz, DMSO-d₆) δ [ppm]=6.99 (d, J=1.1 Hz, 3H, 3Hₐᵣₒₘ), 4.59 (d, J=2.5 Hz, 2H, N1-CH₂), 4.12 (q, J=7.1 Hz, 2H, N7-CH₂), 3.99 (t, J=7.2 Hz, 2H, N3-CH₂), 3.06 (t, J=2.4 Hz, 1H, H_{propargyl}), 3.03 (dd, J=9.1, 6.6 Hz, 2H, CH₂), 2.91 (dd, J=8.9, 6.8 Hz, 2H, CH₂), 2.26 (s, 6H, 2CH₃), 1.74 (p, J=7.4 Hz, 2H, CH₂), 1.51 (tdd, J=19.5, 9.2, 4.3 Hz, 4H, 2CH₂), 1.20 (t, J=7.1 Hz, 3H, N7-CH₂CH₃). ¹³C NMR (151 MHz, DMSO-d₆) δ [ppm]=153.5 (Cₓₐₙₜₕᵢₙₑ), 152.9 (Cₓₐₙₜₕᵢₙₑ), 149.8 (Cₓₐₙₜₕᵢₙₑ), 147.8 (Cₓₐₙₜₕᵢₙₑ), 137.0 (Cₐᵣₒₘ), 136.0 (2Cₐᵣₒₘ), 128.0 (2Cₐᵣₒₘ), 126.0 (Cₐᵣₒₘ), 105.4 (C5), 79.7 (C_{propargyl}), 72.7 (C_{propargyl}), 42.4 (N3-CH₂), 40.1 (N7-CH₂), 29.9 (N1-CH₂), 28.6 (d, ³J_{C,P}=15.6 Hz, CH₂), 27.6 (d, ¹J_{C,P}=135.9 Hz, PCH₂), 27.3 (CH₂), 25.3 (CH₂), 20.2 (d, ²J_{C,P}=4.4 Hz, CH₂), 19.3 (2CH₃), 15.9 (N7-CH₂CH₃). ³¹P NMR (243 MHz, DMSO-d₆) δ [ppm]=26.3. HRMS (ESI-QTOF) calculated for C₂₄H₃₁N₄O₅P [M+H]⁺: 487.2105; found: 487.2120.

B-34) (4-(8-(2,6-Dichlorophenethyl)-7-ethyl-2,6-dioxo-1-(prop-2-yn-1-yl)-1,2,6,7-tetrahydro-3H-purin-3-yl)butyl)phosphonic acid: White solid; m.p. 207-209° C. ¹H NMR (600 MHz, DMSO-d₆) δ [ppm]=7.46 (d, J=8.0 Hz, 2H, 2Hₐᵣₒₘ), 7.30 (t, J=8.0 Hz, 1H, Hₐᵣₒₘ), 4.60 (d, J=2.4 Hz, 2H, N1-CH₂), 4.23 (q, J=7.1 Hz, 2H, N7-CH₂), 3.91 (t, J=7.3 Hz, 2H, N3-CH₂), 3.28 (t, J=7.7 Hz, 2H, CH₂), 3.07 (t, J=2.4 Hz, 1H, H_{propargyl}), 3.04 (t, J=7.7 Hz, 2H, CH₂), 1.67 (p, J=7.4 Hz, 2H, CH₂), 1.56-1.40 (m, 4H, 2CH₂), 1.30 (t, J=7.1 Hz, 3H, N7-CH₂CH₃). ¹³C NMR (151 MHz, DMSO-d₆) δ [ppm]=152.9 (Cₓₐₙₜₕᵢₙₑ), 152.1 (Cₓₐₙₜₕᵢₙₑ), 149.8 (Cₓₐₙₜₕᵢₙₑ), 147.7 (Cₓₐₙₜₕᵢₙₑ), 135.3 (Cₐᵣₒₘ), 134.7 (2Cₐᵣₒₘ), 129.2 (Cₐᵣₒₘ), 128.5 (2Cₐᵣₒₘ), 105.6 (C5), 79.7 (C_{propargyl}), 72.7 (C_{propargyl}), 42.4 (N3-CH₂), 40.1 (N7-CH₂), 30.0 (N1-CH₂), 29.1 (CH₂), 28.5 (d, J=15.6 Hz, CH₂), 27.4 (d, J=136.3 Hz, PCH₂), 24.4 (CH₂), 20.1 (d, J=4.4 Hz, CH₂), 16.0 (N7-CH₂CH₃). ³¹P NMR (243 MHz, DMSO-d₆) δ [ppm]=26.8. HRMS (ESI-QTOF) calculated for C₂₂H₂₅Cl₂N₄O₅P [M+H]⁺: 527.1012; found: 527.0995.

B-35) (4-(8-(4-Bromophenethyl)-7-ethyl-2,6-dioxo-1-(prop-2-yn-1-yl)-1,2,6,7-tetrahydro-3H-purin-3-yl)butyl)phosphonic acid: White solid; m.p. 169-171° C. H NMR (600 MHz, DMSO-d₆) δ [ppm]=7.47-7.44 (m, 2H, 2Hₐᵣₒₘ), 7.26-7.22 (m, 2H, 2Hₐᵣₒₘ), 4.59 (d, J=2.5 Hz, 2H, N1-CH₂), 4.18 (q, J=7.1 Hz, 2H, N7-CH₂), 3.98 (t, J=7.1 Hz, 2H, N3-CH₂), 3.06 (t, J=2.3 Hz, 1H, H_{propargyl}), 3.07 (t, J=8.2 Hz, 2H, CH₂), 3.03 (t, J=8.3 Hz, 2H, CH₂), 1.74 (p, J=7.5 Hz, 2H, CH₂), 1.61-1.54 (m, 2H, CH₂), 1.54-1.46 (m, 2H, CH₂), 1.20 (t, J=7.1 Hz, 3H, N7-CH₂CH₃). ¹³C NMR (126 MHz, DMSO-d₆) δ [ppm]=153.1 (Cₓₐₙₜₕᵢₙₑ), 152.8 (Cₓₐₙₜₕᵢₙₑ), 149.8 (Cₓₐₙₜₕᵢₙₑ), 147.7 (Cₓₐₙₜₕᵢₙₑ), 139.9 (Cₐᵣₒₘ), 131.1 (2Cₐᵣₒₘ), 130.8 (2Cₐᵣₒₘ), 119.2 (Cₐᵣₒₘ), 105.4 (C5), 79.7 (C_{propargyl}), 72.7 (C_{propargyl}), 42.4 (N3-CH₂), 40.1 (N7-CH₂), 32.1 (CH₂), 29.9 (N1-CH₂), 28.5 (d, ³J_{C,P}=15.6 Hz, CH₂), 27.2 (CH₂), 27.2 (d, ¹J_{C,P}=136.4 Hz, PCH₂), 20.0 (d, ²J_{C,P}=4.3 Hz, CH₂), 15.8 (N7-CH₂CH₃). ³¹P NMR (202 MHz, DMSO-d₆) δ [ppm]=27.2.

HRMS (ESI-QTOF) calculated for C₂₂H₂₆BrN₄O₅P [M+H]⁺: 537.0897; found: 537.0892.

B-36) (4-(8-(2-Bromophenethyl)-7-(cyclohexylmethyl)-2,6-dioxo-1-(prop-2-yn-1-yl)-1,2,6,7-tetrahydro-3H-purin-3-yl)butyl)phosphonic acid: White solid; m.p. 143-145° C. ¹H NMR (600 MHz, DMSO-d₆) δ [ppm]=7.59 (dd, J=8.1, 1.3 Hz, 1H, Hₐᵣₒₘ), 7.36 (dd, J=7.7, 1.8 Hz, 1H, Hₐᵣₒₘ), 7.29 (td, J=7.4, 1.3 Hz, 1H, Hₐᵣₒₘ), 7.16 (td, J=7.7, 1.8 Hz, 1H, Hₐᵣₒₘ), 4.58 (d, J=2.4 Hz, 2H, N1-CH₂), 4.02-3.94 (m, 4H, N3-CH₂ and N7-CH₂), 3.18 (t, J=7.5 Hz, 2H, CH₂), 3.08-3.03 (m, 3H, CH₂ and H_{propargyl}), 1.79-1.72 (m, 2H, CH₂), 1.66-1.54 (m, 6H, CH₂ and 4H_{cyclohexane}), 1.54-1.46 (m, 2H, CH₂), 1.44-1.37 (m, 2H, 2H_{cyclohexane}), 1.12-1.03 (m, J=9.0, 7.7 Hz, 3H, 3H_{cyclohexane}), 1.00-0.90 (m, 2H, 2H_{cyclohexane}). ¹³C NMR (151 MHz, DMSO-d₆) δ [ppm]=153.3 (Cₓₐₙₜₕᵢₙₑ), 153.0 (Cₓₐₙₜₕᵢₙₑ), 149.7 (Cₓₐₙₜₕᵢₙₑ), 147.5 (Cₓₐₙₜₕᵢₙₑ), 139.3 (Cₐᵣₒₘ), 132.5 (Cₐᵣₒₘ), 131.2 (Cₐᵣₒₘ), 128.6 (Cₐᵣₒₘ), 127.9 (Cₐᵣₒₘ), 123.8 (Cₐᵣₒₘ), 106.1 (C5), 79.7 (C_{propargyl}), 72.7 (C_{propargyl}), 50.1 (N7-CH₂), 42.4 (N3-CH₂), 38.4 (N7-CH₂CH), 33.1 (CH₂), 30.0 (N1-CH₂), 29.5 (2C_{cyclohexane}), 28.5 (d, ³J_{C,P}=15.6 Hz, CH₂), 27.3 (d, ¹J_{C,P}=136.6 Hz, PCH₂), 26.3 (CH₂), 25.7 (2C_{cyclohexane}), 25.1 (C_{cyclohexane}), 20.1 (d, ²J_{C,P}=4.5 Hz, CH₂). ³¹P NMR (243 MHz, DMSO-d₆) δ [ppm]=27.2. HRMS (ESI-QTOF) calculated for C₂₇H₃₄BrN₄O₅P [M+H]⁺: 605.1523; found: 605.1529.

B-37) (4-(8-(2-Bromophenethyl)-7-(cyclopentylmethyl)-2,6-dioxo-1-(prop-2-yn-1-yl)-1,2,6,7-tetrahydro-3H-purin-3-yl)butyl)phosphonic acid: White solid; m.p. 137-139° C. ¹H NMR (600 MHz, DMSO-d₆) δ [ppm]=7.59 (dd, J=8.0, 1.2 Hz, 1H, Hₐᵣₒₘ), 7.35 (dd, J=7.7, 1.7 Hz, 1H, Hₐᵣₒₘ), 7.29 (td, J=7.5, 1.2 Hz, 1H, Hₐᵣₒₘ), 7.16 (td, J=7.6, 1.8 Hz, 1H, Hₐᵣₒₘ), 4.58 (d, J=2.5 Hz, 2H, N1-CH₂), 4.07 (d, J=7.7 Hz, 2H, N7-CH₂), 3.99 (t, J=7.2 Hz, 2H, N3-CH₂), 3.19 (t, J=7.4 Hz, 2H, CH₂), 3.08 (t, J=7.5 Hz, 2H, CH₂), 3.06 (t, J=2.4 Hz, 1H, H_{propargyl}), 2.23-2.13 (m, 1H, N7-CH₂CH), 1.75 (p, J=7.4 Hz, 2H, CH₂), 1.64-1.55 (m, 4H, 2CH₂), 1.55-1.38 (m, 6H, ⁶H_{cyclopentane}), 1.21-1.12 (m, 2H, 2H_{cyclopentane}). ¹³C NMR (151 MHz, DMSO-d₆) δ [ppm]=153.1 (Cₓₐₙₜₕᵢₙₑ), 153.0 (Cₓₐₙₜₕᵢₙₑ), 149.7 (Cₓₐₙₜₕᵢₙₑ), 147.6 (Cₓₐₙₜₕᵢₙₑ), 139.3 (Cₐᵣₒₘ), 132.5 (Cₐᵣₒₘ), 131.1 (Cₐᵣₒₘ), 128.6 (Cₐᵣₒₘ), 127.9 (Cₐᵣₒₘ), 123.8 (Cₐᵣₒₘ), 105.9 (C5), 79.7 (C_{propargyl}), 72.7 (C_{propargyl}), 48.5 (N7-CH₂), 42.5 (N3-CH₂), 40.1 (N7-CH₂CH), 33.2 (CH₂), 30.0 (N1-CH₂), 29.2 (2C_{cyclopentane}), 28.6 (d, ³J_{C,P}=15.5 Hz, CH₂), 27.5 (d, ¹J_{C,P}=135.5 Hz, PCH₂), 26.4 (CH₂), 24.3 (2C_{cyclopentane}), 20.2 (d, ²J_{C,P}=4.5 Hz, CH₂). ³¹P NMR (243 MHz, DMSO-d₆) δ [ppm]=26.8. HRMS (ESI-QTOF) calculated for C₂₋₆H₃₂BrN₄O₅P [M+Na]⁺: 613.1191; found: 613.1186.

B-38) (4-(8-(4-Bromophenethyl)-7-(cyclopentylmethyl)-2,6-dioxo-1-(prop-2-yn-1-yl)-1,2,6,7-tetrahydro-3H-purin-3-yl)butyl)phosphonic acid: White solid; m.p. 129-131° C. ¹H NMR (600 MHz, DMSO-d₆) δ [ppm]=7.45 (d, J=8.2 Hz, 2H, 2Hₐᵣₒₘ), 7.23 (d, J=8.3 Hz, 2H, 2Hₐᵣₒₘ), 4.58 (d, J=2.5 Hz, 2H, N1-CH₂), 4.11 (d, J=7.7 Hz, 2H, N7-CH₂), 4.00 (t, J=7.1 Hz, 2H, N3-CH₂), 2.24-2.12 (m, 1H, N7-CH₂CH), 1.75 (q, J=7.4 Hz, 2H, CH₂), 1.59 (qd, J=9.4, 8.4, 5.5 Hz, 4H, 2CH₂), 1.55-1.39 (m, 6H, 6H_{cyclopentane}), 1.16 (ddt, J=11.1, 7.9, 4.1 Hz, 2H, ²H_{cyclopentane}). H-1, H-13 and H-14 not visible. ¹³C NMR (151 MHz, DMSO-d₆) δ [ppm]=153.5 (Cₓₐₙₜₕᵢₙₑ), 153.0 (Cₓₐₙₜₕᵢₙₑ), 149.7 (Cₓₐₙₜₕᵢₙₑ), 147.6 (Cₓₐₙₜₕᵢₙₑ), 140.0 (Cₐᵣₒₘ), 131.1 (2Cₐᵣₒₘ), 130.9 (2Cₐᵣₒₘ), 119.2 (Cₐᵣₒₘ), 105.9 (C5), 79.7 (C_{propargyl}), 72.7 (C_{propargyl}), 48.4 (N7-CH₂), 42.4 (N3-CH₂), 40.7 (N7-CH₂CH), 32.0 (CH₂), 30.0 (N1-CH₂), 29.2 (2C_{cyclopentane}), 28.5 (d, ³J_{C,P}=15.6 Hz, CH₂), 27.7 (CH₂), 27.2 (d, ¹J_{C,P}=136.3 Hz, PCH₂), 24.2 (2C_{cyclopentane}), 20.1 (d, ²J_{C,P}=4.6 Hz, CH₂). ³¹P NMR (243 MHz, DMSO-d₆) δ [ppm]=27.2. HRMS (ESI-QTOF) calculated for C₂₋₆H₃₂BrN₄O₅P [M−H]⁺: 589.1210; found; 589.1217.

B-39) (4-(7-(cyclopentylmethyl)-8-(4-iodophenethyl)-2,6-dioxo-1-(prop-2-yn-1-yl)-1,2,6,7-tetrahydro-3H-purin-3-yl)butyl)phosphonic acid: White solid; m.p. 119-121° C. ¹H NMR (600 MHz, DMSO-d₆) δ [ppm]=7.62 (d, J=8.2 Hz, 2H, 2Cₐᵣₒₘ), 7.08 (d, J=8.2 Hz, 2H, 2 Cₐᵣₒₘ), 4.58 (d, J=2.4 Hz, 2H, N1-CH₂), 4.10 (d, J=7.7 Hz, 2H, N7-CH₂), 4.00 (t, J=7.2 Hz, 2H, N3-CH₂), 3.06 (t, J=2.4 Hz, 1H, H_{propargyl}), 3.04 (s, 4H, 2CH₂), 2.11 (p, J=7.7 Hz, 1H, N7-CH₂CH), 1.76 (p, J=7.4 Hz, 2H, CH₂), 1.62-1.55 (m, 4H, 2CH₂), 1.51 (ddt, J=16.3, 11.7, 6.3 Hz, 2H, ²H_{cyclopentane}), 1.48-1.39 (m, 4H, ⁴H_{cyclopentane}), 1.21-1.11 (m, 2H, ²H_{cyclopentane}). ¹³C NMR (151 MHz, DMSO-d₆) δ [ppm]=153.5 (Cₓₐₙₜₕᵢₙₑ), 153.0 (Cₓₐₙₜₕᵢₙₑ), 149.7 (Cₓₐₙₜₕᵢₙₑ), 147.6 (Cₓₐₙₜₕᵢₙₑ), 140.4 (Cₐᵣₒₘ), 137.0 (2Cₐᵣₒₘ), 131.1 (2Cₐᵣₒₘ), 105.8 (C5), 91.8 (Cₐᵣₒₘ), 79.7 (C_{propargyl}), 72.7 (C_{propargyl}), 48.4 (N7-CH₂), 42.4 (N3-CH₂), 40.7 (N7-CH₂CH), 32.2 (CH₂), 30.0 (N1-CH₂), 29.2 (2C_{cyclopentane}), 28.5 (d, ³J_{C,P}=15.5 Hz, CH₂), 27.7 (CH₂), 27.3 (d, ¹J_{C,P}=135.8 Hz, PCH₂), 24.2 (2C_{cyclopentane}), 20.1 (d, ²J_{C,P}=4.4 Hz, CH₂). ³¹P NMR (243 MHz, DMSO-d₆) δ [ppm]=27.1. HRMS (ESI-QTOF) calculated for C₂₋₆H₃₂IN₄O₅P [M+H]⁺: 639.123; found: 639.123.

B-40) (4-(7-(cyclopentylmethyl)-2,6-dioxo-1-(prop-2-yn-1-yl)-8-(4-(trifluoromethyl)phenethyl)-1,2,6,7-tetrahydro-3H-purin-3-yl)butyl)phosphonic acid: White solid; m.p. 101-102° C. ¹H NMR (500 MHz, DMSO-d₆) δ [ppm]=7.63 (d, J=8.0 Hz, 2H, 2Hₐᵣₒₘ), 7.51 (d, J=8.0 Hz, 2H, 2Hₐᵣₒₘ), 4.59 (d, J=2.5 Hz, 2H, N1-CH₂), 4.12 (d, J=7.7 Hz, 2H, N7-CH₂), 4.00 (t, J=7.1 Hz, 2H, N3-CH₂), 3.19 (t, J=7.2 Hz, 2H, CH₂), 3.12 (t, J=7.7 Hz, 2H, CH₂), 3.05 (t, J=2.4 Hz, 1H, H_{propargyl}), 2.15 (p, J=7.5 Hz, 1H, N7-CH₂CH), 1.76 (p, J=7.4 Hz, 2H, CH₂), 1.64-1.55 (m, 4H, 2CH₂), 1.56-1.33 (m, 6H, ⁶H_{cyclopentane}), 1.16 (ddd, J=13.0, 7.6, 3.8 Hz, 2H, ²H_{cyclopentane}). ¹³C NMR (126 MHz, DMSO-d₆) δ [ppm]=153.3 (Cₓₐₙₜₕᵢₙₑ), 153.0 (Cₓₐₙₜₕᵢₙₑ), 149.7 (Cₓₐₙₜₕᵢₙₑ), 147.6 (Cₓₐₙₜₕᵢₙₑ), 145.5 (Cₐᵣₒₘ), 129.4 (2Cₐᵣₒₘ), 126.9 (q, 2J_{C,F}=31.6 Hz, Cₐᵣₒₘ), 125.0 (q, ³J_{C,F}=3.8 Hz, 2Cₐᵣₒₘ), 124.4 (q, ¹J_{C,F}=271.8 Hz, CF₃), 105.9 (C5), 79.7 (C_{propargyl}), 72.7 (C_{propargyl}), 48.5 (N7-CH₂), 42.4 (N3-CH₂), 40.6 (N7-CH₂CH), 32.3 (CH₂), 30.0 (N1-CH₂), 29.1 (2C_{cyclopentane}), 28.5 (d, ³J_{C,P}=15.5 Hz, CH₂), 27.5 (CH₂), 27.2 (d, ¹J_{C,P}=136.4 Hz, PCH₂), 24.2 (2C_{cyclopentane}), 20.0 (d, ²J_{C,P}=4.4 Hz, CH₂). ³¹P NMR (202 MHz, DMSO-d₆) δ [ppm]=27.2. HRMS (ESI-QTOF) calculated for C27H₃₂F3N₄O₅P [M+H]⁺: 581.214; found: 581.212.

B-41) (4-(7-(cyclopentylmethyl)-8-(4-methoxyphenethyl)-2,6-dioxo-1-(prop-2-yn-1-yl)-1,2,6,7-tetra-hydro-3H-purin-3-yl)butyl)phosphonic acid: White solid; m.p. 111-113° C. ¹H NMR (600 MHz, DMSO-d₆) δ [ppm]=7.16 (d, J=8.6 Hz, 2H, 2Hₐᵣₒₘ), 6.83 (d, J=8.5 Hz, 2H, 2Hₐᵣₒₘ), 4.58 (d, J=2.4 Hz, 2H, N1-CH₂), 4.08 (d, J=7.7 Hz, 2H, N7-CH₂), 4.01 (t, J=7.2 Hz, 2H, N3-CH₂), 3.70 (s, 3H, OCH₃), 3.06 (t, J=2.4 Hz, 1H, H_{propargyl}), 3.01 (s, 4H, 2CH₂), 2.13 (p, J=7.7 Hz, 1H, N7-CH₂CH), 1.77 (p, J=7.4 Hz, 2H, CH₂), 1.59 (ddd, J=13.8, 9.9, 6.8 Hz, 4H, 2CH₂), 1.54-1.39 (m, 6H, ⁶H_{cyclopentane}), 1.21-1.11 (m, 2H, ²H_{cyclopentane}). ¹³C NMR (151 MHz, DMSO-d₆) δ [ppm]=157.7 (Cₐᵣₒₘ), 153.8 (Cₓₐₙₜₕᵢₙₑ), 153.0 (Cₓₐₙₜₕᵢₙₑ), 149.7 (Cₓₐₙₜₕᵢₙₑ), 147.6 (Cₓₐₙₜₕᵢₙₑ), 132.4 (Cₐᵣₒₘ), 129.5 (2Cₐᵣₒₘ), 113.7 (2Cₐᵣₒₘ), 105.8 (C5), 79.7 (C_{propargyl}), 72.7 (C_{propargyl}), 55.0 (OCH₃), 48.4 (N7-CH₂), 42.4 (N3-CH₂), 40.7 (N7-CH₂CH), 32.0 (CH₂), 30.0 (N1-CH₂), 29.2 (2C_{cyclopentane}), 28.5 (d, ³J_{C,P}=15.8 Hz, CH₂), 28.4 (CH₂), 27.2 (d, ¹J_{C,P}=136.4 Hz, PCH₂), 24.2 (2C_{cyclopentane}), 20.1 (d, ²J_{C,P}=4.6 Hz, CH₂). ³¹P NMR (243 MHz, DMSO-d₆) δ [ppm]=27.2. HRMS (ESI-QTOF) calculated for C27H₃₅N₄O₆P [M+H]⁺: 543.227; found: 543.227.

B-42) (4-(7-(cyclopentylmethyl)-8-(4-ethylphenethyl)-2,6-dioxo-1-(prop-2-yn-1-yl)-1,2,6,7-tetrahydro-3H-purin-3-yl)butyl)phosphonic acid: White solid; m.p. 133-135° C. ¹H NMR (500 MHz, DMSO-d₆) δ [ppm]=7.15 (d, J=8.1 Hz, 2H, 2Hₐᵣₒₘ), 7.10 (d, J=7.7 Hz, 2H, 2Hₐᵣₒₘ), 4.58 (d, J=2.5 Hz, 2H, N1-CH₂), 4.08 (d, J=7.7 Hz, 2H, N7-CH₂), 4.00 (t, J=7.2 Hz, 2H, N3-CH₂), 3.05 (t, J=2.5 Hz, 1H, H_{propargyl}), 3.03 (s, 4H, 2CH₂), 2.54 (q, J=7.6 Hz, 2H, CH₂CH₃), 2.16 (p, J=7.4 Hz, 1H, N7-CH₂CH), 1.77 (t, J=7.3 Hz, 2H, CH₂), 1.63-1.37 (m, 10H, 2CH₂ and 6H_{cyclopentane}), 1.22-1.15 (m, 2H, 2H_{cyclopentane}), 1.14 (t, J=7.6 Hz, 3H, CH₂CH₃). ¹³C NMR (126 MHz, DMSO-d₆) δ [ppm]=153.8 (Cₓₐₙₜₕᵢₙₑ), 153.0 (Cₓₐₙₜₕᵢₙₑ), 149.7 (Cₓₐₙₜₕᵢₙₑ), 147.6 (Cₓₐₙₜₕᵢₙₑ), 141.5 (Cₐᵣₒₘ), 137.7 (Cₐᵣₒₘ), 128.4 (2Cₐᵣₒₘ), 127.6 (2Cₐᵣₒₘ), 105.8 (C5), 79.7 (C_{propargyl}), 72.7 (C_{propargyl}), 48.4 (N7-CH₂), 42.4 (N3-CH₂), 40.6 (N7-CH₂CH), 32.4 (CH₂), 29.9 (N1-CH₂), 29.2 (2C_{cyclopentane}), 28.5 (d, ³J_{C,P}=15.4 Hz, CH₂), 28.2 (CH₂), 27.7 (CH₂CH₃), 27.3 (d, ¹J_{C,P}=136.3 Hz, PCH₂), 24.2 (2C_{cyclopentane}), 20.1 (d, ²J_{C,P}=4.4 Hz, CH₂), 15.6 (CH₂CH₃). ³¹P NMR (202 MHz, DMSO-d₆) δ [ppm]=27.0. HRMS (ESI-QTOF) calculated for C28H37N405P [M+H]⁺: 541.258; found: 541.251.

B-43) (4-(7-(cyclopentylmethyl)-8-(4-isopropylphenethyl)-2,6-dioxo-1-(prop-2-yn-1-yl)-1,2,6,7-tetra-hydro-3H-purin-3-yl)butyl)phosphonic acid: White solid; m.p. 125-127° C. ¹H NMR (600 MHz, DMSO-d₆) δ [ppm]=7.18-7.11 (m, 4H, 4Hₐᵣₒₘ), 4.58 (d, J=2.5 Hz, 2H, N1-CH₂), 4.07 (d, J=7.7 Hz, 2H, N7-CH₂), 4.01 (t, J=7.2 Hz, 2H, N3-CH₂), 3.06 (t, J=2.4 Hz, 1H, H_{propargyl}), 3.03 (s, 4H, 2CH₂), 2.83 (p, J=6.9 Hz, 1H, CH(CH₃)₂), 2.17 (p, J=7.6 Hz, 1H, N7-CH₂CH), 1.77 (p, J=7.4 Hz, 2H, CH₂), 1.61-1.39 (m, 12H, 2CH₂ and 8H_{cyclopentane}), 1.16 (d, J=6.9 Hz, 6H, CH(CH₃)₂). ¹³C NMR (151 MHz, DMSO-d₆) δ [ppm]=153.8 (Cₓₐₙₜₕᵢₙₑ), 153.0 (Cₓₐₙₜₕᵢₙₑ), 149.8 (Cₓₐₙₜₕᵢₙₑ), 147.6 (Cₓₐₙₜₕᵢₙₑ), 146.2 (Cₐᵣₒₘ), 137.9 (Cₐᵣₒₘ), 128.4 (2Cₐᵣₒₘ), 126.2 (2Cₐᵣₒₘ), 105.8 (C5), 79.7 (C_{propargyl}), 72.7 (C_{propargyl}), 48.4 (N7-CH₂), 42.4 (N3-CH₂), 40.6 (N7-CH₂CH), 33.0 (CH(CH₃)₂), 32.4 (CH₂), 30.0 (N1-CH₂), 29.2 (2C_{cyclopentane}), 28.6 (d, ³J_{C,P}=15.4 Hz, CH₂), 28.2 (CH₂), 27.4 (d, ¹J_{C,P}=136.4 Hz, PCH₂), 24.3 (2C_{cyclopentane}), 23.9 (CH(CH₃)₂), 20.1 (d, ²J_{C,P}=4.4 Hz, CH₂). ³¹P NMR (243 MHz, DMSO-d₆) δ [ppm]=26.9. HRMS (ESI-QTOF) calculated for C29H₃₉N₄O₅P [M+H]⁺: 555.274; found: 555.273.

B-44) (4-(8-(2-([1,1′-biphenyl]-4-yl)ethyl)-7-(cyclopentylmethyl)-2,6-dioxo-1-(prop-2-yn-1-yl)-1,2,6,7-tetrahydro-3H-purin-3-yl)butyl)phosphonic acid: White solid; m.p. 156-158° C. ¹H NMR (500 MHz, DMSO-d₆) δ [ppm]=7.65-7.59 (m, 2H, 2Hₐᵣₒₘ), 7.59-7.55 (m, 2H, 2Hₐᵣₒₘ), 7.44 (t, J=7.7 Hz, 2H, 2Hₐᵣₒₘ), 7.36 (d, J=8.2 Hz, 2H, 2Hₐᵣₒₘ), 7.35-7.30 (m, 1H, Hₐᵣₒₘ), 4.59 (d, J=2.5 Hz, 2H, N1-CH), 4.12 (d, J=7.6 Hz, 2H, N7-CH₂), 4.02 (t, J=7.1 Hz, 2H, N3-CH₂), 3.11 (dt, J=11.5, 5.9 Hz, 4H, 2CH₂), 3.05 (t, J=2.4 Hz, 1H, H_{propargyl}), 2.21-2.09 (m, 1H, N7-CH₂CH), 1.79 (p, J=7.4 Hz, 2H, CH₂), 1.66-1.35 (m, 10H, 2CH₂ and 6H_{cyclopentane}), 1.23-1.11 (m, 2H, ²H_{cyclopentane}). ¹³C NMR (126 MHz, DMSO-d₆) δ [ppm]=153.7 (Cₓₐₙₜₕᵢₙₑ), 153.0 (Cₓₐₙₜₕᵢₙₑ), 149.8 (Cₓₐₙₜₕᵢₙₑ), 147.6 (Cₓₐₙₜₕᵢₙₑ), 140.0 (Cₐᵣₒₘ), 139.8 (Cₐᵣₒₘ), 138.1 (Cₐᵣₒₘ), 129.1 (2Cₐᵣₒₘ), 128.8 (2Cₐᵣₒₘ), 127.2 (Cₐᵣₒₘ), 126.5 (2Cₐᵣₒₘ), 126.4 (2Cₐᵣₒₘ), 105.9 (C5), 79.7 (C_{propargyl}), 72.7 (C_{propargyl}), 48.5 (N7-CH₂), 42.4 (N3-CH₂), 40.7 (N7-CH₂CH), 32.4 (CH₂), 30.0 (N1-CH₂), 29.2 (2C_{cyclopentane}), 28.6 (d, ³J_{C,P}=15.3 Hz, CH₂), 28.0 (CH₂), 27.3 (d, ¹J_{C,P}=136.4 Hz, PCH₂), 24.2 (2C_{cyclopentane}), 20.1 (d, ²J_{C,P}=4.4 Hz, CH₂). ³¹P NMR (202 MHz, DMSO-d₆) δ [ppm]=27.1. HRMS (ESI-QTOF) calculated for C₃₂H₃₇N₄O₅P [M+H]⁺: 589.258; found: 589.258.

B-45) (4-(8-(2-Bromophenethyl)-7-(cyclobutylmethyl)-2,6-dioxo-1-(prop-2-yn-1-yl)-1,2,6,7-tetrahydro-3H-purin-3-yl)butyl)phosphonic acid: White solid; m.p. 167-169° C. ¹H NMR (600 MHz, DMSO-d₆) δ [ppm]=7.60 (dd, J=7.9, 1.2 Hz, 1H, Hₐᵣₒₘ), 7.37 (dd, J=7.6, 1.7 Hz, 1H, Hₐᵣₒₘ), 7.31 (td, J=7.5, 1.2 Hz, 1H, Hₐᵣₒₘ), 7.17 (td, J=7.6, 1.7 Hz, 1H, Hₐᵣₒₘ), 4.59 (d, J=2.5 Hz, 2H, N1-CH₂), 4.20 (d, J=7.4 Hz, 2H, N7-CH₂), 3.98 (t, J=7.2 Hz, 2H, N3-CH₂), 3.18 (t, J=7.5 Hz, 2H, CH₂), 3.08 (t, J=7.6 Hz, 2H, CH₂), 3.06 (t, J=2.4 Hz, 1H, H_{propargyl}), 2.61 (p, J=7.6 Hz, 1H, N7-CH₂CH), 1.88-1.80 (m, 2H, CH₂), 1.80-1.67 (m, 6H, CH₂ and 4H_{cyclobutane}), 1.60-1.54 (m, 2H, CH₂), 1.50 (qd, J=9.0, 7.5, 3.3 Hz, 2H, 2H_{cyclobutane}). ¹³C NMR (151 MHz, DMSO-d₆) δ [ppm]=153.1 (Cₓₐₙₜₕᵢₙₑ), 153.0 (Cₓₐₙₜₕᵢₙₑ), 149.7 (Cₓₐₙₜₕᵢₙₑ), 147.5 (Cₓₐₙₜₕᵢₙₑ), 139.3 (Cₐᵣₒₘ), 132.5 (Cₐᵣₒₘ), 131.1 (Cₐᵣₒₘ), 128.6 (Cₐᵣₒₘ), 127.9 (Cₐᵣₒₘ), 123.8 (Cₐᵣₒₘ), 105.9 (C5), 79.7 (C_{propargyl}), 72.7 (C_{propargyl}), 48.7 (N7-CH₂), 42.4 (N3-CH₂), 35.6 (N7-CH₂CH), 33.2 (CH₂), 30.0 (N1-CH₂), 28.5 (d, ³J_{C,P}=15.5 Hz, CH₂), 27.3 (d, ¹J_{C,P}=136.6 Hz, PCH₂), 26.3 (CH₂), 24.9 (2C_{cyclobuta}ne), 20.1 (d, ²J_{C,P}=4.3 Hz, CH₂), 17.7 (C_{cyclobutane}). ³¹P NMR (243 MHz, DMSO-d₆) δ [ppm]=27.0. HRMS (ESI-QTOF) calculated for C₂₅H₃₀BrN₄O₅P [M+Na]⁺: 599.1029; found: 599.1041.

B-46) (4-(8-(2-Bromophenethyl)-7-isopropyl-2,6-dioxo-1-(prop-2-yn-1-yl)-1,2,6,7-tetrahydro-3H-purin-3-yl)butyl)phosphonic acid: White solid; m.p. 109-111° C. ¹H NMR (600 MHz, DMSO-d₆) δ [ppm]=7.59 (d, J=8.0, 1.2 Hz, 1H, Hₐᵣₒₘ), 7.34 (dd, J=7.7, 1.8 Hz, 1H, 2Hₐᵣₒₘ), 7.30 (td, J=7.5, 1.3 Hz, 1H, Hₐᵣₒₘ), 7.16 (td, J=7.6, 1.8 Hz, 1H, Hₐᵣₒₘ), 4.75-4.66 (m, 1H, N7-CH), 4.61 (d, J=2.5 Hz, 2H, N1-CH₂), 3.97 (t, J=7.3 Hz, 2H, N3-CH₂), 3.18-3.10 (m, 4H, 2CH₂), 3.06 (t, J=2.4 Hz, 1H, H_{propargyl}), 1.75-1.67 (m, 2H, CH₂), 1.58-1.46 (m, 4H, 2CH₂), 1.43 (d, J=6.7 Hz, 6H, N7-CH(CH₃)₂). ¹³C NMR (151 MHz, DMSO-d₆) δ [ppm]=152.7 (Cₓₐₙₜₕᵢₙₑ), 152.4 (Cₓₐₙₜₕᵢₙₑ), 149.7 (Cₓₐₙₜₕᵢₙₑ), 148.8 (Cₓₐₙₜₕᵢₙₑ), 139.2 (Cₐᵣₒₘ), 132.5 (Cₐᵣₒₘ), 131.1 (Cₐᵣₒₘ), 128.6 (Cₐᵣₒₘ), 127.9 (Cₐᵣₒₘ), 123.7 (Cₐᵣₒₘ), 105.6 (C5), 79.8 (C_{propargyl}), 72.7 (C_{propargyl}), 48.9 (N7-CH), 42.5 (N3-CH₂), 33.7 (CH₂), 30.4 (N1-CH₂), 28.6 (d, ³J_{C,P}=15.6 Hz, CH₂), 27.5 (d, ¹J_{C,P}=135.2 Hz, PCH₂), 27.2 (CH₂), 21.2 (N7-CH(CH₃)₂), 20.2 (d, ²J_{C,P}=3.5 Hz, CH₂). ³¹P NMR (243 MHz, DMSO-d₆) δ [ppm]=26.6. HRMS (ESI-QTOF) calculated for C₂₃H₂₈BrN₄O₅P [M+H]⁺: 551.1053; found: 551.1049.

B-47) (4-(8-(2-Iodophenethyl)-7-isopropyl-2,6-dioxo-1-(prop-2-yn-1-yl)-1,2,6,7-tetrahydro-3H-purin-3-yl)butyl)phosphonic acid: White solid; m.p. 123-125° C. ¹H NMR (600 MHz, DMSO-d₆) δ [ppm]=7.84 (d, J=7.8 Hz, 1H, Hₐᵣₒₘ), 7.34-7.29 (m, 2H, 2Hₐᵣₒₘ), 6.97 (ddd, J=7.9, 5.7, 3.3 Hz, 1H, Hₐᵣₒₘ), 4.74-4.67 (m, 1H, N7-CH), 4.61 (d, J=2.5 Hz, 2H, N1-CH₂), 3.99 (t, J=7.3 Hz, 2H, N3-CH₂), 3.14-3.09 (m, 4H, 2CH₂), 3.06 (t, J=2.5 Hz, 1H, H_{propargyl}), 1.73 (p, J=7.3 Hz, 2H, CH₂), 1.60-1.45 (m, 4H, 2CH₂), 1.44 (d, J=6.7 Hz, 6H, N7-CH(CH₃)₂). ¹³C NMR (151 MHz, DMSO-d₆) δ [ppm]=152.7 (Cₓₐₙₜₕᵢₙₑ), 152.4 (Cₓₐₙₜₕᵢₙₑ), 149.7 (Cₓₐₙₜₕᵢₙₑ), 148.8 (Cₓₐₙₜₕᵢₙₑ), 142.5 (Cₐᵣₒₘ), 139.1 (Cₐᵣₒₘ), 130.0 (Cₐᵣₒₘ), 128.6 (Cₐᵣₒₘ), 128.5 (Cₐᵣₒₘ), 105.6 (C5), 100.7 (Cₐᵣₒₘ), 79.8 (C_{propargyl}), 72.7 (C_{propargyl}), 48.9 (N7-CH), 42.5 (N3-CH₂), 38.3 (CH₂), 30.4 (N1-CH₂), 28.6 (d, ³J_{C,P}=15.6 Hz, CH₂), 27.6 (CH₂), 27.4 (d, ¹J_{C,P} 136.5 Hz, PCH₂), 21.2 (N7-CH(CH₃)₂), 20.1 (d, ²J_{C,P}=4.3 Hz, CH₂). ³¹P NMR (243 MHz, DMSO-d₆) δ [ppm]=27.0. HRMS (ESI-QTOF) calculated for C₂₃H₂₈IN₄O₅P [M+H]⁺: 597.0758; found: 597.0763.

B-48) (4-(7-Isopropyl-8-(2-methylphenethyl)-2,6-dioxo-1-(prop-2-yn-1-yl)-1,2,6,7-tetrahydro-3H-purin-3-yl)butyl)phosphonic acid: White solid; m.p. 88-90° C. ¹H NMR (600 MHz, DMSO-d₆) δ [ppm]=7.16-7.12 (m, 2H, 2Hₐᵣₒₘ), 7.11-7.07 (m, 2H, 2Hₐᵣₒₘ), 4.68-4.63 (m, 1H, N7-CH), 4.61 (d, J=2.5 Hz, 2H, N1-CH₂), 3.99 (t, J=7.2 Hz, 2H, N3-CH₂), 3.11 (t, J=7.5 Hz, 2H, CH₂), 3.06 (d, J=2.4 Hz, 1H, H_{propargyl}), 3.00 (t, J=7.5 Hz, 2H, CH₂), 2.27 (s, 3H, CH₃), 1.74 (p, J=7.4 Hz, 2H, CH₂), 1.61-1.54 (m, 2H, CH₂), 1.54-1.46 (m, 2H, CH₂), 1.39 (d, J=6.7 Hz, 6H, N7-CH(CH₃)₂). ¹³C NMR (151 MHz, DMSO-d₆) δ [ppm]=153.5 (Cₓₐₙₜₕᵢₙₑ), 152.4 (Cₓₐₙₜₕᵢₙₑ), 149.7 (Cₓₐₙₜₕᵢₙₑ), 148.8 (Cₓₐₙₜₕᵢₙₑ), 138.4 (Cₐᵣₒₘ), 135.7 (Cₐᵣₒₘ), 130.0 (Cₐᵣₒₘ), 128.9 (Cₐᵣₒₘ), 126.3 (Cₐᵣₒₘ), 125.9 (Cₐᵣₒₘ), 105.5 (C5), 79.8 (C_{propargyl}), 72.7 (C_{propargyl}), 48.8 (N7-CH), 42.4 (N3-CH₂), 30.7 (CH₂), 30.4 (C1-CH₂), 28.5 (d, ³J_{C,P}=15.8 Hz, CH₂), 27.6 (CH₂), 27.3 (d, ¹J_{C,P}=136.4 Hz, PCH₂), 21.2 (N7-CH(CH₃)₂), 20.0 (d, ²J_{C,P}=4.4 Hz, CH₂), 18.9 (CH₃). ³¹P NMR (243 MHz, DMSO-d₆) δ [ppm]=27.2. HRMS (ESI-QTOF) calculated for C₂₄H₃₁N₄O₅P [M−H]⁻: 485.1948; found: 485.1951.

B-49) (4-(8-(2-bromophenethyl)-7-(sec-butyl)-2,6-dioxo-1-(prop-2-yn-1-yl)-1,2,6,7-tetrahydro-3H-purin-3-yl)butyl)phosphonic acid: White solid; m.p. 105-107° C. ¹H NMR (600 MHz, DMSO-d₆) δ [ppm]=7.59 (dd, J=8.0, 1.3 Hz, 1H, Hₐᵣₒₘ), 7.36 (dd, J=7.7, 1.8 Hz, 1H, Hₐᵣₒₘ), 7.30 (td, J=7.5, 1.3 Hz, 1H, Hₐᵣₒₘ), 7.15 (td, J=7.6, 1.7 Hz, 1H, Hₐᵣₒₘ), 4.59 (d, J=2.5 Hz, 2H, N1-CH₂), 3.98 (t, J=7.3 Hz, 2H, N3-CH₂), 3.17-3.10 (m, 4H, 2CH₂), 3.05 (t, J=2.4 Hz, 1H, H_{propargyl}), 1.99-1.91 (m, 1H, N7-CH), 1.82 (dp, J=14.0, 7.2 Hz, 2H, CH₂), 1.72 (p, J=7.4 Hz, 2H, CH₂), 1.58-1.45 (m, 4H, 2CH₂), 1.42 (d, J=6.7 Hz, 3H, N7-CHCH₃), 0.66 (t, J=7.4 Hz, 3H, N7-CHCH₂CH₃). ¹³C NMR (151 MHz, DMSO-d₆) δ [ppm]=153.3 (Cₓₐₙₜₕᵢₙₑ), 152.4 (Cₓₐₙₜₕᵢₙₑ), 149.6 (Cₓₐₙₜₕᵢₙₑ), 148.7 (Cₓₐₙₜₕᵢₙₑ), 139.2 (Cₐᵣₒₘ), 132.5 (Cₐᵣₒₘ), 131.1 (Cₐᵣₒₘ), 128.5 (Cₐᵣₒₘ), 127.9 (Cₐᵣₒₘ), 123.7 (Cₐᵣₒₘ), 105.5 (C5), 79.8 (C_{propargyl}), 72.7 (C_{propargyl}), 54.7 (N7-CH), 42.5 (N3-CH₂), 33.5 (CH₂), 30.4 (N1-CH₂), 28.6 (d, ³J_{C,P}=15.6 Hz, CH₂), 27.7 (d, ¹J_{C,P}=136.5 Hz, PCH₂), 27.6 (CH₂), 27.3 (CH₂), 20.3 (d, ²J_{C,P}=4.5 Hz, CH₂), 19.4 (N7-CHCH₃), 10.8 (N7-CHCH₂CH₃). ³¹P NMR (243 MHz, DMSO-d₆) δ [ppm]=26.4. HRMS (ESI-QTOF) calculated for C₂₄H₃₀BrN₄O₅P [M+H]⁺: 565.1210; found: 565.1202.

B-50) (4-(8-(2-Bromophenethyl)-7-(2-chloroethyl)-2,6-dioxo-1-(prop-2-yn-1-yl)-1,2,6,7-tetrahydro-3H-purin-3-yl)butyl)phosphonic acid: White solid; m.p. 197-199° C. ¹H NMR (600 MHz, DMSO-d₆) δ [ppm]=7.60 (dd, J=8.0, 1.2 Hz, 1H, Hₐᵣₒₘ), 7.40 (dd, J=7.7, 1.7 Hz, 1H, Hₐᵣₒₘ), 7.31 (td, J=7.4, 1.3 Hz, 1H, Hₐᵣₒₘ), 7.17 (td, J=7.6, 1.7 Hz, 1H, Hₐᵣₒₘ), 4.59 (d, J=2.5 Hz, 2H, N1-CH₂), 4.48 (t, J=6.0 Hz, 2H, N7-CH₂CH₂), 3.98 (t, J=7.2 Hz, 2H, N3-CH₂), 3.93 (t, J=5.9 Hz, 2H, N7-CH₂CH₂), 3.21-3.15 (m, 2H, CH₂), 3.16-3.12 (m, 2H, CH₂), 3.08 (t, J=2.4 Hz, 1H, H_{propargyl}), 1.74 (p, J=7.3 Hz, 2H, CH₂), 1.60-1.53 (m, 2H, CH₂), 1.53-1.45 (m, 2H, CH₂). ¹³C NMR (151 MHz, DMSO-d₆) δ [ppm]=154.1 (Cₓₐₙₜₕᵢₙₑ), 153.0 (Cₓₐₙₜₕᵢₙₑ), 149.7 (Cₓₐₜₕᵢₙₑ), 147.8 (Cₓₐₙₜₕᵢₙₑ), 139.3 (Cₐᵣₒₘ), 132.5 (Cₐᵣₒₘ), 131.0 (Cₐᵣₒₘ), 128.5 (Cₐᵣₒₘ), 127.9 (Cₐᵣₒₘ), 123.8 (Cₐᵣₒₘ), 105.7 (C5), 79.6 (C_{propargyl}), 72.8 (C_{propargyl}), 45.9 (N7-CH₂), 43.4 (N7-CH₂CH₂), 42.5 (N3-CH₂), 33.0 (CH₂), 30.0 (N1-CH₂), 28.5 (d, ³J_{C,P}=15.6 Hz, CH₂), 27.3 (d, ¹J_{C,P}=136.5 Hz, PCH₂), 26.4 (CH₂), 20.1 (d, ²J_{C,P}=4.4 Hz, CH₂). ³¹P NMR (243 MHz, DMSO-d₆) δ [ppm]=27.0. HRMS (ESI-QTOF) calculated for C₂₂H₂₅BrClN₄O₅P [M+H]⁺: 571.0507; found: 571.0501.

B-51) (4-(7-(2-Aminoethyl)-8-(2-bromophenethyl)-2,6-dioxo-1-(prop-2-yn-1-yl)-1,2,6,7-tetrahydro-3H-purin-3-yl)butyl)phosphonic acid: White solid; m.p. 159-161° C. ¹H NMR (600 MHz, DMSO-d₆) δ [ppm]=7.61 (dd, J=7.9, 1.2 Hz, 1H, Hₐᵣₒₘ), 7.43 (dd, J=7.7, 1.7 Hz, 1H, Hₐᵣₒₘ), 7.34 (td, J=7.5, 1.3 Hz, 1H, Hₐᵣₒₘ), 7.18 (td, J=7.7, 1.7 Hz, 1H, Hₐᵣₒₘ), 4.62 (d, J=2.5 Hz, 2H, N1-CH₂), 4.36 (t, J=6.3 Hz, 2H, N7-CH₂CH₂), 4.00 (t, J=7.1 Hz, 2H, N3-CH₂), 3.23 (t, J=6.3 Hz, 2H, N7-CH₂CH₂), 3.18 (t, J=7.2 Hz, 2H, CH₂), 3.10 (t, J=7.2 Hz, 2H, CH₂), 3.09 (d, J=2.4 Hz, 1H, H_{propargyl}), 1.74 (p, J=7.4 Hz, 2H, CH₂), 1.58-1.41 (m, 4H, 2CH₂). ¹³C NMR (151 MHz, DMSO-d₆) δ [ppm]=154.0 (Cₓₐₙₜₕᵢₙₑ), 153.2 (Cₓₐₙₜₕᵢₙₑ), 149.7 (Cₓₐₙₜₕᵢₙₑ), 148.0 (Cₓₐₙₜₕᵢₙₑ), 139.4 (Cₐᵣₒₘ), 132.5 (Cₐᵣₒₘ), 131.0 (Cₐᵣₒₘ), 128.6 (Cₐᵣₒₘ), 127.9 (Cₐᵣₒₘ), 123.8 (Cₐᵣₒₘ), 106.1 (C5), 79.6 (C_{propargyl}), 72.9 (C_{propargyl}), 42.4 (N3-CH₂), 42.3 (N7-CH₂CH₂), 40.1 (N7-CH₂CH₂), 32.8 (CH₂), 30.0 (N1-CH₂), 28.3 (d, ³J_{C,P}=15.7 Hz, CH₂), 27.3 (d, ¹J_{C,P}=136.4 Hz, PCH₂), 26.2 (CH₂), 19.9 (d, ¹J_{C,P}=4.4 Hz, CH₂). ³¹P NMR (243 MHz, DMSO-d₆) δ [ppm]=26.7. HRMS (ESI-QTOF) calculated for C₂₂H₂₇BrN₅O₅P [M+H]⁺: 552.1006; found: 552.1021.

B-52) (5-(8-(2-Bromophenethyl)-2,6-dioxo-1-(prop-2-yn-1-yl)-1,2,6,7-tetrahydro-3H-purin-3-yl)pentyl)phosphonic acid: White solid; m.p. 219-221° C. ¹H NMR (600 MHz, DMSO-d₆) δ [ppm]=13.34 (bs, 1H, N7-H), 7.57 (d, J=7.9 Hz, 1H, Hₐᵣₒₘ), 7.27 (d, J=4.4 Hz, 2H, 2Hₐᵣₒₘ), 7.14 (dq, J=8.4, 4.5, 4.0 Hz, 1H, Hₐᵣₒₘ), 4.58 (d, J=2.5 Hz, 2H, N1-CH₂), 3.95 (t, J=7.4 Hz, 2H, N3-CH₂), 3.13 (t, J=7.5 Hz, 2H, CH₂), 3.06 (t, J=2.3 Hz, 1H, H_{propargyl}), 3.02 (t, J=7.6 Hz, 2H, CH₂), 1.63 (p, J=7.6 Hz, 2H, CH₂), 1.46 (ddt, J=25.2, 16.8, 8.2 Hz, 4H, 2CH₂), 1.37-1.28 (m, 2H, CH₂). ¹³C NMR (151 MHz, DMSO-d₆) δ [ppm]=153.4 (Cₓₐₙₜₕᵢₙₑ), 152.9 (Cₓₐₙₜₕᵢₙₑ), 150.0 (Cₓₐₙₜₕᵢₙₑ), 148.0 (Cₓₐₙₜₕᵢₙₑ), 139.3 (Cₐᵣₒₘ), 132.5 (Cₐᵣₒₘ), 130.7 (Cₐᵣₒₘ), 128.5 (Cₐᵣₒₘ), 127.8 (Cₐᵣₒₘ), 123.7 (Cₐᵣₒₘ), 106.0 (C5), 79.8 (C_{propargyl}), 72.7 (C_{propargyl}), 43.0 (N3-CH₂), 33.6 (CH₂), 30.1 (N1-CH₂), 28.3 (CH₂), 28.1 (d, ¹J_{C,P}=136.3 Hz, PCH₂), 27.3 (d, ³J_{C,P}=13.1 Hz, CH₂), 27.2 (CH₂), 22.8 (d, ²J_{C,P}=4.6 Hz, CH₂). ³¹P NMR (243 MHz, DMSO-d₆) δ [ppm]=26.4. HRMS (ESI-QTOF) calculated for C₂₁H₂₄BrN₄O₅P [M+2Na]⁺: 567.0379; found: 567.0304.

B-53) (5-(8-(2-Bromophenethyl)-7-methyl-2,6-dioxo-1-(prop-2-yn-1-yl)-1,2,6,7-tetrahydro-3H-purin-3-yl)pentyl)phosphonic acid: White solid; m.p. 110-112° C. ¹H NMR (600 MHz, DMSO-d₆) δ [ppm]=7.59 (dd, J=8.0, 1.3 Hz, 1H, Hₐᵣₒₘ), 7.34 (dd, J=7.6, 1.8 Hz, 1H, Hₐᵣₒₘ), 7.30 (td, J=7.4, 1.2 Hz, 1H, Hₐᵣₒₘ), 7.17 (td, J=7.6, 1.8 Hz, 1H, Hₐᵣₒₘ), 4.58 (d, J=2.4 Hz, 2H, N1-CH₂), 3.95 (t, J=7.3 Hz, 2H, N3-CH₂), 3.74 (s, 3H, N7-CH₃), 3.14-3.11 (m, 2H, CH₂), 3.09-3.05 (in, 3H, H_{propargyl} and CH₂), 1.61 (q, J=7.5 Hz, 2H, CH₂), 1.55-1.42 (m, 4H, 2CH₂), 1.34 (p, J=7.5 Hz, 2H, CH₂). ¹³C NMR (151 MHz, DMSO-d₆) δ [ppm]=153.5 (Cₓₐₙₜₕᵢₙₑ), 153.3 (Cₓₐₙₜₕᵢₙₑ), 149.7 (Cₓₐₙₜₕᵢₙₑ), 147.3 (Cₓₐₙₜₕᵢₙₑ), 139.3 (Cₐᵣₒₘ), 132.5 (Cₐᵣₒₘ), 131.0 (Cₐᵣₒₘ), 128.6 (Cₐᵣₒₘ), 127.9 (Cₐᵣₒₘ), 123.7 (Cₐᵣₒₘ), 106.4 (C5), 79.7 (C_{propargyl}), 72.7 (C_{propargyl}), 42.5 (N3-CH₂), 32.9 (CH₂), 31.3 (N1-CH₂), 29.9 (CH₂), 27.5 (d, ¹J_{C,P}=135.6 Hz, PCH₂), 27.1 (d, ³J_{C,P}=15.4 Hz, CH₂), 27.1 (CH₂), 26.3 (N7-CH₃), 22.5 (d, ²J_{C,P}=4.6 Hz, CH₂). ³¹P NMR (243 MHz, DMSO-d₆) δ [ppm]=27.4. HRMS (ESI-QTOF) calculated for C₂₂H₂₆BrN₄O₅P [M+Na]⁺: 559.0722; found: 559.0716.

B-54) (5-(8-(2-Bromophenethyl)-7-ethyl-2,6-dioxo-1-(prop-2-yn-1-yl)-1,2,6,7-tetrahydro-3H-purin-3-yl)pentyl)phosphonic acid: White solid; m.p. 146-148° C. ¹H NMR (600 MHz, DMSO-d₆) δ [ppm]=7.59 (dd, J=7.9, 1.2 Hz, 1H, Hₐᵣₒₘ), 7.36 (dd, J=7.6, 1.7 Hz, 1H, Hₐᵣₒₘ), 7.30 (td, J=7.4, 1.2 Hz, 1H, Hₐᵣₒₘ), 7.16 (td, J=7.7, 1.7 Hz, 1H, Hₐᵣₒₘ), 4.59 (d, J=2.5 Hz, 2H, N1-CH₂), 4.19 (q, J=7.1 Hz, 2H, N7-CH₂), 3.96 (t, J=7.4 Hz, 2H, N3-CH₂), 3.72 (bs, 2OH), 3.15 (t, J=7.7 Hz, 2H, CH₂), 3.09 (t, J=7.4 Hz, 2H, CH₂), 3.06 (t, J=2.4 Hz, 1H, H_{propargyl}), 1.63 (p, J=7.7 Hz, 2H, CH₂), 1.50 (qd, J=9.0, 3.1 Hz, 4H, 2CH₂), 1.35 (p, J=6.9 Hz, 2H, CH₂), 1.22 (t, J=7.1 Hz, 3H, N7-CH₂CH₃). ¹³C NMR (151 MHz, DMSO-d₆) δ [ppm]=152.9 (Cₓₐₙₜₕᵢₙₑ), 152.7 (Cₓₐₙₜₕᵢₙₑ), 149.8 (Cₓₐₙₜₕᵢₙₑ), 147.7 (Cₓₐₙₜₕᵢₙₑ), 139.3 (Cₐᵣₒₘ), 132.5 (Cₐᵣₒₘ), 131.0 (Cₐᵣₒₘ), 128.6 (Cₐᵣₒₘ), 127.9 (Cₐᵣₒₘ), 123.8 (Cₐᵣₒₘ), 105.5 (C5), 79.7 (C_{propargyl}), 72.7 (C_{propargyl}), 42.6 (N3-CH₂), 40.1 (N7-CH₂), 33.3 (CH₂), 29.9 (N1-CH₂), 27.6 (d, ¹J_{C,P}=131.7 Hz, PCH₂), 27.1 (2CH₂), 26.1 (CH₂), 22.5 (d, ²J_{C,P}=4.5 Hz, CH₂), 15.9 (N7-CH₂CH₃). ³¹P NMR (243 MHz, DMSO-d₆) δ [ppm]=27.4. HRMS (ESI-QTOF) calculated for C₂₃H₂₈BrN₄O₅P [M+H]⁺: 551.11; found: 551.20. HRMS (ESI-QTOF) calculated for C₂₃H₂₈BrN₄O₅P [M+H]⁺: 551.106; found: 551.105.

B-55) (6-(8-(2-Bromophenethyl)-2,6-dioxo-1-(prop-2-yn-1-yl)-1,2,6,7-tetrahydro-3H-purin-3-yl) hexyl)phosphonic acid: White solid; m.p. 225-227° C. ¹H NMR (500 MHz, DMSO-d₆) δ [ppm]=13.32 (s, 1H, N7-H), 7.58 (d, J=7.9 Hz, 1H, Hₐᵣₒₘ), 7.29-7.26 (m, 2H, 2Hₐᵣₒₘ), 7.18-7.12 (m, 1H, Hₐᵣₒₘ), 4.59 (d, J=2.5 Hz, 2H, N1-CH₂), 3.97 (t, J=7.3 Hz, 2H, N3-CH₂), 3.14 (t, J=7.5 Hz, 2H, CH₂), 3.05 (t, J=2.5 Hz, 1H, H_{propargyl}), 3.02 (t, J=7.6 Hz, 2H, CH₂), 1.64 (p, J=7.5 Hz, 2H, CH₂), 1.51-1.41 (m, 4H, 2CH₂), 1.37 (p, J=6.4 Hz, 2H, CH₂), 1.27 (p, J=7.4, 6.8 Hz, 2H, CH₂). ¹³C NMR (126 MHz, DMSO-d₆) δ [ppm]=153.4 (Cₓₐₙₜₕᵢₙₑ), 152.8 (Cₓₐₙₜₕᵢₙₑ), 149.9 (Cₓₐₙₜₕᵢₙₑ), 148.0 (Cₓₐₙₜₕᵢₙₑ), 139.3 (Cₐᵣₒₘ), 132.5 (Cₐᵣₒₘ), 130.6 (Cₐᵣₒₘ), 128.4 (Cₐᵣₒₘ), 127.8 (Cₐᵣₒₘ), 123.7 (Cₐᵣₒₘ), 105.9 (C5), 79.7 (C_{propargyl}), 72.6 (C_{propargyl}), 42.9 (N3-CH₂), 33.5 (CH₂), 30.0 (N1-CH₂), 29.7 (d, ³J_{C,P}=15.6 Hz, CH₂), 28.2 (CH₂), 27.6 (d, ¹J_{C,P}=136.2 Hz, PCH₂), 27.3 (CH₂), 25.7 (CH₂), 22.7 (d, ²J_{C,P}=4.6 Hz, CH₂). ³¹P NMR (202 MHz, DMSO-d₆) δ [ppm]=27.4. HRMS (ESI-QTOF) calculated for C₂₂H₂₆BrN₄O₅P [M+H]⁺: 537.0897; found: 537.0887.

B-56) (6-(8-(2-Bromophenethyl)-7-methyl-2,6-dioxo-1-(prop-2-yn-1-yl)-1,2,6,7-tetrahydro-3H-purin-3-yl)hexyl)phosphonic acid: White solid; m.p. 221-223° C. ¹H NMR (600 MHz, DMSO-d₆) δ [ppm]=7.59 (dd, J=7.9, 1.2 Hz, 1H, Hₐᵣₒₘ), 7.34 (dd, J=7.6, 1.8 Hz, 1H, Hₐᵣₒₘ), 7.30 (td, J=7.4, 1.3 Hz, 1H, Hₐᵣₒₘ), 7.17 (td, J=7.6, 1.8 Hz, 1H, Hₐᵣₒₘ), 4.58 (d, J=2.5 Hz, 2H, N1-CH₂), 3.96 (t, J=7.2 Hz, 2H, N3-CH₂), 3.74 (s, 3H, N7-CH₃), 3.13 (t, J=7.6 Hz, 2H, CH₂), 3.06 (t, J=2.5 Hz, 1H, H_{propargyl}), 2.88 (d, J=7.6 Hz, 2H, CH₂), 1.62 (p, J=7.4 Hz, 2H, CH₂), 1.51-1.41 (m, J=3.1 Hz, 4H, 2CH₂), 1.35 (p, J=7.6 Hz, 2H, CH₂), 1.25 (p, J=7.6 Hz, 2H, CH₂). ¹³C NMR (151 MHz, DMSO-d₆) δ [ppm]=153.5 (Cₓₐₙₜₕᵢₙₑ), 153.3 (Cₓₐₙₜₕᵢₙₑ), 149.8 (Cₓₐₜₕᵢₙₑ), 147.4 (Cₓₐₙₜₕᵢₙₑ), 139.3 (Cₐᵣₒₘ), 132.5 (Cₐᵣₒₘ), 131.0 (Cₐᵣₒₘ), 128.6 (Cₐᵣₒₘ), 127.9 (Cₐᵣₒₘ), 123.7 (Cₐᵣₒₘ), 106.4 (C5), 79.7 (C_{propargyl}), 72.7 (C_{propargyl}), 42.6 (N3-CH₂), 32.9 (CH₂), 31.3 (N1-CH₂), 29.9 (CH₂), 29.7 (d, ³J_{C,P}=15.9 Hz, CH₂), 27.6 (d, ¹J_{C,P}=136.5 Hz, PCH₂), 27.2 (CH₂), 26.2 (N7-CH₃), 25.7 (CH₂), 22.7 (d, ²J_{C,P}=4.6 Hz, CH₂). ³¹P NMR (243 MHz, DMSO-d₆) δ [ppm]=27.5. HRMS (ESI-QTOF) calculated for C₂₃H₂₈BrN₄O₅P [M+2Na]⁺: 595.0692; found: 595.0682.

B-57) (6-(8-(2-Bromophenethyl)-7-ethyl-2,6-dioxo-1-(prop-2-yn-1-yl)-1,2,6,7-tetrahydro-3H-purin-3-yl)hexyl)phosphonic acid: White solid; m.p. 223-225° C. ¹H NMR (600 MHz, DMSO-d₆) δ [ppm]=7.59 (dd, J=8.0, 1.2 Hz, 1H, Hₐᵣₒₘ), 7.36 (dd, J=7.6, 1.8 Hz, 1H, Hₐᵣₒₘ), 7.30 (td, J=7.4, 1.3 Hz, 1H, Hₐᵣₒₘ), 7.17 (td, J=7.6, 1.7 Hz, 1H, Hₐᵣₒₘ), 4.59 (d, J=2.5 Hz, 2H, N1-CH₂), 4.19 (q, J=7.1 Hz, 2H, N7-CH₂), 3.97 (t, J=7.3 Hz, 2H, N3-CH₂), 3.15 (t, J=7.8 Hz, 2H, CH₂), 3.09 (t, J=7.4 Hz, 2H, CH₂), 3.06 (t, J=2.5 Hz, 1H, H_{propargyl}), 1.64 (p, J=7.5 Hz, 2H, CH₂), 1.52-1.41 (m, 4H, 2CH₂), 1.36 (p, J=7.0 Hz, 2H, CH₂), 1.28 (p, J=8.1 Hz, 2H, CH₂), 1.23 (t, J=7.1 Hz, 3H, N7-CH₂CH₃). ¹³C NMR (151 MHz, DMSO-d₆) δ [ppm]=152.9 (Cₓₐₙₜₕᵢₙₑ), 152.7 (Cₓₐₙₜₕᵢₙₑ), 149.8 (Cₓₐₙₜₕᵢₙₑ), 147.7 (Cₓₐₙₜₕᵢₙₑ), 139.3 (Cₐᵣₒₘ), 132.5 (Cₐᵣₒₘ), 131.0 (Cₐᵣₒₘ), 128.6 (Cₐᵣₒₘ), 127.9 (Cₐᵣₒₘ), 123.8 (Cₐᵣₒₘ), 105.5 (C5), 79.7 (C_{propargyl}), 72.7 (C_{propargyl}), 42.7 (N3-CH₂), 40.1 (N7-CH₂), 33.3 (CH₂), 29.9 (N1-CH₂), 29.7 (d, ³J_{C,P}=15.9 Hz, CH₂), 27.5 (d, ¹J_{C,P}=136.2 Hz, PCH₂), 27.3 (CH₂), 26.0 (CH₂), 25.7 (CH₂), 22.7 (d, ²J_{C,P}=4.5 Hz, CH₂), 15.9 (N7-CH₂CH₃). ³¹P NMR (243 MHz, DMSO-d₆) δ [ppm]=27.5. HRMS (ESI-QTOF) calculated for C₂₄H₃₀BrN₄O₅P [M+H]⁺: 565.1210; found: 565.1220.

B-58) (4-(7-Ethyl-8-(naphthalen-1-ylmethyl)-2,6-dioxo-1-(prop-2-yn-1-yl)-1,2,6,7-tetrahydro-3H-purin-3-yl)butyl)phosphonic acid: White solid; m.p. 138-140° C. ¹H NMR (600 MHz, DMSO-d6) δ[ppm]=7.89-7.83 (m, 3H, 3Hₐᵣₒₘ), 7.78 (s, 1H, Hₐᵣₒₘ), 7.52-7.45 (m, 2H, 2Hₐᵣₒₘ), 7.43 (dd, J=8.6, 1.8 Hz, 1H, Hₐᵣₒₘ), 4.59 (d, J=2.3 Hz, 2H, N1-CH₂), 4.41 (s, 2H, CH₂), 4.26 (q, J=7.1 Hz, 2H, N7-CH₂), 4.00 (t, J=7.1 Hz, 2H, N3-CH₂), 3.06 (t, J=2.5 Hz, 1H, H_{propargyl}), 1.77 (q, J=7.3 Hz, 2H, CH₂), 1.59-1.47 (m, 4H, 2CH₂), 1.08 (t, J=7.1 Hz, 3H, N7-CH₂CH₃). ¹³C NMR (151 MHz, DMSO-d6) δ[ppm]=152.9 (Cₓₐₙₜₕᵢₙₑ), 152.2 (Cₓₐₙₜₕᵢₙₑ), 149.8 (Cₓₐₜₕᵢₙₑ), 147.8 (Cₓₐₙₜₕᵢₙₑ), 134.0 (Cₐᵣₒₘ), 133.0 (Cₐᵣₒₘ), 131.8 (Cₐᵣₒₘ), 128.2 (Cₐᵣₒₘ), 127.5 (Cₐᵣₒₘ), 127.5 (Cₐᵣₒₘ), 126.8 (Cₐᵣₒₘ), 126.7 (Cₐᵣₒₘ), 126.3 (Cₐᵣₒₘ), 125.8 (Cₐᵣₒₘ), 105.9 (C5), 79.7 (C_{propargyl}), 72.7 (C_{propargyl}), 42.6 (N3-CH₂), 40.3 (N7-CH₂), 32.3 (CH₂), 30.0 (N1-CH₂), 28.7 (d, ³J_{C,P}=15.4 Hz, CH₂), 27.5 (d, ¹J_{C,P}=136.3 Hz, PCH₂), 20.2 (d, ²J_{C,P}=3.7 Hz, CH₂), 15.7 (N7-CH₂CH₃). ³¹P NMR (243 MHz, DMSO-d6) δ[ppm]=26.7. HRMS (ESI-QTOF) calculated for C₂₅H₂₇N₄O₅P [M+H]⁺: 495.1792; found: 495.1799.

C-2) Diethyl (4-(8-(2-methoxyphenethyl)-7-methyl-2,6-dioxo-1-(prop-2-yn-1-yl)-1,2,6,7-tetrahydro-3H-purin-3-yl)butyl)phosphonate: White solid; m.p. 124-146° C. ¹H-NMR (600 MHz, DMSO-d₆) δ [ppm]=7.20 (ddd, J=8.2, 7.4, 1.7 Hz, 1H, Hₐᵣₒₘ), 7.11 (dd, J=7.4, 1.7 Hz, 1H, Hₐᵣₒₘ), 6.95 (dd, J=8.3, 1.1 Hz, 1H, Hₐᵣₒₘ), 6.84 (td, J=7.3, 1.1 Hz, 1H, Hₐᵣₒₘ), 4.57 (d, J=2.4 Hz, 2H, N1-CH₂), 4.00-3.89 (m, 6H, N3-CH₂, POCH₂), 3.77 (s, 3H, OCH₃), 3.70 (s, 3H, N7-CH₃), 3.05 (t, J=2.4 Hz, 1H, H_{propargyl}), 2.99-2.92 (m, 4H, CH₂CH₂), 1.81-1.71 (m, 4H, CH₂CH₂), 1.51-1.42 (m, 2H, CH₂), 1.18 (t, J=7.0 Hz, 6H, POCH₂CH₃). ¹³C-NMR (151 MHz, DMSO-d₆) δ [ppm]=157.3 (Cₐᵣₒₘ—OCH₃), 154.5 (C8), 153.4 (CO), 150.0 (CO), 147.6 (C6), 130.0 (Cₐᵣₒₘ), 128.1 (Cₐᵣₒₘ), 128.0 (Cₐᵣₒₘ), 120.4 (Cₐᵣₒₘ), 110.8 (Cₐᵣₒₘ), 106.4 (C5), 79.8 (C_{propargyl}), 72.8 (C_{propargyl}), 60.9 (d, ²J_{C,P}=6.4 Hz, 2C, POCH₂), 55.4 (OCH₃), 42.3 (N3-CH₂), 31.4 (N7-CH₃), 30.0 (N1-CH₂), 28.3 (d, ²J_{C,P}=15.3 Hz, 1C, PCH₂CH₂), 28.0 (CH₂), 26.6 (CH₂), 24.2 (d, ¹J_{C,P}=138.9 Hz, 1C, PCH₂), 19.4 (d, ³J_{C,P}=4.7 Hz, 1C, P(CH₂)₂CH₂), 16.4 (d, ³J_{C,P}=5.6 Hz, POCH₂CH₃). ³¹P-NMR (243 MHz, DMSO-d₆) δ [ppm]=[ppm]=32.8. HRMS (ESI-QTOF) calculated for C₂₋₆H₃₅N₄O₆P [M+H]⁺: 531.2367; found: 531.2361.

C-3) Diethyl (4-(8-(3-bromophenethyl)-2,6-dioxo-1-(prop-2-yn-1-yl)-1,2,6,7-tetrahydro-3H-purin-3-yl)butyl)phosphonate: White solid; m.p. 133-135° C. ¹H-NMR (600 MHz, DMSO-d₆) δ [ppm]=13.28 (br s, 1H), 7.45 (s, 1H, Hₐᵣₒₘ), 7.38 (d, J=7.8 Hz, 1H, Hₐᵣₒₘ), 7.23 (t, J=7.7 Hz, 1H, Hₐᵣₒₘ), 7.20 (d, J=7.7 Hz, 1H, Hₐᵣₒₘ), 4.59 (s, 2H, N1-CH₂), 4.01-3.90 (m, 6H, N3-CH₂ and 2×OCH₂), 3.05 (t, J=2.4 Hz, 1H, H_{propargyl}), 3.05-2.95 (m, 4H, C8-CH₂CH₂), 1.82-1.74 (m, 4H, H_{alkyl}), 1.52-1.42 (m, 2H, H_{alkyl}), 1.19 (t, J=7.0 Hz, 6H, 2×CH₂CH₃). ¹³C-NMR (151 MHz, DMSO-d₆) δ [ppm]=153.7 (C8), 152.9 (CO), 150.0 (CO), 148.0 (C6), 143.3 (Cₐᵣₒₘ), 131.1 (Cₐᵣₒₘ), 130.4 (Cₐᵣₒₘ), 129.0 (Cₐᵣₒₘ), 127.4 (Cₐᵣₒₘ), 121.6 (Cₐᵣₒₘ), 106.0 (C5), 79.7 (C_{propargyl}), 72.7 (C_{propargyl}), 60.8 (d, ²J_{C,P}=6.6 Hz, POCH₂), 42.4 (N3-CH₂), 32.7 (CH₂), 30.1 (N1-CH₂), 29.7 (CH₂), 28.1 (d, ²J_{C,P}=16.4 Hz, 1C, PCH₂CH₂), 24.1 (d, ¹J_{C,P}=139.2 Hz, 1C, PCH₂), 19.3 (d, ³J_{C,P}=5.0 Hz, 1C, P(CH₂)₂CH₂), 16.2 (d, ³J_{C,P}=6.5 Hz, POCH₂CH₃). ³¹P-NMR (243 MHz, DMSO-d₆) δ [ppm]=32.8. HRMS (ESI-QTOF) calculated for C₂₄H₃₀BrN₄O₅P [M+H]⁺: 565.1210; found: 565.1226.

C-4) Diethyl (4-(8-(3-methylphenethyl)-2,6-dioxo-1-(prop-2-yn-1-yl)-1,2,6,7-tetrahydro-3H-purin-3-yl)butyl)phosphonate: White solid; m.p. 111-113° C. ¹H-NMR (600 MHz, DMSO-d₆) δ [ppm]=13.31 (br s, 1H, N7-H), 7.15 (t, J=7.5 Hz, 1H, Hₐᵣₒₘ), 7.04 (s, 1H, Hₐᵣₒₘ), 6.98 (t, J=7.7 Hz, 2H, Hₐᵣₒₘ), 4.59 (d, J=2.2 Hz, 2H, N1-CH₂), 4.02-3.98 (m, 2H, N3-CH₂), 3.97-3.91 (m, 4H, 2×OCH₂), 3.06 (t, J=2.4 Hz, 1H, H_{propargyl}), 2.98 (s, 4H, C8-CH₂CH₂), 2.26 (s, 3H, CₐᵣₒₘCH₃), 1.83-1.74 (m, 4H, H_{alkyl}), 1.53-1.43 (m, 2H, H_{alkyl}), 1.19 (t, J=7.0 Hz, 6H, 2×OCH₂CH₃). ¹³C-NMR (151 MHz, DMSO-d₆) δ [ppm]=154.1 (C8), 152.9 (CO), 150.0 (CO), 148.1 (C4), 140.3 (Cₐᵣₒₘ), 137.3 (Cₐᵣₒₘ), 128.9 (Cₐᵣₒₘ), 128.2 (Cₐᵣₒₘ), 126.8 (Cₐᵣₒₘ), 125.2 (Cₐᵣₒₘ), 105.8 (C5), 79.7 (C_{propargyl}), 72.7 (C_{propargyl}), 60.8 (d, ²J_{C,P}=6.1 Hz, POCH₂), 42.4 (N3-CH₂), 33.2 (CH₂), 30.0 (N1-CH₂), 28.1 (d, ²J_{C,P}=15.4 Hz, 1C, PCH₂CH₂), 24.1 (d, ¹J_{C,P}=138.5 Hz, 1C, PCH₂), 21.0 (CH₂), 19.3 (d, ³J_{C,P}=4.4 Hz, 1C, P(CH₂)₂CH₂), 16.3 (d, ³J_{C,P}=5.6 Hz, POCH₂CH₃). ³¹P-NMR (243 MHz, DMSO-d₆) δ [ppm]=32.8. HRMS (ESI-QTOF) calculated for C₂₅H₃₃N₄O₅P [M+H]⁺: 501.2261; found: 501.2262.

C-5) Diethyl (4-(7-methyl-2,6-dioxo-1-(prop-2-yn-1-yl)-8-(3-(trifluoromethyl)phenethyl)-1,2,6,7-tetrahydro-3H-purin-3-yl)butyl)phosphonate: white solid; m.p. 121-124° C. ¹H-NMR (600 MHz, DMSO-d₆) δ [ppm]=7.62 (t, J=1.7 Hz, 1H, Hₐᵣₒₘ), 7.59-7.49 (m, 3H, Hₐᵣₒₘ), 4.57 (d, J=2.5 Hz, 2H, N1-CH₂), 3.99-3.88 (m, 6H, N3-CH₂, POCH₂), 3.76 (s, 3H, N7-CH₃), 3.13-3.10 (m, 4H, CH₂CH₂), 3.05 (t, J=2.4 Hz, 1H, H_{propargyl}), 1.81-1.69 (m, 4H, CH₂CH₂), 1.50-1.41 (m, 2H, CH₂), 1.18 (t, J=7.0 Hz, 6H, POCH₂CH₃). ¹³C-NMR (151 MHz, DMSO-d₆) δ [ppm]=153.7 (C8), 153.3 (CO), 149.8 (CO), 147.3 (C6), 141.9 (Cₐᵣₒₘ), 132.8 (Cₐᵣₒₘ), 129.2 (Cₐᵣₒₘ), 129.0 (q, ²J_{C,F}=32.1 Hz, 1C, Cₐᵣₒₘ—CF₃), 125.1 (q, ³J_{C,F}=3.9 Hz, 1C, Cₐᵣₒₘ), 124.3 (q, ¹J_{C,F}=272.3 Hz, 1C, CF₃), 122.9 (q, ³J_{C,F}=3.8 Hz, 1C, Cₐᵣₒₘ), 106.3 (C5), 79.6 (C_{propargyl}), 72.7 (C_{propargyl}), 60.8 (d, ²J_{C,P}=6.2 Hz, 2C, POCH₂), 42.0 (N3-CH₂), 32.0 (N7-CH₃), 30.8 (N1-CH₂), 29.9 (CH₂), 28.1 (d, ²J_{C,P}=16.5 Hz, 1C, PCH₂CH₂), 27.3 (CH₂), 24.1 (d, ¹J_{C,P}=137.8 Hz, 1C, PCH₂), 19.2 (d, ³J_{C,P}=4.7 Hz, 1C, P(CH₂)₂CH₂), 16.2 (d, ³J_{C,P}=5.9 Hz, POCH₂CH₃). ³¹P-NMR (243 MHz, DMSO-d₆) δ [ppm]=32.8. HRMS (ESI-QTOF) calculated for C₂₋₆H₃₃F3N₄O₅P [M+H]⁺: 569.2135; found: 569.2103.

C-6) Diethyl (4-(8-(3-methoxyphenethyl)-2,6-dioxo-1-(prop-2-yn-1-yl)-1,2,6,7-tetrahydro-3H-purin-3-yl)butyl)phosphonate: White solid; m.p. 135-137° C.; ¹H-NMR (500 MHz, DMSO-d₆) δ [ppm]=13.29 (s, 1H, N7-H), 7.18 (t, J=7.8 Hz, 1H, Hₐᵣₒₘ), 6.79-6.72 (m, 3H, Hₐᵣₒₘ), 4.59 (d, J=2.5 Hz, 2H, N1-CH₂), 4.01-3.98 (m, 2H, N3-CH₂), 3.98-3.90 (m, 4H, POCH₂), 3.71 (s, 3H, OCH₃), 3.05 (t, J=2.4 Hz, 1H, H_{propargyl}), 3.00 (s, 4H, CH₂CH₂), 1.84-1.70 (m, 4H, CH₂CH₂), 1.54-1.40 (m, 2H, CH₂), 1.19 (t, J=7.0 Hz, 6H, POCH₂CH₃). ¹³C-NMR (126 MHz, DMSO-d₆) δ [ppm]=159.2 (Cₐᵣₒₘ—OCH₃), 154.0 (C8), 152.8 (CO), 150.0 (CO), 148.1 (C4), 142.0 (Cₐᵣₒₘ), 129.3 (Cₐᵣₒₘ), 120.4 (Cₐᵣₒₘ), 113.9 (Cₐᵣₒₘ), 111.6 (Cₐᵣₒₘ), 105.8 (C5), 79.7 (C_{propargyl}), 72.6 (C_{propargyl}), 60.7 (d, ²J_{C,P}=6.3 Hz, 1C, POCH₂), 54.8 (OCH₃), 42.4 (N3-CH₂), 33.2 (CH₂), 30.0 (N1-CH₂), 29.9 (CH₂), 28.2 (d, ²J_{C,P}=15.4 Hz, 1C, PCH₂CH₂), 24.1 (d, ¹J_{C,P}=138.9 Hz, 1C, PCH₂), 19.3 (d, ³J_{C,P}=4.7 Hz, 1C, P(CH₂)₂CH₂), 16.2 (d, ³J_{C,P}=5.7 Hz, 2C, POCH₂CH₃). ³¹P-NMR (243 MHz, DMSO-d₆) δ [ppm]=32.8. HRMS (ESI-QTOF) calculated for C₂₅H₃₃N₄O₆P [M+H]⁺: 517.2210; found: 517.2221.

C-7) Diethyl (4-(1-(cyclobutylmethyl)-8-(3-methoxyphenethyl)-2,6-dioxo-1,2,6,7-tetrahydro-3H-purin-3-yl)butyl)phosphonate: White solid; m.p. 82-84° C. ¹H-NMR (600 MHz, DMSO-d₆) δ [ppm]=13.17 (br s, 1H, N7-H), 7.17 (t, J=8.0 Hz, 1H, Hₐᵣₒₘ), 6.77-6.72 (m, 3H, Hₐᵣₒₘ), 3.97 (t, J=7.0 Hz, 2H, N1-CH₂), 3.96-3.93 (m, 2H, N3-CH₂), 3.93-3.91 (m, 4H, POCH₂), 3.69 (s, 3H, OCH₃), 2.99-2.95 (m, 4H, C8-CH₂CH₂), 2.66-2.58 (m, 1H, H_{alkyl}), 1.91-1.85 (m, 2H, H_{alkyl}), 1.79-1.71 (m, 8H, H_{alkyl}), 1.44 (dq, J=15.0, 8.2, 6.2 Hz, 2H, H_{alkyl}), 1.17 (t, J=7.0 Hz, 6H, H_{alkyl}). ¹³C-NMR (151 MHz, DMSO-d₆) δ [ppm]=159.4 (Cₐᵣₒₘ—OCH₃), 154.2 (C8), 153.7 (CO), 151.0 (CO), 147.9 (C4), 142.2 (Cₐᵣₒₘ), 129.5 (Cₐᵣₒₘ), 120.6 (Cₐᵣₒₘ), 114.0 (Cₐᵣₒₘ), 111.8 (Cₐᵣₒₘ), 106.2 (C5), 60.9 (d, ²J_{C,P}=6.5 Hz, POCH₂), 55.0 (OCH₃), 45.1 (N3-CH₂), 42.4 (Cayi), 34.2 (Cayi), 33.5 (Cayi), 30.1 (N1-CH₂), 28.4 (d, ²J_{C,P}=16.4 Hz, 1C, PCH₂CH₂), 25.7 (2C, C_{cyclobutyle}), 24.3 (d, ¹J_{C,P}=138.6 Hz, 1C, PCH₂), 19.4 (d, ³J_{C,P}=5.4 Hz, 1C, P(CH₂)₂CH₂), 17.9 (Ceycio_{buty}ie), 16.4 (d, ³J_{C,P}=5.9 Hz, POCH₂CH₃). ³¹P-NMR (243 MHz, DMSO-d₆) δ [ppm]=32.8. HRMS (ESI-QTOF) calculated for C27H₃₉N₄O₆P [M+H]⁺: 547.2680; found: 547.2678.

C-8) Diethyl (4-(8-(3,4-dimethoxyphenethyl)-2,6-dioxo-1-(prop-2-yn-1-yl)-1,2,6,7-tetrahydro-3H-purin-3-yl)butyl)phosphonate: White solid; m.p. n.d. ¹H NMR (600 MHz, DMSO-d₆) δ 13.27 (s, 1H, N7-H), 6.83 (d, J=8.2 Hz, 1H, Hₐᵣₒₘ), 6.78 (d, J=2.0 Hz, 1H, Hₐᵣₒₘ), 6.69 (dd, J=8.2, 2.0 Hz, 1H, Hₐᵣₒₘ), 4.59 (d, J=2.5 Hz, 2H, N—CH₂), 4.00 (t, J=6.9 Hz, 2H, N—CH₂), 3.97-3.91 (m, 4H, 2×OCH₂), 3.70 (s, 6H, 2×OCH₃), 3.06 (t, J=2.4 Hz, 1H, H_{propargyl}), 2.99-2.93 (m, 4H, CH₂—CH₂), 1.82-1.74 (m, 4H, H_{alkyl}), 1.52-1.44 (m, 2H, H_{alkyl}), 1.19 (t, J=7.0 Hz, 6H, 2×OCH₂CH₃). ¹³C NMR (151 MHz, DMSO) δ 154.2 (Cₓₐₙₜₕᵢₙₑ), 152.8 (Cₓₐₙₜₕᵢₙₑ), 150.0 (Cₓₐₙₜₕᵢₙₑ), 148.6 (Cₓₐₙₜₕᵢₙₑ), 148.1 (Cₓₐₙₜₕᵢₙₑ), 147.2 (Cₓₐₙₜₕᵢₙₑ), 132.8 (Cₐᵣₒₘ), 120.0 (Cₐᵣₒₘ), 112.2 (Cₐᵣₒₘ), 111.9 (Cₐᵣₒₘ), 105.8 (C5), 79.7 (C_{propargyl}), 72.6 (C_{propargyl}), 60.8 (d, ²J_{C,P}=6.6 Hz, POCH₂), 55.5 (2×OCH₃), 42.4 (C_{alkyl}), 32.9 (C_{alkyl}), 30.3 (C_{alkyl}), 30.0 (C_{alkyl}), 28.2 (d, ²J_{C,P}=16.1 Hz, 1C, PCH₂CH₂), 24.0 (d, ¹J_{C,P}=138.4 Hz, 1C, PCH₂), 19.3 (d, ³J_{C,P}=4.8 Hz, 1C, P(CH₂)₂CH₂), 16.2 (d, ³J_{C,P}=5.6 Hz, POCH₂CH₃). ³¹P NMR (243 MHz, DMSO) δ 32.8.

C-9) Diethyl (4-(1-ethyl-2,6-dioxo-8-(2-phenylcyclopropyl)-1,2,6,7-tetrahydro-3H-purin-3-yl)butyl)phosphonate: Off-white solid; m.p. 124-126° C. ¹H NMR (600 MHz, DMSO-d₆): δ [ppm]=13.10 (s, 1H, N7-H), 7.31-7.26 (m, 2H, Hₐᵣₒₘ), 7.21-7.16 (m, 3H, Hₐᵣₒₘ), 3.99-3.95 (in, 2H, N1-CH₂ or N3-CH₂), 3.95-3.87 (m, 6H, 2×OCH₂ and N1-CH₂ or N3-CH₂), 2.25-2.20 (m, 1H, CH_{cyclopropyle}), 1.84-1.67 (m, 5H, CH_{cyclopropyle} and 2×H_{alkyl}), 1.62-1.54 (m, 1H, CH_{cyclopropyle}), 1.48-1.37 (m, 2H, H_{alkyl}), 1.16 (td, J=7.0, 2.7 Hz, 6H, 2×OCH₂CH₃), 1.10 (t, J=7.0 Hz, 3H, CH₃). ¹³C NMR (151 MHz, DMSO): δ [ppm]=154.7 (Cₓₐₙₜₕᵢₙₑ), 153.5 (Cₓₐₙₜₕᵢₙₑ), 150.6 (Cₓₐₙₜₕᵢₙₑ), 148.1 (Cₓₐₙₜₕᵢₙₑ), 140.7 (Cₓₐₙₜₕᵢₙₑ), 128.6 (Cₐᵣₒₘ), 126.3 (Cₐᵣₒₘ), 125.9 (Cₐᵣₒₘ), 106.1 (C5), 60.9 (d, ²J_{C,P}=6.1 Hz, POCH₂), 42.2 (C_{alkyl}), 35.7 (C_{alkyl}), 28.3 (d, ²J_{C,P}=16.1 Hz, 1C, PCH₂CH₂), 27.1 (C_{cyclopropyle}), 24.1 (d, ¹J_{C,P}=139.0 Hz, 1C, PCH₂), 21.4 (C_{cyclopropyle}), 19.4 (d, ³J_{C,P}=5.1 Hz, 1C, P(CH₂)₂CH₂), 17.5 (C_{cyclopropyle}), 16.4 (d, ³J_{C,P}=6.2 Hz, POCH₂CH₃), 13.3 (CH₃). ³¹P NMR (243 MHz, DMSO): δ [ppm]=32.9. LC-MS: positive mode [m/z]=490.2 ([M+H]⁺). ESI-MS purity: 93.5%.

C-10) Diethyl (4-(1-ethyl-2,6-dioxo-8-(phenoxymethyl)-1,2,6,7-tetrahydro-3H-purin-3-yl)butyl)phosphonate: Off-white solid; m.p. n.d. ¹H NMR (600 MHz, DMSO-d₆) δ 13.80 (s, 1H, N7-H), 7.34-7.29 (m, 1H, Hₐᵣₒₘ), 7.07-7.03 (m, 1H, Hₐᵣₒₘ), 7.00-6.96 (m, 1H, Hₐᵣₒₘ), 5.13 (s, 2H, C8-CH₂), 4.00 (t, J=6.9 Hz, 2H, N—CH₂), 3.98-3.90 (m, 3H, H_{propargyl}), 1.81-1.73 (m, 4H, H_{alkyl}), 1.52-1.44 (m, 2H, H_{alkyl}), 1.18 (t, J=7.0 Hz, 6H, 2×OCH₂CH₃), 1.12 (t, J=7.0 Hz, 3H, CH₂CH₃). ¹³C NMR (151 MHz, DMSO) δ 157.7 (Cₓₐₙₜₕᵢₙₑ), 153.9 (Cₓₐₙₜₕᵢₙₑ), 150.4 (Cₓₐₙₜₕᵢₙₑ), 148.6 (Cₓₐₙₜₕᵢₙₑ), 147.5 (Cₓₐₙₜₕᵢₙₑ), 129.5 (2C, Cₐᵣₒₘ), 121.3 (Cₐᵣₒₘ), 114.8 (2C, Cₐᵣₒₘ), 62.7 (OCH₂), 60.7 (2×OCH₂), 42.3 (C_{alkyl}), 35.7 (C_{alkyl}), 28.3 (C_{alkyl}), 28.2 (d, ²J_{C,P}=15.6 Hz, 1C, PCH₂CH₂), 24.0 (d, ¹J_{C,P}=140.0 Hz, 1C, PCH₂), 19.3 (d, ³J_{C,P}=4.8 Hz, 1C, P(CH₂)₂CH₂), 16.2 (d, ³J_{C,P}=5.7 Hz, POCH₂CH₃), 13.1 (C_{alkyl}). ³¹P NMR (243 MHz, DMSO) δ 32.8.

### Synthesis of Compounds Class D (D-1)-D-3

D-1) Ethyl hydrogen (4-(8-(2-bromophenethyl)-7-methyl-2,6-dioxo-1-(prop-2-yn-1-yl)-1,2,6,7-tetrahydro-3H-purin-3-yl)butyl)phosphonate: White solid; m.p. n.d.; ¹H-NMR (600 MHz, DMSO-d₆) δ [ppm]=7.60 (dd, J=7.9, 1.2 Hz, 1H, Hₐᵣₒₘ), 7.35 (dd, J=7.7, 1.8 Hz, 1H, Hₐᵣₒₘ), 7.31 (td, J=7.4, 1.2 Hz, 1H, Hₐᵣₒₘ), 7.20-7.14 (m, 1H, Hₐᵣₒₘ), 4.59 (d, J=2.5 Hz, 2H, N1-CH₂), 3.97 (t, J=7.0 Hz, 2H, N3-CH₂), 3.92-3.85 (m, 2H, POCH₂), 3.74 (s, 3H, N7-CH₃), 3.13 (t, J=7.2 Hz, 2H, —CH₂), 3.09-3.04 (m, 3H, —CH₂ and H_{propargyl}), 1.77-1.70 (m, 2H, —CH₂), 1.65 (ddd, J=18.0, 9.3, 6.5 Hz, 2H, —CH₂), 1.52-1.41 (m, 2H, —CH₂), 1.18 (t, J=7.0 Hz, 3H, POCH₂CH₃). ¹³C-NMR (151 MHz, DMSO-d₆) δ [ppm]=153.5 (C8 or C4), 153.3 (C8 or C4), 149.8 (CO), 147.4 (CO), 139.3 (Cₐᵣₒₘ), 132.5 (Cₐᵣₒₘ), 131.0 (Cₐᵣₒₘ), 128.6 (Cₐᵣₒₘ), 127.9 (Cₐᵣₒₘ), 123.7 (Cₐᵣₒₘ), 106.4 (C5), 79.6 (C_{propargyl}), 72.7 (C_{propargyl}), 59.8 (d, ²J_{C,P}=6.2 Hz, 1C, POCH₂CH₃), 42.2 (N—CH₂), 32.9 (N—CH₂), 31.3 (N7-CH₃), 29.9 (—CH₂), 28.3 (d, ²J_{C,P}=15.8 Hz, 1C, PCH₂CH₂), 26.3 (—CH₂), 25.4 (d, ¹J_{C,P}=138.2 Hz, 1C, PCH₂), 19.6 (d, ³J_{C,P}=4.4 Hz, PCH₂CH₂CH₂), 16.4 (d, ³J_{C,P}=6.1 Hz, POCH₂CH₃). ³¹P-NMR (243 MHz, DMSO) δ [ppm]=29.7.

D-2) Ethyl hydrogen (4-(8-(2-methoxyphenethyl)-2,6-dioxo-1-(prop-2-yn-1-yl)-1,2,6,7-tetrahydro-3H-purin-3-yl)butyl)phosphonate: White solid; m.p. 172-174° C. ¹H-NMR (600 MHz, DMSO-d₆) δ [ppm]=13.26 (br s, 1H, N7-H), 7.21-7.17 (m, 1H, Hₐᵣₒₘ), 7.16-7.13 (m, 1H, Hₐᵣₒₘ), 6.96 (d, J=8.2 Hz, 1H, Hₐᵣₒₘ), 6.84 (t, J=7.4 Hz, 1H, Hₐᵣₒₘ), 4.58 (d, J=2.5 Hz, 2H, N1-CH₂), 4.17 (q, J=7.1 Hz, 2H, POCH₂), 3.96 (t, J=7.1 Hz, 2H, N3-CH₂), 3.77 (s, 3H, OCH₃), 3.05 (t, J=2.3 Hz, 1H, H_{propargyl}), 2.96 (dt, J=11.4, 5.7 Hz, 4H, C8-CH₂CH₂), 1.73 (p, J=7.4 Hz, 2H, H_{alkyl}), 1.59-1.52 (m, 2H, H_{alkyl}), 1.51-1.45 (m, 2H, H_{alkyl}), 1.23 (t, J=7.1 Hz, 3H, POCH₂CH₃). ¹³C-NMR (151 MHz, DMSO-d₆) δ [ppm]=157.3 (Cₐᵣₒₘ—OCH₃), 153.8 (Cₓₐₙₜₕᵢₙₑ), 153.0 (Cₓₐₙₜₕᵢₙₑ), 150.0 (Cₓₐₙₜₕᵢₙₑ), 147.9 (Cₓₐₙₜₕᵢₙₑ), 130.1 (Cₐᵣₒₘ), 128.1 (Cₐᵣₒₘ), 128.0 (Cₐᵣₒₘ), 120.4 (Cₐᵣₒₘ), 110.8 (Cₐᵣₒₘ), 105.6 (C5), 79.9 (C_{propargyl}), 72.9 (C_{propargyl}), 55.4 (2C, POCH₂ and OCH₃), 42.6 (N3-CH₂), 39.6 (POCH₂), 30.1 (N1-CH₂), 28.7 (d, ²J_{C,P}=16.5 Hz, 1C, PCH₂CH₂), 28.6 (CH₂), 27.4 (d, ¹J_{C,P}=136.0 Hz, 1C, PCH₂), 26.4 (CH₂), 20.2 (d, ³J_{C,P}=4.2 Hz, 1C, PCH₂CH₂CH₂), 16.0 (POCH₂CH₃). ³¹P-NMR (243 MHz, DMSO-d₆) δ [ppm]=27.1. HRMS (ESI-QTOF) calculated for C₂₃H₃₀N₄O₆P [M+H]⁺: 489.1903; found: 489.1904.

D-3) Ethyl hydrogen (4-(8-(3-methoxyphenethyl)-2,6-dioxo-1-(prop-2-yn-1-yl)-1,2,6,7-tetrahydro-3H-purin-3-yl)butyl)phosphonate: White solid; m.p. 118-120° C. ¹H-NMR (600 MHz, DMSO-d₆) δ [ppm]=7.19 (t, J=7.8 Hz, 1H, Hₐᵣₒₘ), 6.85-6.82 (m, 2H, Hₐᵣₒₘ), 6.76 (dd, J=8.2, 2.3 Hz, 1H, Hₐᵣₒₘ), 4.60 (d, J=2.2 Hz, 2H, N1-CH₂), 4.18 (q, J=7.1 Hz, 4H, POCH₂), 4.00 (t, J=7.1 Hz, 2H, N3-CH₂), 3.71 (s, 3H, OCH₃), 3.10-3.08 (m, 1H, H_{propargyl}), 3.08-3.06 (m, 2H, C8-CH₂), 3.02 (t, J=7.4 Hz, 2H, C8-CH₂CH₂), 1.79-1.73 (m, 2H, H_{alkyl}), 1.62-1.55 (m, 2H, H_{alkyl}), 1.51 (dq, J=15.5, 8.6, 7.3 Hz, 2H, H_{alkyl}), 1.20 (t, J=7.1 Hz, 3H, CH₂CH₃). ¹³C-NMR (151 MHz, DMSO-d₆) δ [ppm]=159.8 (Cₐᵣₒₘ OCH₃), 153.9 (Cₓₐₙₜₕᵢₙₑ), 153.3 (Cₓₐₙₜₕᵢₙₑ), 150.3 (Cₓₐₙₜₕᵢₙₑ), 148.2 (Cₓₐₙₜₕᵢₙₑ), 142.5 (Cₐᵣₒₘ), 129.8 (Cₐᵣₒₘ), 121.2 (Cₐᵣₒₘ), 114.6 (Cₐᵣₒₘ), 112.2 (Cₐᵣₒₘ), 105.9 (C5), 80.2 (C_{propargyl}), 73.2 (C_{propargyl}), 55.4 (OCH₃), 42.9 (N3-CH₂), 39.6 (POCH₂), 33.5 (CH₂), 30.4 (N1-CH₂), 29.0 (d, ²J_{C,P}=15.4 Hz, 1C, PCH₂CH₂), 28.0 (CH₂), 27.7 (d, J_{C,P}=135 Hz, 1C, PCH₂), 20.5 (d, ³J_{C,P}=4.7 Hz, 1C, PCH₂CH₂CH₂), 16.3 (POCH₂CH₃). ³¹P-NMR (243 MHz, DMSO-d₆) δ [ppm]=27.2. HRMS (ESI-QTOF) calculated for C₂₃H₃₀N₄O₆P [M+H]⁺: 489.1903; found: 489.1905.

E-1) 4-(8-(2-Bromophenethyl)-2,6-dioxo-1-(prop-2-yn-1-yl)-1,2,6,7-tetrahydro-3H-purin-3-yl)butane-1-sulfonic acid: ¹H NMR (500 MHz, DMSO-d₆) δ 7.58 (d, J=7.9 Hz, 1H, Hₐᵣₒₘ), 7.31-7.29 (m, 2H, Hₐᵣₒₘ), 7.15 (dt, J=8.0, 4.5 Hz, 1H, Hₐᵣₒₘ), 4.60 (d, J=2.5 Hz, 2H, CH₂), 3.97 (t, J=7.2 Hz, 2H, CH₂), 3.19-3.10 (m, 2H, CH₂), 3.07-3.00 (m, 3H, CH₂ and H_{propargyl}), 1.80-1.69 (m, 2H, H_{alkyl}), 1.64-1.54 (m, 2H, H_{alkyl}). ¹³C NMR (126 MHz, DMSO) δ 153.3 (Cₓₐₙₜₕᵢₙₑ), 152.9 (Cₓₐₙₜₕᵢₙₑ), 149.9 (Cₓₐₙₜₕᵢₙₑ), 147.9 (Cₓₐₙₜₕᵢₙₑ), 139.3 (Cₐᵣₒₘ), 132.5 (Cₐᵣₒₘ), 130.7 (Cₐᵣₒₘ), 128.4 (Cₐᵣₒₘ), 127.9 (Cₐᵣₒₘ), 123.7 (Cₐᵣₒₘ), 106.0 (C5), 79.7 (C_{propargyl}), 72.7 (C_{propargyl}), 51.1 (C_{alkyl}), 43.0 (C_{alkyl}), 33.5 (C_{alkyl}), 30.0 (C_{alkyl}), 28.3 (C_{alkyl}), 26.9 (C_{alkyl}), 22.2 (C_{alkyl}).

E-2) 4-(1-Ethyl-8-(3-methoxyphenethyl)-2,6-dioxo-1,2,6,7-tetrahydro-3H-purin-3-yl)butane-1-sulfonic acid: White solid; m.p. 179-181° C. ¹H-NMR (600 MHz, DMSO-d₆) δ [ppm]=7.17 (t, J=7.8 Hz, 1H, Hₐᵣₒₘ), 6.81-6.77 (m, 2H, Hₐᵣₒₘ), 6.74 (dd, J=8.2, 2.5 Hz, 1H, Hₐᵣₒₘ), 3.95 (t, J=7.1 Hz, 2H, N1-CH₂), 3.91 (q, J=7.0 Hz, 2H, N3-CH₂), 3.71 (s, 3H, OCH₃), 3.02-2.96 (m, 4H, CH₂CH₂), 2.49-2.44 (m, 2H, CH₂), 1.78-1.67 (m, 2H, CH₂), 1.58 (q, J=7.7, 7.1 Hz, 2H, CH₂), 1.11 (t, J=7.0 Hz, 3H, CH₃). ¹³C-NMR (151 MHz, DMSO-d₆) δ [ppm]=159.4 (Cₐᵣₒₘ—OCH₃), 153.8 (C8), 153.6 (CO), 150.5 (CO), 147.7 (C6), 142.2 (Cₐᵣₒₘ), 129.5 (Cₐᵣₒₘ), 120.7 (Cₐᵣₒₘ), 114.0 (Cₐᵣₒₘ), 111.8 (Cₐᵣₒₘ), 106.4 (C5), 55.0 (OCH₃), 51.3 (CH₂SO₃H), 43.0 (N3-CH₂), 35.8 (CH₂), 33.5 (CH₂), 30.1 (CH₂), 27.2 (CH₂), 22.5 (CH₂), 13.4 (CH₃). HRMS (ESI-QTOF) calculated for C₂₀H27N₄O₆S [M+H]⁺: 451.1646; found: 451.1639.

E-3) 4-(8-(3-Methoxyphenethyl)-2,6-dioxo-1-(prop-2-yn-1-yl)-1,2,6,7-tetrahydro-3H-purin-3-yl)butane-1-sulfonic acid: White solid; m.p. 175-177° C. ¹H-NMR (500 MHz, DMSO-d₆) δ [ppm]=7.20-7.15 (m, 1H, Hₐᵣₒₘ), 6.81-6.76 (m, 2H, Hₐᵣₒₘ), 6.76-6.72 (m, 1H, Hₐᵣₒₘ), 4.60 (d, J=2.5 Hz, 2H, N1-CH₂), 3.97 (t, J=7.2 Hz, 2H, N3-CH₂), 3.71 (s, 3H, OCH₃), 3.04 (t, J=2.4 Hz, 1H, H_{propargyl}), 3.02-2.99 (m, 4H, CH₂CH₂), 2.50-2.48 (m, 2H, CH₂), 1.78-1.68 (m, 2H, CH₂), 1.64-1.54 (m, 2H, CH₂). ¹³C-NMR (126 MHz, DMSO-d₆) δ [ppm]=159.4 (Cₐᵣₒₘ—OCH₃), 154.1 (C8), 153.0 (CO), 150.1 (CO), 148.1 (C6), 142.2 (Cₐᵣₒₘ), 129.5 (Cₐᵣₒₘ), 120.7 (Cₐᵣₒₘ), 114.0 (Cₐᵣₒₘ), 111.8 (Cₐᵣₒₘ), 106.0 (C5), 79.9 (C_{propargyl}), 72.8 (C_{propargyl}), 55.0 (OCH₃), 51.2 (CH₂SO₃H), 43.2 (N3-CH₂), 33.4 (CH₂), 30.2 (CH₂), 30.1 (CH₂), 27.1 (CH₂), 22.4 (CH₂). HRMS (ESI-QTOF) calculated for C₂₁H₂₅N₄O₆S [M+H]⁺: 561.1489; found: 461.1482.

F-1) 4-(8-(2-Bromophenethyl)-2,6-dioxo-1-(prop-2-yn-1-yl)-1,2,6,7-tetrahydro-3H-purin-3-yl)butane-1-sulfonamide: White solid; m.p. 216-219° C.; ¹H-NMR (500 MHz, DMSO-d₆) δ [ppm]=7.59 (d, J=7.9 Hz, 1H, Hₐᵣₒₘ), 7.31-7.28 (m, 2H, Hₐᵣₒₘ), 7.19-7.13 (m, 1H, Hₐᵣₒₘ), 6.72 (s, 2H, NH₂), 4.60 (d, J=2.5 Hz, 2H, N—CH₂), 4.00 (t, J=7.0 Hz, 2H, N—CH₂), 3.15 (dd, J=8.5, 6.7 Hz, 2H, CH₂), 3.09-2.97 (m, 5H, H_{propargyl}, CH₂), 1.85-1.75 (m, 2H, CH₂), 1.76-1.65 (m, 2H, CH₂). ¹³C-NMR (126 MHz, DMSO-d₆) δ [ppm]=153.7 (C8 or C4), 153.1 (C8 or C4), 150.2 (CO), 148.2 (CO), 139.5 (Cₐᵣₒₘ), 132.6 (Cₐᵣₒₘ), 130.8 (Cₐᵣₒₘ), 128.6 (Cₐᵣₒₘ), 128.0 (Cₐᵣₒₘ), 123.8 (Cₐᵣₒₘ), 106.3 (C5), 79.9 (C_{propargyl}), 72.8 (C_{propargyl}), 54.1 (CH₂), 42.6 (CH₂), 33.7 (CH₂), 30.2 (CH₂), 28.5 (CH₂), 26.4 (CH₂), 20.9 (CH₂).

G-1) 5-(8-(2-Methoxyphenethyl)-2,6-dioxo-1-(prop-2-yn-1-yl)-1,2,6,7-tetrahydro-3H-purin-3-yl)pentanoic acid: White solid; m.p. 196-198° C.; ¹H-NMR (500 MHz, DMSO-d₆) δ [ppm]=7.18 (td, J=7.9, 1.8 Hz, 1H, Hₐᵣₒₘ), 7.08 (dd, J=7.4, 1.7 Hz, 1H, Hₐᵣₒₘ), 6.95 (dd, J=8.3, 1.1 Hz, 1H, Hₐᵣₒₘ), 6.82 (td, J=7.4, 1.1 Hz, 1H, Hₐᵣₒₘ), 4.59 (d, J=2.5 Hz, 2H, N1-CH₂), 3.98 (t, J=7.1 Hz, 2H, N3-CH₂), 3.04 (t, J=2.4 Hz, 1H, H_{propargyl}), 3.01-2.90 (m, 4H, CH₂CH₂), 2.26 (t, J=7.3 Hz, 2H, CH₂), 1.75-1.63 (m, 2H, CH₂), 1.56-1.45 (m, 2H, CH₂). ¹³C-NMR (126 MHz, DMSO-d₆) δ [ppm]=174.2 (COOH), 157.1 (C8), 154.3 (CO), 152.8 (CO), 150.0 (C6), 148.0 (Cₐᵣₒₘ), 129.6 (Cₐᵣₒₘ), 128.1 (Cₐᵣₒₘ), 127.6 (Cₐᵣₒₘ), 120.2 (Cₐᵣₒₘ), 110.6 (Cₐᵣₒₘ), 105.9 (C5), 79.8 (Cₚro_{pargy}), 72.6 (C_{propargyl}), 55.2 (OCH₃), 42.6 (N3-CH₂), 33.2 (CH₂), 30.0 (CH₂), 28.4 (CH₂), 28.3 (CH₂), 27.0 (CH₂), 21.6 (CH₂). HRMS (ESI-QTOF) calculated for C₂₂H₂₅N₄O₅ [M+H]⁺: 425.1819; found: 425.1830.

G-2) 5-(1-(Cyclobutylmethyl)-2,6-dioxo-8-(3-(trifluoromethyl)phenethyl)-1,2,6,7-tetrahydro-3H-purin-3-yl)pentanoic acid: Off-white solid; m.p. 210-212° C. ¹H-NMR (600 MHz, DMSO-d₆) δ [ppm]=13.04 (br s, 1H, COOH), 7.48-7.52 (m, 4H, Hₐᵣₒₘ), 3.97-3.89 (m, 4H, N1-CH₂, N3-CH₂), 3.11 (t, J=7.6 Hz, 2H, CH₂), 3.02 (t, J=7.5 Hz, 2H, CH₂), 2.62 (m, 1H, N1-CH₂CH), 2.23 (t, J=7.4 Hz, 2H, H_{alkyl}), 1.88 (td, J=8.0, 7.4, 3.4 Hz, 2H, H_{alkyl}), 1.73-1.77 (m, 6H, H_{alkyl}), 1.65 (p, J=7.3 Hz, 2H, H_{alkyl}), 1.47 (p, J=7.5 Hz, 2H, Hₐii). ¹³C-NMR (151 MHz, DMSO-d₆) δ [ppm]=174.4 (COOH), 154.2 (C8), 153.3 (CO), 151.0 (CO), 147.8 (C6), 142.1 (Cₐᵣₒₘ), 132.7 (Cₐᵣₒₘ), 129.4 (Cₐᵣₒₘ), 129.1 (q, ²J_{C,F}=31.5 Hz, 1C, Cₐᵣₒₘ—CF3), 124.4 (q, ¹J_{C,F}=275 Hz, 1C, CF₃), 125.1 (q, ³J_{C,F}=4.0 Hz, 1C, Cₐᵣₒₘ), 123.0 (q, J_{C,F}=4.1 Hz, 1C, Cₐᵣₒₘ), 106.3 (C5), 45.1 (N1-CH₂), 42.6 (N3-CH₂), 34.2 (C_{alkyl}), 33.3 (C_{alkyl}), 33.0 (C_{alkyl}), 29.8 (C_{alkyl}), 27.1 (C_{alkyl}), 25.7 (2C, C_{cyclobutyle}), 21.6 (C_{alkyl}), 17.9 (C_{alkyl}). HRMS (ESI-QTOF) calculated for C₂₄H₂₇F₃N₄O₄ [M+H]⁺: 493.2057; found: 493.2077.

G-3) 6-(8-(2-Bromophenethyl)-2,6-dioxo-1-(prop-2-yn-1-yl)-1,2,6,7-tetrahydro-3H-purin-3-yl) hexanoic acid: White solid; m.p. 192-194° C. ¹H NMR (600 MHz, DMSO-d₆) δ [ppm]=13.33 (s, 1H, N7-H), 11.97 (s, 1H, COOH), 7.60-7.57 (m, 1H, Hₐᵣₒₘ), 7.29-7.27 (m, 2H, 2Hₐᵣₒₘ), 7.15 (ddd, J=7.9, 5.9, 3.1 Hz, 1H, Hₐᵣₒₘ), 4.59 (d, J=2.5 Hz, 2H, N1-CH₂), 3.97 (t, J=7.4 Hz, 2H, N3-CH₂), 3.14 (t, J=7.6 Hz, 2H, CH₂), 3.06 (t, J=2.4 Hz, 1H, H_{propargyl}), 3.02 (t, J=7.6 Hz, 2H, CH₂), 2.20 (t, J=7.3 Hz, 2H, CH₂), 1.66 (p, J=7.8 Hz, 2H, CH₂), 1.54 (p, J=7.4 Hz, 2H, CH₂), 1.33-1.25 (m, 2H, CH₂). ¹³C NMR (151 MHz, DMSO-d₆) δ [ppm]=174.3 (COOH), 153.4 (Cₓₐₙₜₕᵢₙₑ), 152.9 (Cₓₐₙₜₕᵢₙₑ), 150.0 (Cₓₐₙₜₕᵢₙₑ), 148.1 (Cₓₐₙₜₕᵢₙₑ), 139.3 (Cₐᵣₒₘ), 132.5 (Cₐᵣₒₘ), 130.6 (Cₐᵣₒₘ), 128.5 (Cₐᵣₒₘ), 127.8 (Cₐᵣₒₘ), 123.7 (Cₐᵣₒₘ), 105.9 (C5), 79.7 (C_{propargyl}), 72.7 (C_{propargyl}), 42.8 (N3-CH₂), 33.5 (CH₂), 33.4 (CH₂), 30.0 (N1-CH₂), 28.3 (CH₂), 27.1 (CH₂), 25.5 (CH₂), 24.1 (CH₂). HRMS (ESI-QTOF) calculated for C₂₂H₂₃BrN₄O₄[M+H]⁺: 487.098; found: 487.094.

H-1) 3-(8-(3-Methoxyphenethyl)-7-methyl-2,6-dioxo-1-(prop-2-yn-1-yl)-1,2,6,7-tetrahydro-3H-purin-3-yl)propyl dihydrogen phosphate: White solid; Yield 60%; ¹H-NMR (600 MHz, DMSO-d₆) δ [ppm]=7.18 (t, J=7.8 Hz, 1H, Hₐᵣₒₘ), 6.87-6.60 (m, 3H, Hₐᵣₒₘ), 4.57 (d, J=2.5 Hz, 2H, N1-CH₂), 4.12-3.95 (m, 2H, N3-CH₂), 3.88 (s, 3H, N8-CH₃), 3.71 (m, 5H, OCH₂, OCH₃), 3.08-2.98 (m, 4H, C8_{alkyl}), 2.97 (m, 1H, H_{propargyl}), 1.96 (m, 2H, H_{alkyl})). ¹³C-NMR (151 MHz, DMSO-d₆) δ [ppm]=159.46 (Cₐᵣₒₘ—OCH₃), 154.32 (C8), 153.42 (C6), 149.95 (C2), 147.40 (C4), 142.13 (Cₐᵣₒₘ) 129.50 (Cₐᵣₒₘ), 120.86 (Cₐᵣₒₘ), 114.17 (C5), 111.97 (Cₐᵣₒₘ), 106.48 (Cₐᵣₒₘ), 79.83 (C_{propargyl}), 72.89 (C_{propargyl}), 63.29 (N7-CH₃), 55.09 (OCH₃), 32.93 (N1-CH₃), 31.46 (N2-CH₃), 30.06, 28.89, 27.92 (C_{alkyls}). ³¹P-NMR (243 MHz, DMSO-d₆) δ [ppm]=0.01. (HRMS (ESI-QTOF) calculated for C₂₁H₂₅N₄O₇P [M+H]⁺: 499.1359; found: 499.1353).

H-2) 3-(8-(3-Methoxyphenethyl)-7-methyl-2,6-dioxo-1-propyl-1,2,6,7-tetrahydro-3H-purin-3-yl)propyl dihydrogen phosphate: White solid; Yield 64%; ¹H-NMR (600 MHz, DMSO-d₆) δ [ppm]=7.17 (t, J=8.0 Hz, 1H, Hₐᵣₒₘ), 6.84-6.78 (m, 2H, Hₐᵣₒₘ), 6.79-6.53 (m, 1H, Hₐᵣₒₘ), 4.03 (t, J=7.3 Hz, 2H, N1-CH₂), 3.86 (q, J=6.6 Hz, 2H, N3-CH₂), 3.84 (s, 3H, N8-CH₃), 3.70-386 (m, 5H, H_{alkyl}, OCH₃), 3.02 (m, 2H, H_{alkyl}), 2.95-2.94 (m, 2H, H_{alkyl}), 1.95-1.93 (m, 2H H_{alkyl}), 1.54 (m, 2H, H_{alkyl}), 0.84 (t, J=7.4 Hz, 3H, CH₃). ¹³C-NMR (151 MHz, DMSO-d₆) δ [ppm]=159.77 (Cₐᵣₒₘ—OCH₃), 154.70 (C8), 154.15 (C6), 150.82 (C2), 147.42 (C4), 142.51 (C5), 129.80, 121.17, 114.46, 112.27, 106.92 (Cₐᵣₒₘ), 63.51 (N7-CH₃), 55.39 (OCH₃), 42.31, 33.26, 31.68, 29.27, 28.22, 21.28, 11.64 (C_{alkyl}). ³¹P-NMR (243 MHz, DMSO-d₆) δ [ppm] 0.22. (HRMS (ESI-QTOF) calculated for C₂₁H29N₄O7P [M+Na]⁺: 503.1672; found: 503.1666).

H-3) (E)-3-(8-(3-Methoxystyryl)-2,6-dioxo-1-(prop-2-yn-1-yl)-1,2,6,7-tetrahydro-3H-purin-3-yl)propyl dihydrogen phosphate: White solid; Yield 41%; ¹H-NMR (600 MHz, DMSO-d₆) δ [ppm]=13.77 (br s, NH), 7.65 (d, J=16.4 Hz, 1H, CH═CH), 7.32-7.30 (m, 1H, Hₐᵣₒₘ), 7.22-7.16 (m, 2H, Hₐᵣₒₘ, CH═CH), 7.12 (d, J=16.4 Hz, 1H, CH═CH), 6.96-6.90 (m, 1H, Hₐᵣₒₘ), 4.61 (d, J=2.5 Hz, 2H, N1-CH₂), 4.14-4.08 (m, 2H, N3-CH₂), 3.92 (q, J=6.7 Hz, 2H, OCH₂), 3.80 (s, 3H, OCH₃), 3.09 (t, J=2.4 Hz, 1H, H_{propargyl}), 2.02 (p, J=6.8 Hz, 2H, H_{alkyl}). ¹³C-NMR (151 MHz, DMSO-d₆) δ [ppm]=159.84 (Cₐᵣₒₘ), 153.06 (C8), 150.20 (C6), 136.98 (C2), 135.68 (C4), 130.15 (C═C), 130.10 (C═C), 119.86 (Cₐᵣₒₘ), 116.25 (Cₐᵣₒₘ), 115.36 (Cₐᵣₒₘ), 112.18 (Cₐᵣₒₘ), 79.84 (C_{propargyl}), 73.00 (C_{propargyl}), 63.43 (OCH₂), 51.23 (OCH₂), 30.36, 28.90 (C_{alkyl}). ³¹P-NMR (243 MHz, DMSO-d₆) δ [ppm] 0.03. HRMS (ESI-QTOF) calculated for C₂₀H₂₁N₄O₇P [M+H]⁺: 461.1226; found: 461.1221).

H-4) (E)-3-(2,6-Dioxo-1-propyl-8-styryl-1,2,6,7-tetrahydro-3H-purin-3-yl)propyl dihydrogen phosphate: White solid; Yield 61%; ¹H NMR (500 MHz, DMSO-d₆) δ 13.56 (s, 1H, 7-NH), 7.82-7.56 (m, 3H, Hₐᵣₒₘ, CH═C—), 7.38 (dt, J=31.0, 7.3 Hz, 3H, Hₐᵣₒₘ), 7.04 (d, J=16.4 Hz, 1H, C═CH), 4.13-4.04 (m, 2H, N1CH₂), 3.91 (q, J=6.7 Hz, 2H, N3CH₂), 3.87-3.81 (m, 2H, OCH₂), 2.01 (p, J=6.7 Hz, 2H, H_{alkyl}), 1.66-1.35 (m, 2H, H_{alkyl}), 0.86 (t, J=7.4 Hz, 3H, CH₃). ¹³C NMR (126 MHz, DMSO-d₆) δ 154.02 (C8), 150.76 (C6), 149.62 (C2), 148.27 (C4), 135.56 (C═C), 135.36 (C═C), 129.23 (C5), 129.10 (Cₐᵣₒₘ), 127.27 (Cₐᵣₒₘ), 115.93 (Cₐᵣₒₘ), 107.42 (Cₐᵣₒₘ), 63.38 (OCH₂), 40.23 (N1_{alkyl}), 28.95 23 (N3_{alkyl}), 28.89, 20.98, 11.33 (C_{alkyl}). ³¹P-NMR (243 MHz, DMSO-d₆) δ [ppm] 0.03. HRMS (ESI-QTOF) calculated for C₁₉H₂₃N₄O₆P [M+H]⁺: 457.1253; found: 457.1247).

H-5) (E)-3-(7-Methyl-2,6-dioxo-1-propyl-8-styryl-1,2,6,7-tetrahydro-3H-purin-3-yl)propyl dihydrogen phosphate: White solid; 42%; ¹H NMR (600 MHz, DMSO-d₆) δ 7.71 (d, J=7.5 Hz, 2H, Hₐᵣₒₘ), 7.58 (d, J=15.7 Hz, 1H, CH═CH), 7.40-7.30 (m, 3H, Hₐᵣₒₘ), 7.23 (d, J=15.7 Hz, 1H, CH═CH), 4.05 (t, J=7.5 Hz, 2H, N1-CH₂), 3.97 (s, 3H, N—CH₃), 3.85 (q, J=6.6 Hz, 2H, N3-CH₂), 3.73 (d, J=2.5 Hz, 2H, OCH₂), 1.97-1.95 (m, 2H, H_{alkyl}), 1.51-1.49 (m, 2H), 0.82 (t, J=7.4 Hz, 3H, CH₃). ¹³C NMR (126 MHz, DMSO-d₆) δ 154.02, (C8) 150.76 (C6), 149.62 (C2), 148.27 (C4), 135.56 (CH═CH), 135.36 (CH═CH), 129.23 (C5), 129.10, 127.27, 115.93, 107.40 (Cₐᵣₒₘ), 63.38 (N—CH₃), 63.34, (OCH₂) 28.95, 28.89, 20.98, 11.33 (C_{alkyl}). ³¹P-NMR (243 MHz, DMSO-d₆) δ [ppm] 0.68. HRMS (ESI-QTOF) calculated for C₂₀H₂₅N₄O₆P [M+Na]⁺: 471.1409; found: 471.1404).

H-6) (E)-3-(8-(3-Methoxystyryl)-7-methyl-2,6-dioxo-1-propyl-1,2,6,7-tetrahydro-3H-purin-3-yl)propyl dihydrogen phosphate: White solid; Yield 52%; ¹H-NMR (600 MHz, DMSO-d₆) δ [ppm]=H NMR (600 MHz, DMSO-d₆) δ 7.63 (d, J=15.1 Hz, 1H, CH═CH), 7.34-7.31 (m, 4H, Hₐᵣₒₘ, CH═CH), 7.00-6.88 (m, 1H, Hₐᵣₒₘ), 4.12-4.11 (m, 2H, N1-CH₂), 4.03 (s, 3H, N7-CH₃), 3.91.3.90 (m, 2H, N3CH₂), 3.81 (s, 3H, OCH₃), 2.02-2.00 (m, 2H, Hₐii), 1.56-1.55 (m, 2H, Hₐii), 0.86 (t, J=7.5 Hz, 3H, CH₃). ¹³C-NMR (151 MHz, DMSO-d₆) δ [ppm]=159.81 (Cₐᵣₒₘ), 154.36 (C8), 150.59 (C6), 149.80 (C2), 147.78 (C4), 137.13 (C═C), 136.87 (C═C), 129.95 (C5), 120.42 (Cₐᵣₒₘ), 115.37 (Cₐᵣₒₘ), 113.24 (Cₐᵣₒₘ), 112.64 (Cₐᵣₒₘ), 107.56 (Cₐᵣₒₘ), 63.44 (N—CH₃), 55.39 (OCH₃), 31.61, 20.98, 11.35 (C_{alkyl}). ³¹P-NMR (243 MHz, DMSO-d₆) δ [ppm] 0.02. HRMS (ESI-QTOF) calculated for C₂₁H₂₇N₄O₇P [M+Na]⁺: 501.1515; found: 501.1510).

H-7) (E)-3-(1-Ethyl-8-(3-methoxystyryl)-2,6-dioxo-1,2,6,7-tetrahydro-3H-purin-3-yl)propyl dihydrogen phosphate: White solid; Yield 46%; ¹H-NMR (600 MHz, DMSO-d₆) δ [ppm]=¹H NMR (600 MHz, DMSO-d₆) δ 7.64 (d, J=15.7 Hz, 1H, CH═CH), 7.40-7.36 (m, 2H, Hₐᵣₒₘ), 7.35-7.29 (m, 2H, Hₐᵣₒₘ, CH═CH), 6.93 (d, J=7.7, 1H, Hₐᵣₒₘ), 4.14-4.06 (m, 2H, N1CH₂), 3.91-390 (m, 4H, N3-CH₂, OCH₂), 3.81 (s, 3H, OCH₃), 2.02-2.01 (m, 2H, H_{alkyl}), 1.12 (t, J=7.0 Hz, 3H, CH₃). ¹³C-NMR (151 MHz, DMSO-d₆) δ [ppm]=159.82 (Cₐᵣₒₘ), 154.17 (C8), 150.40 (C6), 149.81 (C2), 137.14 (C4), 136.90 (C═C), 129.95 (C═C), 120.43 (C5), 115.40 (Cₐᵣₒₘ), 113.28 (Cₐᵣₒₘ), 112.63 (Cₐᵣₒₘ), 107.62 (Cₐᵣₒₘ), 63.46 (OCH₂), 55.42 (OCH₃), 35.72, 31.65, 13.31 (C_{alkyl}). ³¹P-NMR (243 MHz, DMSO-d₆) δ [ppm] 0.20. HRMS (ESI-QTOF) calculated for C₁₉H₂₃N₄O₇P [M+Na]⁺: 473.1202; found: 473.1197).

H-8) 3-(8-(3-Methoxybenzyl)-2,6-dioxo-1-propyl-1,2,6,7-tetrahydro-3H-purin-3-yl)propyl dihydrogen phosphate: White solid; Yield 46%; ¹H NMR (600 MHz, DMSO-d₆) δ 7.12 (d, J=8.1 Hz, 2H, Hₐᵣₒₘ), 6.84 (d, J=8.1 Hz, 2H, Hₐᵣₒₘ), 4.08 (s, 2H, C8CH₂), 4.01 (t, J=7.5 Hz, 2H, N1-CH₂), 3.73 (s, N7-CH₃), 3.67-3.62 (m, 7H, N3-CH₂, OCH₃, OCH₂), 1.59-1.38 (m, 2H, H_{alkyl}), 1.13 (t, J=7.1 Hz, 2H, CH₂), 0.80 (t, J=7.5 Hz, 3H, CH₃). ¹³C NMR (151 MHz, DMSO) δ 158.18 (Cₐᵣₒₘ), 154.39 (C8), 153.08 (C6), 150.41 (C2), 147.14 (C4), 129.56 (C8), 127.81 (Cₐᵣₒₘ), 114.23 (Cₐᵣₒₘ), 106.91 (C5), 55.14 (OCH2), 45.59 (OCH₃), 31.75 (N7-CH₃), 31.37, 20.91, 11.29 (C_{alkyl}). ³¹P-NMR (243 MHz, DMSO-d₆) δ [ppm]0.08. HRMS (ESI-QTOF) calculated for C₂₀H27N₄O₇P [M+Na]⁺: 489.1515; found 489.1510).

H-9): 3-(8-(4-Chlorobenzyl)-7-methyl-2,6-dioxo-1-(prop-2-yn-1-yl)-1,2,6,7-tetrahydro-3H-purin-3-yl)propyl dihydrogen phosphate: White solid; Yield 51%; ¹H-NMR (600 MHz, DMSO-d₆) δ [ppm]=7.35 (d, J=8.0 Hz, 2H, Hₐᵣₒₘ), 7.30 (d, J=8.3 Hz, 2H, Hₐᵣₒₘ), 4.55 (s, 3H, N7-CH₃), 4.02 (t, J=7.4 Hz, 2H, N1-CH₂), 3.83-382 (m, 2H, N3-CH₂), 3.03 (t, J=2.4 Hz, 1H, H_{propargyl}), 1.93-192 (m, 2H, H_{alkyl}). ¹³C-NMR (151 MHz, DMSO-d₆) δ [ppm]=153.19 (C8), 152.54 (C6), 150.09 (C2), 148.21 (C4), 136.02 (Cₐᵣₒₘ), 131.54 02 (Cₐᵣₒₘ), 130.59 02 (Cₐᵣₒₘ), 128.63 (Cₐᵣₒₘ), 79.83 (C_{propargyl}), 72.82 (C_{propargyl}), 62.76 (OCH₂), 33.65 (N7-CH₃), 30.22, 28.95, 28.94 (C_{alkyl}). ³¹P-NMR (243 MHz, DMSO-d₆) δ [ppm] 0.37. HRMS (ESI-QTOF) calculated for C₁₈H₁₈CIN₄O₆P [M+H]⁺: 475.0550; found: 475.0545.

J-1): (E)-(4-(8-(3-Methoxystyryl)-2,6-dioxo-1-(prop-2-yn-1-yl)-1,2,6,7-tetrahydro-3H-purin-3-yl)butyl)phosphonic acid: White solid; Yield 58; ¹H-NMR (600 MHz, DMSO-d₆) δ [ppm]=12.0 (s, 1H, NH), 7.62 (d, 1H, H_{alkyl}), 7.29-7.33 (m, 1H), 7.15-7.24 (m, 2H), 7.07 (d, J=16.35 Hz, 1H, H_{alkyl}), 6.93 (m, 1H, Hₐᵣₒₘ), 4.60 (d, J=2.4 Hz, C_{propargyl}), 4.02 (t, J=6.62, 7.14 Hz, 2H, NCH₂), 3.80 (d, J=8.20, 3H, OCH₃), 3.07 (t, J=2.40, 1H, H_{propargyl}), 1.79 (m, 2H, PCH₂), 1.59 (m, 2H), 1.53 (m, 2H). ¹³C-NMR (151 MHz, DMSO-d₆) δ [ppm]=159.8, 153.0, 150.1, 150.0, 148.7, 136.9, 135.7, 130.1, 119.8, 116.2, 115.4, 112.1, 107.1, 79.8, 72.9, 55.3, 43.0, 30.3, 28.8, 28.0, 26.9, 20.3, 20.2. ³¹P-NMR (243 MHz, DMSO-d₆) δ [ppm] 27.17.

J-2): (4-(8-Cyclopentyl-2,6-dioxo-1-propyl-1,2,6,7-tetrahydro-3H-purin-3-yl)butyl)phosphonic acid: White solid; Yield 13%; ¹H-NMR (600 MHz, DMSO-d₆) δ [ppm]=13.07 (br s, 1H, 7-NH), 3.95 (t, J=7.1 Hz, 2H, N1-CH₂), 3.81 (d, J=6.3 Hz, 2H, N3-CH₂), 3.12 (m, 1H, H_{cyclopentyl}), 2.03-1.89 (m, 2H, H_{alkyl}), 1.83-1.64 (m, 6H, H_{alkyl}, H_{cyclopentyl}), 1.53-1.51 (m, 8H, H_{alkyl}, H_{cyclopentyl}), 0.84 (t, J=7.4 Hz, 3H, CH₃). ¹³C-NMR (151 MHz, DMSO-d₆) δ [ppm]=158.02 (C8), 154.00 (C6), 150.76 (C2), 147.83 (C4), 106.23 (C5), 42.56 (N1-CH₂), 42.12 (N3-CH₂), 32.08 (C_{cyclopentyl}), 28.81, 28.00, 27.09, 25.19, 20.99, 20.21, 11.31 (C-alkyl, C_{cyclopentyl}). ³¹P-NMR (243 MHz, DMSO-d₆) δ [ppm] 29.21. HRMS (ESI-QTOF): n.d.

J-3) (4-(2,6-Dioxo-8-phenyl-1-propyl-1,2,6,7-tetrahydro-3H-purin-3-yl)butyl)phosphonic acid: White solid; Yield 17%; ¹H-NMR (600 MHz, DMSO-d₆) δ [ppm]=¹H NMR (600 MHz, DMSO-d₆) δ 13.82 (br s, 1H, 7-H), 8.25-7.86 (m, 2H, Hₐᵣₒₘ), 7.50-7.48 (m, 3H, Hₐᵣₒₘ), 4.23-3.99 (m, 2H, N1-CH₂), 3.92-3.79 (m, 2H, N3-CH₂), 1.96-1.75 (m, 2H, H_{alkyl}), 1.63-1.47 (m, 6H, H_{alkyl})), 1.08-0.70 (m, 3H, CH₃). ¹³C-NMR (151 MHz, DMSO-d₆) δ [ppm]=¹³C NMR (151 MHz, DMSO-d₆) δ 154.22 (C8), 150.78 (C6), 150.06 (C2), 148.40 (C4), 130.37 (C5), 129.09, 126.65, 107.91 (Cₐᵣₒₘ), 28.80, 27.88, 26.98, 21.00, 20.21, 11.34 (C_{alkyl}). ³¹P-NMR (243 MHz, DMSO-d₆) δ [ppm] 27.21. HRMS (ESI-QTOF) calculated for C₁₈H₂₃N₄O₅P [M+Na]⁺: 429.1304; found: 429.1298).

### Synthesis of Intermediates

### Synthesis of 4-iodobutylphosphonic acid diethyl ester, 5-iodopentylphosphonic acid diethyl ester and 6-iodohexylphosphonic acid diethyl ester

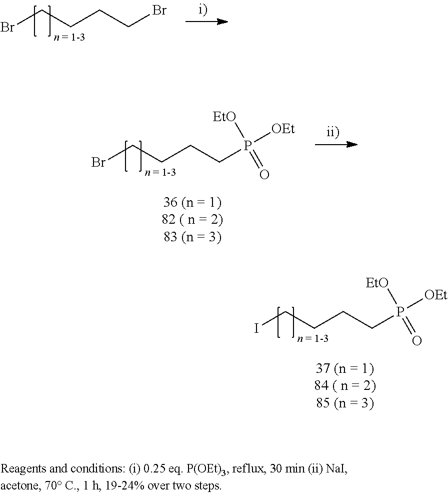

### Synthesis of N-(6-Amino-2,4-dioxo-3-(prop-2-yn-1-yl)-1,2,3,4-tetrahydropyrimidin-5-yl)-3-(2-fluoro-phenyl)propanamide (38a

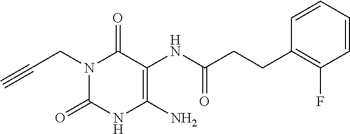 [SMILES: C#CCn1c(=O)[nH]c(N)c(NC(=O)CCc2ccccc2F)c1=O]

Yield: 84%, off-white solid. ¹H NMR (600 MHz, DMSO-d₆) δ [ppm]=10.61 (s, 1H, 1N-H), 8.43 (s, 1H, CONH), 7.34 (td, J=7.7, 1.7 Hz, 1H, Hₐᵣₒₘ), 7.25 (tdd, J=7.4, 5.4, 1.8 Hz, 1H, Hₐᵣₒₘ), 7.17-7.10 (m, 2H, 2Hₐᵣₒₘ), 6.04 (s, 2H, NH₂), 4.42 (d, J=2.4 Hz, 2H, N3-CH₂), 3.02 (t, J=2.4 Hz, 1H, H_{propargyl}), 2.91-2.84 (m, 2H, CH₂), 2.56-2.51 (m, 2H, CH₂). ¹³C NMR (151 MHz, DMSO-d₆) δ [ppm]=171.5 (CONH), 160.47 (d, ¹J_{C,F}=243.4 Hz, Cₐᵣₒₘ), 159.6 (C6), 150.5 (CO), 149.2 (CO), 130.6 (Cₐᵣₒₘ), 128.0 (d, ²J_{C,F}=21.8 Hz, Cₐᵣₒₘ), 128.0 (Cₐᵣₒₘ), 124.4 (d, J=3.3 Hz, Cₐᵣₒₘ), 115.0 (d, J=22.4 Hz, Cₐᵣₒₘ), 86.7 (C5), 80.0 (C_{propargyl}), 72.4 (C_{propargyl}), 35.2 (CH₂), 28.8 (N3-CH₂), 24.0 (d, ³J_{C,F}=2.6 Hz, CH₂). LC-MS: positive mode [m/z]=331.1 [M+H]⁺. Purity: 97%.

### Synthesis of N-(6-Amino-2,4-dioxo-3-(prop-2-yn-1-yl)-1,2,3,4-tetrahydropyrimidin-5-yl)-3-(2-iodo-phenyl)propanamide (38b

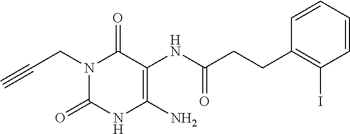 [SMILES: C#CCn1c(=O)[nH]c(N)c(NC(=O)CCc2ccccc2I)c1=O]

Yield: 89%, off-white solid. ¹H NMR (500 MHz, DMSO-d₆) δ [ppm]=10.61 (s, 1H, N1-H), 8.42 (s, 1H, CONH), 7.83 (d, J=7.8 Hz, 1H, Hₐᵣₒₘ), 7.35 (d, J=4.4 Hz, 2H, 2Hₐᵣₒₘ), 6.96 (dt, J=8.4, 4.4 Hz, 1H, Hₐᵣₒₘ), 6.06 (s, 2H, NH₂), 4.42 (d, J=2.5 Hz, 2H, N3-CH₂), 3.01 (t, J=2.3 Hz, 1H, H_{propargyl}), 2.94 (t, J=7.6 Hz, 2H, CH₂), 2.53 (t, J=7.8 Hz, 2H, CH₂). ¹³C NMR (126 MHz, DMSO-d₆) δ [ppm]=171.3 (CONH), 159.6 (C6), 150.6 (CO), 149.3 (CO), 143.8 (Cₐᵣₒₘ), 139.0 (Cₐᵣₒₘ), 129.4 (Cₐᵣₒₘ), 128.6 (Cₐᵣₒₘ), 128.2 (Cₐᵣₒₘ), 100.6 (Cₐᵣₒₘ), 86.8 (C5), 80.0 (C_{propargyl}), 72.3 (C_{propargyl}), 35.7 (CH₂), 35.2 (N3-CH₂), 28.8 (CH₂). LC-MS: positive mode [m/z]=438.9 [M+H]⁺. Purity: 98%.

### Synthesis of N-(6-Amino-2,4-dioxo-3-(prop-2-yn-1-yl)-1,2,3,4-tetrahydropyrimidin-5-yl)-3-(o-tolyl)propanamide (38c

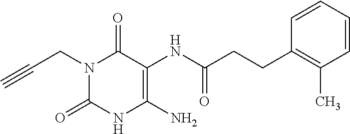 [SMILES: C#CCn1c(=O)[nH]c(N)c(NC(=O)CCc2ccccc2C)c1=O]

Yield: 88%, off-white solid. ¹H NMR (500 MHz, DMSO-d₆) δ [ppm]=10.59 (s, 1H, N1-H), 8.42 (s, 1H, CONH), 7.25-6.95 (m, 4H, 4Hₐᵣₒₘ), 5.98 (s, 2H, NH₂), 4.42 (d, J=2.5 Hz, 2H, N3-CH₂), 3.01 (t, J=2.4 Hz, 1H, H_{propargyl}), 2.87-2.81 (m, 2H, CH₂), 2.50-2.45 (m, 2H, CH₂), 2.29 (s, 3H, CH₃). ¹³C NMR (126 MHz, DMSO-d₆) δ [ppm]=171.9 (CONH), 159.6 (C6), 150.4 (CO), 149.1 (CO), 139.6 (Cₐᵣₒₘ), 135.5 (Cₐᵣₒₘ), 129.9 (Cₐᵣₒₘ), 128.3 (Cₐᵣₒₘ), 125.9 (Cₐᵣₒₘ), 125.9 (Cₐᵣₒₘ), 86.9 (C5), 80.0 (C_{propargyl}), 72.3 (C_{propargyl}), 35.5 (CH₂), 28.8 (N3-CH₂), 28.2 (CH₂), 18.8 (CH₃). LC-MS: positive mode [m/z]=327.2 [M+H]⁺. Purity: 95%.

### Synthesis of N-(6-Amino-2,4-dioxo-3-(prop-2-yn-1-yl)-1,2,3,4-tetrahydropyrimidin-5-yl)-3-(2,6-di-methylphenyl)propanamide (38l

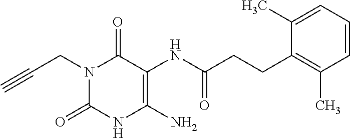 [SMILES: C#CCn1c(=O)[nH]c(N)c(NC(=O)CCc2c(C)cccc2C)c1=O]

Yield: 87%, off-white solid. ¹H NMR (500 MHz, DMSO-d₆) δ [ppm]=10.58 (s, 1H, N1-H), 8.40 (s, 1H, CONH), 6.98 (s, 3H, 3Hₐᵣₒₘ), 6.04 (s, 2H, NH₂), 4.43 (d, J=2.4 Hz, 2H, N3-CH₂), 3.01 (t, J=2.4 Hz, 1H, H_{propargyl}), 2.88-2.81 (m, 2H, CH₂), 2.37-2.31 (m, 2H, CH₂), 2.31 (s, 6H, 2CH₃). ¹³C NMR (126 MHz, DMSO-d₆) δ [ppm]=171.9 (CONH), 159.6 (C6), 150.4 (CO), 149.1 (CO), 138.1 (Cₐᵣₒₘ), 135.6 (2Cₐᵣₒₘ), 127.9 (2Cₐᵣₒₘ), 125.6 (Cₐᵣₒₘ), 86.9 (C5), 80.0 (C_{propargyl}), 72.3 (C_{propargyl}), 34.3 (CH₂), 28.8 (N3-CH₂), 25.0 (CH₂), 19.3 (2CH₃). LC-MS: positive mode [m/z]=341.20 [M+H]⁺. Purity: 83%.

### Synthesis of N-(6-Amino-2,4-dioxo-3-(prop-2-yn-1-yl)-1,2,3,4-tetrahydropyrimidin-5-yl)-3-(2,6-di-chlorophenyl)propanamide (38m

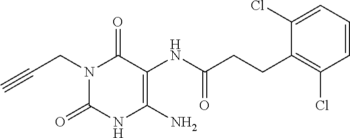 [SMILES: C#CCn1c(=O)[nH]c(N)c(NC(=O)CCc2c(Cl)cccc2Cl)c1=O]

Yield: 79%, off-white solid. ¹H NMR (600 MHz, DMSO-d6) δ[ppm]=10.60 (s, 1H, N1-H), 8.42 (s, 1H, CONH), 7.46 (d, J=8.0 Hz, 2H, 2Hₐᵣₒₘ), 7.28 (t, J=8.1 Hz, 1H, Hₐᵣₒₘ), 6.14 (s, 2H, NH₂), 4.42 (d, J=2.5 Hz, 2H, N3-CH₂), 3.14-3.09 (m, 2H, CH₂), 3.03 (t, J=2.4 Hz, 1H, H_{propargyl}), 2.50-2.44 (m, 2H, CH₂). ¹³C NMR (151 MHz, DMSO-d6) δ[ppm]=171.0 (CONH), 159.6 (C6), 150.6 (CO), 149.2 (CO), 136.8 (Cₐᵣₒₘ), 134.4 (2Cₐᵣₒₘ), 128.8 (Cₐᵣₒₘ), 128.5 (2Cₐᵣₒₘ), 86.7 (C5), 80.0 (C_{propargyl}), 72.3 (C_{propargyl}), 32.9 (CH₂), 28.8 (N3-CH₂), 26.6 (CH₂). LC-MS: positive mode [m/z]=381.1 [M+H]⁺. Purity: 98%.

### Synthesis of N-(6-Amino-2,4-dioxo-3-(prop-2-yn-1-yl)-1,2,3,4-tetrahydropyrimidin-5-yl)-3-(4-bromo-phenyl)propanamide (38d

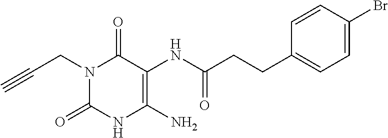 [SMILES: C#CCn1c(=O)[nH]c(N)c(NC(=O)CCc2ccc(Br)cc2)c1=O]

Yield: 79%, off-white solid. ¹H NMR (600 MHz, DMSO-d₆) δ [ppm]=10.62 (s, 1H, N1-H), 8.42 (s, 1H, CONH), 7.49-7.44 (m, 2H, 2Hₐᵣₒₘ), 7.24-7.19 (m, 2H, 2Hₐᵣₒₘ), 6.01 (s, 2H, NH₂), 4.42 (d, J=2.5 Hz, 2H, N3-CH₂), 3.01 (t, J=2.4 Hz, 1H, H_{propargyl}), 2.83 (dd, J=9.2, 6.7 Hz, 2H, CH₂), 2.54-2.51 (m, 2H, CH₂). ¹³C NMR (151 MHz, DMSO-d₆) δ [ppm]=171.7 (CONH), 159.7 (C6), 150.5 (CO), 149.2 (CO), 141.0 (Cₐᵣₒₘ), 131.2 (2Cₐᵣₒₘ), 130.6 (2Cₐᵣₒₘ), 118.9 (Cₐᵣₒₘ), 86.8 (C5), 80.0 (C_{propargyl}), 72.4 (C_{propargyl}), 36.5 (CH₂), 30.2 (N3-CH₂), 28.9 (CH₂). LC-MS: positive mode [m/z]=391.0 [M+H]⁺. Purity: 97%.

### Synthesis of N-(6-Amino-2,4-dioxo-3-(prop-2-yn-1-yl)-1,2,3,4-tetrahydropyrimidin-5-yl)-3-(4-iodo-phenyl)propanamide (38e

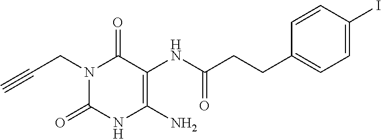 [SMILES: C#CCn1c(=O)[nH]c(N)c(NC(=O)CCc2ccc(I)cc2)c1=O]

Yield: 76%, off-white solid. ¹H NMR (600 MHz, DMSO-d₆) δ [ppm]=10.60 (s, 1H, N1-H), 8.42 (s, 1H, CONH), 7.66-7.60 (m, 2H, 2Hₐᵣₒₘ), 7.09-7.06 (m, 2H, 2Hₐᵣₒₘ), 6.00 (s, 2H, NH₂), 4.42 (d, J=2.4 Hz, 2H, N3-CH₂), 3.02 (t, J=2.4 Hz, 1H, H_{propargyl}), 2.84-2.78 (m, 2H, CH₂), 2.54-2.49 (m, 2H, CH₂). ¹³C NMR (151 MHz, DMSO-d₆) δ [ppm]=171.6 (CONH), 159.6 (C6), 150.4 (CO), 149.1 (CO), 141.4 (Cₐᵣₒₘ), 137.0 (2Cₐᵣₒₘ), 130.7 (2Cₐᵣₒₘ), 91.3 (Cₐᵣₒₘ), 86.8 (C5), 80.0 (C_{propargyl}), 72.4 (C_{propargyl}), 36.4 (CH₂), 30.3 (N3-CH₂), 28.8 (CH₂). LC-MS: positive mode [m/z]=439.1 [M+H]⁺. Purity: 95%.

### Synthesis of N-(6-Amino-2,4-dioxo-3-(prop-2-yn-1-yl)-1,2,3,4-tetrahydropyrimidin-5-yl)-3-(4-(trifluoromethyl)phenyl)propanamide (38f

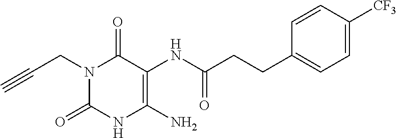 [SMILES: C#CCn1c(=O)[nH]c(N)c(NC(=O)CCc2ccc(C(F)(F)F)cc2)c1=O]

Yield: 68%, off-white solid. ¹H NMR (600 MHz, DMSO-d₆) δ [ppm]=10.62 (s, 1H, N1-H), 8.45 (s, 1H, CONH), 7.64 (d, J=8.0 Hz, 2H, 2Hₐᵣₒₘ), 7.49 (d, J=7.9 Hz, 2H, 2Hₐᵣₒₘ), 6.03 (s, 2H, NH₂), 4.42 (d, J=2.5 Hz, 2H, N3-CH₂), 3.02 (t, J=2.4 Hz, 1H, H_{propargyl}), 2.96 (t, J=7.9 Hz, 2H, CH₂), 2.60-2.54 (m, 2H, CH₂). ¹³C NMR (151 MHz, DMSO-d₆) δ [ppm]=171.5 (CONH), 159.6 (C6), 150.5 (CO), 149.2 (CO), 146.5 (Cₐᵣₒₘ), 129.0 (2Cₐᵣₒₘ), 126.6 (q, ²J_{C,F}=31.8 Hz, Cₐᵣₒₘ), 125.3 (q, ¹J_{C,F}=272.5 Hz, CF₃), 125.1 (q, ³J_{C,F}=3.7 Hz, 2Cₐᵣₒₘ), 86.8 (C5), 80.0 (C_{propargyl}), 72.4 (C_{propargyl}), 36.2 (CH₂), 30.6 (N3-CH₂), 28.8 (CH₂). LC-MS: positive mode [m/z]=381.20 [M+H]⁺. Purity: 97%.

### Synthesis of N-(6-Amino-2,4-dioxo-3-(prop-2-yn-1-yl)-1,2,3,4-tetrahydropyrimidin-5-yl)-3-(4-meth-oxyphenyl)propanamide (38g

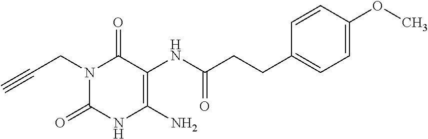 [SMILES: C#CCn1c(=O)[nH]c(N)c(NC(=O)CCc2ccc(OC)cc2)c1=O]

Yield: 48%, off-white solid. ¹H NMR (600 MHz, DMSO-d₆) δ [ppm]=10.61 (s, 1H, N1-H), 8.40 (s, 1H, CONH), 7.15 (d, J=8.5 Hz, 2H, 2Hₐᵣₒₘ), 6.85 (d, J=8.5 Hz, 2H, 2Hₐᵣₒₘ), 5.97 (s, 2H, NH₂), 4.42 (d, J=2.4 Hz, 2H, N3-CH₂), 3.72 (s, 3H, CH₃), 3.02 (t, J=2.4 Hz, 1H, H_{propargyl}), 2.83-2.76 (m, 2H, CH₂), 2.49-2.46 (m, 2H, CH₂). ¹³C NMR (151 MHz, DMSO-d₆) δ [ppm]=171.9 (CONH), 159.6 (C6), 157.5 (Cₐᵣₒₘ), 150.4 (CO), 149.2 (CO), 133.4 (Cₐᵣₒₘ), 129.1 (2Cₐᵣₒₘ), 113.7 (2Cₐᵣₒₘ), 86.9 (C5), 80.0 (C_{propargyl}), 72.4 (C_{propargyl}), 55.0 (CH₃), 37.2 (CH₂), 30.0 (N3-CH₂), 28.8 (CH₂). LC-MS: positive mode [m/z]=343.20 [M+H]⁺. Purity: 99%.

### Synthesis of N-(6-Amino-2,4-dioxo-3-(prop-2-yn-1-yl)-1,2,3,4-tetrahydropyrimidin-5-yl)-3-(4-ethylphenyl)propanamide (38i

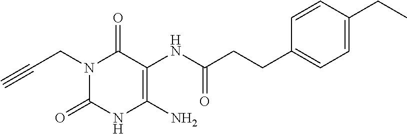 [SMILES: C#CCn1c(=O)[nH]c(N)c(NC(=O)CCc2ccc(CC)cc2)c1=O]

Yield: 62%, off-white solid. ¹H NMR (600 MHz, DMSO-d₆) δ [ppm]=10.59 (s, 1H, N1-H), 8.41 (s, 1H, CONH), 7.15 (d, J=8.1 Hz, 2H, 2Hₐᵣₒₘ), 7.12 (d, J=8.1 Hz, 2H, 2Hₐᵣₒₘ), 5.97 (s, 2H, NH₂), 4.42 (d, J=2.5 Hz, 2H, N3-CH₂), 3.02 (t, J=2.4 Hz, 1H, H_{propargyl}), 2.84-2.79 (m, 2H, CH₂), 2.56 (q, J=7.6 Hz, 2H, CH₂CH₃), 2.53-2.47 (m, 2H, CH₂), 1.16 (t, J=7.6 Hz, 3H, CH₂CH₃). ¹³C NMR (126 MHz, DMSO-d₆) δ [ppm]=171.9 (CONH), 159.6 (C6), 150.4 (CO), 149.2 (CO), 141.1 (Cₐᵣₒₘ), 138.6 (Cₐᵣₒₘ), 128.1 (2Cₐᵣₒₘ), 127.7 (2Cₐᵣₒₘ), 86.9 (C5), 80.0 (C_{propargyl}), 72.3 (C_{propargyl}), 36.9 (CH₂), 30.5 (N3-CH₂), 28.8 (CH₂), 27.7 (CH₂), 15.6 (CH₂CH₃). LC-MS: positive mode [m/z]=341.30 [M+H]⁺. Purity: 99%.

### Synthesis of N-(6-Amino-2,4-dioxo-3-(prop-2-yn-1-yl)-1,2,3,4-tetrahydropyrimidin-5-yl)-3-(4-iso-propylphenyl)propanamide (38i

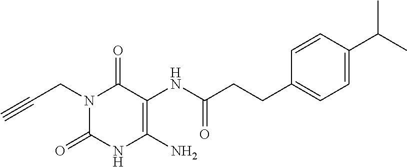 [SMILES: C#CCn1c(=O)[nH]c(N)c(NC(=O)CCc2ccc(C(C)C)cc2)c1=O]

Yield: 55%, off-white solid. ¹H NMR (500 MHz, DMSO-d₆) δ [ppm]=10.58 (s, 1H, N1-H), 8.40 (s, 1H, CONH), 7.15 (s, 4H, 4Hₐᵣₒₘ), 5.94 (s, 2H, NH₂), 4.42 (d, J=2.5 Hz, 2H, N3-CH₂), 3.01 (t, J=2.4 Hz, 1H, H_{propargyl}), 2.89-2.78 (m, 3H, CH₂ and CH(CH₃)₂), 2.53-2.51 (m, 2H, CH₂), 1.19 (d, J=6.8 Hz, 6H, CH(CH₃)₂). ¹³C NMR (126 MHz, DMSO-d₆) δ [ppm]=171.9 (CONH), 159.6 (C6), 150.4 (CO), 149.1 (CO), 145.8 (Cₐᵣₒₘ), 138.8 (Cₐᵣₒₘ), 128.0 (2Cₐᵣₒₘ), 126.2 (2Cₐᵣₒₘ), 86.9 (C5), 80.0 (C_{propargyl}), 72.3 (C_{propargyl}), 36.9 (CH₂), 33.0 (s, CH(CH₃)₂), 30.5 (N3-CH₂), 28.8 (CH₂), 23.9 (CH(CH₃)₂). LC-MS: positive mode [m/z]=355.30 [M+H]⁺. Purity: 85%.

### Synthesis of 3-([1,1′-Biphenyl]-4-yl)-N-(6-amino-2,4-dioxo-3-(prop-2-yn-1-yl)-1,2,3,4-tetrahydro pyrimidin-5-yl)propanamide (38k

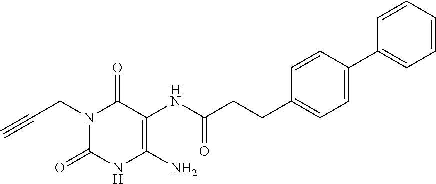 [SMILES: C#CCn1c(=O)[nH]c(N)c(NC(=O)CCc2ccc(-c3ccccc3)cc2)c1=O]

Yield: 81%, off-white solid. ¹H NMR (600 MHz, DMSO-d₆) δ [ppm]=10.61 (s, 1H, N1-H), 8.45 (s, 1H, CONH), 7.66-7.64 (m, 2H, 2Hₐᵣₒₘ), 7.60-7.57 (m, 2H, 2Hₐᵣₒₘ), 7.47-7.43 (m, 2H, 2Hₐᵣₒₘ), 7.36-7.33 (m, 3H, 3Hₐᵣₒₘ), 5.99 (s, 2H, NH₂), 4.43 (d, J=2.5 Hz, 2H, N3-CH₂), 3.02 (t, J=2.4 Hz, 1H, H_{propargyl}), 2.93-2.88 (m, 2H, CH₂), 2.59-2.54 (m, 2H, CH₂). ¹³C NMR (151 MHz, DMSO-d₆) δ [ppm]=171.8 (CONH), 159.6 (C6), 150.5 (CO), 149.2 (CO), 140.8 (Cₐᵣₒₘ), 140.1 (Cₐᵣₒₘ), 137.8 (Cₐᵣₒₘ), 128.9 (2Cₐᵣₒₘ), 128.8 (2Cₐᵣₒₘ), 127.1 (Cₐᵣₒₘ), 126.6 (2Cₐᵣₒₘ), 126.5 (2Cₐᵣₒₘ), 86.9 (C5), 80.0 (C_{propargyl}), 72.4 (C_{propargyl}), 36.7 (CH₂), 30.5 (N3-CH₂), 28.8 (CH₂). LC-MS: positive mode [m/z]=389.30 [M+H]⁺. Purity: 88%.

### Synthesis of N-(6-Amino-2,4-dioxo-3-(prop-2-yn-1-yl)-1,2,3,4-tetrahydropyrimidin-5-yl)-2-(naphthalen-1-yl)acetamide (75

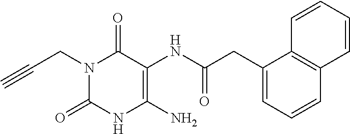 [SMILES: C#CCn1c(=O)[nH]c(N)c(NC(=O)Cc2cccc3ccccc23)c1=O]

Yield: 86%, off-white solid. ¹H NMR (600 MHz, DMSO-d6) δ[ppm]=10.63 (s, 1H, N1-H), 8.70 (s, 1H, CONH), 7.90-7.82 (m, 4H, 4Hₐᵣₒₘ), 7.55-7.44 (m, 3H, 3Hₐᵣₒₘ), 6.11 (s, 2H, NH₂), 4.42 (d, J=2.5 Hz, 2H, N3-CH₂), 3.75 (s, 2H, CH₂), 3.01 (t, J=2.5 Hz, 1H, H_{propargyl}). ¹³C NMR (151 MHz, DMSO-d6) δ [ppm]=170.5 (CONH), 159.6 (C6), 150.5 (CO), 149.2 (CO), 134.1 (Cₐᵣₒₘ), 133.0 (Cₐᵣₒₘ), 131.7 (Cₐᵣₒₘ), 127.9 (Cₐᵣₒₘ), 127.5 (Cₐᵣₒₘ), 127.4 (Cₐᵣₒₘ), 127.4 (Cₐᵣₒₘ), 127.4 (Cₐᵣₒₘ), 125.9 (Cₐᵣₒₘ), 125.4 (Cₐᵣₒₘ), 86.9 (C5), 80.0 (C_{propargyl}), 72.4 (C_{propargyl}), 42.1 (CH₂), 28.8 (N3-CH₂). LC-MS: positive mode [m/z]=349.0 [M+H]⁺. Purity: 95%.

### Synthesis of Diethyl (4-(6-amino-5-(3-(2-fluorophenyl)propanamido)-2,4-dioxo-3-(prop-2-yn-1-yl)-3,4-dihydropyrimidin-1(2H)-yl)butyl)phosphonate (39a

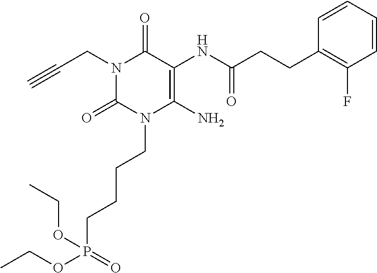 [SMILES: C#CCn1c(=O)c(NC(=O)CCc2ccccc2F)c(N)n(CCCCP(=O)(OCC)OCC)c1=O]

Yield: 58%, light-yellow solid. ¹H NMR (600 MHz, DMSO-d₆) δ [ppm]=8.42 (s, 1H, CONH), 7.35 (td, J=7.7, 1.7 Hz, 1H, Hₐᵣₒₘ), 7.28-7.23 (m, 1H, Hₐᵣₒₘ), 7.16-7.09 (m, 2H, 2Hₐᵣₒₘ), 6.70 (s, 2H, NH₂), 4.48 (d, J=2.5 Hz, 2H, N3-CH₂), 4.01-3.94 (m, 4H, 2OCH₂), 3.86 (dd, J=8.5, 6.7 Hz, 2H, N1-CH₂), 3.01 (t, J=2.4 Hz, 1H, H_{propargyl}), 2.91-2.86 (m, 2H, CH₂), 2.59-2.53 (m, 2H, CH₂), 1.78 (ddd, J=17.9, 8.9, 6.6 Hz, 2H, CH₂), 1.66-1.59 (m, 2H, CH₂), 1.53 (dd, J=17.6, 10.5 Hz, 2H, CH₂), 1.23 (t, J=7.1 Hz, 6H, 2OCH₂CH₃). ¹³C NMR (151 MHz, DMSO-d₆) δ [ppm]=171.8 (CONH), 160.5 (d, ¹J_{C,F}=242.7 Hz, Cₐᵣₒₘ), 158.1 (C6), 151.7 (CO), 149.6 (CO), 130.6 (d, J=4.8 Hz, Cₐᵣₒₘ), 128.1 (d, ²J_{C,F}=15.4 Hz, Cₐᵣₒₘ), 127.9 (d, J=9.2 Hz, Cₐᵣₒₘ), 124.4 (d, J=4.6 Hz, Cₐᵣₒₘ), 115.0 (d, 2J_{C,F}=21.9 Hz, Cₐᵣₒₘ), 87.1 (C5), 79.9 (C_{propargyl}), 72.4 (C_{propargyl}), 60.9 (d, ²J_{C,P}=6.4 Hz, 2OCH₂), 42.1 (N1-CH₂), 35.2 (CH₂), 29.8 (N3-CH₂), 28.1 (d, ³J_{C,P}=15.8 Hz, CH₂), 24.0 (d, ¹J_{C,P}=138.7 Hz, PCH₂), 23.9 (d, ³J_{C,F}=2.8 Hz, CH₂), 19.1 (d, ²J_{C,P}=4.8 Hz, CH₂), 16.3 (d, ³J_{C,P}=5.4 Hz, 2OCH₂CH₃). ³¹P NMR (243 MHz, DMSO-d₆) δ [ppm]=32.7. LC-MS: positive mode [m/z]=523.5 [M+H]⁺. Purity: 91%.

### Synthesis of Diethyl (5-(6-amino-5-(3-(2-bromophenyl)propanamido)-2,4-dioxo-3-(prop-2-yn-1-yl)-3,4-dihydropyrimidin-1(2H)-yl)pentyl)phosphonate (40h

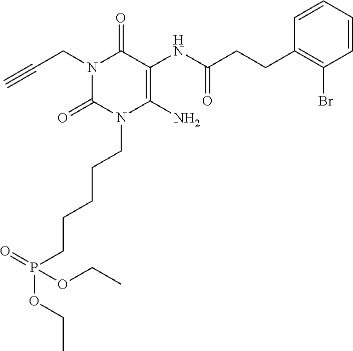 [SMILES: C#CCn1c(=O)c(NC(=O)CCc2ccccc2Br)c(N)n(CCCCCP(=O)(OCC)OCC)c1=O]

Yield: 57%, light-yellow viscous liquid. ¹HNMR (600 MHz, DMSO-d₆) δ [ppm]=8.43 (s, 1H, CONH), 7.59 (dd, J=8.0, 1.2 Hz, 1H, Hₐᵣₒₘ), 7.39 (dd, J=7.7, 1.7 Hz, 1H, Hₐᵣₒₘ), 7.33 (td, J=7.4, 1.3 Hz, 1H, Hₐᵣₒₘ), 7.16 (td, J=7.6, 1.7 Hz, 1H, Hₐᵣₒₘ), 6.67 (d, J=2.7 Hz, 2H, NH₂), 4.48 (d, J=2.5 Hz, 2H, N3-CH₂), 4.02-3.90 (m, 4H, 2OCH₂), 3.85 (t, J=7.7 Hz, 2H, N1-CH₂), 3.02 (t, J=2.4 Hz, 1H, H_{propargyl}), 2.98-2.94 (m, 2H, CH₂), 2.60-2.55 (m, 2H, CH₂), 1.75-1.61 (m, 2H, CH₂), 1.57-1.44 (m, 4H, 2CH₂), 1.37 (dd, J=10.5, 5.1 Hz, 2H, CH₂), 1.23 (t, J=7.0 Hz, 6H, 2OCH₂CH₃). ¹³C NMR (151 MHz, DMSO-d₆) δ [ppm]=171.7 (CONH), 158.1 (C6), 151.8 (CO), 149.7 (CO), 140.5 (Cₐᵣₒₘ), 132.4 (Cₐᵣₒₘ), 130.4 (Cₐᵣₒₘ), 128.1 (Cₐᵣₒₘ), 127.9 (Cₐᵣₒₘ), 123.7 (Cₐᵣₒₘ), 87.2 (C5), 79.9 (C_{propargyl}), 72.5 (C_{propargyl}), 60.8 (d, ²J_{C,P}=6.5 Hz, 2OCH₂), 42.3 (N1-CH₂), 34.9 (CH₂), 31.0 (N3-CH₂), 29.8 (CH₂), 27.0 (CH₂), 26.6 (d, ³J_{C,P}=16.0 Hz, CH₂), 24.4 (d, ¹J_{C,P}=138.4 Hz, PCH₂), 21.8 (d, ²J_{C,P}=5.1 Hz, CH₂), 16.3 (d, ³J_{C,P}=5.7 Hz, 2OCH₂CH₃). ³¹P NMR (243 MHz, DMSO-d₆) δ [ppm]=32.9. LC-MS: positive mode [m/z]=597.2 [M+H]⁺. Purity: 87%.

### Synthesis of Diethyl (6-(6-amino-5-(3-(2-bromophenyl)propanamido)-2,4-dioxo-3-(prop-2-yn-1-yl)-3,4-dihydropyrimidin-1(2H)-yl)hexyl)phosphonate (41h

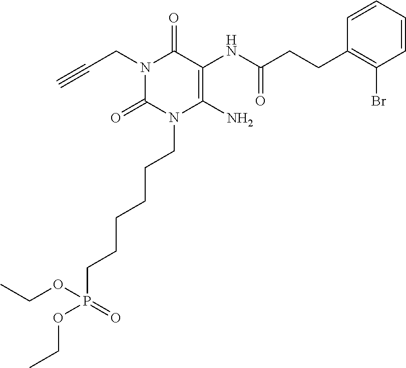 [SMILES: C#CCn1c(=O)c(NC(=O)CCc2ccccc2Br)c(N)n(CCCCCCP(=O)(OCC)OCC)c1=O]

Yield: 43%, light-yellow solid. ¹H NMR (600 MHz, DMSO-d₆) δ [ppm]=8.43 (s, 1H, CONH), 7.59 (dd, J=8.0, 1.2 Hz, 1H, Hₐᵣₒₘ), 7.39 (dd, J=7.7, 1.7 Hz, 1H, Hₐᵣₒₘ), 7.33 (td, J=7.5, 1.2 Hz, 1H, Hₐᵣₒₘ), 7.16 (td, J=7.7, 1.7 Hz, 1H, Hₐᵣₒₘ), 6.67 (s, 2H, NH₂), 4.48 (d, J=2.4 Hz, 2H, N3-CH₂), 4.01-3.91 (m, 4H, 2OCH₂), 3.85 (t, J=7.6 Hz, 2H, N1-CH₂), 3.01 (t, J=2.4 Hz, 1H, H_{propargyl}), 2.98-2.93 (m, 2H, CH₂), 2.60-2.55 (m, 2H, CH₂), 1.74-1.63 (m, 2H, CH₂), 1.52 (q, J=7.5 Hz, 2H, CH₂), 1.50-1.42 (m, 2H, CH₂), 1.37 (p, J=6.9 Hz, 2H, CH₂), 1.34-1.27 (m, 2H, CH₂), 1.22 (t, J=6.9 Hz, 6H, 2OCH₂CH₃). ¹³C NMR (151 MHz, DMSO-d₆) δ [ppm]=171.7 (CONH), 158.1 (C6), 151.8 (CO), 149.6 (CO), 140.5 (Cₐᵣₒₘ), 132.4 (Cₐᵣₒₘ), 130.4 (Cₐᵣₒₘ), 128.1 (Cₐᵣₒₘ), 127.9 (Cₐᵣₒₘ), 123.7 (Cₐᵣₒₘ), 87.2 (C5), 79.9 (C_{propargyl}), 72.4 (C_{propargyl}), 60.7 (d, ²J_{C,P}=5.2 Hz, 2OCH₂), 42.5 (N1-CH₂), 34.9 (CH₂), 31.0 (N3-CH₂), 30.6 (CH₂), 29.8 (CH₂), 29.4 (d, ³J_{C,P}=16.0 Hz, CH₂), 25.3 (CH₂), 24.3 (d, ¹J_{C,P}=138.1 Hz, PCH₂), 21.9 (d, ²J_{C,P}=5.0 Hz, CH₂), 16.3 (d, ¹J_{C,P}=5.7 Hz, 2OCH₂CH₃). ³¹P NMR (243 MHz, DMSO-d₆) δ [ppm]=33.0. LC-MS: positive mode [m/z]=611.4 [M+H]⁺. Purity: 88%.

### Synthesis of Diethyl (4-(6-amino-5-(3-(2-iodophenyl)propanamido)-2,4-dioxo-3-(prop-2-yn-1-yl)-3,4-dihydropyrimidin-1(2H)-yl)butyl)phosphonate (39b

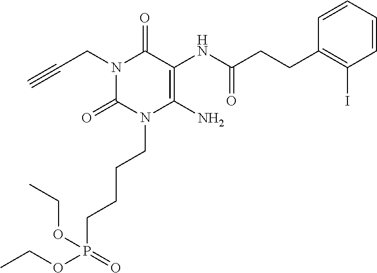 [SMILES: C#CCn1c(=O)c(NC(=O)CCc2ccccc2I)c(N)n(CCCCP(=O)(OCC)OCC)c1=O]

Yield: 43%, off-white solid. ¹H NMR (600 MHz, DMSO-d₆) δ [ppm]=8.43 (s, 1H, CONH), 7.88-7.82 (m, 1H, Hₐᵣₒₘ), 7.39-7.33 (m, 2H, 2Hₐᵣₒₘ), 6.97 (ddd, J=7.9, 6.0, 3.1 Hz, 1H, Hₐᵣₒₘ), 6.71 (s, 2H, NH₂), 4.48 (d, J=2.5 Hz, 2H, N3-CH₂), 4.02-3.94 (m, 4H, 2OCH₂), 3.90-3.83 (t, J=7.4 Hz, 2H, N1-CH₂), 3.01 (t, J=2.4 Hz, 1H, H_{propargyl}), 2.96-2.90 (m, 2H, CH₂), 2.59-2.52 (m, 2H, CH₂), 1.78 (ddd, J=18.0, 9.0, 6.7 Hz, 2H, CH₂), 1.62 (h, J=6.7, 6.2 Hz, 2H, CH₂), 1.59-1.47 (m, 2H, CH₂), 1.23 (t, J=7.0 Hz, 6H, 2OCH₂CH₃). ¹³C NMR (151 MHz, DMSO-d₆) δ [ppm]=171.6 (CONH), 158.1 (C6), 151.8 (CO), 149.7 (CO), 143.8 (Cₐᵣₒₘ), 139.0 (Cₐᵣₒₘ), 129.4 (Cₐᵣₒₘ), 128.6 (Cₐᵣₒₘ), 128.2 (Cₐᵣₒₘ), 100.7 (Cₐᵣₒₘ), 87.2 (C5), 79.9 (C_{propargyl}), 72.4 (C_{propargyl}), 60.9 (d, ²J_{C,P}=6.6 Hz, 2OCH₂), 42.1 (N1-CH₂), 35.7 (CH₂), 35.2 (CH₂), 29.8 (N3-CH₂), 28.1 (d, ³J_{C,P}=16.0 Hz, CH₂), 24.0 (d, ¹J_{C,P}=138.6 Hz, PCH₂), 19.1 (d, ²J_{C,P}=4.4 Hz, CH₂), 16.3 (d, ³J_{C,P}=5.7 Hz, 2OCH₂CH₃). ³¹P NMR (243 MHz, DMSO-d₆) δ [ppm]=32.7. LC-MS: positive mode [m/z]=631.3 [M+H]⁺. Purity: 93%.

### Synthesis of Diethyl (4-(6-amino-2,4-dioxo-3-(prop-2-yn-1-yl)-5-(3-(o-tolyl)propanamido)-3,4-dihydropyrimidin-1(2H)-yl)butyl)phosphonate (39c

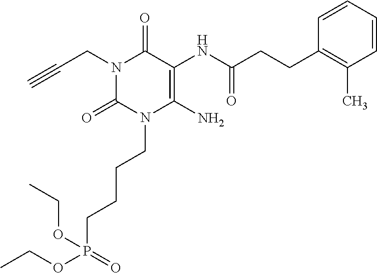 [SMILES: C#CCn1c(=O)c(NC(=O)CCc2ccccc2C)c(N)n(CCCCP(=O)(OCC)OCC)c1=O]

Yield: 42%, colorless solid. ¹H NMR (600 MHz, DMSO-d₆) δ [ppm]=8.40 (s, 1H, CONH), 7.20-6.99 (m, 4H, 4Hₐᵣₒₘ), 6.64 (s, 2H, NH₂), 4.48 (d, J=2.5 Hz, 2H, N3-CH₂), 4.04-3.93 (m, 4H, 2OCH₂), 3.89-3.81 (m, 2H, N1-CH₂), 3.01 (t, J=2.4 Hz, 1H, H_{propargyl}), 2.87-2.81 (m, 2H, CH₂), 2.52-2.49 (m, 2H, CH₂), 2.30 (s, 3H, CH₃), 1.85-1.70 (m, 2H, CH₂), 1.64 (h, J=7.5, 6.6 Hz, 2H, CH₂), 1.58-1.48 (m, 2H, CH₂), 1.23 (t, J=7.1 Hz, 6H, 2OCH₂CH₃). ¹³C NMR (151 MHz, DMSO-d₆) δ [ppm]=172.2 (CONH), 158.1 (C6), 151.7 (CO), 149.6 (CO), 139.7 (Cₐᵣₒₘ), 135.5 (Cₐᵣₒₘ), 129.9 (Cₐᵣₒₘ), 128.3 (Cₐᵣₒₘ), 125.9 (Cₐᵣₒₘ), 125.9 (Cₐᵣₒₘ), 87.2 (C5), 79.9 (C_{propargyl}), 72.4 (C_{propargyl}), 60.8 (d, ²J_{C,P}=5.6 Hz, 2OCH₂), 42.1 (N1-CH₂), 35.5 (CH₂), 29.8 (N3-CH₂), 28.1 (CH₂), 28.1 (d, ³J_{C,P}=15.9 Hz, CH₂), 24.0 (d, ¹J_{C,P}=138.6 Hz, PCH₂), 19.1 (d, ²J_{C,P}=4.7 Hz, CH₂), 18.9 (CH₃), 16.3 (d, ³J_{C,P}=5.5 Hz, 2OCH₂CH₃). ³¹P NMR (243 MHz, DMSO-d₆) δ [ppm]=32.7. LC-MS: positive mode [m/z]=519.40 [M+H]⁺. Purity: 77%.

### Synthesis of Diethyl (4-(6-amino-5-(3-(2,6-dimethylphenyl)propanamido)-2,4-dioxo-3-(prop-2-yn-1-yl)-3,4-dihydropyrimidin-1(2H)-yl)butyl)phosphonate (39l

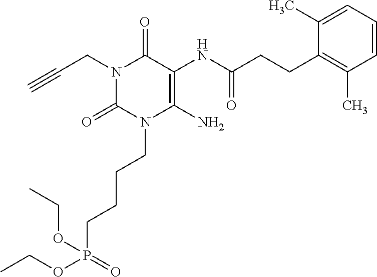 [SMILES: C#CCn1c(=O)c(NC(=O)CCc2c(C)cccc2C)c(N)n(CCCCP(=O)(OCC)OCC)c1=O]

Compound 39l was used for the next step without further purification. LC-MS: positive mode [m/z]=533.1 [M+H]⁺. Light-yellow solid.

### Synthesis of Diethyl (4-(6-amino-5-(3-(2,6-dichlorophenyl)propanamido)-2,4-dioxo-3-(prop-2-yn-1-yl)-3,4-dihydropyrimidin-1(2H)-yl)butyl)phosphonate (39m

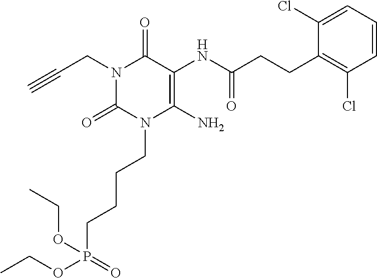 [SMILES: C#CCn1c(=O)c(NC(=O)CCc2c(Cl)cccc2Cl)c(N)n(CCCCP(=O)(OCC)OCC)c1=O]

Compound 39m was used for the next step without further purification. LC-MS: positive mode [m/z]=573.3 [M+H]⁺. Light-yellow solid.

### Synthesis of Diethyl (4-(6-amino-5-(3-(4-bromophenyl)propanamido)-2,4-dioxo-3-(prop-2-yn-1-yl)-3,4-dihydropyrimidin-1(2H)-yl)butyl)phosphonate (39d

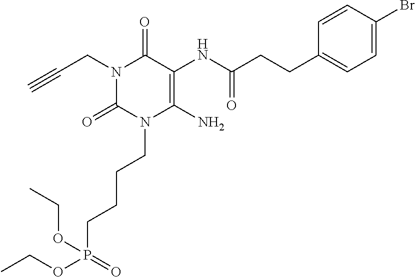 [SMILES: C#CCn1c(=O)c(NC(=O)CCc2ccc(Br)cc2)c(N)n(CCCCP(=O)(OCC)OCC)c1=O]

Yield: 76%, yellow viscous liquid. ¹H NMR (600 MHz, DMSO-d₆) δ [ppm]=8.42 (s, 1H, CONH), 7.46 (dd, J=8.3, 3.4 Hz, 2H, 2Hₐᵣₒₘ), 7.26-7.19 (m, 2H, 2Hₐᵣₒₘ), 6.68 (s, 2H, NH₂), 4.48 (d, J=2.5 Hz, 2H, N3-CH₂), 4.02-3.93 (m, 4H, 2OCH₂), 3.91-3.82 (m, 2H, N1-CH₂), 3.01 (t, J=2.4 Hz, 1H, H_{propargyl}), 2.87-2.81 (m, 2H, CH₂), 2.57-2.50 (m, 2H, CH₂), 1.84-1.73 (m, 2H, CH₂), 1.63 (p, J=7.6 Hz, 2H, CH₂), 1.58-1.47 (m, 2H, CH₂), 1.23 (t, J=7.0 Hz, 6H, 2OCH₂CH₃). ¹³C NMR (151 MHz, DMSO-d₆) δ [ppm]=171.9 (CONH), 158.1 (C6), 151.7 (CO), 149.6 (CO), 141.0 (Cₐᵣₒₘ), 131.1 (2Cₐᵣₒₘ), 130.5 (2Cₐᵣₒₘ), 118.8 (Cₐᵣₒₘ), 87.2 (C5), 79.9 (C_{propargyl}), 72.5 (C_{propargyl}), 60.9 (d, ²J_{C,P}=6.6 Hz, 2OCH₂), 42.1 (N1-CH₂), 36.5 (CH₂), 30.1 (CH₂), 29.8 (N3-CH₂), 28.1 (d, ³J_{C,P}=15.4 Hz, CH₂), 24.0 (d, ¹J_{C,P}=139.0 Hz, PCH₂), 19.1 (d, ²J_{C,P}=4.4 Hz, CH₂), 16.3 (d, ³J_{C,P}=5.0 Hz, 2OCH₂CH₃). ³¹P NMR (243 MHz, DMSO-d₆) δ [ppm]=32.7. LC-MS: positive mode [m/z]=583.1 [M+H]⁺. Purity: 80%.

### Synthesis of Diethyl (4-(6-amino-5-(3-(4-iodophenyl)propanamido)-2,4-dioxo-3-(prop-2-yn-1-yl)-3,4-dihydropyrimidin-1(2H)-yl)butyl)phosphonate (39e

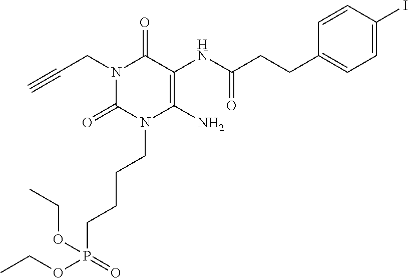 [SMILES: C#CCn1c(=O)c(NC(=O)CCc2ccc(I)cc2)c(N)n(CCCCP(=O)(OCC)OCC)c1=O]

Yield: 67%, light-yellow solid. H NMR (600 MHz, DMSO-d₆) δ [ppm]=8.41 (s, 1H, CONH), 7.63 (d, J=8.3 Hz, 2H, 2Hₐᵣₒₘ), 7.09 (d, J=8.3 Hz, 2H, 2Hₐᵣₒₘ), 6.68 (s, 2H, NH₂), 4.48 (d, J=2.5 Hz, 2H, N3-CH₂), 4.02-3.94 (m, 4H, 2OCH₂), 3.88-3.84 (m, 2H, N1-CH₂), 3.01 (t, J=2.4 Hz, 1H, H_{propargyl}), 2.85-2.79 (m, 2H, CH₂), 2.56-2.50 (m, 2H, CH₂), 1.84-1.73 (m, 2H, CH₂), 1.63 (p, J=7.6 Hz, 2H, CH₂), 1.58-1.48 (m, 2H, CH₂), 1.23 (t, J=7.0 Hz, 6H, 2OCH₂CH₃). ¹³C NMR (151 MHz, DMSO-d₆) δ [ppm]=171.9 (CONH), 158.1 (C6), 151.7 (CO), 149.6 (CO), 141.4 (Cₐᵣₒₘ), 137.0 (2Cₐᵣₒₘ), 130.7 (2Cₐᵣₒₘ), 91.3 (Cₐᵣₒₘ), 87.2 (C5), 79.9 (C_{propargyl}), 72.4 (C_{propargyl}), 60.9 (d, ²J_{C,P}=6.6 Hz, 2OCH₂), 42.1 (N1-CH₂), 40.1 (CH₂), 30.2 (CH₂), 29.8 (N3-CH₂), 28.1 (d, ³J_{C,P}=15.8 Hz, CH₂), 24.0 (d, ¹J_{C,P}=138.4 Hz, PCH₂), 19.1 (d, ²J_{C,P}=4.6 Hz, CH₂), 16.3 (d, ³J_{C,P}=5.7 Hz, 2OCH₂CH₃). ³¹P NMR (243 MHz, DMSO-d₆) δ [ppm]=32.7. LC-MS: positive mode [m/z]=631.40 [M+H]⁺.

### Synthesis of Diethyl (4-(6-amino-2,4-dioxo-3-(prop-2-yn-1-yl)-5-(3-(4-(trifluoromethyl) phenyl)propanamido)-3,4-dihydropyrimidin-1(2H)-yl)butyl)phosphonate (39f

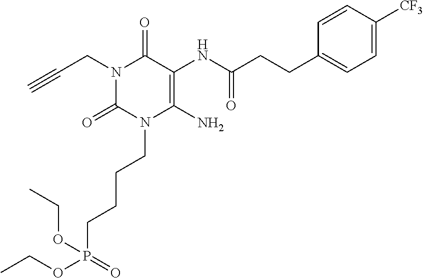 [SMILES: C#CCn1c(=O)c(NC(=O)CCc2ccc(C(F)(F)F)cc2)c(N)n(CCCCP(=O)(OCC)OCC)c1=O]

Yield: 70%, white solid. ¹H NMR (500 MHz, DMSO-d₆) δ [ppm]=8.43 (s, 1H, CONH), 7.64 (d, J=8.1 Hz, 2H, 2Hₐᵣₒₘ), 7.50 (d, J=7.9 Hz, 2H, 2Hₐᵣₒₘ), 6.69 (s, 2H, NH₂), 4.48 (d, J=2.5 Hz, 2H, N3-CH₂), 4.03-3.93 (m, 4H, 2OCH₂), 3.90-3.79 (m, 2H, N1-CH₂), 3.01 (t, J=2.4 Hz, 1H, H_{propargyl}), 2.97 (t, J=7.9 Hz, 2H, CH₂), 2.63-2.56 (m, 2H, CH₂), 1.84-1.72 (m, 2H, CH₂), 1.63 (q, J=8.0 Hz, 2H, CH₂), 1.53 (dt, J=12.6, 7.8 Hz, 2H, CH₂), 1.23 (t, J=7.0 Hz, 6H, 2OCH₂CH₃). ¹³C NMR (126 MHz, DMSO-d₆) δ [ppm]=171.8 (CONH), 158.1 (C6), 151.7 (CO), 149.6 (CO), 146.6 (Cₐᵣₒₘ), 129.0 (2Cₐᵣₒₘ), 126.7 (q, ²J_{C,F}=33.3 Hz, Cₐᵣₒₘ), 125.1 (q, ³J_{C,F}=3.9 Hz, 2Cₐᵣₒₘ), 123.3 (q, ¹J_{C,F}=272.2 Hz, CF₃), 87.1 (C5), 79.9 (C_{propargyl}), 72.4 (C_{propargyl}), 60.8 (d, ²J_{C,P}=6.3 Hz, 2OCH₂), 42.1 (N1-CH₂), 36.2 (CH₂), 30.5 (CH₂), 29.8 (N3-CH₂), 28.0 (d, ³J_{C,P}=15.6 Hz, CH₂), 24.00 (d, ¹J_{C,P}=138.7 Hz, PCH₂) 19.1 (d, ²J_{C,P}=4.9 Hz, CH₂), 16.3 (d, ³J_{C,P}=5.6 Hz, 2OCH₂CH₃). ³¹P NMR (202 MHz, DMSO-d₆) δ [ppm]=32.7. LC-MS: positive mode [m/z]=573.50 [M+H]⁺.

### Synthesis of Diethyl (4-(6-amino-5-(3-(4-methoxyphenyl)propanamido)-2,4-dioxo-3-(prop-2-yn-1-yl)-3,4-dihydropyrimidin-1(2H)-yl)butyl)phosphonate (39g

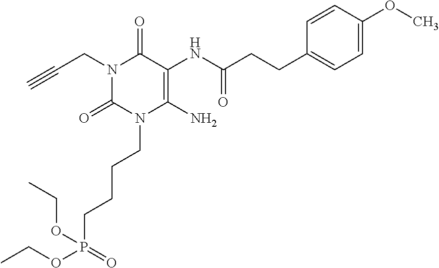 [SMILES: C#CCn1c(=O)c(NC(=O)CCc2ccc(OC)cc2)c(N)n(CCCCP(=O)(OCC)OCC)c1=O]

Yield: 77%, light-yellow solid. H NMR (600 MHz, DMSO-d₆) δ [ppm]=8.39 (s, 1H, CONH), 7.17-7.15 (m, 2H, 2Hₐᵣₒₘ), 6.87-6.84 (m, 2H, 2Hₐᵣₒₘ), 6.63 (s, 2H, NH₂), 4.48 (d, J=2.5 Hz, 2H, N3-CH₂), 4.02-3.92 (m, 4H, 2OCH₂), 3.88-3.84 (m, 2H, N1-CH₂), 3.72 (s, 3H, OCH₃), 3.01 (t, J=2.4 Hz, 1H, H_{propargyl}), 2.84-2.77 (m, 2H, CH₂), 2.53-2.50 (m, 2H, CH₂), 1.84-1.73 (m, 2H, CH₂), 1.63 (p, J=7.7 Hz, 2H, CH₂), 1.53 (dq, J=12.2, 8.0 Hz, 2H, CH₂), 1.23 (t, J=7.0 Hz, 6H, 2OCH₂CH₃). ¹³C NMR (151 MHz, DMSO-d₆) δ [ppm]=172.2 (CONH), 158.1 (C6), 157.4 (Cₐᵣₒₘ), 151.7 (CO), 149.6 (CO), 133.4 (Cₐᵣₒₘ), 129.1 (2Cₐᵣₒₘ), 113.7 (2Cₐᵣₒₘ), 87.2 (C5), 79.9 (C_{propargyl}), 72.4 (C_{propargyl}), 60.8 (d, J=6.5 Hz, 2OCH₂), 55.0 (OCH₃), 42.1 (N1-CH₂), 37.2 (CH₂), 29.9 (CH₂), 29.8 (CH₂), 28.1 (d, ³J_{C,P}=15.8 Hz, CH₂), 24.0 (d, ¹J_{C,P}=138.5 Hz, PCH₂), 19.1 (d, ²J_{C,P}=4.7 Hz, CH₂), 16.3 (d, ³J_{C,P}=4.9 Hz, 2OCH₂CH₃). ³¹P NMR (243 MHz, DMSO-d₆) δ [ppm]=32.7. LC-MS: positive mode [m/z]=535.50 [M+H]⁺.

### Synthesis of Diethyl (4-(6-amino-5-(3-(4-ethylphenyl)propanamido)-2,4-dioxo-3-(prop-2-yn-1-yl)-3,4-dihydropyrimidin-1(2H)-yl)butyl)phosphonate (39i

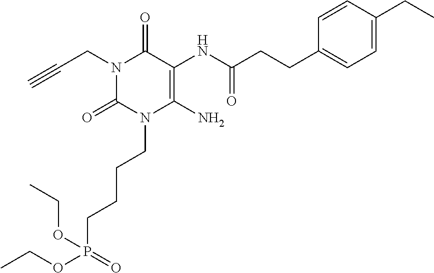 [SMILES: C#CCn1c(=O)c(NC(=O)CCc2ccc(CC)cc2)c(N)n(CCCCP(=O)(OCC)OCC)c1=O]

Yield: 80%, yellow viscous liquid. ¹H NMR (500 MHz, DMSO-d₆) δ [ppm]=8.39 (s, 1H, CONH), 7.15-7.14 (m, 2H, 2Hₐᵣₒₘ), 7.13-7.12 (m, 2H, 2Hₐᵣₒₘ), 6.63 (s, 2H, NH₂), 4.48 (d, J=2.5 Hz, 2H, N3-CH₂), 4.03-3.92 (m, 4H, 2OCH₂), 3.90-3.83 (m, 2H, N1-CH₂), 3.00 (t, J=2.4 Hz, 1H, H_{propargyl}), 2.86-2.79 (m, 2H, CH₂), 2.60-2.52 (m, 4H, CH₂ and CH₂CH₃), 1.85-1.71 (m, 2H, CH₂), 1.63 (dq, J=14.9, 7.2 Hz, 2H, CH₂), 1.59-1.46 (m, 2H, CH₂), 1.23 (t, J=7.0 Hz, 6H, 2OCH₂CH₃), 1.17 (t, J=7.6 Hz, 3H, CH₂CH₃). ¹³C NMR (126 MHz, DMSO-d₆) δ [ppm]=172.2 (CONH), 158.1 (C6), 151.7 (CO), 149.6 (CO), 141.1 (Cₐᵣₒₘ), 138.7 (Cₐᵣₒₘ), 128.0 (2Cₐᵣₒₘ), 127.7 (2Cₐᵣₒₘ), 87.3 (C5), 79.9 (C_{propargyl}), 72.4 (C_{propargyl}), 60.8 (d, ²J_{C,P}=6.4 Hz, 2OCH₂), 42.1 (N1-CH₂), 37.0 (CH₂), 30.4 (CH₂), 29.8 (CH₂), 28.1 (d, ³J_{C,P}=15.5 Hz, CH₂), 27.7 (CH₂CH₃), 24.0 (d, ¹J_{C,P}=138.8 Hz, PCH₂), 19.1 (d, ²J_{C,P}=4.8 Hz, CH₂), 16.3 (d, ³J_{C,P}=5.6 Hz, 2OCH₂CH₃), 15.6 (CH₂CH₃). ³¹P NMR (202 MHz, DMSO-d₆) δ [ppm]=32.7. LC-MS: positive mode [m/z]=533.3 [M+H]⁺.

### Synthesis of Diethyl (4-(6-amino-5-(3-(4-isopropylphenyl)propanamido)-2,4-dioxo-3-(prop-2-yn-1-yl)-3,4-dihydropyrimidin-1(2H)-yl)butyl)phosphonate (39j

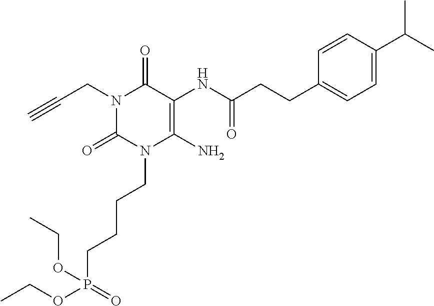 [SMILES: C#CCn1c(=O)c(NC(=O)CCc2ccc(C(C)C)cc2)c(N)n(CCCCP(=O)(OCC)OCC)c1=O]

Yield: 43%, light-yellow solid. ¹H NMR (500 MHz, DMSO-d₆) δ [ppm]=8.39 (s, 1H, CONH), 7.16 (s, 4H, 4Hₐᵣₒₘ), 6.64 (s, 2H, NH₂), 4.48 (d, J=2.5 Hz, 2H, N3-CH₂), 4.04-3.92 (m, 4H, 2OCH₂), 3.87 (t, J=7.6 Hz, 2H, N1-CH₂), 3.00 (t, J=2.4 Hz, 1H, H_{propargyl}), 2.85-2.79 (m, 3H, CH₂ and CH(CH₃)₂), 2.55-2.52 (m, 2H, CH₂), 1.86-1.71 (m, 2H, CH₂), 1.69-1.60 (m, 2H, CH₂), 1.58-1.48 (m, 2H, CH₂), 1.23 (t, J=7.0 Hz, 6H, 2OCH₂CH₃), 1.19 (d, J=7.0 Hz, 6H, CH(CH₃)₂). ¹³C NMR (126 MHz, DMSO-d₆) δ [ppm]=172.2 (CONH), 158.1 (C6), 151.7 (CO), 149.6 (CO), 145.8 (Cₐᵣₒₘ), 138.9 (Cₐᵣₒₘ), 128.0 (2Cₐᵣₒₘ), 126.2 (2Cₐᵣₒₘ), 87.3 (C5), 79.9 (C_{propargyl}), 72.4 (C_{propargyl}), 60.9 (d, ²J_{C,P}=6.3 Hz, OCH₂), 42.1 (N1-CH₂), 37.0 (CH₂), 33.0 (CH(CH₃)₂), 29.8 (CH₂), 28.1 (d, ³J_{C,P}=15.6 Hz, CH₂), 24.0 (d, J_{C,P}=138.7 Hz, PCH₂), 23.9 (CH(CH₃)₂), 19.1 (d, ²J_{C,P}=5.0 Hz, CH₂), 16.3 (d, ³J_{C,P}=5.7 Hz, 2OCH₂CH₃). ³¹P NMR (202 MHz, DMSO-d₆) δ [ppm]=32.7. LC-MS: positive mode [m/z]=547.4 [M+H]⁺.

### Synthesis of Diethyl (4-(5-(3-([1,1′-biphenyl]-4-yl)propanamido)-6-amino-2,4-dioxo-3-(prop-2-yn-1-yl)-3,4-dihydropyrimidin-1(2H)-yl)butyl)phosphonate (39k

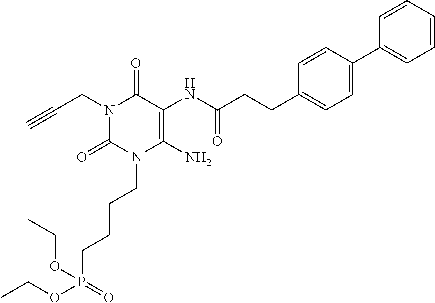 [SMILES: C#CCn1c(=O)c(NC(=O)CCc2ccc(-c3ccccc3)cc2)c(N)n(CCCCP(=O)(OCC)OCC)c1=O]

Yield: 45%, light-yellow solid. ¹H NMR (500 MHz, DMSO-d₆) δ [ppm]=8.43 (s, 1H, CONH), 7.67-7.63 (m, 2H, 2Hₐᵣₒₘ), 7.61-7.57 (m, 2H, 2Hₐᵣₒₘ), 7.49-7.41 (m, 2H, 2Hₐᵣₒₘ), 7.37-7.33 (m, 2H, 2Hₐᵣₒₘ), 6.66 (s, 2H, NH₂), 4.49 (d, J=2.5 Hz, 2H, N3-CH₂), 4.04-3.93 (m, 4H, 2OCH₂), 3.90-3.81 (m, 2H, N1-CH₂), 3.01 (t, J=2.4 Hz, 1H, H_{propargyl}), 2.94-2.89 (m, 2H, CH₂), 2.63-2.57 (m, 2H, CH₂), 1.87-1.72 (m, 2H, CH₂), 1.63 (p, J=7.5 Hz, 2H, CH₂), 1.53 (dt, J=12.4, 7.7 Hz, 2H, CH₂), 1.23 (t, J=7.1 Hz, 6H, 2OCH₂CH₃). ¹³C NMR (126 MHz, DMSO-d₆) δ [ppm]=172.1 (CONH), 158.1 (C6), 151.7 (CO), 149.6 (CO), 140.9 (Cₐᵣₒₘ), 140.1 (Cₐᵣₒₘ), 137.8 (Cₐᵣₒₘ), 128.8 (2Cₐᵣₒₘ), 128.8 (2Cₐᵣₒₘ), 127.1 (Cₐᵣₒₘ), 126.6 (2Cₐᵣₒₘ), 126.5 (2Cₐᵣₒₘ), 87.3 (C5), 79.9 (C_{propargyl}), 72.4 (C_{propargyl}), 60.9 (d, ²J_{C,P}=6.4 Hz, 2OCH₂), 42.1 (N1-CH₂), 36.8 (CH₂), 30.4 (CH₂), 29.8 (CH₂), 28.1 (d, ³J_{C,P}=15.6 Hz, CH₂), 24.0 (d, J_{C,P}=138.6 Hz, PCH₂), 19.1 (d, ²J_{C,P}=4.9 Hz, CH₂), 16.3 (d, ³J_{C,P}=5.5 Hz, 2OCH₂CH₃). ³¹P NMR (202 MHz, DMSO-d₆) δ [ppm]=32.7. LC-MS: positive mode [m/z]=581.3 [M+H]⁺.

### Synthesis of Diethyl (4-(6-amino-5-(2-(naphthalen-1-yl)acetamido)-2,4-dioxo-3-(prop-2-yn-1-yl)-3,4-dihydropyrimidin-1(2H)-yl)butyl)phosphonate (76

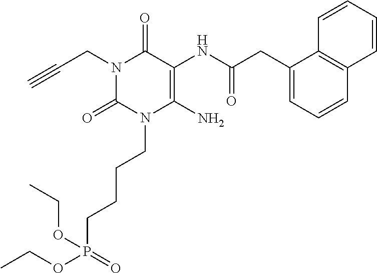 [SMILES: C#CCn1c(=O)c(NC(=O)Cc2cccc3ccccc23)c(N)n(CCCCP(=O)(OCC)OCC)c1=O]

Yield: 75%, off-white solid. ¹H NMR (600 MHz, DMSO-d₆) δ [ppm]=8.69 (s, 1H, CONH), 7.90-7.83 (m, 4H, 4Hₐᵣₒₘ), 7.57-7.43 (m, 3H, 3Hₐᵣₒₘ), 6.77 (s, 2H, NH₂), 4.48 (d, J=2.5 Hz, 2H, N3-CH₂), 4.02-3.93 (m, 4H, 2OCH₂), 3.87 (t, J=7.7 Hz, 2H, N1-CH₂), 3.77 (s, 2H, CH₂), 3.00 (t, J=2.4 Hz, 1H, H_{propargyl}), 1.86-1.71 (m, 2H, CH₂), 1.63 (dt, J=15.0, 7.6 Hz, 2H, CH₂), 1.53 (dt, J=20.5, 7.8 Hz, 2H, CH₂), 1.22 (t, J=7.0 Hz, 6H, 2OCH₂CH₃). ¹³C NMR (151 MHz, DMSO-d₆) δ [ppm]=170.8 (CONH), 158.1 (C6), 151.8 (CO), 149.6 (CO), 134.1 (Cₐᵣₒₘ), 133.0 (Cₐᵣₒₘ), 131.7 (Cₐᵣₒₘ), 128.0 (Cₐᵣₒₘ), 127.5 (Cₐᵣₒₘ), 127.4 (Cₐᵣₒₘ), 127.4 (Cₐᵣₒₘ), 127.3 (Cₐᵣₒₘ), 125.9 (Cₐᵣₒₘ), 125.4 (Cₐᵣₒₘ), 87.2 (C5), 79.9 (C_{propargyl}), 72.5 (C_{propargyl}), 60.8 (d, ²J_{C,P}=6.4 Hz, 2OCH₂), 42.2 (CH₂), 42.1 (CH₂), 29.8 (N3-CH₂), 28.1 (d, ³J_{C,P} 16.0 Hz, CH₂), 24.0 (d, ¹J_{C,P}=138.8 Hz, PCH₂), 19.1 (d, ²J_{C,P}=4.7 Hz, CH₂), 16.3 (d, ³J_{C,P}=5.6 Hz, 2OCH₂CH₃). ³¹P NMR (243 MHz, DMSO-d₆) δ [ppm]=32.7. LC-MS: positive mode [m/z]=541.4 [M+H]⁺.

### Synthesis of Ethyl 6-(6-amino-5-(3-(2-bromophenyl)propanamido)-2,4-dioxo-3-(prop-2-yn-1-yl)-3,4-dihydropyrimidin-1(2H)-yl)hexanoate (80

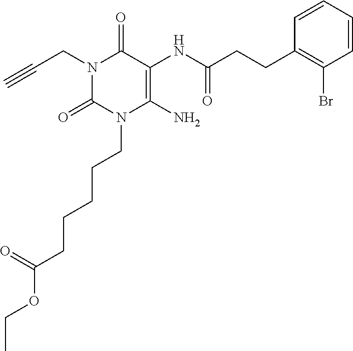 [SMILES: C#CCn1c(=O)c(NC(=O)CCc2ccccc2Br)c(N)n(CCCCCC(=O)OCC)c1=O]

Yield: 47%, light-yellow liquid. ¹H NMR (600 MHz, DMSO-d₆) δ [ppm]=8.42 (s, 1H, CONH), 7.59 (dd, J=8.0, 1.2 Hz, 1H, Hₐᵣₒₘ), 7.39 (dd, J=7.7, 1.7 Hz, 1H, Hₐᵣₒₘ), 7.33 (td, J=7.5, 1.3 Hz, 1H, Hₐᵣₒₘ), 7.16 (td, J=7.7, 1.8 Hz, 1H, Hₐᵣₒₘ), 6.67 (s, 2H, NH₂), 4.48 (d, J=2.5 Hz, 2H, N3-CH₂), 4.05 (q, J=7.1 Hz, 2H, OCH₂), 3.85 (t, J=7.7 Hz, 2H, N1-CH₂), 3.01 (t, J=2.4 Hz, 1H, H_{propargyl}), 2.98-2.94 (m, 2H, CH₂), 2.59-2.54 (m, 2H, CH₂), 2.29 (t, J=7.3 Hz, 2H, CH₂), 1.53 (ddt, J=19.1, 11.4, 5.5 Hz, 4H, 2CH₂), 1.35-1.26 (m, 2H, CH₂), 1.18 (t, J=7.1 Hz, 3H, OCH₂CH₃). ¹³C NMR (151 MHz, DMSO-d₆) δ [ppm]=172.8 (COOCH₂), 171.7 (CONH), 158.1 (C6), 151.8 (CO), 149.7 (CO), 140.5 (Cₐᵣₒₘ), 132.4 (Cₐᵣₒₘ), 130.4 (Cₐᵣₒₘ), 128.2 (Cₐᵣₒₘ), 127.9 (Cₐᵣₒₘ), 123.7 (Cₐᵣₒₘ), 87.2 (C5), 79.9 (C_{propargyl}), 72.4 (C_{propargyl}), 59.7 (OCH₂), 42.4 (N1-CH₂), 35.8 (CH₂), 33.4 (CH₂), 31.0 (N3-CH₂), 30.8 (CH₂), 27.2 (CH₂), 25.3 (CH₂), 24.1 (CH₂), 14.1 (OCH₂CH₃). LC-MS: positive mode [m/z]=533.2 [M+H]⁺. Purity: 97%.

### Synthesis of Diethyl (4-(8-(2-fluorophenethyl)-2,6-dioxo-1-(prop-2-yn-1-yl)-1,2,6,7-tetrahydro-3H-purin-3-yl)butyl)phosphonate (42a

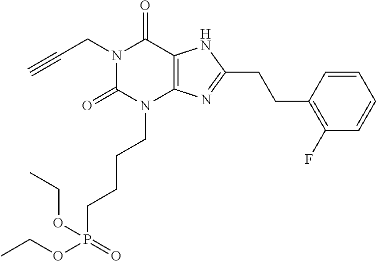 [SMILES: C#CCn1c(=O)c2[nH]c(CCc3ccccc3F)nc2n(CCCCP(=O)(OCC)OCC)c1=O]

Yield: 54%, light-yellow solid. ¹H NMR (600 MHz, DMSO-d₆) δ [ppm]=13.32 (s, 1H, N7-H), 7.29-7.21 (m, 2H, 2Hₐᵣₒₘ), 7.17-7.07 (m, 2H, 2Hₐᵣₒₘ), 4.59 (d, J=2.5 Hz, 2H, N1-CH₂), 3.98 (t, J=7.2 Hz, 2H, N3-CH₂), 3.99-3.88 (m, 4H, 2OCH₂), 3.08-3.03 (m, 3H, H_{propargyl} and CH₂), 3.00 (t, J=7.7 Hz, 2H, CH₂), 1.76 (ddt, J=14.9, 10.6, 7.2 Hz, 4H, 2CH₂), 1.52-1.40 (m, 2H, CH₂), 1.19 (t, J=7.0 Hz, 6H, 2OCH₂CH₃). ¹³C NMR (151 MHz, DMSO-d₆) δ [ppm]=160.5 (d, ¹J_{C,F}=243.5 Hz, Cₐᵣₒₘ), 153.6 (Cₓₐₙₜₕᵢₙₑ), 152.9 (Cₓₐₙₜₕᵢₙₑ), 150.0 (Cₓₐₙₜₕᵢₙₑ), 148.1 (Cₓₐₙₜₕᵢₙₑ), 130.7 (d, J=5.1 Hz, Cₐᵣₒₘ), 128.3 (d, J=8.2 Hz, Cₐᵣₒₘ), 126.9 (d, J=15.4 Hz, Cₐᵣₒₘ), 124.4 (d, J=3.5 Hz, Cₐᵣₒₘ), 115.1 (d, J=21.7 Hz, Cₐᵣₒₘ), 105.9 (C5), 79.7 (C_{propargyl}), 72.7 (C_{propargyl}), 60.8 (d, ²J_{C,P}=6.7 Hz, 2OCH₂), 42.4 (N3-CH₂), 30.1 (CH₂), 28.6 (CH₂), 28.1 (d, ³J_{C,P}=15.7 Hz, CH₂), 26.7 (CH₂), 24.1 (d, ¹J_{C,P}=138.7 Hz, PCH₂), 19.3 (d, ²J_{C,P}=4.9 Hz, CH₂), 16.2 (d, ³J_{C,P}=5.6 Hz, 2OCH₂CH₃). ³¹P NMR (243 MHz, DMSO-d₆) δ [ppm]=32.8. LC-MS: positive mode [m/z]=505.4 [M+H]⁺. Purity: 96%.

### Synthesis of Diethyl (5-(8-(2-bromophenethyl)-2,6-dioxo-1-(prop-2-yn-1-yl)-1,2,6,7-tetrahydro-3H-purin-3-yl)pentyl)phosphonate (43h

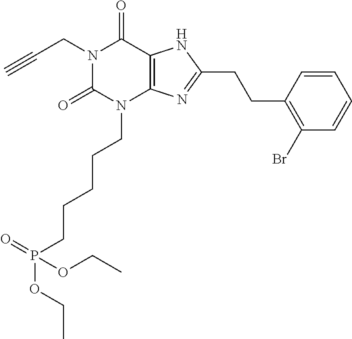 [SMILES: C#CCn1c(=O)c2[nH]c(CCc3ccccc3Br)nc2n(CCCCCP(=O)(OCC)OCC)c1=O]

Yield: 51%, white solid. ¹H NMR (600 MHz, DMSO-d₆) δ [ppm]=13.34 (s, 1H, N7-H), 7.61-7.55 (m, 1H, Hₐᵣₒₘ), 7.30-7.26 (m, 2H, 2Hₐᵣₒₘ), 7.17-7.11 (m, 1H, Hₐᵣₒₘ), 4.59 (d, J=2.5 Hz, 2H, N1-CH₂), 4.00-3.91 (m, 6H, N3-CH₂ and 2OCH₂), 3.14 (t, J=7.5 Hz, 2H, CH₂), 3.07 (t, J=2.4 Hz, 1H, H_{propargyl}), 3.02 (t, J=7.5 Hz, 2H, CH₂), 1.74-1.63 (m, 4H, 2CH₂), 1.55-1.46 (m, 2H, CH₂), 1.36 (t, J=7.3 Hz, 2H, CH₂), 1.20 (t, J=7.0 Hz, 6H, 2OCH₂CH₃). ¹³C NMR (151 MHz, DMSO-d₆) δ [ppm]=153.4 (Cₓₐₙₜₕᵢₙₑ), 152.9 (Cₓₐₙₜₕᵢₙₑ), 150.0 (Cₓₐₜₕᵢₙₑ), 148.1 (Cₓₐₙₜₕᵢₙₑ), 139.3 (Cₐᵣₒₘ), 132.5 (Cₐᵣₒₘ), 130.6 (Cₐᵣₒₘ), 128.4 (Cₐᵣₒₘ), 127.8 (Cₐᵣₒₘ), 123.7 (Cₐᵣₒₘ), 105.9 (C5), 79.7 (C_{propargyl}), 72.7 (C_{propargyl}), 60.7 (d, ²J_{C,P}=6.1 Hz, 2OCH₂), 42.7 (N3-CH₂), 33.5 (CH₂), 30.0 (N1-CH₂), 28.2 (CH₂), 26.9 (CH₂), 26.6 (d, ³J_{C,P}=16.2 Hz, CH₂), 24.3 (d, ¹J_{C,P}=138.2 Hz, PCH₂), 21.7 (d, ²J_{C,P}=4.9 Hz, CH₂), 16.3 (d, ³J_{C,P}=5.5 Hz, 2OCH₂CH₃). ³¹P NMR (243 MHz, DMSO-d₆) δ [ppm]=32.9. LC-MS: positive mode [m/z]=579.2 [M+H]⁺. Purity: 97%.

### Synthesis of Diethyl (6-(8-(2-bromophenethyl)-2,6-dioxo-1-(prop-2-yn-1-yl)-1,2,6,7-tetrahydro-3H-purin-3-yl)hexyl)phosphonate (44h

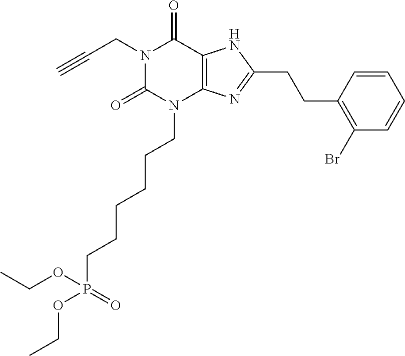 [SMILES: C#CCn1c(=O)c2[nH]c(CCc3ccccc3Br)nc2n(CCCCCCP(=O)(OCC)OCC)c1=O]

Yield: 54%, light-yellow solid. H NMR (600 MHz, DMSO-d₆) δ [ppm]=13.33 (s, 1H, N7-H), 7.59 (d, J=7.9 Hz, 1H, Hₐᵣₒₘ), 7.30-7.26 (m, 2H, 2Hₐᵣₒₘ), 7.15 (ddd, J=8.0, 5.9, 3.2 Hz, 1H, Hₐᵣₒₘ), 4.59 (d, J=2.4 Hz, 2H, N1-CH₂), 3.98 (d, J=6.8 Hz, 2H, N3-CH₂), 3.98-3.90 (m, 4H, 2OCH₂), 3.14 (t, J=7.6 Hz, 2H, CH₂), 3.06 (t, J=2.4 Hz, 1H, H_{propargyl}), 3.02 (t, J=7.6 Hz, 2H, CH₂), 1.72-1.60 (m, 4H, 2CH₂), 1.46 (dtd, J=11.9, 8.8, 5.6 Hz, 2H, CH₂), 1.38 (dt, J=14.8, 6.8 Hz, 2H, CH₂), 1.28 (h, J=7.3, 6.6 Hz, 2H, CH₂), 1.20 (t, J=7.0 Hz, 6H, 2OCH₂CH₃). ¹³C NMR (151 MHz, DMSO-d₆) δ [ppm]=153.4 (Cₓₐₙₜₕᵢₙₑ), 152.9 (Cₓₐₙₜₕᵢₙₑ), 150.0 (Cₓₐₙₜₕᵢₙₑ), 148.1 (Cₓₐₙₜₕᵢₙₑ), 139.3 (Cₐᵣₒₘ), 132.5 (Cₐᵣₒₘ), 130.6 (Cₐᵣₒₘ), 128.5 (Cₐᵣₒₘ), 127.8 (Cₐᵣₒₘ), 123.7 (Cₐᵣₒₘ), 105.9 (C5), 79.7 (C_{propargyl}), 72.6 (C_{propargyl}), 60.7 (d, ²J_{C,P}=6.3 Hz, 2OCH₂), 42.9 (N3-CH₂), 33.5 (CH₂), 30.0 (N1-CH₂), 29.3 (d, ³J_{C,P}=16.5 Hz, CH₂), 28.3 (CH₂), 27.2 (CH₂), 25.5 (CH₂), 24.4 (d, ¹J_{C,P}=138.7 Hz, PCH₂), 21.9 (d, ²J_{C,P}=5.1 Hz, CH₂), 16.2 (d, ³J_{C,P}=6.1 Hz, 2OCH₂CH₃). ³¹P NMR (243 MHz, DMSO-d₆) δ [ppm]=33.0. LC-MS: positive mode [m/z]=593.6 [M+H]⁺. Purity: 90%.

### Synthesis of Diethyl (4-(8-(2-iodophenethyl)-2,6-dioxo-1-(prop-2-yn-1-yl)-1,2,6,7-tetrahydro-3H-purin-3-yl)butyl)phosphonate (42b

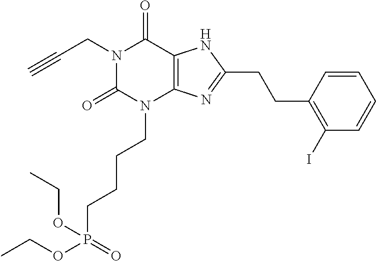 [SMILES: C#CCn1c(=O)c2[nH]c(CCc3ccccc3I)nc2n(CCCCP(=O)(OCC)OCC)c1=O]

Yield: 74%, off-white solid. ¹H NMR (600 MHz, DMSO-d₆) δ [ppm]=13.35 (s, 1H, N7-H), 7.84 (dd, J=7.9, 1.2 Hz, 1H, Hₐᵣₒₘ), 7.31 (td, J=7.5, 1.3 Hz, 1H, Hₐᵣₒₘ), 7.25 (dd, J=7.7, 1.7 Hz, 1H, Hₐᵣₒₘ), 6.96 (td, J=7.6, 1.7 Hz, 1H, Hₐᵣₒₘ), 4.59 (d, J=2.5 Hz, 2H, N1-CH₂), 4.03-3.95 (m, 2H, N3-CH₂), 3.98-3.89 (m, 4H, 2OCH₂), 3.12 (t, J=7.7 Hz, 2H, CH₂), 3.06 (t, J=2.4 Hz, 1H, H_{propargyl}), 2.99 (t, J=7.6 Hz, 2H, CH₂), 1.82-1.73 (m, 4H, 2CH₂), 1.55-1.42 (m, 2H, CH₂), 1.19 (t, J=7.0 Hz, 6H, 2OCH₂CH₃). ¹³C NMR (151 MHz, DMSO-d₆) δ [ppm]=153.3 (Cₓₐₙₜₕᵢₙₑ), 152.9 (Cₓₐₙₜₕᵢₙₑ), 150.0 (Cₓₐₙₜₕᵢₙₑ), 148.1 (Cₓₐₙₜₕᵢₙₑ), 142.6 (Cₐᵣₒₘ), 139.1 (Cₐᵣₒₘ), 129.6 (Cₐᵣₒₘ), 128.5 (Cₐᵣₒₘ), 128.5 (Cₐᵣₒₘ), 105.9 (C5), 100.6 (Cₐᵣₒₘ), 79.7 (C_{propargyl}), 72.7 (C_{propargyl}), 60.8 (d, ²J_{C,P}=6.5 Hz, 2OCH₂), 42.4 (N3-CH₂), 38.0 (CH₂), 30.0 (N1-CH₂), 28.6 (CH₂), 28.2 (d, ³J_{C,P}=15.8 Hz, CH₂), 24.1 (d, ¹J_{C,P}=138.8 Hz, PCH₂), 19.3 (d, ¹J_{C,P}=4.8 Hz, CH₂), 16.3 (d, ³J_{C,P}=5.5 Hz, 2OCH₂CH₃). ³¹P NMR (243 MHz, DMSO-d₆) δ [ppm]=32.8. LC-MS: positive mode [m/z]=613.3 [M+H]⁺. Purity: 88%.

### Synthesis of Diethyl (4-(8-(2-methylphenethyl)-2,6-dioxo-1-(prop-2-yn-1-yl)-1,2,6,7-tetrahydro-3H-purin-3-yl)butyl)phosphonate (42c

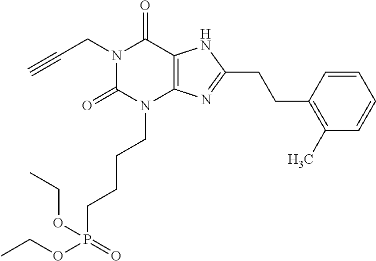 [SMILES: C#CCn1c(=O)c2[nH]c(CCc3ccccc3C)nc2n(CCCCP(=O)(OCC)OCC)c1=O]

Yield: 37%, off-white solid. ¹H NMR (600 MHz, DMSO-d₆) δ [ppm]=13.32 (s, 1H, N7-H), 7.16-7.06 (m, 4H, 4Hₐᵣₒₘ), 4.60 (d, J=2.5 Hz, 2H, N1-CH₂), 4.00 (t, J=7.0 Hz, 2H, N3-CH₂), 3.98-3.91 (m, 4H, 2OCH₂), 3.06 (t, J=2.4 Hz, 1H, H_{propargyl}), 3.00 (ddd, J=8.9, 6.5, 1.9 Hz, 2H, CH₂), 2.95 (ddd, J=8.7, 6.5, 1.9 Hz, 2H, CH₂), 2.29 (s, 3H, CH₃), 1.84-1.71 (m, 4H, 2CH₂), 1.56-1.41 (m, 2H, CH₂), 1.19 (t, J=7.0 Hz, 6H, 2OCH₂CH₃). ¹³C NMR (151 MHz, DMSO-d₆) δ [ppm]=154.1 (Cₓₐₙₜₕᵢₙₑ), 152.8 (Cₓₐₙₜₕᵢₙₑ), 150.0 (Caxntine), 148.1 (Cₓₐₙₜₕᵢₙₑ), 138.5 (Cₐᵣₒₘ), 135.7 (Cₐᵣₒₘ), 130.0 (Cₐᵣₒₘ), 128.5 (Cₐᵣₒₘ), 126.2 (Cₐᵣₒₘ), 125.9 (Cₐᵣₒₘ), 105.8 (C5), 79.7 (C_{propargyl}), 72.6 (C_{propargyl}), 60.7 (d, ²J_{C,P}=5.4 Hz, 2OCH₂), 42.4 (N3-CH₂), 30.8 (CH₂), 30.0 (N1-CH₂), 28.8 (CH₂), 28.2 (d, ³J_{C,P}=16.0 Hz, CH₂), 24.1 (d, ¹J_{C,P}=138.7 Hz, PCH₂), 19.2 (d, ²J_{C,P}=4.5 Hz, CH₂), 18.8 (CH₃), 16.2 (d, ³J_{C,P}=5.6 Hz, 2OCH₂CH₃). ³¹P NMR (243 MHz, DMSO-d₆) δ [ppm]=32.8. LC-MS: positive mode [m/z]=499.30 [M+H]⁺. Purity: 99%.

### Synthesis of Diethyl (4-(8-(2,6-dimethylphenethyl)-2,6-dioxo-1-(prop-2-yn-1-yl)-1,2,6,7-tetrahydro-3H-purin-3-yl)butyl)phosphonate (42l

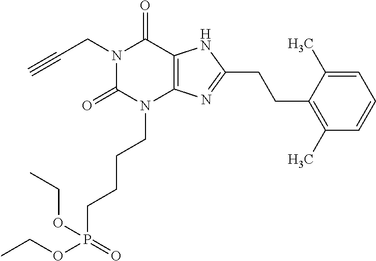 [SMILES: C#CCn1c(=O)c2[nH]c(CCc3c(C)cccc3C)nc2n(CCCCP(=O)(OCC)OCC)c1=O]

Compound 42l was used for the next step without further purification. LC-MS: positive mode [m/z]=515.1 [M+H]⁺. Off-white solid.

### Synthesis of Diethyl (4-(8-(2,6-dichlorophenethyl)-2,6-dioxo-1-(prop-2-yn-1-yl)-1,2,6,7-tetrahydro-3H-purin-3-yl)butyl)phosphonate (42m

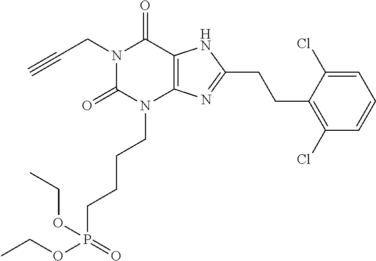 [SMILES: C#CCn1c(=O)c2[nH]c(CCc3c(Cl)cccc3Cl)nc2n(CCCCP(=O)(OCC)OCC)c1=O]

Compound 42m was used for the next step without further purification. ¹H NMR (600 MHz, DMSO-d6) δ [ppm]=13.42 (s, 1H, N7-H), 7.46 (d, J=8.0 Hz, 2H, 2Hₐᵣₒₘ), 7.29 (t, J=8.1 Hz, 1H, Hₐᵣₒₘ), 4.60 (d, J=2.5 Hz, 2H, N1-CH₂), 3.98-3.91 (m, 6H, N3-CH₂ and 2OCH₂), 3.28 (t, J=8.2 Hz, 2H, CH₂), 3.07 (t, J=2.5 Hz, 1H, H_{propargyl}), 2.96 (t, J=7.8 Hz, 2H, CH₂), 1.75 (ddt, J=18.0, 14.9, 7.6 Hz, 4H, 2CH₂), 1.45 (ddd, J=15.4, 9.8, 6.5 Hz, 2H, CH₂), 1.19 (t, J=7.1 Hz, 6H, 2OCH₂CH₃). ¹³C NMR (151 MHz, DMSO-d6) δ[ppm]=152.9 (Cₓₐₙₜₕᵢₙₑ), 152.9 (Cₓₐₙₜₕᵢₙₑ), 150.0 (Cₓₐₙₜₕᵢₙₑ), 148.2 (Cₓₐₙₜₕᵢₙₑ), 135.5 (Cₐᵣₒₘ), 134.7 (2Cₐᵣₒₘ), 129.1 (Cₐᵣₒₘ), 128.5 (2Cₐᵣₒₘ), 106.0 (C5), 79.7 (C_{propargyl}), 72.7 (C_{propargyl}), 60.7 (d, ²J_{C,P}=6.3 Hz, 2OCH₂), 42.3 (N3-CH₂), 30.1 (N1-CH₂), 29.3 (CH₂), 28.1 (d, ³J_{C,P}=15.6 Hz, CH₂), 26.6 (CH₂), 24.1 (d, ¹J_{C,P}=138.8 Hz, PCH₂), 19.2 (d, ²J_{C,P}=4.6 Hz, CH₂), 16.2 (d, ³J_{C,P}=5.7 Hz, 2OCH₂CH₃). ³¹P NMR (243 MHz, DMSO-d6) δ[ppm]=32.7. LC-MS: positive mode [m/z]=555.6 [M+H]⁺. Off-white solid.

### Synthesis of Diethyl (4-(8-(4-bromophenethyl)-2,6-dioxo-1-(prop-2-yn-1-yl)-1,2,6,7-tetrahydro-3H-purin-3-yl)butyl)phosphonate (42d

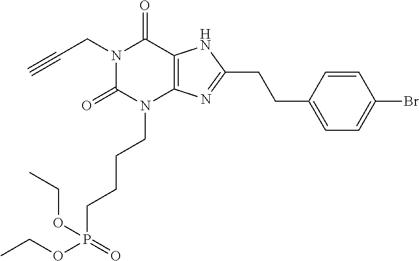 [SMILES: C#CCn1c(=O)c2[nH]c(CCc3ccc(Br)cc3)nc2n(CCCCP(=O)(OCC)OCC)c1=O]

Yield: 45%, off-white solid. ¹H NMR (600 MHz, DMSO-d₆) δ [ppm]=13.30 (s, 1H, N7-H), 7.48-7.43 (m, 2H, 2Hₐᵣₒₘ), 7.19-7.13 (m, 2H, 2Hₐᵣₒₘ), 4.59 (d, J=2.5 Hz, 2H, N1-CH₂), 3.99 (t, J=6.9 Hz, 2H, N3-CH₂), 3.97-3.89 (m, 4H, 2OCH₂), 3.06 (t, J=2.4 Hz, 1H, H_{propargyl}), 3.03-2.96 (m, 4H, 2CH₂), 1.82-1.71 (m, 4H, 2CH₂), 1.47 (dtd, J=12.9, 6.9, 6.4, 2.7 Hz, 2H, CH₂), 1.19 (t, J=7.0 Hz, 6H, 2OCH₂CH₃). ¹³C NMR (151 MHz, DMSO-d₆) δ [ppm]=153.7 (Cₓₐₙₜₕᵢₙₑ), 152.8 (Cₓₐₙₜₕᵢₙₑ), 150.0 (Cₓₐₙₜₕᵢₙₑ), 148.0 (Cₓₐₙₜₕᵢₙₑ), 139.8 (Cₐᵣₒₘ), 131.1 (2Cₐᵣₒₘ), 130.6 (2Cₐᵣₒₘ), 119.2 (Cₐᵣₒₘ), 105.9 (C5), 79.7 (C_{propargyl}), 72.7 (C_{propargyl}), 60.7 (d, ²J_{C,P}=5.4 Hz, 2OCH₂), 42.4 (N3-CH₂), 32.5 (CH₂), 30.0 (N1-CH₂), 29.7 (CH₂), 28.2 (d, ³J_{C,P}=15.8 Hz, CH₂), 24.1 (d, ¹J_{C,P}=138.6 Hz, PCH₂), 19.3 (d, ²J_{C,P}=4.8 Hz, CH₂), 16.2 (d, ³J_{C,P}=5.6 Hz, 2OCH₂CH₃). ³¹P NMR (243 MHz, DMSO-d₆) δ [ppm]=32.8. LC-MS: positive mode [m/z]=565.2 [M+H]⁺. Purity: 96%.

### Synthesis of Diethyl (4-(8-(4-iodophenethyl)-2,6-dioxo-1-(prop-2-yn-1-yl)-1,2,6,7-tetrahydro-3H-purin-3-yl)butyl)phosphonate (42e

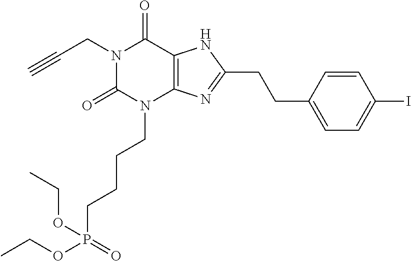 [SMILES: C#CCn1c(=O)c2[nH]c(CCc3ccc(I)cc3)nc2n(CCCCP(=O)(OCC)OCC)c1=O]

Yield: 66%, off-white solid. H NMR (600 MHz, DMSO-d₆) δ [ppm]=13.30 (s, 1H, N7-H), 7.64-7.58 (m, 2H, 2Hₐᵣₒₘ), 7.04-7.00 (m, 2H, 2Hₐᵣₒₘ), 4.59 (d, J=2.5 Hz, 2H, N1-CH₂), 3.99 (t, J=6.8 Hz, 2H, N3-CH₂), 3.97-3.91 (m, 4H, 2OCH₂), 3.06 (t, J=2.4 Hz, 1H, H_{propargyl}), 2.98 (s, 4H, 2CH₂), 1.77 (tdd, J=14.6, 10.3, 7.1 Hz, 4H, 2CH₂), 1.52-1.43 (m, 2H, CH₂), 1.19 (t, J=7.0 Hz, 6H, 2OCH₂CH₃). ¹³C NMR (151 MHz, DMSO-d₆) δ [ppm]=153.7 (Cₓₐₙₜₕᵢₙₑ), 152.8 (Cxantimne), 150.0 (Cₓₐₙₜₕᵢₙₑ), 148.0 (Cₓₐₙₜₕᵢₙₑ), 140.2 (Cₐᵣₒₘ), 137.0 (2Cₐᵣₒₘ), 130.8 (2Cₐᵣₒₘ), 105.9 (C5), 91.8 (Cₐᵣₒₘ), 79.7 (C_{propargyl}), 72.7 (C_{propargyl}), 60.7 (d, ²J_{C,P}=6.3 Hz, OCH₂), 42.4 (N3-CH₂), 32.6 (CH₂), 30.0 (N1-CH₂), 29.7 (CH₂), 28.2 (d, J_{C,P}=15.7 Hz, CH₂), 24.1 (d, ¹J_{C,P}=138.7 Hz, PCH₂), 19.3 (d, ²J_{C,P}=4.7 Hz, CH₂), 16.2 (d, ³J_{C,P}=5.7 Hz, OCH₂CH₃). ³¹P NMR (243 MHz, DMSO-d₆) δ [ppm]=32.8. LC-MS: positive mode [m/z]=613.20 [M+H]⁺. Purity: 99%.

### Synthesis of Diethyl (4-(2,6-dioxo-1-(prop-2-yn-1-yl)-8-(4-(trifluoromethyl)phenethyl)-1,2,6,7-tetra-hydro-3H-purin-3-yl)butyl)phosphonate (42f

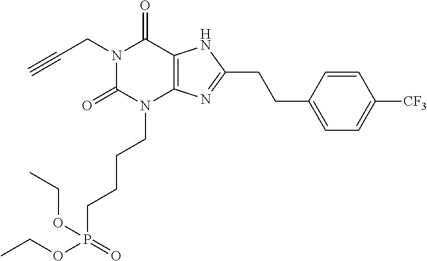 [SMILES: C#CCn1c(=O)c2[nH]c(CCc3ccc(C(F)(F)F)cc3)nc2n(CCCCP(=O)(OCC)OCC)c1=O]

Yield: 58%, off-white solid. ¹H NMR (600 MHz, DMSO-d₆) δ [ppm]=13.33 (s, 1H, N7-H), 7.64 (d, J=8.0 Hz, 2H, 2Hₐᵣₒₘ), 7.43 (d, J=8.0 Hz, 2H, 2Hₐᵣₒₘ), 4.59 (d, J=2.5 Hz, 2H, N1-CH₂), 3.99 (t, J=7.0 Hz, 2H, N3-CH₂), 3.97-3.91 (m, 4H, 2OCH₂), 3.13 (t, J=7.6 Hz, 2H, CH₂), 3.06 (t, J=2.5 Hz, 1H, H_{propargyl}), 3.04 (t, J=7.4 Hz, 2H, CH₂), 1.81-1.73 (m, 4H, 2CH₂), 1.51-1.41 (m, 2H, CH₂), 1.19 (t, J=7.0 Hz, 6H, 2OCH₂CH₃). ¹³C NMR (151 MHz, DMSO-d₆) δ [ppm]=153.6 (Cₓₐₙₜₕᵢₙₑ), 152.9 (Cₓₐₙₜₕᵢₙₑ), 150.0 (Cₓₐₙₜₕᵢₙₑ), 148.0 (Cₓₐₙₜₕᵢₙₑ), 145.3 (Cₐᵣₒₘ), 129.2 (2Cₐᵣₒₘ), 126.9 (q, 2J_{C,F}=31.8 Hz, Cₐᵣₒₘ), 125.1 (q, ³J_{C,F}=3.7 Hz, 2Cₐᵣₒₘ), 124.4 (q, ¹J_{C,F}=271.7 Hz, CF₃), 105.9 (C5), 79.7 (C_{propargyl}), 72.7 (C_{propargyl}), 60.7 (d, ²J_{C,P}=6.3 Hz, OCH₂), 42.4 (N3-CH₂), 32.9 (CH₂), 30.0 (N1-CH₂), 29.5 (CH₂), 28.2 (d, ³J_{C,P}=15.8 Hz, CH₂), 24.1 (d, ¹J_{C,P}=138.6 Hz, PCH₂), 19.3 (d, ²J_{C,P}=4.8 Hz, CH₂), 16.2 (d, ³J_{C,P}=5.7 Hz, OCH₂CH₃). ³¹P NMR (243 MHz, DMSO-d₆) δ [ppm]=32.8. LC-MS: positive mode [m/z]=555.30 [M+H]⁺. Purity: 99%.

### Synthesis of Diethyl (4-(8-(4-methoxyphenethyl)-2,6-dioxo-1-(prop-2-yn-1-yl)-1,2,6,7-tetrahydro-3H-purin-3-yl)butyl)phosphonate (42g

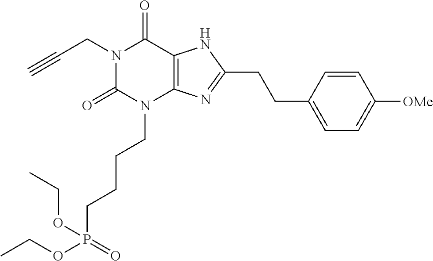 [SMILES: C#CCn1c(=O)c2[nH]c(CCc3ccc(OC)cc3)nc2n(CCCCP(=O)(OCC)OCC)c1=O]

Yield: 40%, off-white solid. ¹H NMR (500 MHz, DMSO-d₆) δ [ppm]=13.27 (s, 1H, N7-H), 7.12-7.08 (m, 2H, 2Hₐᵣₒₘ), 6.85-6.81 (m, 2H, 2Hₐᵣₒₘ), 4.59 (d, J=2.5 Hz, 2H, N1-CH₂), 4.00 (t, J=7.0 Hz, 2H, N3-CH₂), 3.98-3.91 (m, 4H, 2OCH₂), 3.70 (s, 3H, OCH₃), 3.05 (t, J=2.4 Hz, 1H, H_{propargyl}), 2.96 (s, 4H, 2CH₂), 1.82-1.73 (m, 4H, 2CH₂), 1.48 (dtt, J=12.7, 9.5, 6.2 Hz, 2H, CH₂), 1.19 (t, J=7.0 Hz, 6H, 2OCH₂CH₃). ¹³C NMR (126 MHz, DMSO-d₆) δ [ppm]=157.6 (Cₐᵣₒₘ), 154.1 (Cₓₐₙₜₕᵢₙₑ), 152.8 (Cₓₐₙₜₕᵢₙₑ), 150.0 (Cₓₐₙₜₕᵢₙₑ), 148.1 (Cₓₐₙₜₕᵢₙₑ), 132.3 (Cₐᵣₒₘ), 129.2 (2Cₐᵣₒₘ), 113.7 (2Cₐᵣₒₘ), 105.8 (C5), 79.7 (C_{propargyl}), 72.6 (C_{propargyl}), 60.7 (d, ²J_{C,P}=6.3 Hz, OCH₂), 54.9 (OCH₃), 42.4 (N3-CH₂), 32.4 (CH₂), 30.3 (N1-CH₂), 30.0 (CH₂), 28.2 (d, ³J_{C,P}=15.7 Hz, CH₂), 24.1 (d, ¹J_{C,P}=138.7 Hz, PCH₂), 19.3 (d, ²J_{C,P}=4.8 Hz, CH₂), 16.2 (d, ³J_{C,P}=5.7 Hz, OCH₂CH₃). ³¹P NMR (202 MHz, DMSO-d₆) δ [ppm]=32.8. LC-MS: positive mode [m/z]=517.30 [M+H]⁺. Purity: 99%.

### Synthesis of Diethyl (4-(8-(4-ethylphenethyl)-2,6-dioxo-1-(prop-2-yn-1-yl)-1,2,6,7-tetrahydro-3H-purin-3-yl)butyl)phosphonate (42i

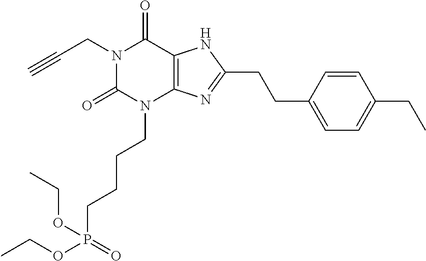 [SMILES: C#CCn1c(=O)c2[nH]c(CCc3ccc(CC)cc3)nc2n(CCCCP(=O)(OCC)OCC)c1=O]

Yield: 47%, off-white solid. ¹H NMR (500 MHz, DMSO-d₆) δ [ppm]=13.28 (s, 1H, N7-H), 7.10 (s, 4H, 4Hₐᵣₒₘ), 4.59 (d, J=2.5 Hz, 2H, N1-CH₂), 4.00 (t, J=7.0 Hz, 2H, N3-CH₂), 3.98-3.91 (m, 4H, 2OCH₂), 3.05 (t, J=2.4 Hz, 1H, H_{propargyl}), 2.98 (s, 4H, 2CH₂), 2.55 (q, J=7.6 Hz, 2H, CH₂CH₃), 1.78 (ddt, J=12.7, 8.4, 6.1 Hz, 4H, 2CH₂), 1.48 (dtd, J=11.8, 6.3, 5.2, 3.1 Hz, 2H, CH₂), 1.19 (t, J=7.0 Hz, 6H, OCH₂CH₃), 1.14 (t, J=7.6 Hz, 3H, CH₂CH₃). ¹³C NMR (126 MHz, DMSO-d₆) δ [ppm]=154.1 (Cₓₐₙₜₕᵢₙₑ), 152.8 (Cₓₐₙₜₕᵢₙₑ), 150.0 (Cₓₐₙₜₕᵢₙₑ), 148.1 (Cₓₐₙₜₕᵢₙₑ), 141.4 (Cₐᵣₒₘ), 137.6 (Cₐᵣₒₘ), 128.1 (2Cₐᵣₒₘ), 127.7 (2Cₐᵣₒₘ), 105.8 (C5), 79.7 (Cₓₐₙₜₕᵢₙₑ), 72.6 (Cₓₐₙₜₕᵢₙₑ), 60.7 (d, ²J_{C,P}=6.3 Hz, OCH₂), 42.4 (N3-CH₂), 32.8 (CH₂), 30.1 (N1-CH₂), 30.0 (CH₂), 28.2 (d, ³J_{C,P}=15.7 Hz, CH₂), 27.7 (CH₂CH₃), 24.1 (d, ¹J_{C,P}=138.7 Hz, PCH₂), 19.3 (d, ²J_{C,P}=4.8 Hz, CH₂), 16.2 (d, ³J_{C,P}=5.6 Hz, OCH₂CH₃), 15.5 (CH₂CH₃). ³¹P NMR (202 MHz, DMSO-d₆) δ [ppm]=32.8. LC-MS: positive mode [m/z]=515.3 [M+H]⁺. Purity: 97%.

### Synthesis of Diethyl (4-(8-(4-isopropylphenethyl)-2,6-dioxo-1-(prop-2-yn-1-yl)-1,2,6,7-tetrahydro-3H-purin-3-yl)butyl)phosphonate (42j

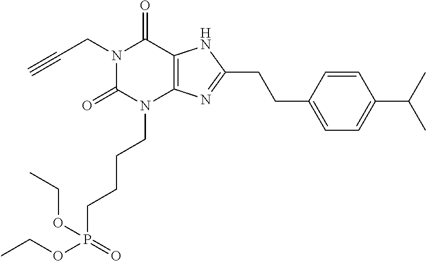 [SMILES: C#CCn1c(=O)c2[nH]c(CCc3ccc(C(C)C)cc3)nc2n(CCCCP(=O)(OCC)OCC)c1=O]

Yield: 66%, off-white solid. ¹H NMR (500 MHz, DMSO-d₆) δ [ppm]=13.28 (s, 1H, N7-H), 7.16-7.10 (m, 4H, 4Hₐᵣₒₘ), 4.60 (d, J=2.5 Hz, 2H, N1-CH₂), 4.00 (t, J=6.9 Hz, 2H, N3-CH₂), 3.98-3.90 (m, 4H, 2OCH₂), 3.05 (t, J=2.4 Hz, 1H, H_{propargyl}), 2.98 (s, 4H, 2CH₂), 2.83 (p, J=6.9 Hz, 1H, CH(CH₃)₂), 1.78 (ddt, J=13.4, 8.8, 6.4 Hz, 4H, 2CH₂), 1.54-1.42 (m, 2H, CH₂), 1.22-1.15 (m, 12H, 2OCH₂CH₃ and CH(CH₃)₂). ¹³C NMR (126 MHz, DMSO-d₆) δ [ppm]=154.1 (Cₓₐₙₜₕᵢₙₑ), 152.8 (Cₓₐₙₜₕᵢₙₑ), 150.0 (Cₓₐₙₜₕᵢₙₑ), 148.1 (Cₓₐₙₜₕᵢₙₑ), 146.1 (Cₐᵣₒₘ), 137.7 (Cₐᵣₒₘ), 128.1 (2Cₐᵣₒₘ), 126.2 (2Cₐᵣₒₘ), 105.8 (C5), 79.7 (C_{propargyl}), 72.6 (C_{propargyl}), 60.7 (d, ²J_{C,P}=6.2 Hz, OCH₂), 42.4 (N3-CH₂), 33.0 (CH(CH₃)₂), 32.8 (CH₂), 30.1 (CH₂), 30.0 (N1-CH₂), 28.2 (d, ³J_{C,P}=15.7 Hz, CH₂), 24.1 (d, ¹J_{C,P}=138.8 Hz, PCH₂), 23.9 (CH(CH₃)₂), 19.3 (d, ²J_{C,P}=4.9 Hz, CH₂), 16.2 (d, ³J_{C,P}=5.6 Hz, OCH₂CH₃). ³¹P NMR (202 MHz, DMSO-d₆) δ [ppm]=32.7. LC-MS: positive mode [m/z]=529.4 [M+H]⁺. Purity: 95%.

### Synthesis of Diethyl (4-(8-(2-([1,1′-biphenyl]-4-yl)ethyl)-2,6-dioxo-1-(prop-2-yn-1-yl)-1,2,6,7-tetra-hydro-3H-purin-3-yl)butyl)phosphonate (42k

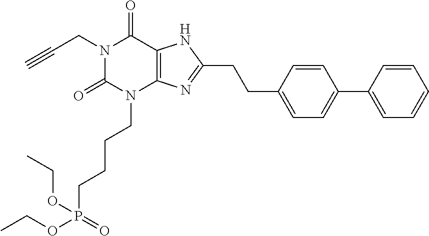 [SMILES: C#CCn1c(=O)c2[nH]c(CCc3ccc(-c4ccccc4)cc3)nc2n(CCCCP(=O)(OCC)OCC)c1=O]

Yield: 52%, off-white solid. ¹H NMR (600 MHz, DMSO-d₆) δ [ppm]=13.33 (s, 1H, N7-H), 7.63 (dd, J=7.9, 1.4 Hz, 2H, 2Hₐᵣₒₘ), 7.59-7.56 (m, 2H, 2Hₐᵣₒₘ), 7.44 (t, J=7.7 Hz, 2H, 2Hₐᵣₒₘ), 7.34 (td, J=7.3, 1.3 Hz, 1H, Hₐᵣₒₘ), 7.32-7.27 (m, 2H, 2Hₐᵣₒₘ), 4.60 (d, J=2.5 Hz, 2H, N1-CH₂), 4.01 (t, J=6.9 Hz, 2H, N3-CH₂), 3.97-3.90 (m, 4H, 2OCH₂), 3.06 (dt, J=11.2, 7.0 Hz, 5H, 2CH₂ and H_{propargyl}), 1.83-1.71 (m, 4H, 2CH₂), 1.48 (dddd, J=11.9, 8.3, 6.2, 2.5 Hz, 2H, CH₂), 1.18 (t, J=7.0 Hz, 6H, 2OCH₂CH₃). ¹³C NMR (151 MHz, DMSO-d₆) δ [ppm]=154.0 (Cₓₐₙₜₕᵢₙₑ), 152.9 (Cₓₐₙₜₕᵢₙₑ), 150.0 (Cₓₐₙₜₕᵢₙₑ), 148.1 (Cₓₐₙₜₕᵢₙₑ), 139.9 (Cₐᵣₒₘ), 139.7 (Cₐᵣₒₘ), 138.0 (Cₐᵣₒₘ), 128.9 (Cₐᵣₒₘ), 127.2 (4Cₐᵣₒₘ), 126.6 (2Cₐᵣₒₘ), 126.4 (2Cₐᵣₒₘ), 105.9 (C5), 79.7 (C_{propargyl}), 72.7 (C_{propargyl}), 60.7 (d, ²J_{C,P}=5.4 Hz, OCH₂), 42.4 (N3-CH₂), 32.8 (CH₂), 30.0 (N1-CH₂), 29.9 (CH₂), 28.2 (d, ³J_{C,P}=15.6 Hz, CH₂), 24.1 (d, ¹J_{C,P}=138.6 Hz, PCH₂), 19.3 (d, ²J_{C,P}=4.8 Hz, CH₂), 16.2 (d, ³J_{C,P}=5.5 Hz, OCH₂CH₃). ³¹P NMR (243 MHz, DMSO-d₆) δ [ppm]=32.8. LC-MS: positive mode [m/z]=563.3 [M+H]⁺. Purity: 93%.

### Synthesis of Diethyl (4-(8-(naphthalen-1-ylmethyl)-2,6-dioxo-1-(prop-2-yn-1-yl)-1,2,6,7-tetrahydro-3H-purin-3-yl)butyl)phosphonate (77

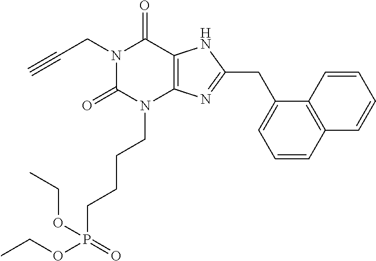 [SMILES: C#CCn1c(=O)c2[nH]c(Cc3cccc4ccccc34)nc2n(CCCCP(=O)(OCC)OCC)c1=O]

Yield: 53%, off-white solid. ¹H NMR (600 MHz, DMSO-d6) δ[ppm]=13.58 (s, 1H, N7-H), 7.89-7.85 (m, 3H, 3Hₐᵣₒₘ), 7.79-7.76 (m, 1H, Hₐᵣₒₘ), 7.51-7.43 (m, 3H, 3Hₐᵣₒₘ), 4.59 (d, J=2.5 Hz, 2H, N1-CH₂), 4.24 (s, 2H, CH₂), 3.98 (t, J=7.0 Hz, 2H, N3-CH₂), 3.95-3.88 (m, 4H, 2OCH₂), 3.06 (t, J=2.4 Hz, 1H, H_{propargyl}), 1.80-1.70 (m, 4H, 2CH₂), 1.51-1.41 (m, 2H, CH₂), 1.16 (t, J=7.0 Hz, 6H, 2OCH₂CH₃). ¹³C NMR (151 MHz, DMSO-d6) δ[ppm]=153.0 (Cₓₐₙₜₕᵢₙₑ), 152.9 (Cₓₐₙₜₕᵢₙₑ), 150.0 (Cₓₐₙₜₕᵢₙₑ), 148.3 (Cₓₐₙₜₕᵢₙₑ), 134.4 (Cₐᵣₒₘ), 133.0 (Cₐᵣₒₘ), 131.8 (Cₐᵣₒₘ), 128.1 (Cₐᵣₒₘ), 127.5 (2Cₐᵣₒₘ), 127.0 (Cₐᵣₒₘ), 126.9 (Cₐᵣₒₘ), 126.2 (Cₐᵣₒₘ), 125.7 (Cₐᵣₒₘ), 106.3 (C5), 79.7 (C_{propargyl}), 72.6 (C_{propargyl}), 60.7 (d, ²J_{C,P}=6.2 Hz, 2OCH₂), 42.5 (N3-CH₂), 34.5 (C8-CH₂), 30.1 (N1-CH₂), 28.2 (d, ³J_{C,P}=15.5 Hz, CH₂), 24.1 (d, ¹J_{C,P}=138.5 Hz, PCH₂), 19.3 (d, ²J_{C,P}=4.9 Hz, CH₂), 16.2 (d, ³J_{C,P}=5.7 Hz, 2OCH₂CH₃). ³¹P NMR (243 MHz, DMSO-d6) δ[ppm]=32.7. LC-MS: positive mode [m/z]=523.6 [M+H]⁺. Purity: 83%.

### Synthesis of Diethyl (4-(8-(2-fluorophenethyl)-7-methyl-2,6-dioxo-1-(prop-2-yn-1-yl)-1,2,6,7-tetrahydro-3H-purin-3-yl)butyl)phosphonate (48a

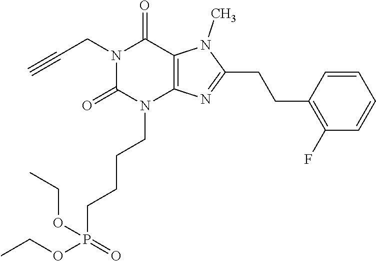 [SMILES: C#CCn1c(=O)c2c(nc(CCc3ccccc3F)n2C)n(CCCCP(=O)(OCC)OCC)c1=O]

Compound 48a was used for the next step without further purification. ¹H NMR (600 MHz, DMSO-d₆) δ [ppm]=7.33 (td, J=7.7, 1.8 Hz, 1H, Hₐᵣₒₘ), 7.26 (ddd, J=9.7, 4.7, 2.0 Hz, 1H, Hₐᵣₒₘ), 7.18-7.09 (m, 2H, 2Hₐᵣₒₘ), 4.58 (d, J=2.4 Hz, 2H, N1-CH₂), 4.00-3.90 (m, 6H, 2OCH₂ and N3-CH₂), 3.76 (s, 3H, N7-CH₃), 3.07 (t, J=2.5 Hz, 1H, H_{propargyl}), 3.05 (bs, 4H, 2CH₂), 1.82-1.68 (m, 4H, 2CH₂), 1.51-1.41 (m, 2H, CH₂), 1.19 (t, J=7.0 Hz, 6H, 2OCH₂CH₃). ¹³C NMR (151 MHz, DMSO-d₆) δ [ppm]=160.6 (d, ¹J_{C,F}=243.4 Hz, Cₐᵣₒₘ), 153.7 (Cₓₐₙₜₕᵢₙₑ), 153.3 (Cₓₐₙₜₕᵢₙₑ), 149.8 (Cₓₐₙₜₕᵢₙₑ), 147.3 (Cₓₐₙₜₕᵢₙₑ), 131.0 (d, J=4.4 Hz, Cₐᵣₒₘ), 128.4 (d, J=8.0 Hz, Cₐᵣₒₘ), 127.0 (d, J=15.8 Hz, Cₐᵣₒₘ), 124.4 (d, J=3.4 Hz, Cₐᵣₒₘ), 115.1 (d, J=21.9 Hz, Cₐᵣₒₘ), 106.3 (C5), 79.6 (C_{propargyl}), 72.7 (C_{propargyl}), 60.7 (d, ²J_{C,P}=6.3 Hz, 2OCH₂), 42.1 (N3-CH₂), 31.3 (CH₂), 29.9 (CH₂), 28.1 (d, ³J_{C,P}=15.8 Hz, CH₂), 26.4 (N7-CH₃), 25.8 (d, ³J_{C,F}=1.5 Hz, CH₂), 24.1 (d, ¹J_{C,P}=138.7 Hz, PCH₂), 19.2 (d, ²J_{C,P}=4.4 Hz, CH₂), 16.2 (d, ³J_{C,P}=5.5 Hz, 2OCH₂CH₃). ³¹P NMR (243 MHz, DMSO-d₆) δ [ppm]=32.8. LC-MS: positive mode [m/z]=519.1 [M+H]⁺. Purity: 94%; light-yellow solid.

### Synthesis of Diethyl (5-(8-(2-bromophenethyl)-7-methyl-2,6-dioxo-1-(prop-2-yn-1-yl)-1,2,6,7-tetra-hydro-3H-purin-3-yl)pentyl)phosphonate (57h

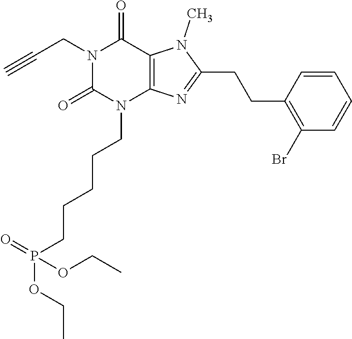 [SMILES: C#CCn1c(=O)c2c(nc(CCc3ccccc3Br)n2C)n(CCCCCP(=O)(OCC)OCC)c1=O]

Compound 57h was used for the next step without further purification. ¹H NMR (600 MHz, DMSO-d₆) δ [ppm]=7.59 (dd, J=7.9, 1.2 Hz, 1H, Hₐᵣₒₘ), 7.35 (dd, J=7.7, 1.8 Hz, 1H, Hₐᵣₒₘ), 7.31 (td, J=7.4, 1.2 Hz, 1H, Hₐᵣₒₘ), 7.18 (dd, J=7.6, 1.8 Hz, 1H, Hₐᵣₒₘ), 4.58 (d, J=2.4 Hz, 2H, N1-CH₂), 4.03-3.89 (m, 6H, 2OCH₂ and N3-CH₂), 3.75 (s, 3H, N7-CH₃), 3.16-3.10 (m, 2H, CH₂), 3.10-3.02 (m, 3H, H_{propargyl} and CH₂), 1.75-1.60 (m, 4H, 2CH₂), 1.55-1.46 (m, 2H, CH₂), 1.35 (dd, J=10.4, 4.9 Hz, 2H, CH₂), 1.20 (t, J=7.0 Hz, 6H, 2OCH₂CH₃). ¹³C NMR (151 MHz, DMSO-d₆) δ [ppm]=153.5 (Cₓₐₙₜₕᵢₙₑ), 153.3 (Cₓₐₙₜₕᵢₙₑ), 149.8 (Cₓₐₙₜₕᵢₙₑ), 147.4 (Cₓₐₙₜₕᵢₙₑ), 139.3 (Cₐᵣₒₘ), 132.5 (Cₐᵣₒₘ), 131.0 (Cₐᵣₒₘ), 128.6 (Cₐᵣₒₘ), 127.9 (Cₐᵣₒₘ), 123.7 (Cₐᵣₒₘ), 106.4 (C5), 79.7 (C_{propargyl}), 72.7 (C_{propargyl}), 60.7 (d, ²J_{C,P}=6.3 Hz, 2OCH₂), 42.4 (N3-CH₂), 32.9 (CH₂), 31.3 (CH₂), 29.9 (CH₂), 26.8 (N7-CH₃), 26.6 (d, ³J_{C,P}=16.3 Hz, CH₂), 26.2 (CH₂), 24.3 (d, ¹J_{C,P}=138.6 Hz, PCH₂), 21.7 (d, ²J_{C,P}=5.0 Hz, CH₂), 16.3 (d, ³J_{C,P}=5.5 Hz, 2OCH₂CH₃). ³¹P NMR (243 MHz, DMSO-d₆) δ [ppm]=33.0. LC-MS: positive mode [m/z]=593.0 [M+H]⁺. Purity: 96%; light-yellow solid.

### Synthesis of Diethyl (6-(8-(2-bromophenethyl)-7-methyl-2,6-dioxo-1-(prop-2-yn-1-yl)-1,2,6,7-tetra-hydro-3H-purin-3-yl)hexyl)phosphonate (59h

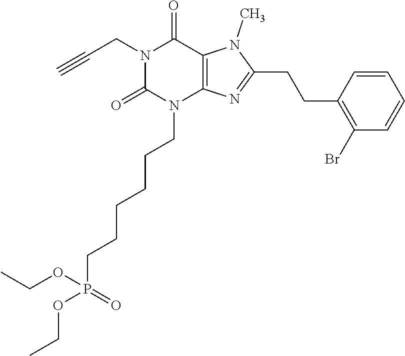 [SMILES: C#CCn1c(=O)c2c(nc(CCc3ccccc3Br)n2C)n(CCCCCCP(=O)(OCC)OCC)c1=O]

Yield: 54%, light-yellow solid. H NMR (600 MHz, DMSO-d₆) δ [ppm]=7.59 (dd, J=8.0, 1.2 Hz, 1H, Hₐᵣₒₘ), 7.35 (dd, J=7.7, 1.8 Hz, 1H, Hₐᵣₒₘ), 7.30 (td, J=7.5, 1.3 Hz, 1H, Hₐᵣₒₘ), 7.17 (td, J=7.6, 1.8 Hz, 1H, Hₐᵣₒₘ), 4.58 (d, J=2.4 Hz, 2H, N1-CH₂), 3.99-3.90 (m, 6H, 2OCH₂ and N3-CH₂), 3.75 (s, 3H, N7-CH₃), 3.13 (t, J=7.0 Hz, 2H, CH₂), 3.07 (t, J=7.0 Hz, 2H, CH₂), 3.06 (t, J=2.5 Hz, 1H, H_{propargyl}), 1.72-1.57 (m, 4H, 2CH₂), 1.45 (ddt, J=15.4, 11.6, 7.2 Hz, 2H, CH₂), 1.37 (dq, J=14.8, 8.0, 7.5 Hz, 2H, CH₂), 1.31-1.22 (m, 2H, CH₂), 1.20 (t, J=7.1 Hz, 6H, 2OCH₂CH₃). ¹³C NMR (151 MHz, DMSO-d₆) δ [ppm]=153.5 (Cₓₐₙₜₕᵢₙₑ), 153.3 (Cₓₐₙₜₕᵢₙₑ), 149.7 (Cₓₐₙₜₕᵢₙₑ), 147.4 (Cₓₐₙₜₕᵢₙₑ), 139.3 (Cₐᵣₒₘ), 132.5 (Cₐᵣₒₘ), 130.9 (Cₐᵣₒₘ), 128.6 (Cₐᵣₒₘ), 127.9 (Cₐᵣₒₘ), 123.7 (Cₐᵣₒₘ), 106.4 (C5), 79.7 (C_{propargyl}), 72.6 (C_{propargyl}), 60.7 (d, ²J_{C,P}=6.3 Hz, 2OCH₂), 42.5 (N3-CH₂), 32.9 (CH₂), 31.3 (CH₂), 29.8 (CH₂), 29.3 (d, ³J_{C,P}=15.9 Hz, CH₂), 27.1 (CH₂), 26.2 (N7-CH₃), 25.5 (CH₂), 24.4 (d, ¹J_{C,P}=138.5 Hz, PCH₂), 21.9 (d, ²J_{C,P}=4.4 Hz, CH₂), 16.3 (d, ³J_{C,P}=5.7 Hz, 2OCH₂CH₃). ³¹P NMR (243 MHz, DMSO-d₆) δ [ppm]=33.0. LC-MS: positive mode [m/z]=607.4 [M+H]⁺. Purity: 93%.

### Synthesis of Diethyl (4-(8-(4-bromophenethyl)-7-methyl-2,6-dioxo-1-(prop-2-yn-1-yl)-1,2,6,7-tetra-hydro-3H-purin-3-yl)butyl)phosphonate (48d

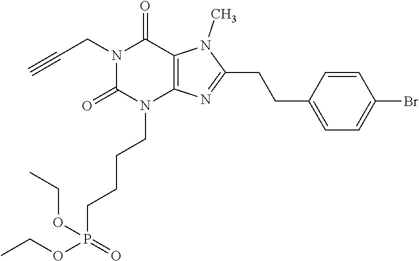 [SMILES: C#CCn1c(=O)c2c(nc(CCc3ccc(Br)cc3)n2C)n(CCCCP(=O)(OCC)OCC)c1=O]

Compound 48d was used for the next step without further purification. ¹H NMR (600 MHz, DMSO-d₆) δ [ppm]=7.49-7.44 (m, 2H, 2Hₐᵣₒₘ), 7.24-7.20 (m, 2H, 2Hₐᵣₒₘ), 4.58 (d, J=2.5 Hz, 2H, N1-CH₂), 3.98 (t, J=6.8 Hz, 2H, N3-CH₂), 3.98-3.89 (m, 4H, 2OCH₂), 3.76 (s, 3H, N7-CH₃), 3.06 (t, J=2.5 Hz, 1H, H_{propargyl}), 3.06-3.03 (m, 2H, CH₂), 3.02-2.97 (m, 2H, CH₂), 1.83-1.67 (m, 4H, 2CH₂), 1.46 (dtd, J=12.8, 6.8, 6.2, 2.9 Hz, 2H, CH₂), 1.19 (t, J=7.0 Hz, 6H, 2OCH₂CH₃). ¹³C NMR (151 MHz, DMSO-d₆) δ [ppm]=153.8 (Cₓₐₙₜₕᵢₙₑ), 153.3 (Cₓₐₙₜₕᵢₙₑ), 149.8 (Cₓₐₙₜₕᵢₙₑ), 147.3 (Cₓₐₙₜₕᵢₙₑ), 139.9 (Cₐᵣₒₘ), 131.1 (2Cₐᵣₒₘ), 130.8 (2Cₐᵣₒₘ), 119.2 (Cₐᵣₒₘ), 106.3 (C5), 79.6 (C_{propargyl}), 72.7 (C_{propargyl}), 60.8 (d, ²J_{C,P}=6.3 Hz, 2OCH₂), 42.1 (N3-CH₂), 31.7 (CH₂), 31.3 (CH₂), 29.9 (CH₂), 28.1 (d, ³J_{C,P}=16.0 Hz, CH₂), 27.4 (N7-CH₃), 24.1 (d, ¹J_{C,P}=138.8 Hz, PCH₂), 19.3 (d, ²J_{C,P}=4.8 Hz, CH₂), 16.2 (d, ³J_{C,P}=5.6 Hz, 2OCH₂CH₃). ³¹P NMR (243 MHz, DMSO-d₆) δ [ppm]=32.8. LC-MS: positive mode [m/z]=579.1 [M+H]⁺. Purity: 95%, light-yellow solid.

### Synthesis of Diethyl (4-(7-ethyl-8-(2-fluorophenethyl)-2,6-dioxo-1-(prop-2-yn-1-yl)-1,2,6,7-tetrahydro-3H-purin-3-yl)butyl)phosphonate (49a

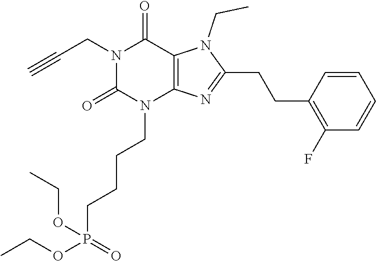 [SMILES: C#CCn1c(=O)c2c(nc(CCc3ccccc3F)n2CC)n(CCCCP(=O)(OCC)OCC)c1=O]

Compound 49a was used for the next step without further purification. H NMR (600 MHz, DMSO-d₆) δ [ppm]=7.35 (td, J=7.7, 1.8 Hz, 1H, Hₐᵣₒₘ), 7.26 (tdd, J=7.5, 5.4, 1.8 Hz, 1H, Hₐᵣₒₘ), 7.17-7.09 (m, 2H, 2Hₐᵣₒₘ), 4.59 (d, J=2.4 Hz, 2H, N1-CH₂), 4.21 (q, J=7.2 Hz, 2H, N7-CH₂), 4.01-3.89 (m, 6H, 2OCH₂ and N3-CH₂), 3.10-3.04 (m, 5H, 2CH₂ and H_{propargyl}), 1.84-1.71 (m, 4H, 2CH₂), 1.47 (ddt, J=18.8, 15.0, 7.8 Hz, 2H, CH₂), 1.23 (t, J=7.2 Hz, 3H, N7-CH₂CH₃), 1.19 (t, J=7.0 Hz, 6H, 2OCH₂CH₃). ¹³C NMR (151 MHz, DMSO-d₆) δ [ppm]=160.6 (d, ¹J_{C,F}=243.5 Hz, Cₐᵣₒₘ), 152.9 (Cₓₐₙₜₕᵢₙₑ), 152.9 (Cₓₐₙₜₕᵢₙₑ), 149.8 (Cₓₐₙₜₕᵢₙₑ), 147.7 (Cₓₐₙₜₕᵢₙₑ), 131.0 (d, J=4.7 Hz, Cₐᵣₒₘ), 128.4 (d, J=8.1 Hz, Cₐᵣₒₘ), 126.9 (d, ²J_{,F}=15.7 Hz, Cₐᵣₒₘ), 124.4 (d, J=3.3 Hz, Cₐᵣₒₘ), 115.1 (d, J=21.8 Hz, Cₐᵣₒₘ), 105.5 (C5), 79.6 (C_{propargyl}), 72.7 (C_{propargyl}), 60.7 (d, ²J_{C,P}=6.2 Hz, 2OCH₂), 42.1 (N3-CH₂), 40.1 (N7-CH₂), 35.7 (CH₂), 30.7 (CH₂), 29.9 (N1-CH₂), 28.1 (d, ³J_{C,P}=15.9 Hz, CH₂), 26.2 (d, ²J_{C,P}=5.9 Hz, CH₂), 24.1 (d, ¹J_{C,P}=138.7 Hz, PCH₂), 19.3 (d, ³J_{C,P}=5.0 Hz, 2OCH₂CH₃), 15.8 (N7-CH₂CH₃). ³¹P NMR (243 MHz, DMSO-d₆) δ [ppm]=32.7. LC-MS: positive mode [m/z]=533.2 [M+H]⁺. Purity: 80%; light-yellow solid.

### Synthesis of Diethyl (5-(8-(2-bromophenethyl)-7-ethyl-2,6-dioxo-1-(prop-2-yn-1-yl)-1,2,6,7-tetrahydro-3H-purin-3-yl)pentyl)phosphonate (58h

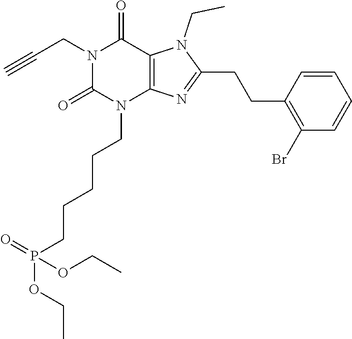 [SMILES: C#CCn1c(=O)c2c(nc(CCc3ccccc3Br)n2CC)n(CCCCCP(=O)(OCC)OCC)c1=O]

Compound 58h was used for the next step without further purification. ¹H NMR (600 MHz, DMSO-d₆) δ [ppm]=7.60 (dd, J=8.0, 1.2 Hz, 1H, Hₐᵣₒₘ), 7.37 (dd, J=7.7, 1.7 Hz, 1H, Hₐᵣₒₘ), 7.30 (td, J=7.4, 1.2 Hz, 1H, Hₐᵣₒₘ), 7.17 (td, J=7.6, 1.7 Hz, 1H, Hₐᵣₒₘ), 4.59 (d, J=2.4 Hz, 2H, N1-CH₂), 4.20 (q, J=7.1 Hz, 2H, N7-CH₂), 3.96 (tdd, J=7.9, 5.9, 4.0 Hz, 6H, 2OCH₂ and N3-CH₂), 3.16 (t, J=7.2 Hz, 2H, CH₂), 3.09 (d, J=7.7 Hz, 2H, CH₂), 3.07 (t, J=2.4 Hz, 1H, H_{propargyl}), 1.74-1.59 (m, 4H, 2CH₂), 1.55-1.46 (m, 2H, CH₂), 1.40-1.33 (m, 2H, CH₂), 1.23 (d, J=7.1 Hz, 3H, N7-CH₂CH₃), 1.20 (t, J=7.1 Hz, 6H, 2OCH₂CH₃). ¹³C NMR (151 MHz, DMSO-d₆) δ [ppm]=152.9 (Cₓₐₙₜₕᵢₙₑ), 152.7 (Cₓₐₙₜₕᵢₙₑ), 149.8 (Cₓₐₙₜₕᵢₙₑ), 147.7 (Cₓₐₙₜₕᵢₙₑ), 139.3 (Cₐᵣₒₘ), 132.5 (Cₐᵣₒₘ), 131.0 (Cₐᵣₒₘ), 128.6 (Cₐᵣₒₘ), 127.9 (Cₐᵣₒₘ), 123.7 (Cₐᵣₒₘ), 105.5 (C5), 79.7 (C_{propargyl}), 72.7 (C_{propargyl}), 60.7 (d, ²J_{C,P}=6.1 Hz, 2OCH₂), 42.4 (N3-CH₂), 40.1 (N7-CH₂), 33.3 (CH₂), 29.9 (N1-CH₂), 26.8 (CH₂), 26.6 (d, ³J_{C,P}=16.1 Hz, CH₂), 26.0 (CH₂), 24.28 (d, ¹J_{C,P}=138.4 Hz, PCH₂), 21.7 (d, ²J_{C,P}=5.1 Hz, CH₂), 16.3 (d, ³J_{C,P}=5.7 Hz, 2OCH₂CH₃), 15.9 (N7-CH₂CH₃). ³¹P NMR (243 MHz, DMSO-d₆) δ [ppm]=33.0. LC-MS: positive mode [m/z]=607.3 [M+H]⁺. Purity: 91%; light-yellow solid.

### Synthesis of Diethyl (6-(8-(2-bromophenethyl)-7-ethyl-2,6-dioxo-1-(prop-2-yn-1-yl)-1,2,6,7-tetrahydro-3H-purin-3-yl)hexyl)phosphonate (60h

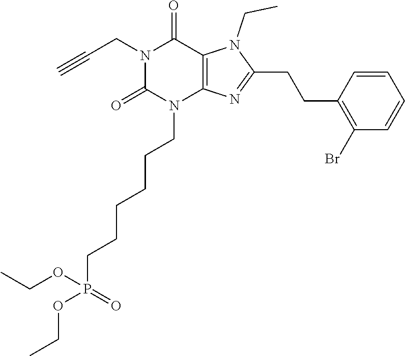 [SMILES: C#CCn1c(=O)c2c(nc(CCc3ccccc3Br)n2CC)n(CCCCCCP(=O)(OCC)OCC)c1=O]

Yield: 76%, light-yellow solid. H NMR (600 MHz, DMSO-d₆) δ [ppm]=7.60 (dd, J=8.0, 1.2 Hz, 1H, Hₐmm), 7.37 (dd, J=7.7, 1.8 Hz, 1H, Hₐmm), 7.30 (td, J=7.5, 1.3 Hz, 1H, Hₐmm), 7.17 (td, J=7.7, 1.7 Hz, 1H, Hₐᵣₒₘ), 4.59 (d, J=2.5 Hz, 2H, N1-CH₂), 4.20 (q, J=7.1 Hz, 2H, N7-CH₂), 4.01-3.89 (m, 6H, 2OCH₂ and N3-CH₂), 3.16 (t, J=7.3 Hz, 2H, CH₂), 3.09 (t, J=7.4 Hz, 2H, CH₂), 3.06 (t, J=2.4 Hz, 1H, H_{propargyl}), 1.72-1.60 (m, 4H, 2CH₂), 1.50-1.42 (m, 2H, CH₂), 1.42-1.36 (m, 2H, CH₂), 1.28 (p, J=8.1 Hz, 2H, CH₂), 1.23 (t, J=7.1 Hz, 3H, N7-CH₂CH₃), 1.20 (t, J=7.0 Hz, 6H, 2OCH₂CH₃). ¹³C NMR (151 MHz, DMSO-d₆) δ [ppm]=152.9 (Cₓₐₙₜₕᵢₙₑ), 152.7 (Cₓₐₙₜₕᵢₙₑ), 149.8 (Cₓₐₙₜₕᵢₙₑ), 147.7 (Cₓₐₙₜₕᵢₙₑ), 139.3 (Cₐᵣₒₘ), 132.5 (Cₐᵣₒₘ), 130.9 (Cₐᵣₒₘ), 128.5 (Cₐᵣₒₘ), 127.9 (Cₐᵣₒₘ), 123.7 (Cₐᵣₒₘ), 105.5 (C5), 79.7 (C_{propargyl}), 72.7 (C_{propargyl}), 60.7 (d, ²J_{C,P}=6.9 Hz, 2OCH₂), 42.6 (N3-CH₂), 40.1 (N7-CH₂), 33.3 (CH₂), 29.9 (N1-CH₂), 29.3 (d, ³J_{C,P}=15.8 Hz, CH₂), 27.2 (CH₂), 26.0 (CH₂), 25.5 (CH₂), 24.4 (d, ¹J_{C,P}=138.3 Hz, PCH₂), 21.9 (d, ²J_{C,P}=4.8 Hz, CH₂), 16.3 (d, ³J_{C,P}=5.8 Hz, 2OCH₂CH₃), 15.9 (N7-CH₂CH₃). ³¹P NMR (243 MHz, DMSO-d₆) δ [ppm]=33.0. LC-MS: positive mode [m/z]=621.4 [M+H]⁺. Purity: 90%.

### Synthesis of Diethyl (4-(7-ethyl-8-(2-iodophenethyl)-2,6-dioxo-1-(prop-2-yn-1-yl)-1,2,6,7-tetrahydro-3H-purin-3-yl)butyl)phosphonate (49b

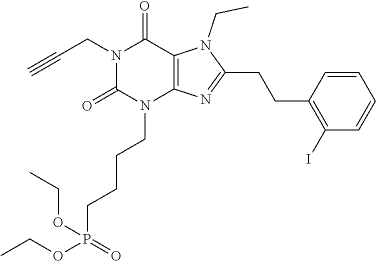 [SMILES: C#CCn1c(=O)c2c(nc(CCc3ccccc3I)n2CC)n(CCCCP(=O)(OCC)OCC)c1=O]

Yield: 47%, light-yellow solid. ¹H NMR (600 MHz, DMSO-d₆) δ [ppm]=7.85 (d, J=7.9 Hz, 1H, Hₐᵣₒₘ), 7.35-7.32 (m, 2H, 2Hₐᵣₒₘ), 6.98 (ddd, J=8.0, 5.5, 3.5 Hz, 1H, Hₐᵣₒₘ), 4.59 (d, J=2.4 Hz, 2H, N1-CH₂), 4.20 (q, J=7.1 Hz, 2H, N7-CH₂), 4.00 (t, J=6.8 Hz, 2H, N3-CH₂), 4.00-3.88 (m, 4H, 2OCH₂), 3.14 (t, J=7.6 Hz, 2H, CH₂), 3.07 (t, J=2.4 Hz, 1H, H_{propargyl}), 3.05 (t, J=7.7 Hz, 2H, CH₂), 1.78 (ddt, J=14.9, 11.8, 7.2 Hz, 4H, 2CH₂), 1.48 (dtt, J=12.7, 9.0, 6.1 Hz, 2H, CH₂), 1.23 (t, J=7.1 Hz, 3H, N7-CH₂CH₃), 1.19 (t, J=7.0 Hz, 6H, 2OCH₂CH₃). ¹³C NMR (151 MHz, DMSO-d₆) δ [ppm]=152.9 (Cₓₐₙₜₕᵢₙₑ), 152.6 (Cₓₐₙₜₕᵢₙₑ), 149.8 (Cₓₐₙₜₕᵢₙₑ), 147.7 (Cₓₐₙₜₕᵢₙₑ), 142.6 (Cₐᵣₒₘ), 139.1 (Cₐᵣₒₘ), 129.9 (Cₐᵣₒₘ), 128.6 (Cₐᵣₒₘ), 128.6 (Cₐᵣₒₘ), 105.5 (C5), 100.7 (Cₐᵣₒₘ), 79.6 (C_{propargyl}), 72.7 (C_{propargyl}), 60.8 (d, ²J_{C,P}=6.4 Hz, 2OCH₂), 42.1 (N3-CH₂), 40.2 (N7-CH₂), 37.8 (CH₂), 29.9 (N1-CH₂), 28.1 (d, ³J_{C,P}=15.9 Hz, CH₂), 26.4 (CH₂), 24.1 (d, ¹J_{C,P}=138.8 Hz, PCH₂), 19.3 (d, ²J_{C,P}=4.8 Hz, CH₂), 16.3 (d, ³J_{C,P}=5.7 Hz, 2OCH₂CH₃), 15.9 (N7-CH₂CH₃). ³¹P NMR (243 MHz, DMSO-d₆) δ [ppm]=32.8. LC-MS: positive mode [m/z]=641.3 [M+H]⁺. Purity: 89%.

### Synthesis of Diethyl (4-(7-ethyl-8-(2-methylphenethyl)-2,6-dioxo-1-(prop-2-yn-1-yl)-1,2,6,7-tetrahydro-3H-purin-3-yl)butyl)phosphonate (49c

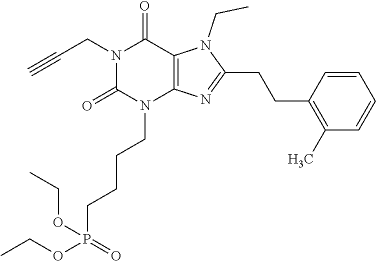 [SMILES: C#CCn1c(=O)c2c(nc(CCc3ccccc3C)n2CC)n(CCCCP(=O)(OCC)OCC)c1=O]

Yield: 99%, colorless oil. ¹H NMR (600 MHz, DMSO-d₆) δ [ppm]=7.15 (dt, J=6.0, 3.2 Hz, 2H, 2Hₐᵣₒₘ), 7.10 (dd, J=5.6, 3.4 Hz, 2H, 2Hₐᵣₒₘ), 4.59 (d, J=2.5 Hz, 2H, N1-CH₂), 4.15 (q, J=7.1 Hz, 2H, N7-CH₂), 4.01 (t, J=6.9 Hz, 2H, N3-CH₂), 3.98-3.90 (m, 4H, 2OCH₂), 3.07 (t, J=2.4 Hz, 1H, H_{propargyl}), 3.03 (s, 4H, 2CH₂), 2.30 (s, 3H, CH₃), 1.84-1.73 (m, 4H, 2CH₂), 1.48 (dtd, J=11.7, 6.4, 5.4, 3.2 Hz, 2H, CH₂), 1.19 (t, J=7.0 Hz, 6H, 2OCH₂CH₃), 1.18 (t, J=7.1 Hz, 3H, N7-CH₂CH₃). ¹³C NMR (151 MHz, DMSO-d₆) δ [ppm]=153.4 (Cₓₐₙₜₕᵢₙₑ), 152.8 (Cₓₐₙₜₕᵢₙₑ), 149.8 (Cₓₐₙₜₕᵢₙₑ), 147.8 (Cₓₐₙₜₕᵢₙₑ), 138.5 (Cₐᵣₒₘ), 135.7 (Cₐᵣₒₘ), 130.0 (Cₐᵣₒₘ), 128.8 (Cₐᵣₒₘ), 126.3 (Cₐᵣₒₘ), 125.9 (Cₐᵣₒₘ), 105.38 (C5), 79.6 (C_{propargyl}), 72.7 (C_{propargyl}), 60.7 (d, ²J_{C,P}=6.3 Hz, 2OCH₂), 42.1 (N3-CH₂), 40.0 (N7-CH₂), 30.3 (CH₂), 29.9 (CH₂), 28.2 (d, ³J_{C,P}=15.9 Hz, CH₂), 26.5 (CH₂), 24.1 (d, ¹J_{C,P}=138.7 Hz, PCH₂), 19.3 (d, ²J_{C,P}=4.8 Hz, CH₂), 18.8 (CH₃), 16.2 (d, ³J_{C,P}=5.7 Hz, 2OCH₂CH₃), 15.8 (N7-CH₂CH₃). ³¹P NMR (243 MHz, DMSO-d₆) δ [ppm]=32.8. LC-MS: positive mode [m/z]=529.40 [M+H]⁺. Purity: 93%.

### Synthesis of Diethyl (4-(8-(2,6-dimethylphenethyl)-7-ethyl-2,6-dioxo-1-(prop-2-yn-1-yl)-1,2,6,7-tetra-hydro-3H-purin-3-yl)butyl)phosphonate (49l

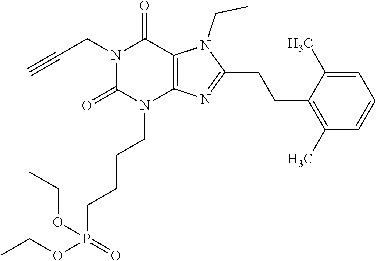 [SMILES: C#CCn1c(=O)c2c(nc(CCc3c(C)cccc3C)n2CC)n(CCCCP(=O)(OCC)OCC)c1=O]

Compound 491 was used for the next step without further purification. Light-yellow viscous liquid.

### Synthesis of Diethyl (4-(8-(2,6-dichlorophenethyl)-7-ethyl-2,6-dioxo-1-(prop-2-yn-1-yl)-1,2,6,7-tetra-hydro-3H-purin-3-yl)butyl)phosphonate (49m

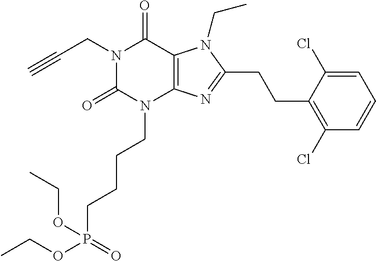 [SMILES: C#CCn1c(=O)c2c(nc(CCc3c(Cl)cccc3Cl)n2CC)n(CCCCP(=O)(OCC)OCC)c1=O]

Yield: 99%, light-yellow solid. ¹H NMR (600 MHz, DMSO-d6) δ[ppm]=7.48 (d, J=8.2 Hz, 2H, 2Hₐᵣₒₘ), 7.31 (t, J=8.1 Hz, 1H, Hₐᵣₒₘ), 4.60 (d, J=2.5 Hz, 2H, N1-CH₂), 4.24 (q, J=7.1 Hz, 2H, N7-CH₂), 3.98-3.92 (m, 6H, 2OCH₂ and N3-CH₂), 3.28 (t, J=7.6 Hz, 2H, CH₂), 3.08 (t, J=2.4 Hz, 1H, H_{propargyl}), 3.02 (t, J=7.4 Hz, 2H, CH₂), 1.81-1.68 (m, 4H, 2CH₂), 1.45 (dtd, J=12.3, 6.7, 5.7, 3.5 Hz, 2H, CH₂), 1.31 (t, J=7.1 Hz, 3H, N7-CH₂CH₃), 1.19 (t, J=7.1 Hz, 6H, 2OCH₂CH₃). ¹³C NMR (151 MHz, DMSO-d6) δ[ppm]=152.9 (Cₓₐₙₜₕᵢₙₑ), 152.1 (Cₓₐₙₜₕᵢₙₑ), 149.8 (Cₓₐₙₜₕᵢₙₑ), 147.8 (Cₓₐₙₜₕᵢₙₑ), 135.3 (Cₐᵣₒₘ), 134.7 (2Cₐᵣₒₘ), 129.2 (Cₐᵣₒₘ), 128.5 (2Cₐᵣₒₘ), 105.6 (C5), 79.6 (C_{propargyl}), 72.7 (C_{propargyl}), 60.7 (d, ²J_{C,P}=6.3 Hz, 2OCH₂), 42.0 (N3-CH₂), 40.2 (N7-CH₂), 30.0 (CH₂), 29.1 (CH₂), 28.1 (d, ³J_{C,P}=16.0 Hz, CH₂), 24.4 (CH₂), 24.1 (d, ¹J_{C,P}=138.6 Hz, PCH₂), 19.2 (d, ²J_{C,P}=4.9 Hz, CH₂), 16.2 (d, ³J_{C,P}=5.1 Hz, 2OCH₂CH₃), 16.0 (N7-CH₂CH₃). ³¹P NMR (243 MHz, DMSO-d6) δ[ppm]=32.7. LC-MS: positive mode [m/z]=583.4 [M+H]⁺. Purity: 94%.

### Synthesis of Diethyl (4-(8-(4-bromophenethyl)-7-ethyl-2,6-dioxo-1-(prop-2-yn-1-yl)-1,2,6,7-tetrahydro-3H-purin-3-yl)butyl)phosphonate (49d

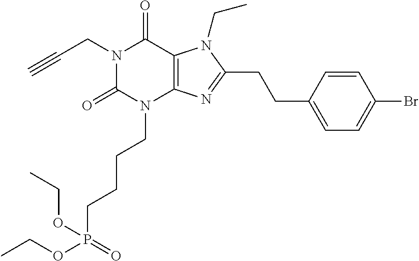 [SMILES: C#CCn1c(=O)c2c(nc(CCc3ccc(Br)cc3)n2CC)n(CCCCP(=O)(OCC)OCC)c1=O]

Compound 49d was used for the next step without further purification. ¹H NMR (600 MHz, DMSO-d₆) δ [ppm]=7.50-7.45 (m, 2H, 2Hₐᵣₒₘ), 7.28-7.22 (m, 2H, 2Hₐᵣₒₘ), 4.60 (d, J=2.4 Hz, 2H, N1-CH₂), 4.20 (q, J=7.1 Hz, 2H, N7-CH₂), 4.00 (t, J=6.8 Hz, 2H, N3-CH₂), 3.98-3.93 (m, 4H, 2OCH₂), 3.11-3.06 (m, 3H, H_{propargyl} and CH₂), 3.04 (dd, J=8.2, 5.5 Hz, 2H, CH₂), 1.85-1.71 (m, 4H, 2CH₂), 1.54-1.44 (m, 2H, CH₂), 1.22 (t, J=7.2 Hz, 3H, N7-CH₂CH₃), 1.20 (t, J=6.9 Hz, 6H, 2OCH₂CH₃). ¹³C NMR (151 MHz, DMSO-d₆) δ [ppm]=153.0 (Cₓₐₙₜₕᵢₙₑ), 152.8 (Cₓₐₙₜₕᵢₙₑ), 149.8 (Cₓₐₙₜₕᵢₙₑ), 147.7 (Cₓₐₙₜₕᵢₙₑ), 139.9 (Cₐᵣₒₘ), 131.1 (2Cₐᵣₒₘ), 130.8 (2Cₐᵣₒₘ), 119.2 (Cₐᵣₒₘ), 105.4 (C5), 79.6 (C_{propargyl}), 72.7 (C_{propargyl}), 60.8 (d, ²J_{C,P}=6.2 Hz, 2OCH₂), 42.1 (N3-CH₂), 40.1 (N7-CH₂), 32.1 (CH₂), 29.9 (N1-CH₂), 28.2 (d, ³J_{C,}=15.7 Hz, CH₂), 27.2 (CH₂), 24.1 (d, ¹J_{C,P}=138.7 Hz, PCH₂), 19.3 (d, ³J_{C,P}=5.0 Hz, CH₂), 16.2 (d, ²J_{C,P}=5.9 Hz, 2OCH₂CH₃), 15.8 (N7-CH₂CH₃). ³¹P NMR (243 MHz, DMSO-d₆) δ [ppm]=32.8. LC-MS: positive mode [m/z]=593.0 [M+H]⁺. Purity: 93%; light-yellow solid.

### Synthesis of Diethyl (4-(7-ethyl-8-(naphthalen-1-ylmethyl)-2,6-dioxo-1-(prop-2-yn-1-yl)-1,2,6,7-tetra-hydro-3H-purin-3-yl)butyl)phosphonate (78

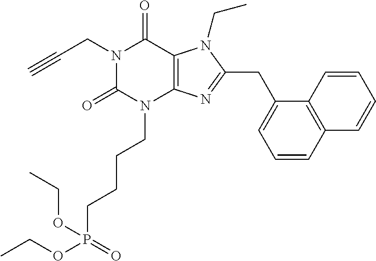 [SMILES: C#CCn1c(=O)c2c(nc(Cc3cccc4ccccc34)n2CC)n(CCCCP(=O)(OCC)OCC)c1=O]

Yield: 90%, light-yellow solid. ¹H NMR (600 MHz, DMSO-d6) δ[ppm]=7.90-7.85 (m, 3H, 3Hₐᵣₒₘ), 7.78 (d, J=1.7 Hz, 1H, Hₐᵣₒₘ), 7.52-7.46 (m, 2H, 2Hₐᵣₒₘ), 7.43 (dd, J=8.5, 1.8 Hz, 1H, Hₐᵣₒₘ), 4.60 (d, J=2.4 Hz, 2H, N1-CH₂), 4.41 (s, 2H, CH₂), 4.27 (q, J=7.0 Hz, 2H, N7-CH₂), 4.02 (t, J=6.9 Hz, 2H, N3-CH₂), 3.97-3.88 (m, 4H, 2OCH₂), 3.07 (t, J=2.4 Hz, 1H, H_{propargyl}), 1.78 (dq, J=18.1, 8.4, 7.8 Hz, 4H, 2CH₂), 1.50 (dtd, J=15.6, 6.4, 5.8, 2.5 Hz, 2H, CH₂), 1.17 (t, J=7.0 Hz, 6H, 2OCH₂CH₃), 1.09 (t, J=7.1 Hz, 3H, N7-CH₂CH₃). ¹³C NMR (151 MHz, DMSO-d6) δ[ppm]=152.9 (Cₓₐₙₜₕᵢₙₑ), 152.2 (Cₓₐₙₜₕᵢₙₑ), 149.8 (Cₓₐₙₜₕᵢₙₑ), 147.8 (Cₓₐₙₜₕᵢₙₑ), 134.0 (Cₐᵣₒₘ), 133.0 (Cₐᵣₒₘ), 131.9 (Cₐᵣₒₘ), 128.2 (Cₐᵣₒₘ), 127.5 (2Cₐᵣₒₘ), 126.8 (Cₐᵣₒₘ), 126.7 (Cₐᵣₒₘ), 126.3 (Cₐᵣₒₘ), 125.8 (Cₐᵣₒₘ), 105.9 (C5), 79.6 (C_{propargyl}), 72.7 (C_{propargyl}), 60.7 (d, ²J_{C,P}=6.3 Hz, 2OCH₂), 42.3 (N3-CH₂), 40.3 (N7-CH₂), 32.3 (CH₂), 30.0 (N1-CH₂), 28.2 (d, ³J_{C,P}=15.4 Hz, CH₂), 24.1 (d, ¹J_{C,P}=138.7 Hz, PCH₂), 19.4 (d, ²J_{C,P}=4.9 Hz, CH₂), 16.2 (d, J_{C,P}=5.6 Hz, 2OCH₂CH₃), 15.7 (N7-CH₂CH₃). ³¹P NMR (243 MHz, DMSO-d6) δ[ppm]=32.7. LC-MS: positive mode [m/z]=551.3 [M+H]⁺. Purity: 93%.

### Synthesis of Diethyl (4-(8-(2-bromophenethyl)-7-(cyclohexylmethyl)-2,6-dioxo-1-(prop-2-yn-1-yl)-1,2,6,7-tetrahydro-3H-purin-3-yl)butyl)phosphonate (50h

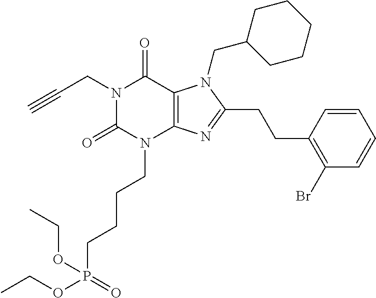 [SMILES: C#CCn1c(=O)c2c(nc(CCc3ccccc3Br)n2CC2CCCCC2)n(CCCCP(=O)(OCC)OCC)c1=O]

Yield: 55%, light-yellow solid. ¹H NMR (600 MHz, DMSO-d₆) δ [ppm]=7.60 (dd, J=8.0, 1.2 Hz, 1H, Hₐmm), 7.36 (dd, J=7.7, 1.8 Hz, 1H, Hₐmm), 7.31 (td, J=7.4, 1.2 Hz, 1H, Hₐmm), 7.17 (td, J=7.6, 1.8 Hz, 1H, Hₐᵣₒₘ), 4.58 (d, J=2.5 Hz, 2H, N1-CH₂), 4.01 (t, J=7.1 Hz, 2H, N3-CH₂), 3.98 (d, J=7.5 Hz, 2H, N7-CH₂), 3.94 (dt, J=8.0, 6.9 Hz, 4H, 2OCH₂), 3.18 (t, J=7.5 Hz, 2H, CH₂), 3.07 (t, J=2.4 Hz, 1H, H_{propargyl}), 3.04 (t, J=7.6 Hz, 2H, CH₂), 1.84-1.74 (m, 4H, 2CH₂), 1.61 (dd, J=11.3, 5.3 Hz, 2H, 2H_{cyclohexane}), 1.54-1.45 (m, 2H, CH₂), 1.41 (d, J=11.7 Hz, 2H, 2H_{cyclohexane}), 1.24 (d, J=5.3 Hz, 2H, 2H_{cyclohexane}), 1.18 (t, J=7.0 Hz, 6H, 2OCH₂CH₃), 1.12-1.03 (m, 2H, 2H_{cyclohexane}), 0.98 (dd, J=17.1, 7.3 Hz, 2H, 2H_{cyclohexane}). ¹³C NMR (151 MHz, DMSO-d₆) δ [ppm]=153.3 (Cₓₐₙₜₕᵢₙₑ), 153.0 (Cₓₐₙₜₕᵢₙₑ), 149.8 (Cₓₐₙₜₕᵢₙₑ), 147.6 (Cₓₐₙₜₕᵢₙₑ), 139.3 (Cₐᵣₒₘ), 132.5 (Cₐᵣₒₘ), 131.1 (Cₐᵣₒₘ), 128.6 (Cₐᵣₒₘ), 127.9 (Cₐᵣₒₘ), 123.7 (Cₐᵣₒₘ), 106.1 (C5), 79.6 (C_{propargyl}), 72.7 (C_{propargyl}), 60.7 (d, ²J_{C,P}=6.6 Hz, 2OCH₂), 50.0 (N7-CH₂), 42.1 (N3-CH₂), 38.4 (N7-CH₂CH), 33.1 (CH₂), 30.0 (N1-CH₂), 29.5 (2C_{cyclohexane}), 28.2 (d, ³J_{C,P}=15.8 Hz, CH₂), 26.3 (CH₂), 25.7 (2C_{cyclohexane}), 25.1 (C_{cyclohexane}), 24.1 (d, ¹J_{C,P}=138.6 Hz, PCH₂), 19.3 (d, ²J_{C,P}=4.8 Hz, CH₂), 16.2 (d, ³J_{C,P}=5.8 Hz, 2OCH₂CH₃). ³¹P NMR (243 MHz, DMSO-d₆) δ [ppm]=32.8. LC-MS: positive mode [m/z]=661.5 [M+H]⁺. Purity: 92%.

### Synthesis of Diethyl (4-(8-(2-bromophenethyl)-7-(cyclopentylmethyl)-2,6-dioxo-1-(prop-2-yn-1-yl)-1,2,6,7-tetrahydro-3H-purin-3-yl)butyl)phosphonate (51h

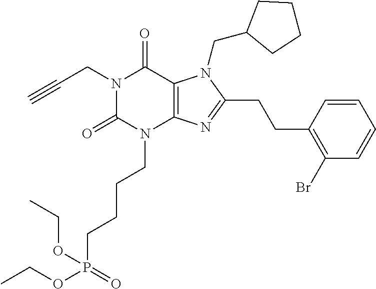 [SMILES: C#CCn1c(=O)c2c(nc(CCc3ccccc3Br)n2CC2CCCC2)n(CCCCP(=O)(OCC)OCC)c1=O]

Yield: 24%, light-yellow viscous liquid. ¹H NMR (600 MHz, DMSO-d₆) δ [ppm]=7.60 (dd, J=8.0, 1.2 Hz, 1H, Hₐᵣₒₘ), 7.35 (dd, J=7.6, 1.8 Hz, 1H, Hₐᵣₒₘ), 7.31 (td, J=7.4, 1.2 Hz, 1H, Hₐᵣₒₘ), 7.17 (td, J=7.7, 1.8 Hz, 1H, Hₐᵣₒₘ), 4.59 (d, J=2.5 Hz, 2H, N1-CH₂), 4.09 (d, J=7.7 Hz, 2H, N7-CH₂), 4.01 (t, J=6.9 Hz, 2H, N3-CH₂), 3.99-3.90 (m, 4H, 2OCH₂), 3.19 (t, J=7.5 Hz, 2H, CH₂), 3.07 (t, J=7.7 Hz, 2H, CH₂), 3.06 (t, J=2.5 Hz, 1H, H_{propargyl}), 2.19 (dt, J=15.1, 7.5 Hz, 1H, N7-CH₂CH), 1.86-1.73 (m, 4H, 2CH₂), 1.59 (dqd, J=10.9, 7.4, 6.2, 3.3 Hz, 2H, CH₂), 1.47 (ddq, J=23.9, 9.0, 6.3, 4.5 Hz, 8H, ⁸H_{cyclopentane}), 1.19 (t, J=7.0 Hz, 6H, 2OCH₂CH₃). ¹³C NMR (151 MHz, DMSO-d₆) δ [ppm]=153.1 (Cₓₐₙₜₕᵢₙₑ), 153.0 (Cₓₐₙₜₕᵢₙₑ), 149.8 (Cₓₐₙₜₕᵢₙₑ), 147.6 (Cₓₐₙₜₕᵢₙₑ), 139.3 (Cₐᵣₒₘ), 132.5 (Cₐᵣₒₘ), 131.0 (Cₐᵣₒₘ), 128.6 (Cₐᵣₒₘ), 127.9 (Cₐᵣₒₘ), 123.7 (Cₐᵣₒₘ), 105.9 (C5), 79.6 (C_{propargyl}), 72.7 (C_{propargyl}), 60.7 (d, ²J_{C,P}=6.1 Hz, 2OCH₂), 48.5 (N7-CH₂), 42.1 (N3-CH₂), 40.7 (N7-CH₂CH), 33.2 (CH₂), 30.0 (N1-CH₂), 29.2 (2CH₂), 28.2 (d, ³J_{C,P}=15.7 Hz, CH₂), 26.4 (CH₂), 24.2 (2CH₂), 24.1 (d, ¹J_{C,P}=138.7 Hz, PCH₂), 19.3 (d, ²J_{C,P}=4.9 Hz, CH₂), 16.2 (d, ³J_{C,P}=5.5 Hz, 2OCH₂CH₃). ³¹P NMR (243 MHz, DMSO-d₆) δ [ppm]=32.8. LC-MS: positive mode [m/z]=647.4 [M+H]⁺. Purity: 98%.

### Synthesis of Diethyl (4-(8-(4-bromophenethyl)-7-(cyclopentylmethyl)-2,6-dioxo-1-(prop-2-yn-1-yl)-1,2,6,7-tetrahydro-3H-purin-3-yl)butyl)phosphonate (51d

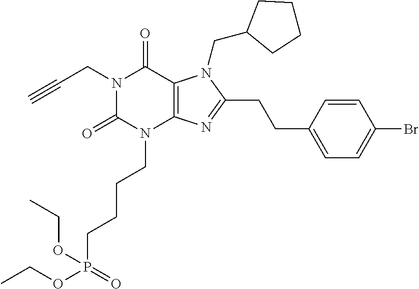 [SMILES: C#CCn1c(=O)c2c(nc(CCc3ccc(Br)cc3)n2CC2CCCC2)n(CCCCP(=O)(OCC)OCC)c1=O]

Compound 51d was used for the next step without further purification. ¹H NMR (600 MHz, DMSO-d₆) δ [ppm]=7.45 (d, J=8.3 Hz, 2H, 2Hₐᵣₒₘ), 7.22 (d, J=8.3 Hz, 2H, 2Hₐᵣₒₘ), 4.58 (d, J=2.5 Hz, 2H, N1-CH₂), 4.11 (d, J=7.7 Hz, 2H, N7-CH₂), 4.02 (dt, J=7.1 Hz, 2H, N3-CH₂), 3.98-3.90 (m, 4H, 2OCH₂), 3.06 (s, 4H, 2CH₂), 2.16-2.08 (m, 1H, N7-CH₂CH), 1.79 (m, 4H, 2CH₂), 1.58 (ddd, J=13.5, 6.6, 3.6 Hz, 2H, CH₂), 1.46 (m, 8H, ⁸H_{cyclopentane}), 1.18 (t, J=6.9 Hz, 6H, 2OCH₂CH₃). ¹³C NMR (151 MHz, DMSO-d₆) δ [ppm]=153.5 (Cₓₐₙₜₕᵢₙₑ), 153.0 (Cₓₐₙₜₕᵢₙₑ), 149.8 (Cₓₐₙₜₕᵢₙₑ), 147.6 (Cₓₐₙₜₕᵢₙₑ), 140.0 (Cₐᵣₒₘ), 131.1 (Cₐᵣₒₘ), 130.9 (Cₐᵣₒₘ), 119.3 (Cₐᵣₒₘ), 105.9 (C5), 79.7 (C_{propargyl}), 72.7 (C_{propargyl}), 60.8 (d, ²J_{C,P}=6.2 Hz, 2OCH₂), 48.5 (N7-CH₂), 42.2 (N3-CH₂), 40.7 (N7-CH₂CH), 32.0 (CH₂), 30.0 (N1-CH₂), 29.2 (2CH₂), 28.2 (d, ³J_{C,P}=16.0 Hz, CH₂), 27.7 (CH₂), 24.2 (2CH₂), 24.1 (d, ¹J_{C,P}=138.7 Hz, PCH₂), 19.3 (d, ²J_{C,P}=4.0 Hz, CH₂), 16.3 (d, ³J_{C,P}=5.4 Hz, 2OCH₂CH₃). ³¹P NMR (243 MHz, DMSO-d₆) δ [ppm]=32.8. LC-MS: positive mode [m/z]=647.5 [M+H]⁺. Purity: 92%; light-yellow solid.

### Synthesis of Diethyl (4-(7-(cyclopentylmethyl)-8-(4-iodophenethyl)-2,6-dioxo-1-(prop-2-yn-1-yl)-1,2,6,7-tetrahydro-3H-purin-3-yl)butyl)phosphonate (51e

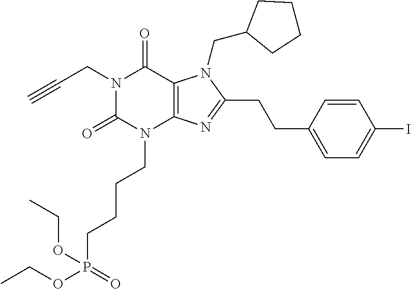 [SMILES: C#CCn1c(=O)c2c(nc(CCc3ccc(I)cc3)n2CC2CCCC2)n(CCCCP(=O)(OCC)OCC)c1=O]

Yield: 55%, colorless solid. H NMR (600 MHz, DMSO-d₆) δ [ppm]=7.62 (d, J=8.2 Hz, 2H, 2Hₐᵣₒₘ), 7.08 (d, J=8.3 Hz, 2H, 2Hₐᵣₒₘ), 4.58 (d, J=2.5 Hz, 2H, N1-CH₂), 4.11 (d, J=7.7 Hz, 2H, N7-CH₂), 4.01 (t, J=6.9 Hz, 2H, N3-CH₂), 3.99-3.90 (m, 4H, 2OCH₂), 3.06 (t, J=2.4 Hz, 1H, H_{propargyl}), 3.05-3.03 (m, 4H, 2CH₂), 2.14-2.08 (m, 1H, N7-CH₂CH), 1.79 (tdd, J=14.6, 10.2, 7.1 Hz, 4H, 2CH₂), 1.63-1.55 (m, 2H, CH₂), 1.54-1.39 (m, 6H, ⁶H_{cyclopentane}), 1.19 (t, J=7.0 Hz, 6H, 2OCH₂CH₃), 1.16-1.13 (m, 2H, ²H_{cyclopentane}). ¹³C NMR (151 MHz, DMSO-d₆) δ [ppm]=153.5 (Cₓₐₙₜₕᵢₙₑ), 153.0 (Cₓₐₙₜₕᵢₙₑ), 149.8 (Cₓₐₙₜₕᵢₙₑ), 147.6 (Cₓₐₙₜₕᵢₙₑ), 140.3 (Cₐᵣₒₘ), 137.0 (2Cₐᵣₒₘ), 131.0 (2Cₐᵣₒₘ), 105.9 (C5), 91.8 (Cₐᵣₒₘ), 79.6 (Cₓₐₙₜₕᵢₙₑ), 72.7 (Cₓₐₙₜₕᵢₙₑ), 60.8 (d, ²J_{C,P}=6.4 Hz, 2OCH₂), 48.4 (N7-CH₂), 42.1 (N3-CH₂), 40.7 (N7-CH₂CH), 32.1 (CH₂), 30.0 (N1-CH₂), 29.2 (2C_{cyclopentane}), 28.2 (d, ³J_{C,P}=15.9 Hz, CH₂), 27.7 (CH₂), 24.2 (2C_{cyclopentane}), 24.1 (d, ¹J_{C,P}=138.8 Hz, PCH₂), 19.3 (d, ²J_{C,P}=4.7 Hz, CH₂), 16.2 (d, ³J_{C,P}=5.9 Hz, 2OCH₂CH₃). ³¹P NMR (243 MHz, DMSO-d₆) δ [ppm]=32.8. LC-MS: positive mode [m/z]=695.30 [M+H]⁺. Purity: 96%.

### Synthesis of Diethyl (4-(7-(cyclopentylmethyl)-2,6-dioxo-1-(prop-2-yn-1-yl)-8-(4-(trifluoromethyl) phenethyl)-1,2,6,7-tetrahydro-3H-purin-3-yl)butyl)phosphonate (51f

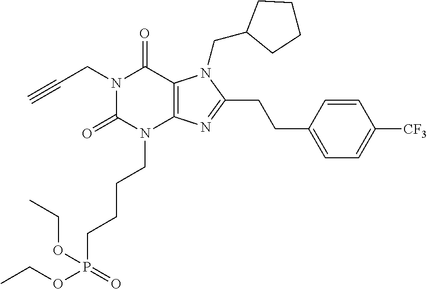 [SMILES: C#CCn1c(=O)c2c(nc(CCc3ccc(C(F)(F)F)cc3)n2CC2CCCC2)n(CCCCP(=O)(OCC)OCC)c1=O]

Yield: 88%, light-yellow viscous liquid. H NMR (600 MHz, DMSO-d₆) δ [ppm]=7.63 (d, J=8.1 Hz, 2H, 2Hₐᵣₒₘ), 7.50 (d, J=8.0 Hz, 2H, 2Hₐᵣₒₘ), 4.58 (d, J=2.5 Hz, 2H, N1-CH₂), 4.13 (d, J=7.7 Hz, 2H, N7-CH₂), 4.01 (t, J=6.9 Hz, 2H, N3-CH₂), 3.98-3.90 (m, 4H, 2OCH₂), 3.19 (t, J=7.3 Hz, 2H, CH₂), 3.12 (t, J=7.0 Hz, 2H, CH₂), 3.06 (t, J=2.4 Hz, 1H, H_{propargyl}), 2.15 (p, J=7.6 Hz, 1H, N7-CH₂CH), 1.83-1.74 (m, 4H, 2CH₂), 1.58 (ddd, J=8.0, 4.1, 1.9 Hz, 2H, CH₂), 1.53-1.38 (m, 6H, ⁶C_{cyclopentane}), 1.18 (t, J=7.0 Hz, 6H, 2OCH₂CH₃), 1.16-1.13 (m, 2H, 2C_{cyclopentane}). ¹³C NMR (151 MHz, DMSO-d₆) δ [ppm]=153.3 (Cₓₐₙₜₕᵢₙₑ), 153.0 (Cₓₐₙₜₕᵢₙₑ), 149.8 (Cₓₐₙₜₕᵢₙₑ), 147.6 (Cₓₐₙₜₕᵢₙₑ), 145.5 (Cₐᵣₒₘ), 129.4 (2Cₐᵣₒₘ), 126.9 (q, ²J_{C,F}=32.5 Hz, Cₐᵣₒₘ), 125.1 (q, ³J_{C,F}=3.5 Hz, 2Cₐᵣₒₘ), 124.4 (q, ¹J_{C,F}=271.7 Hz, CF₃), 105.9 (C5), 79.6 (C_{propargyl}), 72.7 (C_{propargyl}), 60.8 (d, ²J_{C,P}=6.2 Hz, 2OCH₂), 48.5 (N7-CH₂), 42.2 (N3-CH₂), 40.1 (N7-CH₂CH), 32.3 (CH₂), 30.0 (N1-CH₂), 29.1 (2C_{cyclopentane}), 28.2 (d, ³J_{C,P}=16.1 Hz, CH₂), 27.5 (CH₂), 24.2 (2C_{cyclopentane}), 24.1 (d, ¹J_{C,P}=139.0 Hz, PCH₂), 19.3 (d, ²J_{C,P}=4.8 Hz, CH₂), 16.2 (d, ³J_{C,P}=5.8 Hz, 2OCH₂CH₃). ³¹P NMR (243 MHz, DMSO-d₆) δ [ppm]=32.8. LC-MS: positive mode [m/z]=637.4 [M+H]⁺. Purity: 93%.

### Synthesis of Diethyl (4-(7-(cyclopentylmethyl)-8-(4-methoxyphenethyl)-2,6-dioxo-1-(prop-2-yn-1-yl)-1,2,6,7-tetrahydro-3H-purin-3-yl)butyl)phosphonate (51g

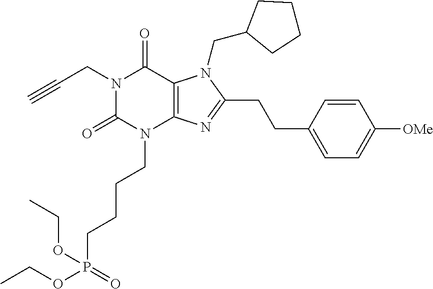 [SMILES: C#CCn1c(=O)c2c(nc(CCc3ccc(OC)cc3)n2CC2CCCC2)n(CCCCP(=O)(OCC)OCC)c1=O]

Yield: 55%, light-yellow viscous liquid. H NMR (600 MHz, DMSO-d₆) δ [ppm]=7.15 (d, J=8.6 Hz, 2H, 2Hₐᵣₒₘ), 6.83 (d, J=8.6 Hz, 2H, 2Hₐᵣₒₘ), 4.58 (d, J=2.4 Hz, 2H, N1-CH₂), 4.09 (d, J=7.7 Hz, 2H, N7-CH₂), 4.02 (t, J=6.9 Hz, 2H, N3-CH₂), 3.99-3.90 (m, 4H, 2OCH₂), 3.70 (s, 3H, OCH₃), 3.06 (t, J=2.4 Hz, 1H, H_{propargyl}), 3.01 (s, 4H, 2CH₂), 2.14 (p, J=7.6 Hz, 1H, N7-CH₂CH), 1.85-1.76 (m, 4H, 2CH₂), 1.61-1.55 (m, 2H, CH₂), 1.53-1.40 (m, 8H, ⁸H_{cyclopentane}), 1.19 (t, J=7.0 Hz, 6H, 2OCH₂CH₃). ¹³C NMR (126 MHz, DMSO-d₆) δ [ppm]=157.7 (Cₐᵣₒₘ), 153.8 (Cₓₐₙₜₕᵢₙₑ), 153.0 (Cₓₐₙₜₕᵢₙₑ), 149.8 (Cₓₐₙₜₕᵢₙₑ), 147.6 (Cₓₐₙₜₕᵢₙₑ), 132.3 (Cₐᵣₒₘ), 129.4 (2Cₐᵣₒₘ), 113.7 (2Cₐᵣₒₘ), 105.8 (C5), 79.6 (C_{propargyl}), 72.6 (C_{propargyl}), 60.7 (d, ²J_{C,P}=6.3 Hz, 2OCH₂), 54.9 (OCH₃), 48.4 (N7-CH₂), 42.1 (N3-CH₂), 40.7 (N7-CH₂CH), 32.0 (CH₂), 29.9 (N1-CH₂), 29.2 (2C_{cyclopentane}), 28.3 (CH₂), 28.2 (d, ³J_{C,P}=15.9 Hz, CH₂), 24.2 (2C_{cyclopentane}), 24.1 (d, ¹J_{C,P}=138.7 Hz, PCH₂), 19.3 (d, ²J_{C,P}=5.1 Hz, CH₂), 16.2 (d, ³J_{C,P}=5.6 Hz, 2OCH₂CH₃). ³¹P NMR (243 MHz, DMSO-d₆) δ [ppm]=32.8. LC-MS: positive mode [m/z]=599.40 [M+H]⁺. Purity: 96%.

### Synthesis of Diethyl (4-(7-(cyclopentylmethyl)-8-(4-ethylphenethyl)-2,6-dioxo-1-(prop-2-yn-1-yl)-1,2,6,7-tetrahydro-3H-purin-3-yl)butyl)phosphonate (51i

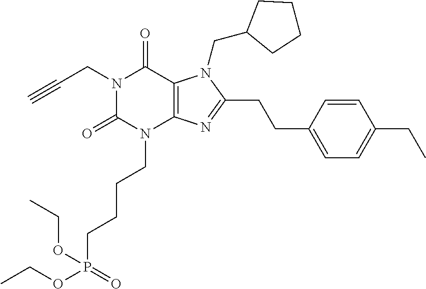 [SMILES: C#CCn1c(=O)c2c(nc(CCc3ccc(CC)cc3)n2CC2CCCC2)n(CCCCP(=O)(OCC)OCC)c1=O]

Compound 51i was used for the next step without further purification. Light-yellow viscous liquid.

### Synthesis of Diethyl (4-(7-(cyclopentylmethyl)-8-(4-isopropylphenethyl)-2,6-dioxo-1-(prop-2-yn-1-yl)-1,2,6,7-tetrahydro-3H-purin-3-yl)butyl)phosphonate (51j

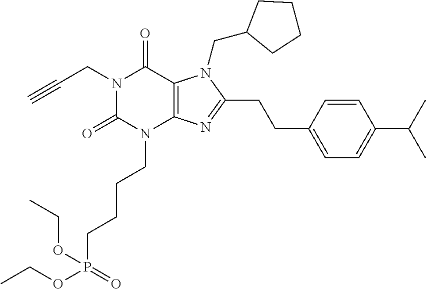 [SMILES: C#CCn1c(=O)c2c(nc(CCc3ccc(C(C)C)cc3)n2CC2CCCC2)n(CCCCP(=O)(OCC)OCC)c1=O]

Compound 51j was used for the next step without further purification. Light-yellow viscous liquid.

### Synthesis of Diethyl (4-(8-(2-([1,1′-biphenyl]-4-yl)ethyl)-7-(cyclopentylmethyl)-2,6-dioxo-1-(prop-2-yn-1-yl)-1,2,6,7-tetrahydro-3H-purin-3-yl)butyl)phosphonate (51k

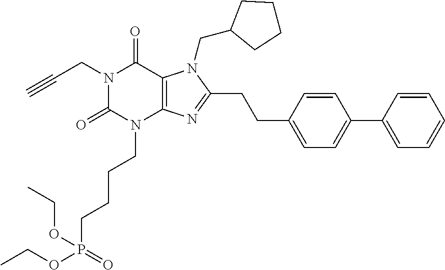 [SMILES: C#CCn1c(=O)c2c(nc(CCc3ccc(-c4ccccc4)cc3)n2CC2CCCC2)n(CCCCP(=O)(OCC)OCC)c1=O]

Compound 51k was used for the next step without further purification. Light-yellow viscous liquid.

### Synthesis of Diethyl (4-(8-(2-bromophenethyl)-7-(cyclobutylmethyl)-2,6-dioxo-1-(prop-2-yn-1-yl)-1,2,6,7-tetrahydro-3H-purin-3-yl)butyl)phosphonate (52h

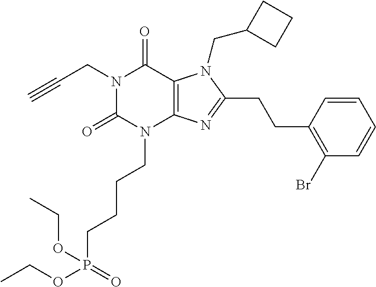 [SMILES: C#CCn1c(=O)c2c(nc(CCc3ccccc3Br)n2CC2CCC2)n(CCCCP(=O)(OCC)OCC)c1=O]

Yield: 71%, light-yellow viscous liquid. ¹H NMR (600 MHz, DMSO-d₆) δ [ppm]=7.61 (dd, J=7.9, 1.2 Hz, 1H, Hₐᵣₒₘ), 7.37 (dd, J=7.6, 1.8 Hz, 1H, Hₐᵣₒₘ), 7.32 (td, J=7.5, 1.3 Hz, 1H, Hₐᵣₒₘ), 7.18 (td, J=7.6, 1.8 Hz, 1H, Hₐᵣₒₘ), 4.59 (d, J=2.5 Hz, 2H, N1-CH₂), 4.22 (d, J=7.4 Hz, 2H, N7-CH₂), 4.00 (t, J=6.9 Hz, 2H, N3-CH₂), 3.97-3.90 (m, 4H, 2OCH₂), 3.18 (t, J=7.6 Hz, 2H, CH₂), 3.10-3.04 (m, 2H, CH₂), 3.07 (d, J=2.4 Hz, 1H, H_{propargyl}), 2.62 (p, J=7.7 Hz, 1H, N7-CH₂CH) 1.87-1.74 (m, 8H, 2CH₂ and 4H_{cyclobutane}), 1.73-1.66 (m, 2H, CH₂), 1.48 (dtd, J=15.6, 8.6, 7.6, 4.1 Hz, 2H, 2H_{cyclobutane}), 1.19 (t, J=7.1 Hz, 6H, 2OCH₂CH₃). ¹³C NMR (151 MHz, DMSO-d₆) δ [ppm]=153.1 (Cₓₐₙₜₕᵢₙₑ), 153.0 (Cₓₐₙₜₕᵢₙₑ), 149.8 (Cₓₐₙₜₕᵢₙₑ), 147.6 (Cₓₐₙₜₕᵢₙₑ), 139.3 (Cₐᵣₒₘ), 132.5 (Cₐᵣₒₘ), 131.0 (Cₐᵣₒₘ), 128.6 (Cₐᵣₒₘ), 127.9 (Cₐᵣₒₘ), 123.8 (Cₐᵣₒₘ), 105.9 (C5), 79.6 (C_{propargyl}), 72.7 (C_{propargyl}), 60.8 (d, ²J_{C,P}=6.2 Hz, 2OCH₂), 48.6 (N7-CH₂), 42.2 (N3-CH₂), 35.6 (N7-CH₂CH), 33.2 (CH₂), 30.0 (N1-CH₂), 28.1 (d, ³J_{C,P}=15.9 Hz, CH₂), 26.3 (CH₂), 24.9 (2CH₂), 24.1 (d, ¹J_{C,P}=138.6 Hz, PCH₂), 19.3 (d, ²J_{C,P}=4.9 Hz, CH₂), 17.7 (CH₂), 16.2 (d, ³J_{C,P}=5.7 Hz, 2OCH₂CH₃). ³¹P NMR (243 MHz, DMSO-d₆) δ [ppm]=32.8. LC-MS: positive mode [m/z]=633.4 [M+H]⁺. Purity: 94%.

### Synthesis of Diethyl (4-(8-(2-bromophenethyl)-7-(2-chloroethyl)-2,6-dioxo-1-(prop-2-yn-1-yl)-1,2,6,7-tetrahydro-3H-purin-3-yl)butyl)phosphonate (55h

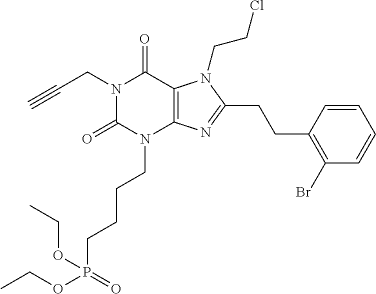 [SMILES: C#CCn1c(=O)c2c(nc(CCc3ccccc3Br)n2CCCl)n(CCCCP(=O)(OCC)OCC)c1=O]

Yield: 85%, off-white solid. ¹H NMR (600 MHz, DMSO-d₆) δ [ppm]=7.60 (dd, J=8.0, 1.2 Hz, 1H, Hₐᵣₒₘ), 7.40 (dd, J=7.6, 1.7 Hz, 1H, Hₐᵣₒₘ), 7.32 (td, J=7.4, 1.3 Hz, 1H, Hₐᵣₒₘ), 7.18 (td, J=7.7, 1.7 Hz, 1H, Hₐᵣₒₘ), 4.59 (d, J=2.5 Hz, 2H, N1-CH₂), 4.50 (t, J=5.9 Hz, 2H, N7-CH₂CH₂), 4.00 (t, J=6.9 Hz, 2H, N3-CH₂), 3.95 (qd, J=6.9, 1.8 Hz, 6H, 2OCH₂ and N7-CH₂CH₂), 3.18 (ddd, J=8.1, 6.5, 1.9 Hz, 2H, CH₂), 3.13 (ddd, J=9.0, 6.6, 1.8 Hz, 2H, CH₂), 3.08 (t, J=2.4 Hz, 1H, H_{propargyl}), 1.83-1.73 (m, 4H, 2CH₂), 1.48 (dtd, J=12.8, 6.6, 5.8, 3.6 Hz, 2H, CH₂), 1.19 (t, J=7.0 Hz, 6H, 2OCH₂CH₃). ¹³C NMR (151 MHz, DMSO-d₆) δ [ppm]=154.1 (Cₓₐₙₜₕᵢₙₑ), 153.1 (Cₓₐₙₜₕᵢₙₑ), 149.8 (Cₓₐₙₜₕᵢₙₑ), 147.9 (Cₓₐₙₜₕᵢₙₑ), 139.3 (Cₐᵣₒₘ), 132.5 (Cₐᵣₒₘ), 130.9 (Cₐᵣₒₘ), 128.6 (Cₐᵣₒₘ), 127.9 (Cₐᵣₒₘ), 123.7 (Cₐᵣₒₘ), 105.7 (C5), 79.5 (C_{propargyl}), 72.8 (C_{propargyl}), 60.8 (d, ²J_{C,P}=6.3 Hz, 2OCH₂), 45.9 (N7-CH₂), 43.5 (N7-CH₂CH₂), 42.2 (N3-CH₂), 33.0 (CH₂), 30.0 (N1-CH₂), 28.2 (d, ³J_{C,P}=15.9 Hz, CH₂), 26.4 (CH₂), 24.1 (d, ¹J_{C,P}=138.7 Hz, PCH₂), 19.3 (d, ²J_{C,P}=4.7 Hz, CH₂), 16.2 (d, ³J_{C,P}=4.9 Hz, 2OCH₂CH₃). ³¹P NMR (243 MHz, DMSO-d₆) δ [ppm]=32.7. LC-MS: positive mode [m/z]=627.4 [M+H]⁺. Purity: 91%.

### Synthesis of tert-Butyl (2-(8-(2-bromophenethyl)-3-(4-(diethoxyphosphoryl)butyl)-2,6-dioxo-1-(prop-2-yn-1-yl)-1,2,3,6-tetrahydro-7H-purin-7-yl)ethyl)carbamate (56h

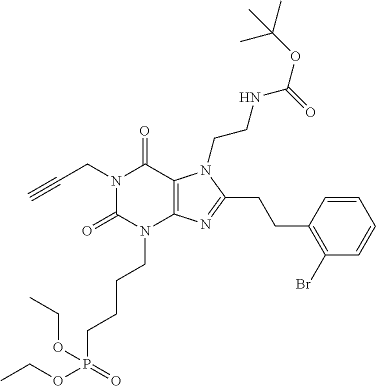 [SMILES: C#CCn1c(=O)c2c(nc(CCc3ccccc3Br)n2CCNC(=O)OC(C)(C)C)n(CCCCP(=O)(OCC)OCC)c1=O]

Compound 56h was used for the next step without further purification. ¹H NMR (600 MHz, DMSO-d₆) δ [ppm]=7.60 (dd, J=8.1, 1.2 Hz, 1H, Hₐᵣₒₘ), 7.38 (dd, J=7.7, 1.9 Hz, 1H, Hₐᵣₒₘ), 7.32 (t, J=7.5 Hz, 1H, Hₐᵣₒₘ), 7.18 (td, J=7.7, 1.7 Hz, 1H, Hₐᵣₒₘ), 6.91 (t, J=6.1 Hz, 1H, CONH), 4.60 (d, J=2.5 Hz, 2H, N1-CH₂), 4.17 (t, J=5.5 Hz, 2H, N7-CH₂), 4.00 (t, J=7.8 Hz, 2H, N3-CH₂), 4.00-3.90 (m, 4H, 2OCH₂), 3.29-3.23 (m, 2H, N7-CH₂CH₂), 3.16 (t, J=7.8 Hz, 2H, CH₂), 3.08 (t, J=2.4 Hz, 1H, H_{propargyl}), 3.00 (t, J=7.9 Hz, 2H, CH₂), 1.83-1.72 (m, 4H, 2CH₂), 1.54-1.41 (m, 2H, CH₂), 1.24 (s, 9H, C(CH₃)₃), 1.20 (t, J=7.0 Hz, 6H, 2OCH₂CH₃). ¹³C NMR (151 MHz, DMSO-d₆) δ [ppm]=155.5 (CO), 153.7 (Cₓₐₙₜₕᵢₙₑ), 153.0 (Cₓₐₙₜₕᵢₙₑ), 149.8 (Cₓₐₙₜₕᵢₙₑ), 147.7 (Cₓₐₙₜₕᵢₙₑ), 139.4 (Cₐᵣₒₘ), 132.5 (Cₐᵣₒₘ), 130.9 (Cₐᵣₒₘ), 128.5 (Cₐᵣₒₘ), 127.9 (Cₐᵣₒₘ), 123.7 (Cₐᵣₒₘ), 106.0 (C5), 79.6 (C_{propargyl}), 77.8 (C(CH₃)₃), 72.8 (C_{propargyl}), 60.8 (d, ²J_{C,P}=6.3 Hz, 2OCH₂), 44.6 (N7-CH₂CH₂), 42.1 (N3-CH₂), 40.1 (N7-CH₂CH₂), 33.1 (CH₂), 29.9 (N1-CH₂), 28.2 (d, ³J_{C,P}=15.9 Hz, CH₂), 27.9 (C(CH₃)₃), 26.1 (CH₂), 24.1 (d, ¹J_{C,P}=138.4 Hz, PCH₂), 19.2 (d, ²J_{C,P}=4.8 Hz, CH₂), 16.2 (d, ³J_{C,P}=5.5 Hz, 2OCH₂CH₃). ³¹P NMR (243 MHz, DMSO-d₆) δ [ppm]=32.8. LC-MS: positive mode [m/z]=708.5 [M+H]⁺. Purity: 91%; colorless liquid.

### Synthesis of Diethyl (4-(8-(2-bromophenethyl)-7-isopropyl-2,6-dioxo-1-(prop-2-yn-1-yl)-1,2,6,7-tetrahydro-3H-purin-3-yl)butyl)phosphonate (53h

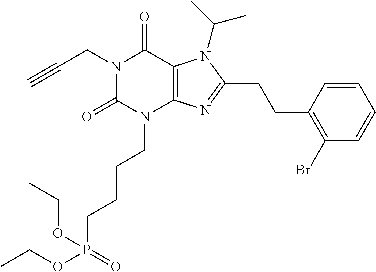 [SMILES: C#CCn1c(=O)c2c(nc(CCc3ccccc3Br)n2C(C)C)n(CCCCP(=O)(OCC)OCC)c1=O]

Yield: 66%, colorless liquid. H NMR (600 MHz, DMSO-d₆) δ [ppm]=7.59 (dd, J=8.1, 1.2 Hz, 1H, Hₐᵣₒₘ), 7.34 (dd, J=7.7, 1.8 Hz, 1H, Hₐᵣₒₘ), 7.30 (td, J=7.4, 1.2 Hz, 1H, Hₐᵣₒₘ), 7.16 (td, J=7.6, 1.9 Hz, 1H, Hₐᵣₒₘ), 4.79-4.65 (m, 1H, N7-CH), 4.60 (d, J=2.5 Hz, 2H, N1-CH₂), 3.99 (t, J=6.9 Hz, 2H, N3-CH₂), 3.97-3.90 (m, 4H, 2OCH₂), 3.16-3.11 (m, 4H, 2CH₂), 3.05 (t, J=2.4 Hz, 1H, H_{propargyl}), 1.82-1.70 (m, 4H, 2CH₂), 1.51-1.45 (m, 2H, CH₂), 1.43 (d, J=6.7 Hz, 6H, N7-CH(CH₃)₂), 1.18 (t, J=7.0 Hz, 6H, 2OCH₂CH₃). ¹³C NMR (151 MHz, DMSO-d₆) δ [ppm]=152.7 (Cₓₐₙₜₕᵢₙₑ), 152.4 (Cₓₐₙₜₕᵢₙₑ), 149.7 (Cₓₐₙₜₕᵢₙₑ), 148.8 (Cₓₐₙₜₕᵢₙₑ), 139.2 (Cₐᵣₒₘ), 132.5 (Cₐᵣₒₘ), 131.0 (Cₐᵣₒₘ), 128.6 (Cₐᵣₒₘ), 127.9 (Cₐᵣₒₘ), 123.7 (Cₐᵣₒₘ), 105.6 (C5), 79.7 (C_{propargyl}), 72.7 (C_{propargyl}), 60.7 (d, ²J_{C,P}=6.3 Hz, 2OCH₂), 48.9 (N7-CH), 42.1 (N3-CH₂), 33.7 (CH₂), 30.4 (N1-CH₂), 28.1 (d, ³J_{C,P}=15.9 Hz, CH₂), 27.2 (CH₂), 24.1 (d, ¹J_{C,P}=138.7 Hz, PCH₂), 21.2 (N7-CH(CH₃)₂), 19.3 (d, ²J_{C,P}=4.9 Hz, CH₂), 16.2 (d, ³J_{C,P}=5.6 Hz, 2OCH₂CH₃). ³¹P NMR (243 MHz, DMSO-d₆) δ [ppm]=32.7. LC-MS: positive mode [m/z]=607.4 [M+H]⁺. Purity: 93%.

### Synthesis of Diethyl (4-(8-(2-iodophenethyl)-7-isopropyl-2,6-dioxo-1-(prop-2-yn-1-yl)-1,2,6,7-tetra-hydro-3H-purin-3-yl)butyl)phosphonate (53b

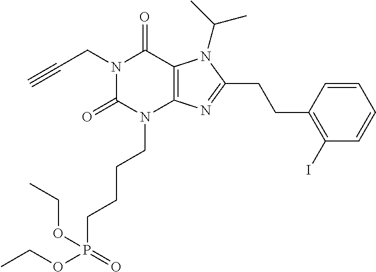 [SMILES: C#CCn1c(=O)c2c(nc(CCc3ccccc3I)n2C(C)C)n(CCCCP(=O)(OCC)OCC)c1=O]

Yield: 87%, colorless viscous liquid. ¹H NMR (600 MHz, DMSO-d₆) δ [ppm]=7.85 (dd, J=7.9, 1.1 Hz, 1H, Hₐᵣₒₘ), 7.35-7.29 (m, 2H, 2Hₐᵣₒₘ), 6.97 (ddd, J=7.9, 6.7, 2.3 Hz, 1H, Hₐᵣₒₘ), 4.77-4.64 (m, 1H, N7-CH), 4.61 (d, J=2.5 Hz, 2H, N1-CH₂), 4.00 (t, J=6.9 Hz, 2H, N3-CH₂), 3.99-3.91 (m, 4H, 2OCH₂), 3.13-3.10 (m, 4H, 2CH₂), 3.06 (t, J=2.4 Hz, 1H, H_{propargyl}), 1.85-1.71 (m, 4H, 2CH₂), 1.53-1.46 (m, 2H, CH₂), 1.44 (d, J=6.7 Hz, 6H, N7-CH(CH₃)₂), 1.19 (t, J=7.1 Hz, 6H, 2OCH₂CH₃). ¹³C NMR (151 MHz, DMSO-d₆) δ [ppm]=152.6 (Cₓₐₙₜₕᵢₙₑ), 152.4 (Cₓₐₙₜₕᵢₙₑ), 149.7 (Cₓₐₙₜₕᵢₙₑ), 148.8 (Cₓₐₙₜₕᵢₙₑ), 142.5 (Cₐᵣₒₘ), 139.1 (Cₐᵣₒₘ), 129.9 (Cₐᵣₒₘ), 128.6 (Cₐᵣₒₘ), 128.6 (Cₐᵣₒₘ), 105.6 (C5), 100.7 (Cₐᵣₒₘ), 79.7 (C_{propargyl}), 72.7 (C_{propargyl}), 60.8 (d, ²J_{C,P}=6.0 Hz, 2OCH₂), 48.9 (N7-CH), 42.2 (N3-CH₂), 38.2 (CH₂), 30.4 (N1-CH₂), 28.2 (d, ³J_{C,P}=15.9 Hz, CH₂), 27.6 (CH₂), 24.1 (d, ¹J_{C,P}=138.6 Hz, PCH₂), 21.2 (N7-CH(CH₃)₂), 19.3 (d, ²J_{C,P}=4.2 Hz, CH₂), 16.3 (d, ³J_{C,P}=5.5 Hz, 2OCH₂CH₃). ³¹P NMR (243 MHz, DMSO-d₆) δ [ppm]=32.8. LC-MS: positive mode [m/z]=655.40 [M+H]⁺. Purity: 87%.

### Synthesis of Diethyl (4-(7-isopropyl-8-(2-methylphenethyl)-2,6-dioxo-1-(prop-2-yn-1-yl)-1,2,6,7-tetrahydro-3H-purin-3-yl)butyl)phosphonate (53c

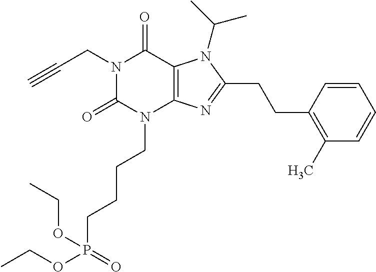 [SMILES: C#CCn1c(=O)c2c(nc(CCc3ccccc3C)n2C(C)C)n(CCCCP(=O)(OCC)OCC)c1=O]

Yield: 50%, colorless liquid. H NMR (500 MHz, DMSO-d₆) δ [ppm]=7.14 (p, J=3.9 Hz, 2H, 2Hₐᵣₒₘ), 7.11-7.07 (m, 2H, 2Hₐᵣₒₘ), 4.72-4.62 (m, 1H, N7-CH), 4.61 (d, J=2.4 Hz, 2H, N1-CH₂), 4.01 (t, J=6.9 Hz, 2H, N3-CH₂), 3.99-3.90 (m, 4H, 2OCH₂), 3.11 (t, J=7.5 Hz, 2H, CH₂), 3.05 (t, J=2.4 Hz, 1H, H_{propargyl}), 3.01 (t, J=7.5 Hz, 2H, CH₂), 2.28 (s, 3H, CH₃), 1.84-1.72 (m, 4H, 2CH₂), 1.54-1.44 (m, 2H, CH₂), 1.40 (d, J=6.7 Hz, 6H, N7-CH(CH₃)₂), 1.19 (t, J=7.1 Hz, 6H, 2OCH₂CH₃). ¹³C NMR (126 MHz, DMSO-d₆) δ [ppm]=153.4 (Cₓₐₙₜₕᵢₙₑ), 152.3 (Cₓₐₙₜₕᵢₙₑ), 149.7 (Cₓₐₙₜₕᵢₙₑ), 148.8 (Cₓₐₙₜₕᵢₙₑ), 138.4 (Cₐᵣₒₘ), 135.6 (Cₐᵣₒₘ), 130.0 (Cₐᵣₒₘ), 128.8 (Cₐᵣₒₘ), 126.3 (Cₐᵣₒₘ), 125.9 (Cₐᵣₒₘ), 105.5 (C5), 79.7 (C_{propargyl}), 72.6 (C_{propargyl}), 60.7 (d, ²J_{C,P}=6.3 Hz, 2OCH₂), 48.8 (N7-CH), 42.1 (N3-CH₂), 30.7 (CH₂), 30.4 (N1-CH₂), 28.1 (d, ³J_{C,P}=15.8 Hz, CH₂), 27.6 (CH₂), 24.1 (d, ¹J_{C,P}=138.6 Hz, PCH₂), 21.1 (N7-CH(CH₃)₂), 19.3 (d, ²J_{C,P}=4.9 Hz, CH₂), 18.8 (CH₃), 16.2 (d, ³J_{C,P}=5.7 Hz, 2OCH₂CH₃). ³¹P NMR (202 MHz, DMSO-d₆) δ [ppm]=32.7. LC-MS: positive mode [m/z]=543.40 [M+H]⁺. Purity: 94%.

### Synthesis of Diethyl (4-(8-(2-bromophenethyl)-7-(sec-butyl)-2,6-dioxo-1-(prop-2-yn-1-yl)-1,2,6,7-tetrahydro-3H-purin-3-yl)butyl)phosphonate (54h

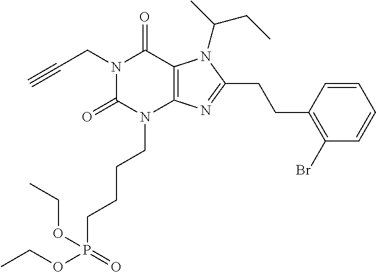 [SMILES: C#CCn1c(=O)c2c(nc(CCc3ccccc3Br)n2C(C)CC)n(CCCCP(=O)(OCC)OCC)c1=O]

Yield: 41%, colorless liquid. H NMR (500 MHz, DMSO-d₆) δ [ppm]=7.60 (dd, J=7.9, 1.4 Hz, 1H, Hₐᵣₒₘ), 7.36 (dt, J=7.6, 1.6 Hz, 1H, Hₐᵣₒₘ), 7.31 (tt, J=7.5, 1.4 Hz, 1H, Hₐᵣₒₘ), 7.17 (tt, J=7.6, 1.6 Hz, 1H, Hₐᵣₒₘ), 4.60 (d, J=2.1 Hz, 2H, N1-CH₂), 4.01 (t, J=6.9 Hz, 2H, N3-CH₂), 3.99-3.90 (m, 4H, 2OCH₂), 3.19-3.12 (m, 4H, 2CH₂), 3.05 (t, J=2.1 Hz, 1H, H_{propargyl}), 2.02-1.90 (m, 1H, N7-CH), 1.87-1.71 (m, 6H, 2CH₂ and N7-CHCH₂), 1.49 (dq, J=14.9, 8.7, 6.7 Hz, 2H, CH₂), 1.43 (d, J=6.9 Hz, 3H, N7-CHCH₃), 1.19 (td, J=7.1, 1.4 Hz, 6H, 2OCH₂CH₃), 0.67 (td, J=7.5, 1.4 Hz, 3H, N7-CHCH₂CH₃). ¹³C NMR (126 MHz, DMSO-d₆) δ [ppm]=153.3 (Cₓₐₙₜₕᵢₙₑ), 152.4 (Cₓₐₙₜₕᵢₙₑ), 149.7 (Cₓₐₙₜₕᵢₙₑ), 148.8 (Cₓₐₙₜₕᵢₙₑ), 139.2 (Cₐᵣₒₘ), 132.5 (Cₐᵣₒₘ), 131.0 (Cₐᵣₒₘ), 128.5 (Cₐᵣₒₘ), 127.9 (Cₐᵣₒₘ), 123.7 (Cₐᵣₒₘ), 105.5 (C5), 79.7 (C_{propargyl}), 72.6 (C_{propargyl}), 60.7 (d, ²J_{C,P}=6.2 Hz, 2OCH₂), 54.7 (N7-CH), 42.2 (N3-CH₂), 33.5 (CH₂), 30.4 (N1-CH₂), 28.1 (d, ³J_{C,P}=15.8 Hz, CH₂), 27.6 (CH₂), 27.2 (CH₂), 24.1 (d, ¹J_{C,P}=138.7 Hz, PCH₂), 19.4 (N7-CHCH₃), 19.3 (d, ²J_{C,P}=4.9 Hz, CH₂), 16.2 (d, ³J_{C,P}=5.7 Hz, 2OCH₂CH₃), 10.7 (N7-CHCH₂CH₃). ³¹P NMR (202 MHz, DMSO-d₆) δ [ppm]=32.7. LC-MS: positive mode [m/z]=621.3 [M+H]⁺. Purity: 99%.

### Synthesis of Diethyl (5-bromopentyl)phosphonate (82

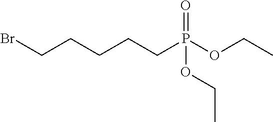

1,5-Dibromopentane (3.4 mL, 26.0 mmol, 1.0 equiv) in triethylphosphite (P(OEt)₃, 0.3 equiv) was stirred for 30 minutes at 155° C. After cooling to rt, excess of 1,5-dibromopentane and P(OEt)₃ were removed under reduced pressure at 70° C. The oily colorless residue was purified by flash column chromatography on silica gel 60 (eluent:dichloromethane/methanol, 97:3) to yield 82 (1.65 g, 5.7 mmol, 22%) as a light-yellow liquid. ¹H NMR (600 MHz, DMSO-d₆) δ [ppm]=4.01-3.93 (m, 4H, 2OCH₂), 3.52 (t, J=6.7 Hz, 2H, BrCH₂), 1.80 (p, J=6.8 Hz, 2H, CH₂), 1.75-1.66 (m, 2H, CH₂), 1.53-1.42 (m, 4H, 2CH₂), 1.22 (t, J=7.0 Hz, 6H, 2OCH₂CH₃). ¹³C NMR (151 MHz, DMSO-d₆) δ [ppm]=60.7 (d, ²J_{C,P}=6.3 Hz, 2OCH₂), 34.9 (BrCH₂), 31.7 (CH₂), 28.3 (d, ³J_{C,P}=15.7 Hz, CH₂), 24.3 (d, ¹J_{C,P}=138.5 Hz, PCH₂), 21.3 (d, ²J_{C,P}=5.6 Hz, CH₂), 16.3 (d, ³J_{C,P}=5.4 Hz, 2OCH₂CH₃). ³¹P NMR (243 MHz, DMSO-d₆) δ [ppm]=32.9. LC-MS: positive mode [m/z]=286.8 [M+H]⁺.

### Synthesis of Diethyl (6-bromohexyl)phosphonate (83

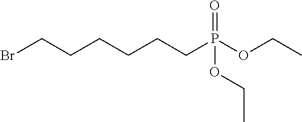

1,6-Dibromohexane (3.7 mL, 24.6 mmol, 1.0 equiv) in triethylphosphite (P(OEt)₃, 0.3 equiv) was stirred for 30 minutes at 155° C. After cooling to rt, excess of 1,6-dibromohexane and P(OEt)₃ were removed under reduced pressure at 70° C. The oily colorless residue was purified by flash column chromatography on silica gel 60 (eluent:dichloromethane/methanol, 95:5) to yield 83 (1.89 g, 6.3 mmol, 25%) as a light-yellow liquid. ¹H NMR (500 MHz, DMSO-d₆) δ [ppm]=4.04-3.86 (m, 4H, 2OCH₂), 3.52 (t, J=6.7 Hz, 2H, BrCH₂), 1.78 (p, J=6.7 Hz, 2H, CH₂), 1.69 (dt, J=18.1, 7.9 Hz, 2H, CH₂), 1.46 (ddt, J=11.9, 8.3, 5.4 Hz, 2H, CH₂), 1.38 (h, J=4.4, 3.7 Hz, 4H, 2CH₂), 1.22 (t, J=7.1 Hz, 6H, 2OCH₂CH₃). ¹³C NMR (126 MHz, DMSO-d₆) δ [ppm]=60.7 (d, ²J_{C,P}=6.3 Hz, 2OCH₂), 35.0 (BrCH₂), 32.0 (CH₂), 28.7 (d, ³J_{C,P}=15.8 Hz, CH₂), 26.9 (CH₂), 24.4 (d, ¹J_{C,P}=138.6 Hz, PCH₂), 21.8 (d, ²J_{C,P}=5.1 Hz, CH₂), 16.2 (d, ³J_{C,P}=5.7 Hz, 2OCH₂CH₃). ³¹P NMR (202 MHz, DMSO-d₆) δ [ppm]=33.0. LC-MS: positive mode [m/z]=300.8 [M+H]⁺.

### Synthesis of Diethyl (5-iodopentyl)phosphonate (84

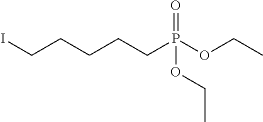

For the Finkelstein reaction, 82 (1.65 g, 5.7 mmol, 1.0 equiv) and NaI (1.2 equiv) was dissolved in acetone (0.1M) and stirred for 1 h at 65° C. The resulting precipitate was filtered off, washed with acetone and the volume of the filtrate was reduced in vacuo. The remaining residue was purified by flash column chromatography on silica gel 60 (eluent:dichloromethane/methanol, 98:2) to yield 84 (1.74 g, 5.2 mmol, 91%) as colorless liquid. ¹H NMR (600 MHz, DMSO-d₆) δ [ppm]=4.01-3.94 (m, 4H, 2OCH₂), 3.28-3.23 (m, 2H, ICH₂), 1.76 (p, J=7.0 Hz, 2H, CH₂), 1.70 (m, 2H, CH₂), 1.48 (m, 2H, CH₂), 1.45-1.38 (m, 2H, CH₂), 1.22 (t, J=7.1 Hz, 6H, 2OCH₂CH₃). ¹³C NMR (151 MHz, DMSO-d₆) δ [ppm]=60.7 (d, ²J_{C,P}=6.7 Hz, 2OCH₂), 32.3 (CH₂), 30.6 (d, ³J_{C,P}=15.1 Hz, CH₂), 24.3 (d, ¹J_{C,P}=138.5 Hz, PCH₂), 21.0 (d, ²J_{C,P}=5.5 Hz, CH₂), 16.3 (d, ³J_{C,P}=6.3 Hz, 2OCH₂CH₃), 8.6 (ICH₂). ³¹P NMR (243 MHz, DMSO-d₆) δ [ppm]=32.9. LC-MS: positive mode [m/z]=334.8 [M+H]⁺.

### Synthesis of Diethyl (6-iodohexyl)phosphonate (85

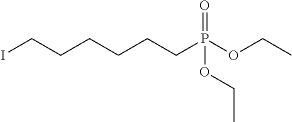

For the Finkelstein reaction, 83 (1.88 g, 6.2 mmol, 1.0 equiv) and NaI (1.2 equiv) was dissolved in acetone (0.1M) and stirred for 1 h at 65° C. The resulting precipitate was filtered off, washed with acetone and the volume of the filtrate was reduced in vacuo. The remaining residue was purified by flash column chromatography on silica gel 60 (eluent:dichloromethane/methanol, 98:2) to yield 85 (2.08 g, 5.9 mmol, 96%) as colorless liquid. ¹H NMR (600 MHz, DMSO-d₆) δ [ppm]=4.01-3.93 (m, 4H, 2OCH₂), 3.27 (t, J=6.9 Hz, 2H, ICH₂), 1.79-1.63 (m, 4H, 2CH₂), 1.50-1.41 (m, 2H, CH₂), 1.35 (tt, J=7.3, 3.5 Hz, 4H, 2CH₂), 1.22 (t, J=7.0 Hz, 6H, 2OCH₂CH₃). ¹³C NMR (151 MHz, DMSO-d₆) δ [ppm]=60.7 (d, ²J_{C,P}=6.3 Hz, 2OCH₂), 32.6 (CH₂), 29.3 (CH₂), 28.5 (d, ³J_{C,P}=15.2 Hz, CH₂), 24.4 (d, ¹J_{C,P}=137.6 Hz, PCH₂), 21.8 (d, ²J_{C,P}=5.1 Hz, CH₂), 16.3 (d, J_{C,P}=5.5 Hz, 2OCH₂CH₃), 8.8 (ICH₂). ³¹P NMR (243 MHz, DMSO-d₆) δ [ppm]=33.0. LC-MS: positive mode [m/z]=348.7 [M+H]⁺.

### Synthesis of tert-Butyl (2-bromoethyl)carbamate (86

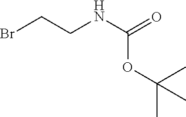

To a suspension of 2-bromoethylamine hydrobromide (122 mg, 600 μmol, 1.0 equiv) in THF, di-tert-butyl dicarbonate (Boc₂O, 158 mg, 720 umol, 1.2 equiv) was added. The mixture was cooled to 0° C. and DIPEA added dropwise. After stirring for 3 hours at room temperature, the solvent was removed in vacuo, the residue dissolved in ethyl acetate, washed with an aqueous solution of K₂CO₃ (10%) and the layers were separated. The aqueous phase was extracted with ethyl acetate (3×) and the combined organic extracts washed with brine, dried over MgSO₄ and the solvent removed in vacuo. The crude product was purified by flash column chromatography on silica gel 60 (eluent:cyclohexane/ethyl acetate, 95:5) yielding 86 (87 mg, 388 μmol, 65%) as a light-yellow liquid. ¹H NMR (600 MHz, DMSO-d₆) δ [ppm]=7.08 (t, J=5.6 Hz, 1H, NH), 3.42 (t, J=6.6 Hz, 2H, CH₂), 3.29 (t, J=6.4 Hz, 2H, CH₂), 1.38 (s, 9H, C(CH₃)₃). ¹³C NMR (151 MHz, DMSO-d₆) δ [ppm]=155.4 (CO), 78.0 (C(CH₃)₃), 41.9 (CH₂), 32.3 (CH₂), 28.1 (C(CH₃)₃). LC-MS: positive mode [m/z]=233.8 [M+H]⁺.

The potency of the xanthine derivatives was determined at the human wild-type MRGPRX4 and, in addition, at its rare variant containing a single point mutation, 83S and 83L, in β-arrestin recruitment as well as calcium mobilization assays (Tables 1-4). For β-arrestin recruitment assays, recombinant Chinese hamster ovary (CHO) cells expressing the human MRGPRX4 were used (DiscoverX/Eurofins) which allowed to measure β-galactosidase complementation upon β-arrestin recruitment. For calcium mobilization assays, recombinant astrocytes expressing MRGPX4-83L, or LN229 glioblastoma cells natively expressing the MRGPRX4-83S, were used.

The assays were performed as previously described for other rhodopsin-like GPCRs (Müller, C.E.; 8-Benzamidochromen-4-one-2-carboxylic acids: potent and selective agonists for the orphan G protein-coupled receptor GPR35. J. Med. Chem. 2013, 56:5182-97; Müller, C.E. Molecular Recognition of Agonists and Antagonists by the Nucleotide-Activated G Protein-Coupled P2Y₂ Receptor. J. Med. Chem. 2017, 60:8425-8440).

TABLE 1
Potency of selected compounds in activating the human MRGPRX4-83L

| | β-arrestin assay | | | | | |
|---|---|---|---|---|---|---|
| | (CHO β-arrestin ARMS1-ProLink ™ 2 | | | Calcium assay | | |
| | human MRGPRX4 83L) | | | (human MRGPRX4 83L) | | |
| | EC₅₀ ± SEM | | IC₅₀ ± SEM | EC₅₀ ± SEM | | IC₅₀ ± SEM |
| | (μM)^{a} | Eₘₐₓ^{b} (%) | (μM)^{a,c} | (μM)^{a} | Eₘₐₓ^{d} (%) | (μM)^{a,e} |
| A-1) | >10 | n.d. | >10 | >10 | n.d. | >10 |
| B-1) | >10 | n.d. | >10 | 3.25 ± 0.61 | 110 | n.d. |
| B-2) | 5.71 ± 1.23 | 91 | n.d. | 0.435 ± 0.078 | 102 | n.d. |
| B-3) | 7.94 ± 1.45 | 89 | n.d. | 0.601 ± 0.103 | 110 | n.d. |
| B-4) | 2.14 ± 0.85 | 103 | n.d. | 0.0808 ± 0.0168 | 100 | n.d. |
| B-5) | 15.2 ± 0.52 | 99 | n.d. | 0.492 ± 0.038 | 91 | n.d. |
| B-6) | 11.9 ± 1.8 | 114 | n.d. | 0.267 ± 0.066 | 99 | n.d. |
| B-7) | 3.12 ± 1.20 | 103 | n.d. | 0.397 ± 0.032 | 108 | n.d. |
| B-8) | 0.243 ± 0.045 | 98 | n.d. | 0.0227 ± 0.0072 | 114 | n.d. |
| B-9) | 1.26 ± 0.27 | 107 | n.d. | 0.0552 ± 0.00170 | 107 | n.d. |
| B-10) | 14.5 ± 2.4 | 100 | n.d. | 0.425 ± 0.187 | 113 | n.d. |
| B-11) | >10 | n.d. | 50.5 ± 1.4 | 28.0 ± 3.3 | 100 | n.d. |
| B-12) | >10 | n.d. | >10 (−27 ± 9) | 3.80 ± 0.19 | 104 | n.d. |
| B-13) | 48.3 ± 14.1 | 96 | n.d. | 1.60 ± 0.46 | 94 | n.d. |
| B-14) | >10 | n.d. | >10 (3 ± 15) | 9.45 ± 3.23 | 99 | n.d. |
| B-15) | 6.28 ± 1.38 | 141 | n.d. | 0.333 ± 0.007 | 113 | n.d. |
| B-16) | 0.370 ± 0.050 | 100 | n.d. | 0.0324 ± 0.0056 | 100 | n.d. |
| B-17) | 0.169 ± 0.060 | 114 | n.d. | 0.00223 ± 0.00013 | 109 | n.d. |
| B-18) | 0.0935 ± 0.0054 | 132 | n.d. | 0.00635 ± 0.00167 | 112 | n.d. |
| B-19) | 0.0259 ± 0.0079 | 144 | n.d. | 0.00426 ± 0.00056 | 115 | n.d. |
| B-20) | 0.0329 ± 0.0075 | 124 | n.d. | 0.00186 ± 0.00045 | 103 | n.d. |
| B-21) | 0.0898 ± 0.0209 | 163 | n.d. | 0.00202 ± 0.00062 | 109 | n.d. |
| B-22) | 0.208 ± 0.049 | 176 | n.d. | 0.0178 ± 0.0062 | 119 | n.d. |
| B-23) | 0.0599 ± 0.0128 | 164 | n.d. | 0.00503 ± 0.00159 | 103 | n.d. |
| C-1) | >10 | n.d. | >10 | >10 | n.d. | >10 |
| C-2) | >10 | n.d. | >10 | >10 | n.d. | >10 |
| C-3) | >10 | n.d. | >10 | >10 | n.d. | >10 |
| C-4) | >10 | n.d. | >10 | >10 | n.d. | >10 |
| C-5) | >10 | n.d. | >10 | >10 | n.d. | >10 |
| C-6) | >10 | n.d. | >10 | >10 | n.d. | >10 |
| C-7) | >10 | n.d. | >10 | >10 | n.d. | >10 |
| C-8) | >10 | n.d. | >10 | >10 | n.d. | >10 |
| C-9) | >10 | n.d. | >10 | >10 | n.d. | >10 |
| C-10) | >10 | n.d. | >10 | >10 | n.d. | >10 |
| D-1) | 0.395 ± 0.126 | 108 | n.d. | 0.0555 ± 0.0090 | 115 | n.d. |
| D-2) | 0.113 ± 0.033 | 122 | n.d. | 3.32 ± 0.61 | 103 | n.d. |
| D-3) | 0.407 ± 0.71 | 112 | n.d. | 0.0363 ± 0.0018 | 114 | n.d. |
| E-1) | >10 | n.d. | >10 | >10 | n.d. | >10 |
| E-2) | >10 | n.d. | >10 | >10 | n.d. | >10 |
| E-3) | >10 | n.d. | >10 | >10 | n.d. | >10 |
| F-1) | >10 | n.d. | >10 | >10 | n.d. | >10 |
| G-1) | >10 | n.d. | >10 | >10 | n.d. | >10 |
| G-2) | >10 | n.d. | >10 | >10 | n.d. | >10 |
| H-1) | 13.0 ± 4.1 | 97 | n.d. | 0.574 ± 0.179 | 107 | n.d. |
| H-2) | >10 | n.d. | >10 | 2.50 ± 0.73 | 113 | n.d. |
| H-3) | >10 | n.d. | >10 | >10 | n.d. | >10 |
| H-4) | n.d. | n.d. | n.d. | n.d. | n.d. | n.d. |
| H-5) | n.d. | n.d. | n.d. | n.d. | n.d. | n.d. |
| H-6) | n.d. | n.d. | n.d. | n.d. | n.d. | n.d. |
| H-7) | n.d. | n.d. | n.d. | n.d. | n.d. | n.d. |
| H-8) | n.d. | n.d. | n.d. | n.d. | n.d. | n.d. |
| H-9) | n.d. | n.d. | n.d. | n.d. | n.d. | n.d. |
| H-10) | n.d. | n.d. | n.d. | n.d. | n.d. | n.d. |
| J-1) | >10 | n.d. | inhibition | >10 | n.d. | inhibition |
| J-2) | >10 | n.d. | >10 | >50 | n.d. | >50 |
| J-3) | >10 | n.d. | >10 | >10 | n.d. | >10 |

^{a}The results represent means of 3-4 independent experiments.
^{b}Efficacies are based on the maximal effect of 100 nM B-16 (set as 100%).
^{c}Inhibition of the effect of 2.6 μM B-16 (corresponding to its EC₈₀ value)
^{d}Efficacy based on the maximal effect of 1 μM B-16 (set as 100%).
^{e}Inhibition of the effect of 100 nM B-16 (corresponding to its EC₈₀ value)

TABLE 2
Effects of compounds on MRGPRX4-83S determined in β-arrestin recruitment and
calcium mobilization assays.

| | | | | Calcium assay | | |
|---|---|---|---|---|---|---|
| | β-arrestin assay (CHO β-arrestin | | | (human glioblastoma LN229 cells | | |
| | human MRGPRX4-83S) | | | expressing the MRGPRX4-83S) | | |
| | EC₅₀ ± SEM | | IC₅₀ ± SEM | EC₅₀ ± SEM | | |
| | (μM)^{a} | Eₘₐₓ^{b} (%) | (μM)^{a,c} | (μM)^{a} | Eₘₐₓ (%)^{d} | IC₅₀ (%)^{a,e} |
| A-1) | n.d. | | n.d. | >10 | | >10 |
| B-1) | 0.399 ± 0.115 | 84 | n.d. | 0.134 ± 0.017 | 92 | n.d. |
| B-2) | 0.0320 ± 0.0110 | 98 | n.d. | 0.0112 ± 0.0013 | 99 | n.d. |
| B-3) | 0.0339 ± 0.0051 | 93 | n.d. | 0.0631 ± 0.0186 | 118 | n.d. |
| B-4) | 0.00324 ± 0.00116 | 110 | n.d. | 0.00469 ± 0.00158 | 109 | n.d. |
| B-5) | 0.0343 ± 0.0076 | 103 | n.d. | 0.0181 ± 0.0015 | 96 | n.d. |
| B-6) | 0.0205 ± 0.0024 | 100 | n.d. | 0.0382 ± 0.0109 | 150 | n.d. |
| B-7) | 0.00894 ± 0.00138 | 101 | n.d. | 0.0103 ± 0.0013 | 132 | n.d. |
| B-8) | 0.00315 ± 0.00082 | 143 | n.d. | n.d. | n.d. | n.d. |
| B-9) | 0.00345 ± 0.00028 | 116 | n.d. | n.d. | n.d. | n.d. |
| B-10) | 0.0714 ± 0.0193 | 87 | n.d. | 0.0306 ± 0.0031 | 82 | n.d. |
| B-11) | n.d. | n.d. | 9.72 ± 1.05 | n.d. | n.d. | n.d. |
| B-12) | 1.19 ± 0.05 | 102 | n.d. | 0.214 ± 0.051 | 94 | n.d. |
| B-13) | 0.0689 ± 0.0170 | 93 | n.d. | 0.0599 ± 0.0107 | 91 | n.d. |
| B-14) | 0.735 ± 0.131 | 81 | n.d. | 0.500 ± 0.113 | 74 | n.d. |
| B-15) | 0.0300 ± 0.0058 | 140 | n.d. | 0.0192 ± 0.0035 | 132 | n.d. |
| B-16) | 0.00228 ± 0.00016 | 100 | n.d. | 0.00317 ± 0.00060 | 137 | n.d. |
| B-18) | 0.000296 ± 0.000033 | 128 | n.d. | 0.000501 ± 0.000076 | 108 | n.d. |
| C-1) | >10 | n.d. | n.d. | n.d. | n.d. | n.d. |
| C-2) | >10 | n.d. | n.d. | n.d. | n.d. | n.d. |
| C-3) | >10 | n.d. | n.d. | n.d. | n.d. | n.d. |
| C-4) | >10 | n.d. | n.d. | n.d. | n.d. | n.d. |
| C-5) | >10 | n.d. | n.d. | n.d. | n.d. | n.d. |
| C-6) | >10 | n.d. | n.d. | n.d. | n.d. | n.d. |
| C-7) | >10 | n.d. | n.d. | n.d. | n.d. | n.d. |
| C-8) | >10 | n.d. | n.d. | n.d. | n.d. | n.d. |
| C-9) | >10 | n.d. | n.d. | n.d. | n.d. | n.d. |
| C-10) | >10 | n.d. | n.d. | n.d. | n.d. | n.d. |
| D-1) | 0.114 ± 0.023 | 120 | n.d. | 0.134 ± 0.012 | 105 | n.d. |
| D-2) | 0.000493 ± 0.000104 | 141 | n.d. | n.d | n.d | n.d |
| D-3) | 0.00580 ± 0.00141 | 110 | n.d. | 0.00265 ± 0.00072 | 111 | n.d. |
| E-1) | >10 | n.d. | n.d. | n.d. | n.d. | n.d. |
| E-2) | >10 | n.d. | n.d. | n.d. | n.d. | n.d. |
| E-3) | >10 | n.d. | n.d. | n.d. | n.d. | n.d. |
| F-1) | >10 | n.d. | n.d. | n.d | n.d. | n.d. |
| G-1) | >10 | n.d. | n.d. | n.d | n.d. | n.d. |
| G-2) | >10 | n.d. | n.d. | n.d | n.d. | n.d. |
| H-1) | 0.374 ± 0.111 | 86 | n.d. | 0.238 ± 0.011 | 98 | n.d. |
| H-2) | 0.674 ± 0.050 | n.d. | n.d. | 0.801 ± 0.233 | 88 | n.d. |
| H-3) | >10 | n.d. | n.d. | >10 | n.d. | >10 |
| J-1) | 0.200 ± 0.059 | 65 | n.d. | 0.0116 ± 0.0013 | | n.d. |
| J-2) | 6.36 ± 1.63 | 76 | n.d. | >10 | | 4.45 ± 0.68 |
| J-3) | >10 | | >10 | >10 | | >10 |

^{a}The results represent means of 3-4 independent experiments.
^{b}Efficacy based on 100 nM B-16 (set as 100%)
^{c}Inhibition of the effect of 6 nM B-16 (corresponding to its EC₈₀ value) or of 500 nM H-1 (corresponding to its EC₈₀ value).
^{d}Efficacy based on 30 μM H-1 (set as 100%)
^{e}Inhibition of 1 μM H-1 (corresponding to its EC₈₀).

TABLE 3
Potency of selected compounds in activating the human MRGPRX4-83S

| | β-arrestin assays (CHO β-arrestin | | | Calcium assays (human glioblastoma LN229 cells | | |
|---|---|---|---|---|---|---|
| | human MRGPRX4-83S) | | | expressing MPGPRX4-83S | | |
| | EC₅₀ ± SEM | | IC₅₀ ± SEM | EC₅₀ ± SEM | | IC₅₀ ± SEM |
| | (μM)^{a} | Eₘₐₓ^{b} (%) | (μM)^{a,c} | (μM)^{a} | Eₘₐₓ^{d} (%) | (μM)^{a,e} |
| B-24 | n.d. | n.d. | n.d. | n.d. | n.d. | n.d. |
| B-25 | n.d. | n.d. | n.d. | n.d. | n.d. | n.d. |
| B-26 | n.d. | n.d. | n.d. | n.d. | n.d. | n.d. |
| B-27 | 0.0367 ± 0.0028 | 68 | n.d. | n.d. | n.d. | n.d. |
| B-28 | 0.00148 ± 0.00018 | 78 | n.d. | 0.000284 ± 0.000074 | 92 | n.d. |
| B-29 | n.d. | n.d. | n.d. | n.d. | n.d. | n.d. |
| B-30 | n.d. | n.d. | n.d. | n.d. | n.d. | n.d. |
| B-31 | 0.0000937 ± 0.0000523 | 89 | n.d. | n.d. | n.d. | n.d. |
| B-32 | 0.0000424 ± 0.0000150 | 97 | n.d. | 0.000441 ± 0.000157 | 111 | n.d. |
| B-33 | 0.0000487 ± 0.0000201 | 113 | n.d. | 0.000543 ± 0.000186 | 113 | n.d. |
| B-34 | 0.000146 ± 0.000033 | 110 | n.d. | 0.000363 ± 0.000109 | 104 | n.d. |
| B-35 | n.d. | n.d. | n.d. | n.d. | n.d. | n.d. |
| B-36 | 0.00428 ± 0.00083 | 84 | n.d. | 0.000787 ± 0.000164 | 112 | n.d. |
| B-37 | n.d. | n.d. | n.d. | n.d. | n.d. | n.d. |
| B-38 | 0.0134 ± 0.0008 | 79 | n.d. | 0.00581 ± 0.00058 | 6 | 0.00523 ± 0.00220 |
| B-39 | 0.000773 ± 0.000106 | 76 | n.d. | 0.143 ± 0.066 | 23 | 0.0161 ± 0.0084 |
| B-40 | 0.0183 ± 0.0061 | 77 | n.d. | 0.0237 ± 0.0007 | 8 | 0.0130 ± 0.0014 |
| B-41 | 0.00367 ± 0.00121 | 96 | n.d. | 0.0514 ± 0.0085 | 57 | n.d. |
| B-42 | 0.0125 ± 0.0013 | 92 | n.d. | n.d. | n.d. | n.d. |
| B-43 | 0.0107 ± 0.0071 | 53 | n.d. | >10 (2%) | n.d. | 0.0104 ± 0.002.4 |
| B-44 | 0.00212 ± 0.00085 | 43 | n.d. | >10 (2%) | n.d. | 0.0000873 ± 0.0000120 |
| B-45 | 0.000298 ± 0.000050 | 93 | n.d. | 0.00032 ± 0.000087 | 97 | n.d. |
| B-46 | 0.0000750 ± 0.0000184 | 91 | n.d. | 0.00079 ± 0.000262 | 109 | n.d. |
| B-47 | n.d. | n.d. | n.d. | n.d. | n.d. | n.d. |
| B-48 | 0.0000705 ± 0.0000198 | 102 | n.d. | 0.000984 ± 0.000054 | 154 | n.d. |
| B-49 | 0.0000751 ± 0.0000229 | 103 | n.d. | 0.000491 ± 0.000192 | 119 | n.d. |
| B-50 | 0.000301 ± 0.000021 | 84 | n.d. | 0.000095 ± 0.0000427 | 111 | n.d. |
| B-51 | 0.000932 ± 0.000177 | 99 | n.d. | 0.00148 ± 0.00040 | 107 | n.d. |
| B-52 | n.d. | n.d. | n.d. | n.d. | n.d. | n.d. |
| B-53 | n.d. | n.d. | n.d. | n.d. | n.d. | n.d. |
| B-54 | n.d. | n.d. | n.d. | n.d. | n.d. | n.d. |
| B-55 | n.d. | n.d. | n.d. | n.d. | n.d. | n.d. |
| B-56 | n.d. | n.d. | n.d. | n.d. | n.d. | n.d. |
| B-57 | n.d. | n.d. | n.d. | n.d. | n.d. | n.d. |
| B-58 | >100 (21%) | n.d. | n.d. | >10 (8%) | n.d. | 0.0317 ± 0.0191 |
| G-3 | >100 (41%) | n.d. | n.d. | >10 (0%) | n.d. | n.d. |

^{a}The results represent means of 3-4 independent experiments.
^{b}Efficacy based on 100 nM B-16 (set as 100%).
^{c}Inhibition of the effect of 6 nM B-16 (corresponding to its EC₈₀ value) or of 500 nM H-1 (corresponding to its EC₈₀ value).
^{d}Efficacy based on 30 μM H-1 (set as 100%).
^{e}Inhibition of 1 μM H-1 (corresponding to its EC₈₀).

TABLE 4
Potency of selected compounds in activating the human MRGPRX4 83L

| | β-arrestin assay (CHO β-arrestin | | | | | |
|---|---|---|---|---|---|---|
| | ARMS1-ProLink ™ 2 | | | Calcium assay | | |
| | human MRGPRX4-83L) | | | (human MRGPRX4-83L) | | |
| | EC₅₀ ± SEM | | IC₅₀ ± SEM | EC₅₀ ± SEM | | IC₅₀ ± SEM |
| | (μM)^{a} | Eₘₐₓ^{b} (%) | (μM)^{a,c} | (μM)^{a} | Eₘₐₓ^{d} (%) | (μM)^{a,e} |
| B-24 | 8.19 ± 1.81 | 98 | n.d. | 1.42 ± 0.29 | 100 | n.d. |
| B-25 | 0.381 ± 0.112 | 96 | n.d. | 0.0431 ± 0.0094 | 103 | n.d. |
| B-26 | 2.74 ± 0.58 | 70 | n.d. | 0.187 ± 0.039 | 79 | n.d. |
| B-27 | n.d. | n.d. | n.d. | n.d. | n.d. | n.d. |
| B-28 | 0.113 ± 0.039 | 118 | n.d. | 0.0397 ± 0.0096 | 62 | n.d. |
| B-29 | 2.44 ± 0.75 | 100 | n.d. | 0.134 ± 0.013 | 45 | n.d. |
| B-30 | 0.262 ± 0.097 | 96 | n.d. | 0.0155 ± 0.0125 | 97 | n.d. |
| B-31 | 0.0874 ± 0.0242 | 94 | n.d. | 0.000389 ± 0.000092 | 103 | n.d. |
| B-32 | n.d. | n.d. | n.d. | n.d. | n.d. | n.d. |
| B-33 | n.d. | n.d. | n.d. | n.d. | n.d. | n.d. |
| B-34 | 0.00902 ± 0.00149 | 95 | n.d. | 0.00200 ± 0.00094 | 97 | n.d. |
| B-35 | 0.521 ± 0.142 | 84 | n.d. | 0.306 ± 0.111 | 41 | n.d. |
| B-36 | 1.25 ± 0.41 | 95 | n.d. | 0.0252 ± 0.0030 | 109 | n.d. |
| B-37 | 0.169 ± 0.023 | 87 | n.d. | 0.0892 ± 0.0255 | 84 | n.d. |
| B-38 | 0.00144 ± 0.00032 | 92 | n.d. | 0.267 ± 0.153 | 9 | 0.949 ± 0.308 |
| B-39 | n.d. | n.d. | n.d. | n.d. | n.d. | n.d. |
| B-40 | n.d. | n.d. | n.d. | n.d. | n.d. | n.d. |
| B-41 | n.d. | n.d. | n.d. | n.d. | n.d. | n.d. |
| B-42 | n.d. | n.d. | n.d. | n.d. | n.d. | n.d. |
| B-43 | n.d. | n.d. | n.d. | n.d. | n.d. | n.d. |
| B-44 | n.d. | n.d | n.d. | n.d. | n.d. | n.d. |
| B-45 | 0.0851 ± 0.0279 | 99 | n.d. | 0.0127 ± 0.0460 | 101 | n.d. |
| B-46 | 0.0136 ± 0.0061 | 116 | n.d. | 0.000684 ± 0.000073 | 108 | n.d. |
| B-47 | 0.00685 ± 0.00718 | 97 | n.d. | 0.00116 ± 0.00026 | 118 | n.d. |
| B-48 | n.d. | n.d. | n.d. | n.d. | n.d. | n.d. |
| B-49 | n.d. | n.d. | n.d. | n.d. | n.d. | n.d. |
| B-50 | 0.0243 ± 0.0032 | 91 | n.d. | 0.00228 ± 0.00075 | 118 | n.d. |
| B-51 | 0.566 ± 0.106 | 93 | n.d. | 0.0798 ± 0.0367 | 102 | n.d. |
| B-52 | 1.89 ± 0.28 | 93 | n.d. | 0.604 ± 0.275 | 61 | n.d. |
| B-53 | 1.07 ± 0.35 | 94 | n.d. | 0.0155 ± 0.0010 | 113 | n.d. |
| B-54 | 0.534 ± 0.17 | 96 | n.d. | 0.0201 ± 0.0041 | 103 | n.d. |
| B-55 | 0.857 ± 0.22 | 98 | n.d. | 0.0578 ± 0.0189 | 94 | n.d. |
| B-56 | 0.576 ± 0.083 | 91 | n.d. | 0.0578 ± 0.0189 | 102 | n.d. |
| B-57 | 0.766 ± 0.357 | 97 | n.d. | 0.474 ± 0.152 | 98 | n.d. |
| B-58 | >10 (0%) | n.d. | n.d. | >10 (0%) | n.d. | n.d. |
| G-3 | n.d. | n.d. | n.d. | n.d. | n.d. | n.d. |

^{a}The results represent means of 3-4 independent experiments.
bEfficacies are based on the maximal effect of 100 nM B-16 (set as 100%).
^{c}Inhibition of the effect of 2.6 μM B-16 (corresponding to its EC₈₀ value).
^{d}Efficacy based on the maximal effect of 1 μM B-16 (set as 100%).
^{e}Inhibition of the effect of 100 nM B-16 (corresponding to its EC₈₀ value).

1: A compound according to general Formula 1 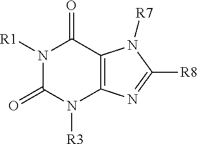
wherein
R1 represents —H, —C₁₋₁₀-alkyl, —C₁₋₁₀-alkyl-C₃₋₁₀-cycloalkyl, —C₁₋₁₀-alkyl-aryl, —C₁₋₁₀-alkyl-heteroaryl, —C₃₋₁₀-cycloalkyl, —C₁₋₁₀-alkyl-O-aryl, or —C₁₋₁₀-alkyl-O-heteroaryl;
R3 represents —C₁₋₁₀-alkyl-P(═O)(OC₁₋₁₀-alkyl)₂, —C₁₋₁₀-alkyl-P(═O)(OH)(OC₁₋₁₀-alkyl), —C₁₋₁₀-alkyl-S(═O)₂(OH), —C₁₋₁₀-alkyl-S(═O)₂(NH₂), —C₁₋₁₀-alkyl-C(═O)(OH), or —C₁₋₁₀-alkyl-P(═O)(OH)₂;
R7 represents —H, —C₁₋₁₀-alkyl, —C₁₋₁₀-alkyl-C₃₋₁₀-cycloalkyl, —C₁₋₁₀-alkyl-aryl, —C₁₋₁₀-alkyl-heteroaryl, —C₃₋₁₀-cycloalkyl, —C₁₋₁₀-alkyl-O-aryl, or —C₁₋₁₀-alkyl-O-heteroaryl; and
R8 represents —H, —C₁₋₁₀-alkyl, —C₁₋₁₀-alkyl-C₃₋₁₀-cycloalkyl, —C₁₋₁₀-alkyl-aryl, —C₁₋₁₀-alkyl-heteroaryl, —C₃₋₁₀-cycloalkyl, —C₁₋₁₀-alkyl-O-aryl, —C₃₋₆-cycloalkyl-aryl, —C₃₋₆-cycloalkyl-heteroaryl, or —C₁₋₁₀-alkyl-O-heteroaryl;
wherein in each case “C₁₋₁₀-alkyl” may be linear or branched, unless expressly stated otherwise saturated or unsaturated, unsubstituted or monosubstituted with a substituent selected from —C≡CH, —C(═O)OH, —C(═O)O—C₁₋₁₀-alkyl, —OH, —O—C₁₋₁₀-alkyl, —NH₂, —NH—C₁₋₁₀-alkyl, —N(C₁₋₁₀-alkyl)₂, —N₃, —F, —Cl, —Br, and —I;
wherein in each case “C₃₋₁₀-cycloalkyl” may be linear or branched, unless expressly stated otherwise saturated or unsaturated, unsubstituted or monosubstituted with a substituent selected from —C≡CH, —C(═O)OH, —C(═O)O—C₁₋₆-alkyl, —OH, —O—C₁₋₁₀-alkyl, —NH₂, —NH—C₁₋₁₀-alkyl, —N(C₁₋₁₀-alkyl)₂, —N₃, —F, —Cl, —Br, and —I;
wherein in each case “aryl” is a 6-14-membered aryl moiety which may be unsubstituted, mono- or disubstituted with a substituent independently selected from —C≡CH, —CH₃, —CH₂—CH₃, —CH(CH₃)₂, -phenyl, —CF₃, —CCl₃, —CBr₃, —Cl₃, —C(═O)OH, C(═O)O—C₁₋₁₀-alkyl, —OH, —O—C₁₋₁₀-alkyl, —NH₂, —NH—C₁₋₁₀-alkyl, —N(C₁₋₁₀-alkyl)₂, —N₃, —F, —Cl, —Br, and —I;
wherein in each case “heteroaryl” is a 5-14-membered heteroaryl moiety which may be unsubstituted, mono- or disubstituted with a substituent independently selected from —C≡CH, —CF₃, —CCl₃, —CBr₃, —Cl₃, —C(═O)OH, —C(═O)O—C₁₋₁₀-alkyl, —OH, —O—C₁₋₁₀-alkyl, —NH₂, —NH—C₁₋₁₀-alkyl, —N(C₁₋₁₀-alkyl)₂, —N₃, —F, —Cl, —Br, and —I;
or a physiologically acceptable salt thereof;
with the proviso that the compound is not a compound selected from the group consisting of compounds J-1 to J-3:

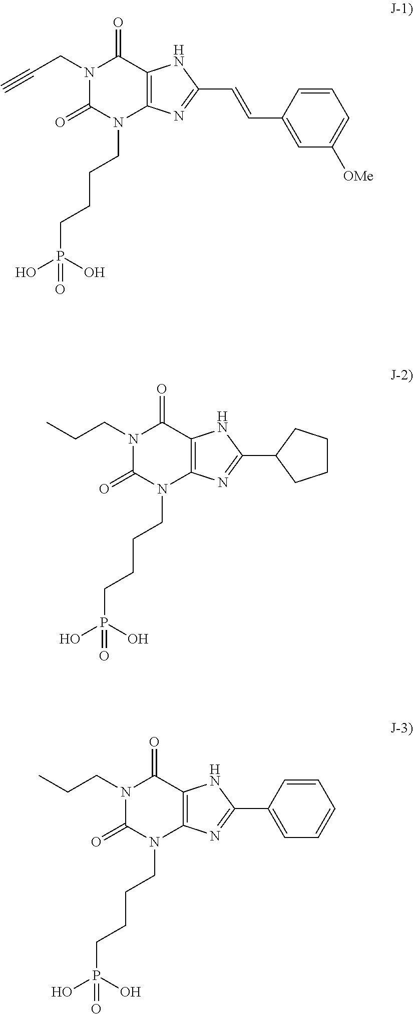 [SMILES: C#CCn1c(=O)c2[nH]c(/C=C/c3cccc(OC)c3)nc2n(CCCCP(=O)(O)O)c1=O ; CCCn1c(=O)c2[nH]c(C3CCCC3)nc2n(CCCCP(=O)(O)O)c1=O ; CCCn1c(=O)c2[nH]c(-c3ccccc3)nc2n(CCCCP(=O)(O)O)c1=O]

2: The compound according to claim 1, wherein in each case “aryl” is a 6-14-membered aryl moiety which may be unsubstituted, mono- or disubstituted with a substituent independently selected from —C≡CH, —CF₃, —CCl₃, —CBr₃, —Cl₃, —C(═O)OH, —C(═O)O—C₁₋₁₀-alkyl, —OH, —O—C₁₋₁₀-alkyl, —NH₂, —NH—C₁₋₁₀-alkyl, —N(C₁₋₁₀-alkyl)₂, —N₃, —F, —Cl, —Br, and —I.

3: The compound according to claim 1,
wherein
(i) R3 represents —C₁₋₁₀-alkyl-P(═O)(OH)₂ and R8 represents —C₃₋₆-cycloalkyl-aryl, —C₃₋₆-cycloalkyl-heteroaryl, —C₁₋₁₀-alkyl-aryl or —C₁₋₁₀-alkyl-heteroaryl (wherein in case of —C₂-alkyl-aryl, the alkyl-moiety is saturated); and
wherein in each case R1 and R7 independently from one another are selected from —H, —C₁₋₁₀-alkyl, —C₁₋₁₀-alkyl-C₃₋₁₀-cycloalkyl, —C₁₋₁₀-alkyl-aryl, and —C₁₋₁₀-alkyl-heteroaryl; or
wherein
(ii) R3 represents —C₁₋₁₀-alkyl-P(═O)(OC₁₋₁₀-alkyl)₂; or
R3 represents —C₁₋₁₀-alkyl-P(═O)(OH)(OC₁₋₁₀-alkyl); or
R3 represents —C₁₋₁₀-alkyl-S(═O)₂(OH); or
R3 represents —C₁₋₁₀-alkyl-S(═O)₂(NH₂); or
R3 represents —C₁₋₁₀-alkyl-C(═O)(OH); and wherein in each case R1, R7, and R8 independently from one another are selected from —H, —C₁₋₁₀-alkyl, —C₁₋₁₀-alkyl-C₃₋₁₀-cycloalkyl, —C₁₋₁₀-alkyl-aryl, —C₁₋₁₀-alkyl-heteroaryl, —C₃₋₁₀-cycloalkyl, —C₁₋₁₀-alkyl-O-aryl, and —C₁₋₁₀-alkyl-O-heteroaryl; or
wherein
(iii) R3 represents —C₁₋₁₀-alkyl-P(═O)(OC₁₋₁₀-alkyl)₂; or
R3 represents —C₁₋₁₀-alkyl-P(═O)(OH)(OC₁₋₁₀-alkyl); or
R3 represents —C₁₋₁₀-alkyl-S(═O)₂(OH); or
R3 represents —C₁₋₁₀-alkyl-S(═O)₂(NH₂); or
R3 represents —C₁₋₁₀-alkyl-C(═O)(OH); and
wherein in each case R1 and R7 independently from one another are selected from —H, —C₁₋₁₀-alkyl, —C₁₋₁₀-alkyl-C₃₋₁₀-cycloalkyl, —C₁₋₁₀-alkyl-aryl, —C₁₋₁₀-alkyl-heteroaryl, —C₃₋₁₀-cycloalkyl, —C₁₋₁₀-alkyl-O-aryl, and —C₁₋₁₀-alkyl-O-heteroaryl and R8 represents —C₃-6-cycloalkyl-aryl, —C₁₋₁₀-alkyl-aryl (wherein in case of —C₂-alkyl-aryl, the alkyl-moiety is saturated); or
wherein
(iv) R3 represents —C₁₋₁₀-alkyl-P(═O)(OC₁₋₁₀-alkyl)₂; or
R3 represents —C₁₋₁₀-alkyl-P(═O)(OH)(OC₁₋₁₀-alkyl); and
wherein in each case R1, R7, and R8 independently from one another are selected from —H, —C₁₋₁₀-alkyl, —C₁₋₁₀-alkyl-C₃₋₁₀-cycloalkyl, —C₁₋₁₀-alkyl-aryl, —C₁₋₁₀-alkyl-heteroaryl, —C₃₋₁₀-cycloalkyl, —C₁₋₁₀-alkyl-O-aryl, and —C₁₋₁₀-alkyl-O-heteroaryl.

4: The compound according to claim 1,
wherein
R1 represents —C₁₋₁₀-alkyl, optionally substituted with —C≡CH; or —C₁₋₁₀-alkyl-C₃₋₁₀-cycloalkyl;
preferably —C₁₋₆-alkyl, optionally substituted with —C≡CH; or —C₁₋₆-alkyl-C₃₋₆-cycloalkyl;
more preferably —CH₂CH₃, —CH₂CH₂CH₃, —CH₂C≡CH, or —CH₂-cyclobutyl; and/or
R3 represents —C₃₋₅-alkyl-P(═O)(OH)₂; —C₃₋₅-alkyl-O—P(═O)(OH)₂; —C₃₋₅-alkyl-P(═O)(OC₁₋₆-alkyl)₂; —C₃₋₅-alkyl-P(═O)(OH)(OC₁₋₆-alkyl); —C₃₋₅-alkyl-S(═O)₂(OH); —C₃₋₅-alkyl-S(═O)₂(NH₂); or —C₃₋₅-alkyl-C(═O)(OH);
preferably —C₃₋₅-alkyl-P(═O)(OH)₂;
more preferably —C₄-alkyl-P(═O)(OH)₂; and/or
R7 represents —H; —C₁₋₁₀-alkyl, optionally substituted with —OH; —C₁₋₁₀-alkyl-C₃₋₁₀-cycloalkyl; or —C₁₋₁₀-alkyl-aryl;
preferably —H; —C₁₋₆-alkyl, optionally substituted with —OH; —C₁₋₆-alkyl-C₃₋₆-cycloalkyl; or —C₁₋₆-alkyl-aryl;
more preferably —H, —CH₃, —CH₂CH₃, —CH₂CH₂—OH, —CH₂CH₂CH₃, —CH₂-cyclopropyl, or —CH₂-phenyl; and/or
R8 represents —C₃₋₁₀-cycloalkyl, —C₁₋₁₀-alkyl-C₃₋₁₀-cycloalkyl, -aryl, —C₁₋₁₀-alkyl-aryl, —C₃₋₆-cycloalkyl-aryl, or —C₁₋₁₀-alkyl-O-aryl;
preferably -phenyl, optionally substituted with —F, —Br, —Cl, —CH₃, —CF₃, —OCH₃; —CH₂-phenyl, optionally substituted with —F, —Br, —Cl, —CH₃, —CF₃, —OCH₃; —CH₂CH₂-phenyl, optionally substituted with one or two substituents independently of one another selected from —F, —Br, —Cl, —CH₃, —CF₃, —OCH₃; —CH═CH-phenyl, optionally substituted with —F, —Br, —Cl, —CH₃, —CF₃, —OCH₃; —CH₂—O-phenyl, optionally substituted with —F, —Br, —Cl, —CH₃, —CF₃, —OCH₃; -cyclopropyl-phenyl, optionally substituted with —F, —Br, —Cl, —CH₃, —CF₃, —OCH₃; or —C₃₋₆-cycloalkyl;
more preferably -phenyl, optionally substituted with —OCH₃; —CH₂-phenyl, optionally substituted with —Cl; —CH₂CH₂-phenyl, optionally substituted with one or two substituents independently of one another selected from —F, —Br, —Cl, —CH₃, —CF₃, —OCH₃; —CH═CH-phenyl, optionally substituted with —OCH₃; —CH₂—O-phenyl; -cyclopropyl-phenyl; or -cyclopentyl.

5: The compound according to claim 1,
wherein
(i) R3 represents —C₁₋₆-alkyl-P(═O)(OH)₂ and R8 represents —C₃₋₆-cycloalkyl-phenyl or —C₁₋₆-alkyl-phenyl (wherein in case of —C₂-alkyl-phenyl, the alkyl-moiety is saturated); and wherein in each case R1 and R7 independently from one another are selected from —H, —C₁₋₆-alkyl, —C₁₋₆-alkyl-C₃₋₆-cycloalkyl, —C₁₋₆-alkyl-aryl, and —C₁₋₆-alkyl-heteroaryl; or
wherein
(ii) R3 represents —C₁₋₆-alkyl-P(═O)(OC₁₋₆-alkyl)₂; or
R3 represents —C₁₋₆-alkyl-P(═O)(OH)(OC₁₋₆-alkyl); or
R3 represents —C₁₋₆-alkyl-S(═O)₂(OH); or
R3 represents —C₁₋₆-alkyl-S(═O)₂(NH₂); or
R3 represents —C₁₋₆-alkyl-C(═O)(OH); and
wherein in each case R1, R7, and R8 independently from one another are selected from —H, —C₁₋₆-alkyl, —C₁₋₆-alkyl-C₃₋₆-cycloalkyl, —C₁₋₆-alkyl-aryl, —C₁₋₆-alkyl-heteroaryl, —C₃₋₆-cycloalkyl, —C₁₋₆—O-aryl, and —C₁₋₆—O-heteroaryl;
wherein in each case “C₁₋₆-alkyl” may be linear or branched, unless expressly stated otherwise saturated or unsaturated, unsubstituted or monosubstituted with a substituent selected from —C≡CH, —C(═O)OH, —C(═O)O—C₁₋₁₀-alkyl, —OH, —O—C₁₋₁₀-alkyl, —NH₂, —NH—C₁₋₁₀-alkyl, —N(C₁₋₁₀-alkyl)₂, —N₃, —F, —Cl, —Br, and —I;
wherein in each case “C₃₋₆-cycloalkyl” may be linear or branched, unless expressly stated otherwise saturated or unsaturated, unsubstituted or monosubstituted with a substituent selected from —C≡CH, —C(═O)OH, —C(═O)O—C₁₋₆-alkyl, —OH, —O—C₁₋₁₀-alkyl, —NH₂, —NH—C₁₋₁₀-alkyl, —N(C₁₋₁₀-alkyl)₂, —N₃, —F, —Cl, —Br, and —I; or
a physiologically acceptable salt thereof.

6: The compound according to claim 1,
wherein
(i) R3 represents —C₄-alkyl-P(═O)(OH)₂ and R8 represents —C₃-cycloalkyl-aryl or —C₁-6-alkyl-phenyl (wherein in case of —C₂-alkyl-phenyl, the alkyl-moiety is saturated);
and wherein in each case R1 and R7 are independently from one another are selected from —H, —C₁₋₆-alkyl, —C₁₋₆-alkyl-C₃₋₆-cycloalkyl, —C₁₋₆-alkyl-aryl, and —C₁₋₆-alkyl-heteroaryl; or
wherein
(ii) R3 represents —C₄-alkyl-P(═O)(OC₁₋₆-alkyl)₂; or
R3 represents —C₄-alkyl-P(═O)(OH)(OC₁₋₆-alkyl); or
R3 represents —C₄-alkyl-S(═O)₂(OH); or
R3 represents —C₄-alkyl-S(═O)₂(NH₂); or
R3 represents —C₄-alkyl-C(═O)(OH); and
wherein in each case R1, R7, and R8 independently from one another are selected from —H, —C₁₋₆-alkyl, —C₁₋₆-alkyl-C₃₋₆-cycloalkyl, —C₁₋₆-alkyl-aryl, —C₁₋₆-alkyl-heteroaryl, —C₃₋₆-cycloalkyl, —C₁₋₆—O-aryl, and —C₁₋₆—O-heteroaryl;
wherein in each case “C₁₋₆-alkyl” may be linear or branched, unless expressly stated otherwise saturated or unsaturated, unsubstituted or monosubstituted with a substituent selected from —C≡CH, —C(═O)OH, —C(═O)O—C₁₋₁₀-alkyl, —OH, —O—C₁₋₁₀-alkyl, —NH₂, —NH—C₁₋₁₀-alkyl, —N(C₁₋₁₀-alkyl)₂, —N₃, —F, —Cl, —Br, and —I;
wherein in each case “C₃₋₆-cycloalkyl” may be linear or branched, unless expressly stated otherwise saturated or unsaturated, unsubstituted or monosubstituted with a substituent selected from —C≡CH, —C(═O)OH, —C(═O)O—C₁₋₆-alkyl, —OH, —O—C₁₋₁₀-alkyl, —NH₂, —NH—C₁₋₁₀-alkyl, —N(C₁₋₁₀-alkyl)₂, —N₃, —F, —Cl, —Br, and —I; or
a physiologically acceptable salt thereof.

7: The compound to claim 1,
wherein
(i) R3 represents —CH₂CH₂CH₂CH₂—P(═O)(OH)₂ and R8 represents -cyclopropyl-aryl, —CH₂-phenyl or —CH₂CH₂-phenyl (wherein in case of —CH₂CH₂-phenyl, the ethyl-moiety is saturated); and
wherein in each case R1 and R7 independently from one another are selected from —H, —C₁₋₆-alkyl, —C₁₋₆-alkyl-C₃₋₆-cycloalkyl, —C₁₋₆-alkyl-aryl, and —C₁₋₆-alkyl-heteroaryl; or
wherein
(ii) R3 represents —CH₂CH₂CH₂CH₂—P(═O)(OC₁₋₆-alkyl)₂; or
R3 represents —CH₂CH₂CH₂CH₂—P(═O)(OH)(OC₁₋₆-alkyl); or
R3 represents —CH₂CH₂CH₂CH₂—S(═O)₂(OH); or
R3 represents —CH₂CH₂CH₂CH₂—S(═O)₂(NH₂); or
R3 represents —CH₂CH₂CH₂CH₂—C(═O)(OH); and
wherein in each case R1, R7, and R8 independently from one another are selected from —H, —C₁₋₆-alkyl, —C₁₋₆-alkyl-C₃₋₆-cycloalkyl, —C₁₋₆-alkyl-aryl, —C₁₋₆-alkyl-heteroaryl, —C₃₋₆-cycloalkyl, —C₁₋₆—O-aryl, and —C₁₋₆—O-heteroaryl; or
wherein
(iii) R3 represents —CH₂CH₂CH₂CH₂—P(═O)(OC₁₋₆-alkyl)₂; or
R3 represents —CH₂CH₂CH₂CH₂—P(═O)(OH)(OC₁₋₆-alkyl); or
R3 represents —CH₂CH₂CH₂CH₂—S(═O)₂(OH); or
R3 represents —CH₂CH₂CH₂CH₂—S(═O)₂(NH₂); or
R3 represents —CH₂CH₂CH₂CH₂—C(═O)(OH); and
wherein in each case R1 and R7 independently from one another are selected from —H, —C₁₋₆-alkyl, —C₁₋₆-alkyl-C₃₋₆-cycloalkyl, —C₁₋₆-alkyl-aryl, —C₁₋₆-alkyl-heteroaryl, —C₃-6-cycloalkyl, —C₁₋₆—O-aryl, and —C₁₋₆—O-heteroaryl, and R8 represents -cyclopropyl-aryl, —CH₂-phenyl or —CH₂CH₂-phenyl (wherein in case of —CH₂CH₂-phenyl, the ethyl-moiety is saturated); or
wherein
(iv) R3 represents —CH₂CH₂CH₂CH₂—P(═O)(OC₁₋₆-alkyl)₂; or
R3 represents —CH₂CH₂CH₂CH₂—P(═O)(OH)(OC₁₋₆-alkyl); and
wherein in each case R1, R7, and R8 independently from one another are selected from —H, —C₁₋₆-alkyl, —C₁₋₆-alkyl-C₃₋₆-cycloalkyl, —C₁₋₆-alkyl-aryl, —C₁₋₆-alkyl-heteroaryl, —C₃₋₆-cycloalkyl, —C₁₋₆—O-aryl, and —C₁₋₆—O-heteroaryl;
wherein in each case “C₁₋₆-alkyl” may be linear or branched, unless expressly stated otherwise saturated or unsaturated, unsubstituted or monosubstituted with a substituent selected from —C≡CH, —C(═O)OH, —C(═O)O—C₁₋₁₀-alkyl, —OH, —O—C₁₋₁₀-alkyl, —NH₂, —NH—C₁₋₁₀-alkyl, —N(C₁₋₁₀-alkyl)₂, —N₃, —F, —Cl, —Br, and —I;
wherein in each case “C₃₋₆-cycloalkyl” may be linear or branched, unless expressly stated otherwise saturated or unsaturated, unsubstituted or monosubstituted with a substituent selected from —C≡CH, —C(═O)OH, —C(═O)O—C₁₋₆-alkyl, —OH, —O—C₁₋₁₀-alkyl, —NH₂, —NH—C₁₋₁₀-alkyl, —N(C₁₋₁₀-alkyl)₂, —N₃, —F, —Cl, —Br, and —I; or
a physiologically acceptable salt thereof.

8: The compound according to claim 1, wherein
R1 represents —CH₂—C≡CH, —CH₂—CH₃, or —CH₂-cyclobutyl, unsubstituted;
R3 represents (—CH₂—)₄₋₆—P(═O)(OH)₂;
R7 represents (i) —H; (ii) —CH₃, —CH₂—CH₃, —CH₂—CH₂—CH₃, —CH(CH₃)₂, —CH(CH₃)(CH₂—CH₃), —CH₂—CH₂—OH, —CH₂—CH₂—Cl, —CH₂—CH₂—NH₂, —CH₂-cyclopropyl, unsubstituted, —CH₂-cyclobutyl, unsubstituted, —CH₂-cyclopentyl, unsubstituted, —CH₂-cyclohexyl, unsubstituted; or (iii) —CH₂-phenyl, unsubstituted;
R8 represents (i) —CH₂—CH₂-phenyl, unsubstituted; (ii) —CH₂—CH₂-phenyl, mono-, di- or trisubstituted with —OCH₃, —CH₃, —CH₂—CH₃, —CH(CH₃)₂, —CF₃, —F, —Cl, —Br, —I and/or unsubstituted-phenyl; or (iii) —CH₂-naphthyl, unsubstituted; or
R1 represents —CH₂—C≡CH, —CH₂—CH₃, or —CH₂-cyclobutyl, unsubstituted;
R3 represents (—CH₂—)₄—P(═O)(OCH₂CH₃)₂;
R7 represents —H or —CH₃;
R8 represents (i) —CH₂—O-phenyl, unsubstituted; (ii) —CH₂—CH₂-phenyl, mono-, di- or trisubstituted with —OCH₃, —CH₃, —CF₃ and/or —Br; or (iii) -cyclopropyl-phenyl, unsubstituted; or
R1 represents —CH₂—C≡CH;
R3 represents (—CH₂—)₄—P(═O)(OH)(OCH₂CH₃);
R7 represents —H or —CH₃;
R8 represents —CH₂—CH₂-phenyl, mono-, di- or trisubstituted with —OCH₃ and/or —Br; or
R1 represents —CH₂—C≡CH or —CH₂—CH₃;
R3 represents (—CH₂—)₄—SO₂—OH;
R7 represents —H;
R8 represents —CH₂—CH₂-phenyl, mono-, di- or trisubstituted with —OCH₃ and/or —Br; or
R1 represents —CH₂—C≡CH;
R3 represents (—CH₂—)₄—SO₂—NH₂;
R7 represents —H;
R8 represents —CH₂—CH₂-phenyl, mono-, di- or trisubstituted with —Br; or
R1 represents —CH₂—C≡CH or CH₂-cyclobutyl, unsubstituted;
R3 represents (—CH₂—)₄₋₅—CO₂H;
R7 represents —H;
R8 represents —CH₂—CH₂-phenyl, mono-, di- or trisubstituted with —OCH₃, CF₃ and/or —Br.

9: The compound according to claim 1 which is selected from compounds:
(I) B-1 to B-58 and the physiologically acceptable salts thereof: 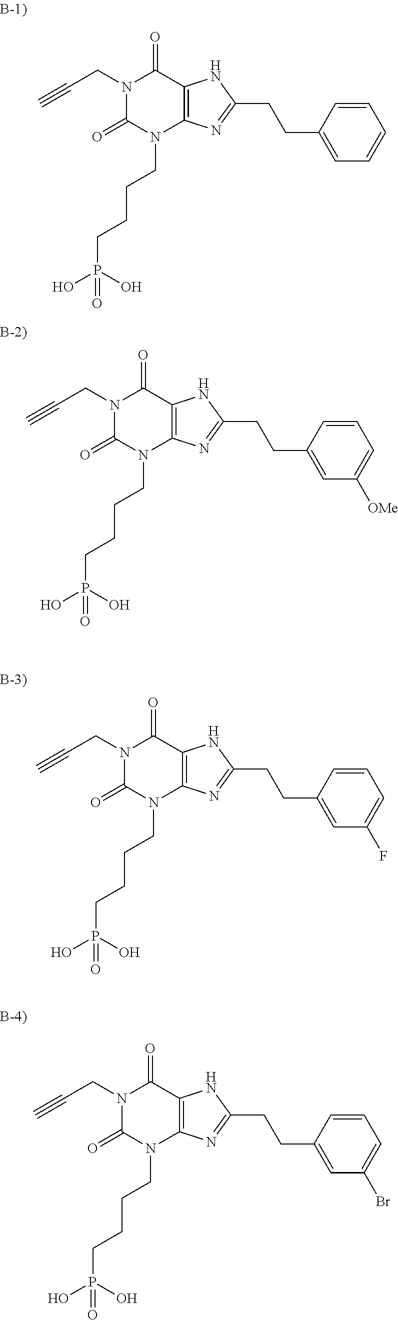 [SMILES: C#CCn1c(=O)c2[nH]c(CCc3ccccc3)nc2n(CCCCP(=O)(O)O)c1=O ; C#CCn1c(=O)c2[nH]c(CCc3cccc(OC)c3)nc2n(CCCCP(=O)(O)O)c1=O ; C#CCn1c(=O)c2[nH]c(CCc3cccc(F)c3)nc2n(CCCCP(=O)(O)O)c1=O ; C#CCn1c(=O)c2[nH]c(CCc3cccc(Br)c3)nc2n(CCCCP(=O)(O)O)c1=O] 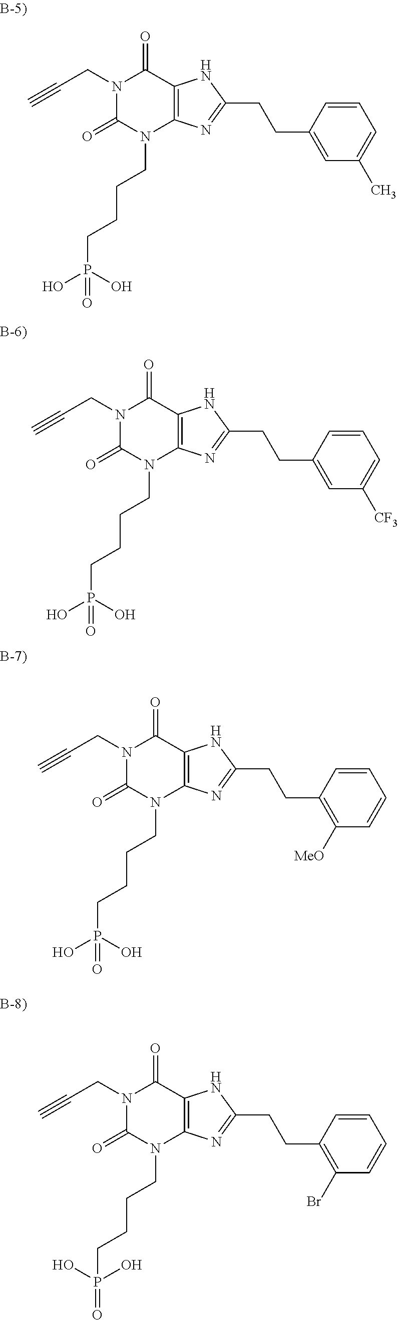 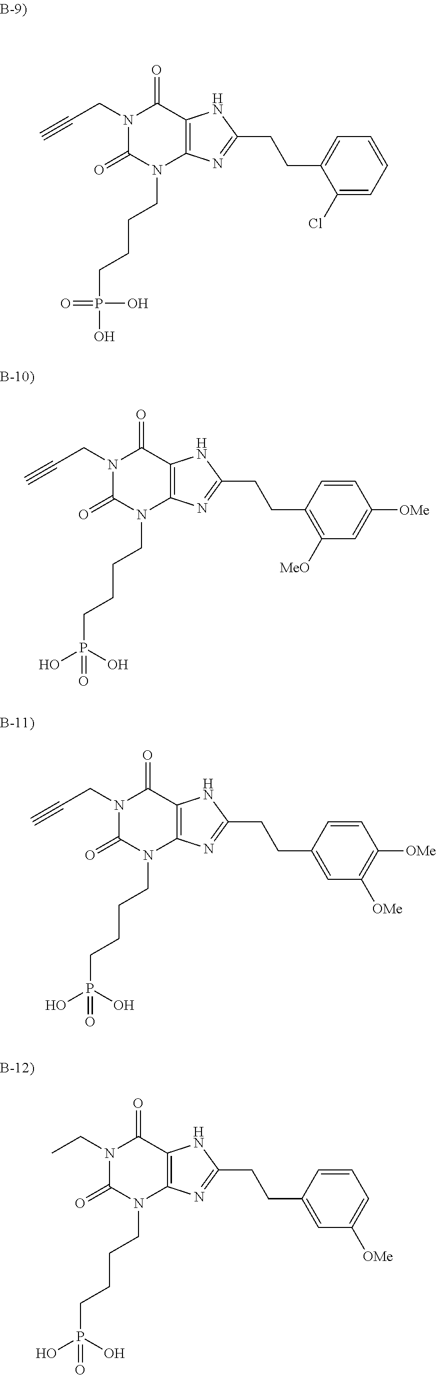 [SMILES: C#CCn1c(=O)c2[nH]c(CCc3ccccc3Cl)nc2n(CCCCP(=O)(O)O)c1=O ; C#CCn1c(=O)c2[nH]c(CCc3ccc(OC)cc3OC)nc2n(CCCCP(=O)(O)O)c1=O ; C#CCn1c(=O)c2[nH]c(CCc3ccc(OC)c(OC)c3)nc2n(CCCCP(=O)(O)O)c1=O ; CCn1c(=O)c2[nH]c(CCc3cccc(OC)c3)nc2n(CCCCP(=O)(O)O)c1=O] 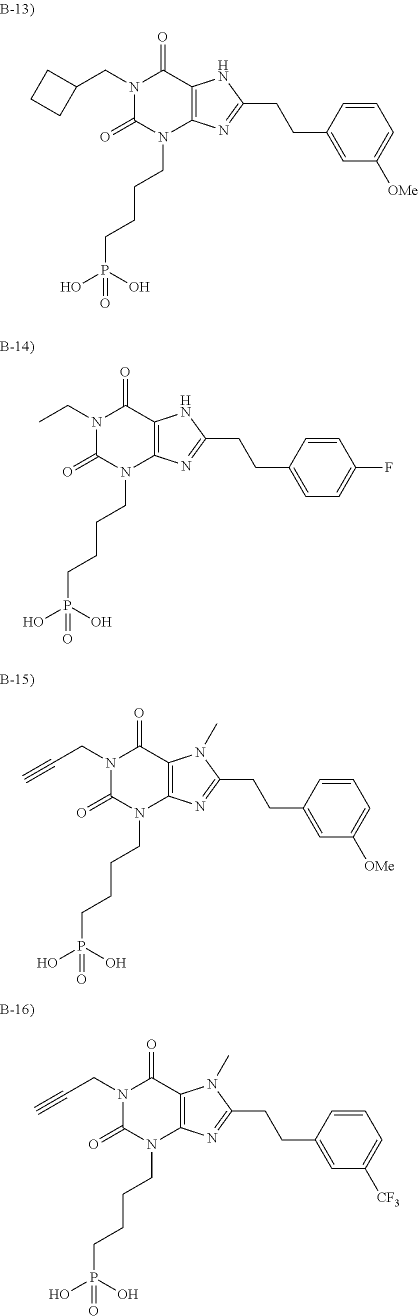 [SMILES: COc1cccc(CCc2nc3c([nH]2)c(=O)n(CC2CCC2)c(=O)n3CCCCP(=O)(O)O)c1 ; CCn1c(=O)c2[nH]c(CCc3ccc(F)cc3)nc2n(CCCCP(=O)(O)O)c1=O ; C#CCn1c(=O)c2c(nc(CCc3cccc(OC)c3)n2C)n(CCCCP(=O)(O)O)c1=O ; C#CCn1c(=O)c2c(nc(CCc3cccc(C(F)(F)F)c3)n2C)n(CCCCP(=O)(O)O)c1=O] 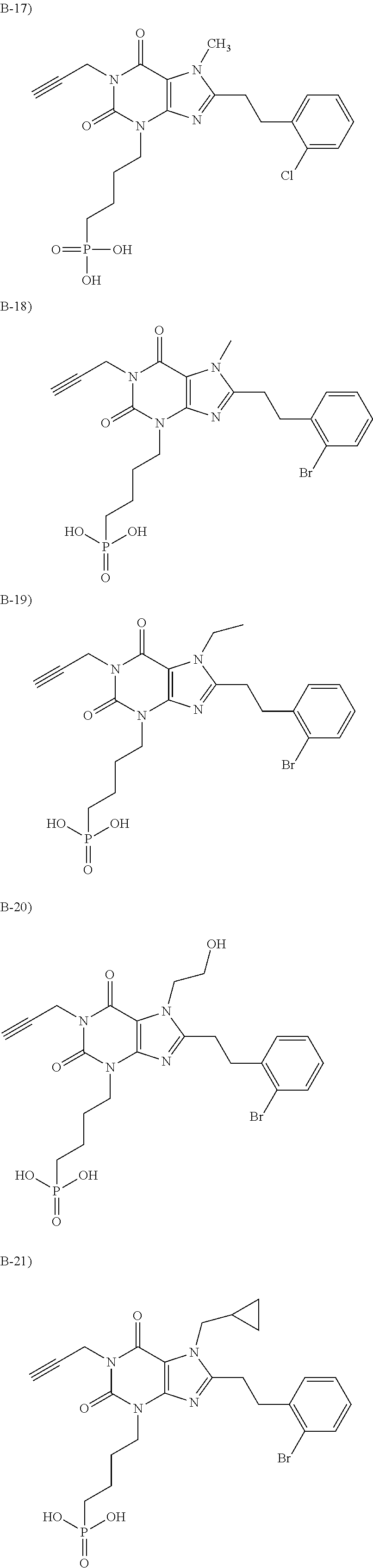 [SMILES: C#CCn1c(=O)c2c(nc(CCc3ccccc3Cl)n2C)n(CCCCP(=O)(O)O)c1=O ; C#CCn1c(=O)c2c(nc(CCc3ccccc3Br)n2C)n(CCCCP(=O)(O)O)c1=O ; C#CCn1c(=O)c2c(nc(CCc3ccccc3Br)n2CC)n(CCCCP(=O)(O)O)c1=O ; C#CCn1c(=O)c2c(nc(CCc3ccccc3Br)n2CCO)n(CCCCP(=O)(O)O)c1=O ; C#CCn1c(=O)c2c(nc(CCc3ccccc3Br)n2CC2CC2)n(CCCCP(=O)(O)O)c1=O] 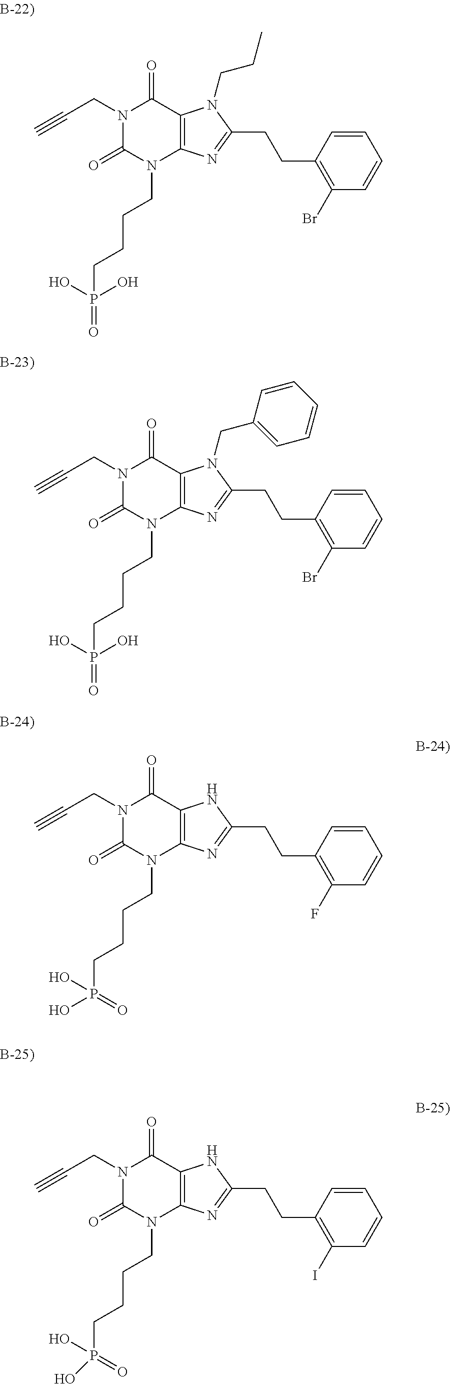 [SMILES: C#CCn1c(=O)c2c(nc(CCc3ccccc3Br)n2CCC)n(CCCCP(=O)(O)O)c1=O ; C#CCn1c(=O)c2c(nc(CCc3ccccc3Br)n2Cc2ccccc2)n(CCCCP(=O)(O)O)c1=O ; C#CCn1c(=O)c2[nH]c(CCc3ccccc3F)nc2n(CCCCP(=O)(O)O)c1=O ; C#CCn1c(=O)c2[nH]c(CCc3ccccc3I)nc2n(CCCCP(=O)(O)O)c1=O] 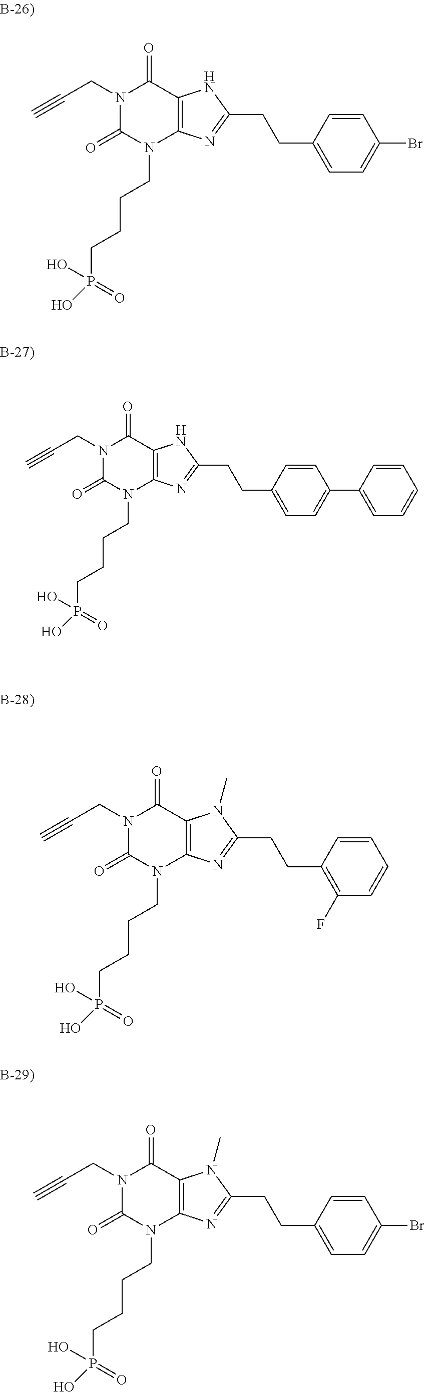 [SMILES: C#CCn1c(=O)c2[nH]c(CCc3ccc(Br)cc3)nc2n(CCCCP(=O)(O)O)c1=O ; C#CCn1c(=O)c2[nH]c(CCc3ccc(-c4ccccc4)cc3)nc2n(CCCCP(=O)(O)O)c1=O ; C#CCn1c(=O)c2c(nc(CCc3ccccc3F)n2C)n(CCCCP(=O)(O)O)c1=O ; C#CCn1c(=O)c2c(nc(CCc3ccc(Br)cc3)n2C)n(CCCCP(=O)(O)O)c1=O] 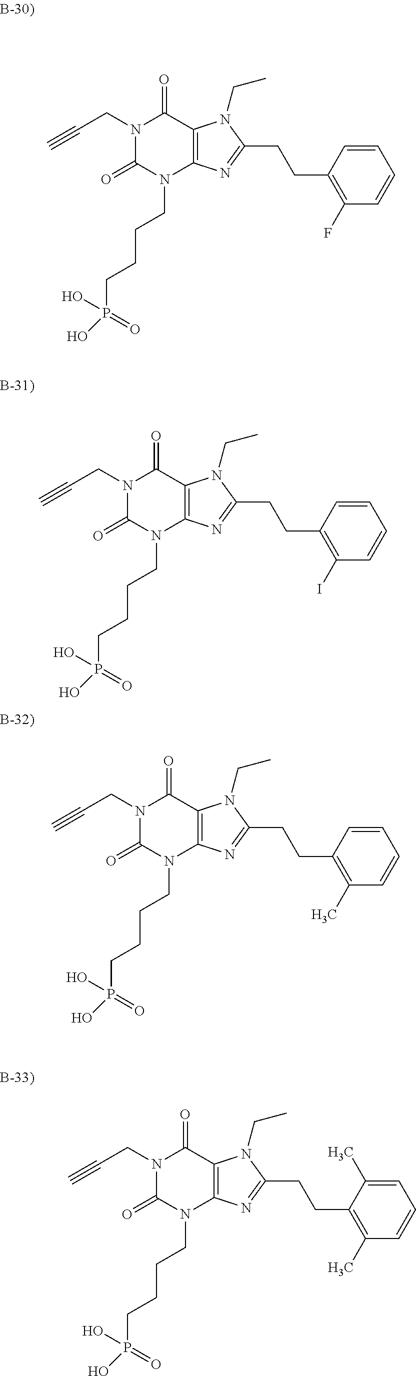 [SMILES: C#CCn1c(=O)c2c(nc(CCc3ccccc3F)n2CC)n(CCCCP(=O)(O)O)c1=O ; C#CCn1c(=O)c2c(nc(CCc3ccccc3I)n2CC)n(CCCCP(=O)(O)O)c1=O ; C#CCn1c(=O)c2c(nc(CCc3ccccc3C)n2CC)n(CCCCP(=O)(O)O)c1=O ; C#CCn1c(=O)c2c(nc(CCc3c(C)cccc3C)n2CC)n(CCCCP(=O)(O)O)c1=O] 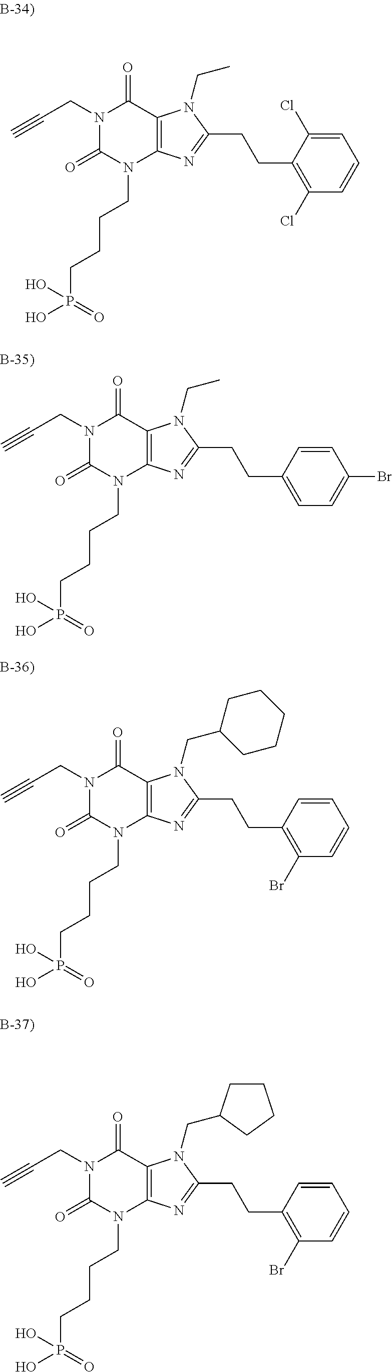 [SMILES: C#CCn1c(=O)c2c(nc(CCc3c(Cl)cccc3Cl)n2CC)n(CCCCP(=O)(O)O)c1=O ; C#CCn1c(=O)c2c(nc(CCc3ccc(Br)cc3)n2CC)n(CCCCP(=O)(O)O)c1=O ; C#CCn1c(=O)c2c(nc(CCc3ccccc3Br)n2CC2CCCCC2)n(CCCCP(=O)(O)O)c1=O ; C#CCn1c(=O)c2c(nc(CCc3ccccc3Br)n2CC2CCCC2)n(CCCCP(=O)(O)O)c1=O] 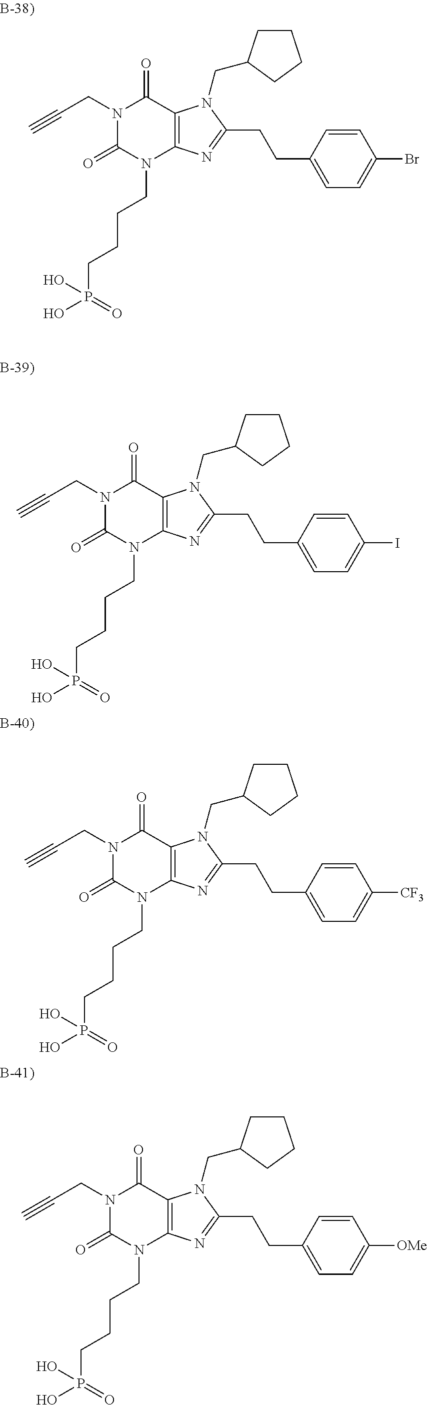 [SMILES: C#CCn1c(=O)c2c(nc(CCc3ccc(Br)cc3)n2CC2CCCC2)n(CCCCP(=O)(O)O)c1=O ; C#CCn1c(=O)c2c(nc(CCc3ccc(I)cc3)n2CC2CCCC2)n(CCCCP(=O)(O)O)c1=O ; C#CCn1c(=O)c2c(nc(CCc3ccc(C(F)(F)F)cc3)n2CC2CCCC2)n(CCCCP(=O)(O)O)c1=O ; C#CCn1c(=O)c2c(nc(CCc3ccc(OC)cc3)n2CC2CCCC2)n(CCCCP(=O)(O)O)c1=O] 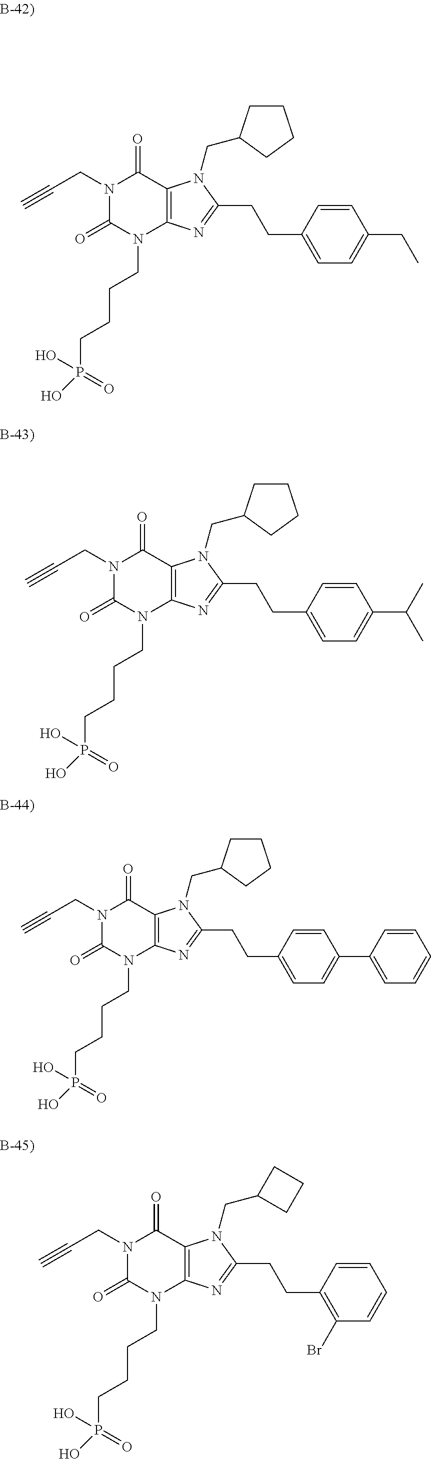 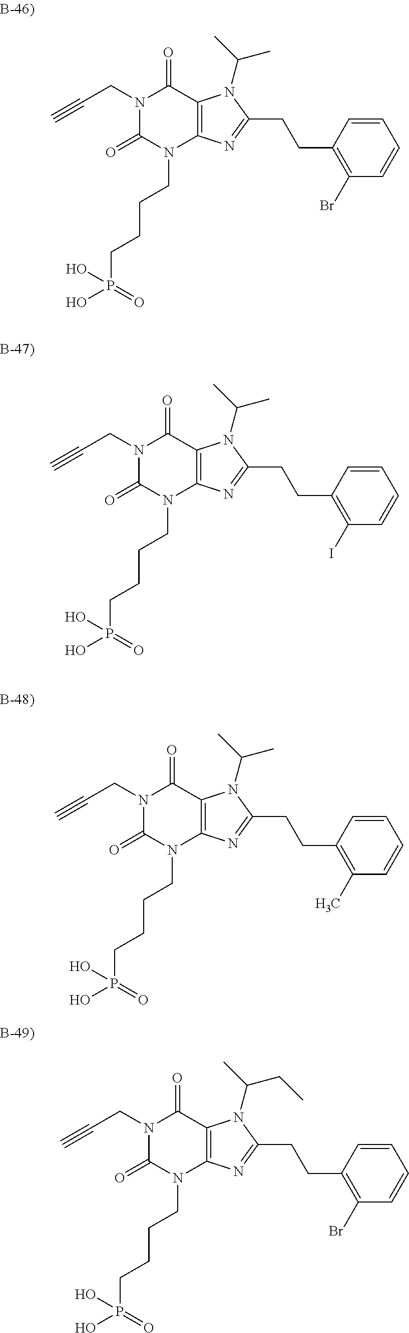 [SMILES: C#CCn1c(=O)c2c(nc(CCc3ccccc3Br)n2C(C)C)n(CCCCP(=O)(O)O)c1=O ; C#CCn1c(=O)c2c(nc(CCc3ccccc3I)n2C(C)C)n(CCCCP(=O)(O)O)c1=O ; C#CCn1c(=O)c2c(nc(CCc3ccccc3C)n2C(C)C)n(CCCCP(=O)(O)O)c1=O ; C#CCn1c(=O)c2c(nc(CCc3ccccc3Br)n2C(C)CC)n(CCCCP(=O)(O)O)c1=O] 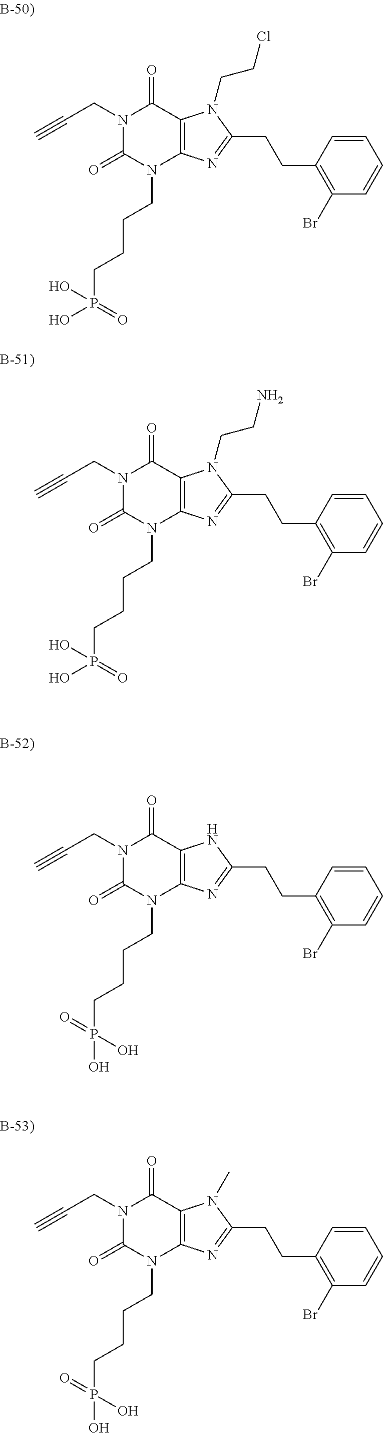 [SMILES: C#CCn1c(=O)c2c(nc(CCc3ccccc3Br)n2CCCl)n(CCCCP(=O)(O)O)c1=O ; C#CCn1c(=O)c2c(nc(CCc3ccccc3Br)n2CCN)n(CCCCP(=O)(O)O)c1=O ; C#CCn1c(=O)c2[nH]c(CCc3ccccc3Br)nc2n(CCCCP(=O)(O)O)c1=O ; C#CCn1c(=O)c2c(nc(CCc3ccccc3Br)n2C)n(CCCCP(=O)(O)O)c1=O] 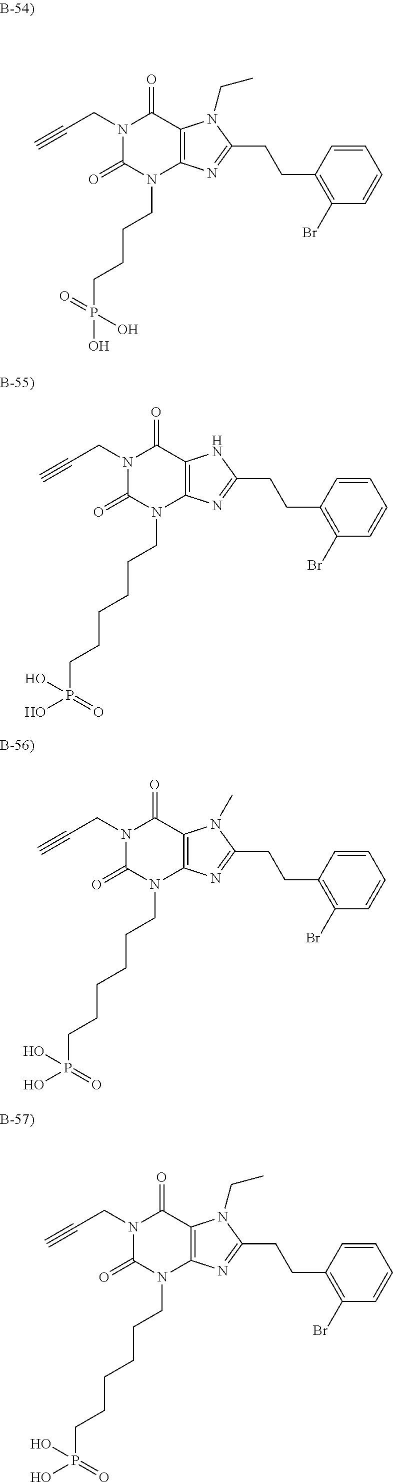 [SMILES: C#CCn1c(=O)c2c(nc(CCc3ccccc3Br)n2CC)n(CCCCP(=O)(O)O)c1=O ; C#CCn1c(=O)c2[nH]c(CCc3ccccc3Br)nc2n(CCCCCCP(=O)(O)O)c1=O ; C#CCn1c(=O)c2c(nc(CCc3ccccc3Br)n2C)n(CCCCCCP(=O)(O)O)c1=O ; C#CCn1c(=O)c2c(nc(CCc3ccccc3Br)n2CC)n(CCCCCCP(=O)(O)O)c1=O] 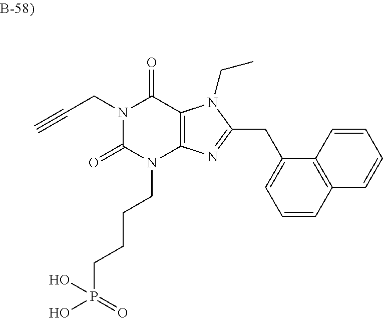 [SMILES: C#CCn1c(=O)c2c(nc(Cc3cccc4ccccc34)n2CC)n(CCCCP(=O)(O)O)c1=O]
and
(II) C-1 to C-10 and the physiologically acceptable salts thereof: 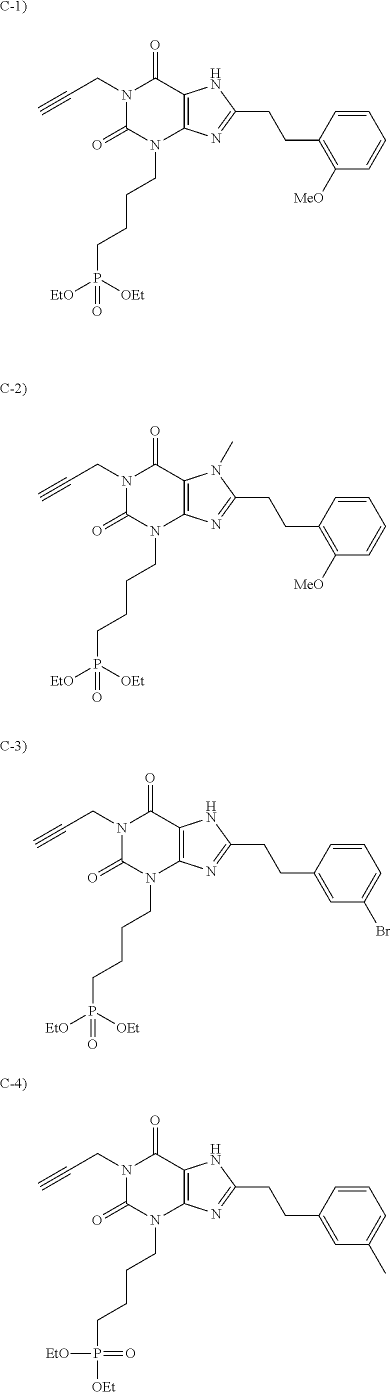 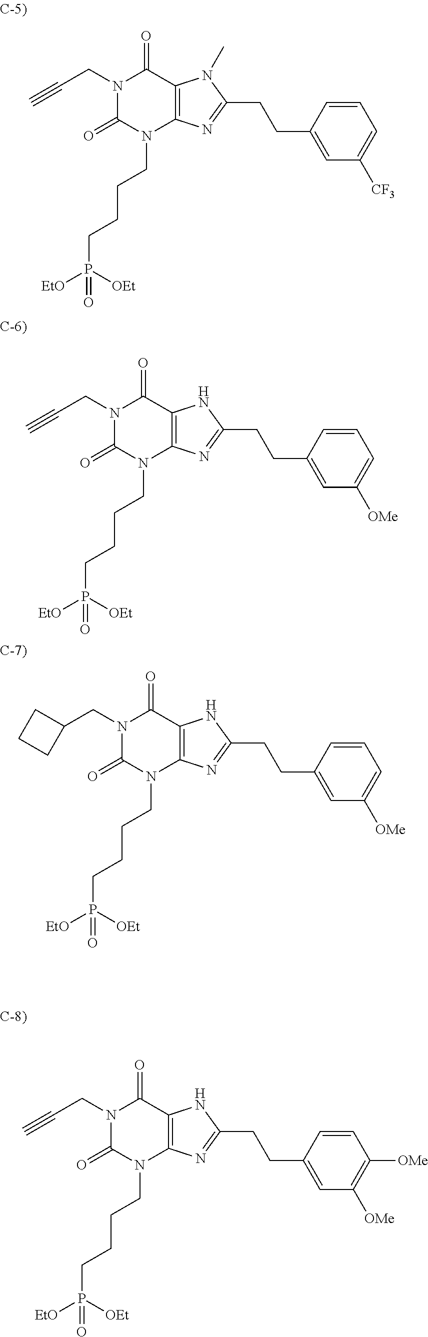 [SMILES: C#CCn1c(=O)c2c(nc(CCc3cccc(C(F)(F)F)c3)n2C)n(CCCCP(=O)(OCC)OCC)c1=O ; C#CCn1c(=O)c2[nH]c(CCc3cccc(OC)c3)nc2n(CCCCP(=O)(OCC)OCC)c1=O ; CCOP(=O)(CCCCn1c(=O)n(CC2CCC2)c(=O)c2[nH]c(CCc3cccc(OC)c3)nc21)OCC ; C#CCn1c(=O)c2[nH]c(CCc3ccc(OC)c(OC)c3)nc2n(CCCCP(=O)(OCC)OCC)c1=O] 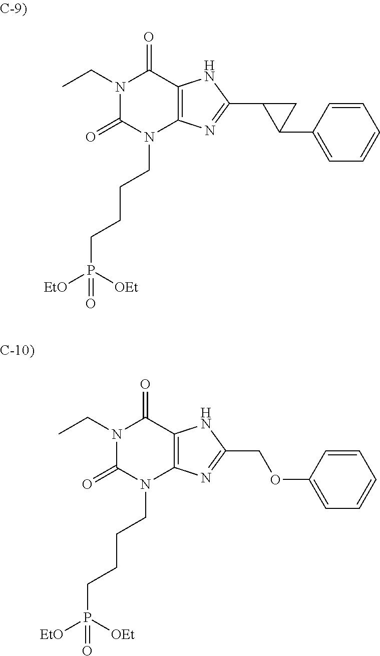 [SMILES: CCOP(=O)(CCCCn1c(=O)n(CC)c(=O)c2[nH]c(C3CC3c3ccccc3)nc21)OCC ; CCOP(=O)(CCCCn1c(=O)n(CC)c(=O)c2[nH]c(COc3ccccc3)nc21)OCC]
and
(III) D-1 to D-3 and the physiologically acceptable salts thereof: 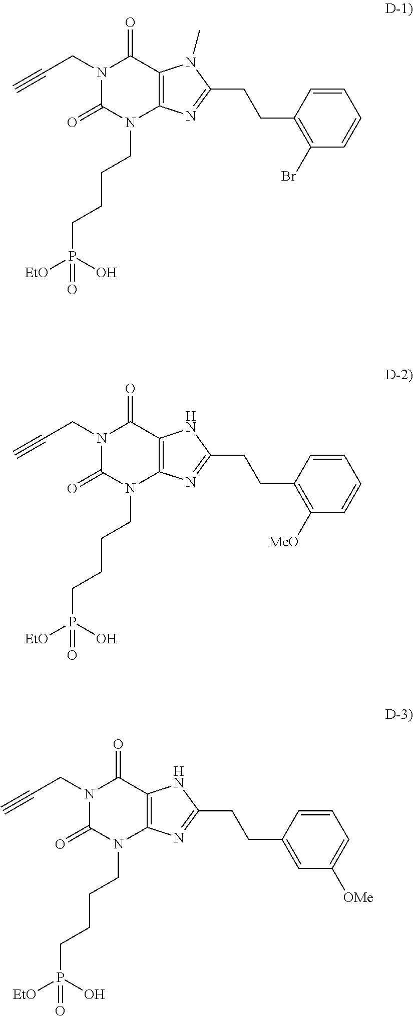 [SMILES: C#CCn1c(=O)c2c(nc(CCc3ccccc3Br)n2C)n(CCCCP(=O)(O)OCC)c1=O ; C#CCn1c(=O)c2[nH]c(CCc3ccccc3OC)nc2n(CCCCP(=O)(O)OCC)c1=O ; C#CCn1c(=O)c2[nH]c(CCc3cccc(OC)c3)nc2n(CCCCP(=O)(O)OCC)c1=O]
and
(IV) E-1 to E-3 and the physiologically acceptable salts thereof: 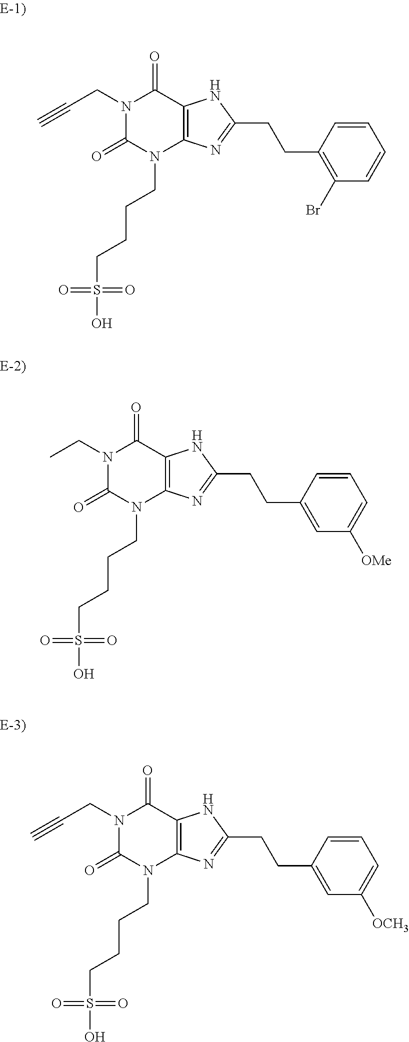 [SMILES: C#CCn1c(=O)c2[nH]c(CCc3ccccc3Br)nc2n(CCCCS(=O)(=O)O)c1=O ; CCn1c(=O)c2[nH]c(CCc3cccc(OC)c3)nc2n(CCCCS(=O)(=O)O)c1=O ; C#CCn1c(=O)c2[nH]c(CCc3cccc(OC)c3)nc2n(CCCCS(=O)(=O)O)c1=O]
and
(V) F-1 and the physiologically acceptable salts thereof: 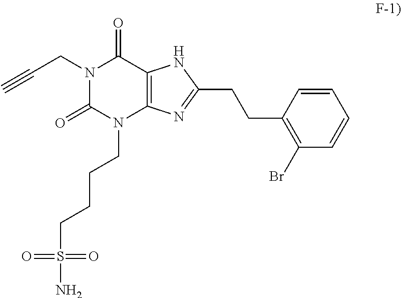 [SMILES: C#CCn1c(=O)c2[nH]c(CCc3ccccc3Br)nc2n(CCCCS(N)(=O)=O)c1=O]
and
(VI) G-1 to G-3 and the physiologically acceptable salts thereof:

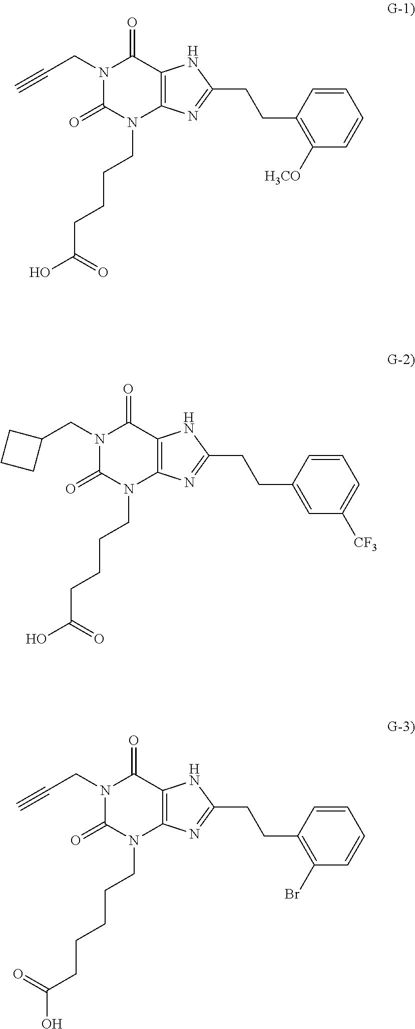 [SMILES: C#CCn1c(=O)c2[nH]c(CCc3ccccc3OC)nc2n(CCCCC(=O)O)c1=O ; O=C(O)CCCCn1c(=O)n(CC2CCC2)c(=O)c2[nH]c(CCc3cccc(C(F)(F)F)c3)nc21 ; C#CCn1c(=O)c2[nH]c(CCc3ccccc3Br)nc2n(CCCCCC(=O)O)c1=O]

10: The compound according to claim 1 for use as a medicament.

11: The compound according to claim 1 for use in the prevention or treatment of a condition, disease or disorder that is associated with the MRGPRX4 receptor, preferably associated with the wildtype of the MRGPRX4 receptor.

12: A compound according to general Formula 1: 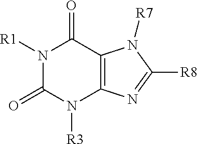
wherein
R1 represents —H, —C₁₋₁₀-alkyl, —C₁₋₁₀-alkyl-C₃₋₁₀-cycloalkyl, —C₁₋₁₀-alkyl-aryl, —C₁₋₁₀-alkyl-heteroaryl, —C₃₋₁₀-cycloalkyl, —C₁₋₁₀-alkyl-O-aryl, or —C₁₋₁₀-alkyl-O-heteroaryl;
R3 represents —C₁₋₁₀-alkyl-P(═O)(OC₁₋₁₀-alkyl)₂, —C₁₋₁₀-alkyl-P(═O)(OH)(OC₁₋₁₀-alkyl), —C₁₋₁₀-alkyl-S(═O)₂(OH), —C₁₋₁₀-alkyl-S(═O)₂(NH₂), —C₁₋₁₀-alkyl-C(═O)(OH), or —C₁₋₁₀-alkyl-P(═O)(OH)₂;
R7 represents —H, —C₁₋₁₀-alkyl, —C₁₋₁₀-alkyl-C₃₋₁₀-cycloalkyl, —C₁₋₁₀-alkyl-aryl, —C₁₋₁₀-alkyl-heteroaryl, —C₃₋₁₀-cycloalkyl, —C₁₋₁₀-alkyl-O-aryl, or —C₁₋₁₀-alkyl-O-heteroaryl; and
R8 represents —H, —C₁₋₁₀-alkyl, —C₁₋₁₀-alkyl-C₃₋₁₀-cycloalkyl, —C₁₋₁₀-alkyl-aryl, —C₁₋₁₀-alkyl-heteroaryl, —C₃₋₁₀-cycloalkyl, —C₁₋₁₀-alkyl-O-aryl, —C₃₋₆-cycloalkyl-aryl, —C₃₋₆-cycloalkyl-heteroaryl, or —C₁₋₁₀-alkyl-O-heteroaryl;
wherein in each case “C₁₋₁₀-alkyl” may be linear or branched, unless expressly stated otherwise saturated or unsaturated, unsubstituted or monosubstituted with a substituent selected from —C≡CH, —C(═O)OH, —C(═O)O—C₁₋₁₀-alkyl, —OH, —O—C₁₋₁₀-alkyl, —NH₂, —NH—C₁₋₁₀-alkyl, —N(C₁₋₁₀-alkyl)₂, —N₃, —F, —Cl, —Br, and —I;
wherein in each case “C₃₋₁₀-cycloalkyl” may be linear or branched, unless expressly stated otherwise saturated or unsaturated, unsubstituted or monosubstituted with a substituent selected from —C≡CH, —C(═O)OH, —C(═O)O—C₁₋₆-alkyl, —OH, —O—C₁₋₁₀-alkyl, —NH₂, —NH—C₁₋₁₀-alkyl, —N(C₁₋₁₀-alkyl)₂, —N₃, —F, —Cl, —Br, and —I;
wherein in each case “aryl” is a 6-14-membered aryl moiety which may be unsubstituted, mono- or disubstituted with a substituent independently selected from —C≡CH, —CH₃, —CH₂—CH₃, —CH(CH₃)₂, -phenyl, —CF₃, —CCl₃, —CBr₃, —Cl₃, —C(═O)OH, —C(═O)—C₁₋₁₀-alkyl, —OH, —O—C₁₋₁₀-alkyl, —NH₂, —NH—C₁₋₁₀-alkyl, —N(C₁₋₁₀-alkyl)₂, —N₃, —F, —Cl, —Br, and —I;
wherein in each case “heteroaryl” is a 5-14-membered heteroaryl moiety which may be unsubstituted, mono- or disubstituted with a substituent independently selected from —C≡CH, —CF₃, —CCl₃, —CBr₃, —Cl₃, —C(═O)OH, —C(═O)O—C₁₋₁₀-alkyl, —OH, —O—C₁₋₁₀-alkyl, —NH₂, —NH—C₁₋₁₀-alkyl, —N(C₁₋₁₀-alkyl)₂, —N₃, —F, —Cl, —Br, and —I; or
a physiologically acceptable salt thereof,
for use in the prevention or treatment of a condition, disease or disorder that is associated with the MRGPRX4 receptor, preferably associated with the wildtype of the MRGPRX4 receptor.

13: The compound for use according to claim 12, wherein the compound is according to claim 3.

14: The compound according to claim 12,
wherein
(iii) R3 represents —C₁₋₁₀-alkyl-P(═O)(OH)₂ and R8 represents —C₃₋₆-cycloalkyl, -aryl, -heteroaryl, —C₁₋₁₀-alkyl-aryl, or —C₁₋₁₀-alkyl-heteroaryl (wherein in case of —C₂-alkyl-aryl, the alkyl-moiety is unsaturated);
and wherein in each case R1 and R7 independently from one another are selected from —H, —C₁₋₁₀-alkyl, —C₁₋₁₀-alkyl-C₃₋₁₀-cycloalkyl, —C₁₋₁₀-alkyl-aryl, and —C₁₋₁₀-alkyl-heteroaryl; or
wherein
(iv) R3 represents —C₁₋₁₀-alkyl-O—C(═O)C₁₋₁₀-alkyl;
and wherein in each case R1, R7, and R8 independently from one another are selected from —H, —C₁₋₁₀-alkyl, —C₁₋₁₀-alkyl-C₃₋₁₀-cycloalkyl, —C₁₋₁₀-alkyl-aryl, —C₁₋₁₀-alkyl-heteroaryl, —C₃₋₁₀-cycloalkyl, —C₁₋₁₀-alkyl-O-aryl, and —C₁₋₁₀-alkyl-O-heteroaryl;
for use in the prevention or treatment of a condition, disease or disorder that is associated with the MRGPRX4 receptor, preferably associated with the wildtype of the MRGPRX4 receptor.

15: The compound for use according to claim 12,
wherein
R1 represents —CH₂—C≡CH, —CH₂—CH₃, —CH₂—CH₂—CH₃, or —CH₂-cyclobutyl, unsubstituted;
R3 represents (—CH₂—)₄₋₆—P(═O)(OH)₂;
R7 represents (i) —H; (ii) —CH₃, —CH₂—CH₃, —CH₂—CH₂—CH₃, —CH(CH₃)₂, —CH(CH₃)(CH₂—CH₃), —CH₂—CH₂—OH, —CH₂—CH₂—Cl, —CH₂—CH₂—NH₂, —CH₂-cyclopropyl, unsubstituted, —CH₂-cyclobutyl, unsubstituted, —CH₂-cyclopentyl, unsubstituted, —CH₂-cyclohexyl, unsubstituted; or (iii) —CH₂-phenyl, unsubstituted;
R8 represents (i) -phenyl or —CH₂—CH₂-phenyl, in either case unsubstituted; (ii) —CH₂—CH₂-phenyl, mono-, di- or trisubstituted with —OCH₃, —CH₃, —CH₂—CH₃, —CH(CH₃)₂, —CF₃, —F, —Cl, —Br, —I and/or unsubstituted-phenyl; (iii) —CH═CH-phenyl, mono-, di- or trisubstituted with —OCH₃; (iv) -cyclopentyl, unsubstituted; or (v) —CH₂-naphthyl, unsubstituted; or
R1 represents —CH₂—C≡CH, —CH₂—CH₃, or —CH₂-cyclobutyl, unsubstituted;
R3 represents (—CH₂—)₄—P(═O)(OCH₂CH₃)₂;
R7 represents —H or —CH₃;
R8 represents (i) —CH₂—O-phenyl, unsubstituted; (ii) —CH₂—CH₂-phenyl, mono-, di- or trisubstituted with —OCH₃, —CH₃, —CF₃ and/or —Br; or (iii) -cyclopropyl-phenyl, unsubstituted; or
R1 represents —CH₂—C≡CH;
R3 represents (—CH₂—)₄—P(═O)(OH)(OCH₂CH₃);
R7 represents —H or —CH₃;
R8 represents —CH₂—CH₂-phenyl, mono-, di- or trisubstituted with —OCH₃ and/or —Br; or
R1 represents —CH₂—C≡CH or —CH₂—CH₃;
R3 represents (—CH₂—)₄—SO₂—OH;
R7 represents —H;
R8 represents —CH₂—CH₂-phenyl, mono-, di- or trisubstituted with —OCH₃ and/or —Br; or
R1 represents —CH₂—C≡CH;
R3 represents (—CH₂—)₄—SO₂—NH₂;
R7 represents —H;
R8 represents —CH₂—CH₂-phenyl, mono-, di- or trisubstituted with —Br; or
R1 represents —CH₂—C≡CH or CH₂-cyclobutyl, unsubstituted;
R3 represents (—CH₂—)₄₋₅—CO₂H;
R7 represents —H;
R8 represents —CH₂—CH₂-phenyl, mono-, di- or trisubstituted with —OCH₃, CF₃ and/or —Br.

16: The compound for use according to claim 12, which is selected from compounds;
J-1 to J-3 and the physiologically acceptable salts thereof:

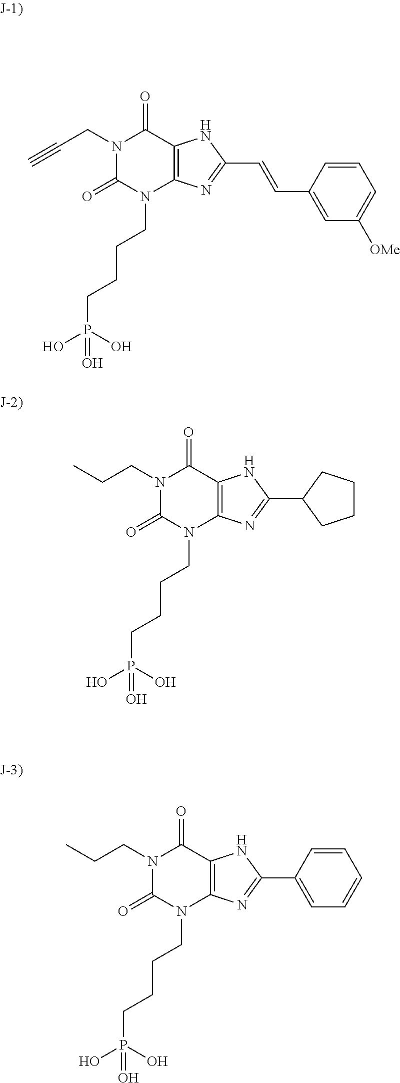 [SMILES: C#CCn1c(=O)c2[nH]c(/C=C/c3cccc(OC)c3)nc2n(CCCCP(=O)(O)O)c1=O ; CCCn1c(=O)c2[nH]c(C3CCCC3)nc2n(CCCCP(=O)(O)O)c1=O ; CCCn1c(=O)c2[nH]c(-c3ccccc3)nc2n(CCCCP(=O)(O)O)c1=O]

17: The compound for use according to claim 10, wherein the disease or disorder that is associated with the MRGPRX4 receptor, preferably associated with the wildtype of the MRGPRX4 receptor, is selected from:
open wounds; preferably selected from the group consisting of incisions or incised wounds, lacerations, abrasions (grazes), avulsions, puncture wounds, penetration wounds and gunshot wounds; and
closed wounds; preferably selected from the group consisting of hematomas and crash injuries; and
painful states, itching, neuropathic pain, chronic pain.

18: The compound for use according to claim 10, wherein the compound is administered topically and/or locally.
